(19) United States
(12) Patent Application Publication
(10) Pub. No.: US 2024/0002354 A1
(43) Pub. Date: Jan. 4, 2024

(54) ANTIDIABETIC COMPOUNDS AND COMPOSITIONS

(71) Applicant: REZUBIO PHARMACEUTICALS CO., LTD, Zhuhai, Guangdong (CN)

(72) Inventors: Yusheng XIONG, Zhuhai, Guangdong (CN); Hongping GUAN, Zhuhai, Guangdong (CN)

(21) Appl. No.: 18/040,596

(22) PCT Filed: Jul. 30, 2021
(86) PCT No.: PCT/CN2021/109496

§ 371 Date: Feb. 3, 2023

(30) Foreign Application Priority Data
Aug. 5, 2020 (WO) PCT/CN2020/107047

(51) Int. Cl. C07D 249/04; C07D 405/14; C07D 405/04; A61K 45/06

(52) U.S. Cl. CPC C07D 249/04; A61K 45/06; C07D 405/04; C07D 405/14

(57) ABSTRACT

Provided herein are novel compounds (e.g., Formula I), pharmaceutical compositions, and methods of using related to GPR40. The compounds herein are typically GPR40 agonists, which can be used for treating a variety of disorders, conditions or diseases such as Type 2 diabetes.

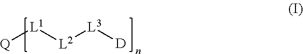

### CROSS REFERENCE TO RELATED APPLICATIONS

This application claims priority to International Patent Application No. PCT/CN2020/107047, filed Aug. 5, 2020, the content of which is incorporated herein by reference in its entirety.

In various embodiments, the present disclosure generally relates to novel antidiabetic compounds, pharmaceutical compositions, and methods of using the same, such as for treating Type 2 diabetes mellitus.

### BACKGROUND

Type 2 diabetes mellitus is a form of diabetes that is characterized by high blood sugar, insulin resistance, and relative lack of insulin. There are several available treatments for Type 2 diabetes, each of which has its own limitations and potential risks. Pharmacologic treatments for diabetes have largely focused on: (1) hepatic glucose production (biguanides, such as phenformin and metformin), (2) insulin resistance (PPAR agonists, such as rosiglitazone, troglitazone, engliazone, balaglitazone, MCC-555, netoglitazone, T-131, LY-300512, LY-818 and pioglitazone), (3) insulin secretion (sulfonylureas, such as tolbutamide, glipizide and glimipiride); (4) incretin hormone mimetics (GLP-1 derivatives and analogs, such as exenatide, liraglutide, dulaglutide, semaglutide, lixisenatide, albiglutide and taspoglutide); (5) inhibitors of incretin hormone degradation (DPP-4 inhibitors, such as sitagliptin, alogliptin, vildagliptin, linagliptin, denagliptin and saxagliptin); and (6) SGLT2 inhibitors (canagliflozin, dapagliflozin, empagliflozin, and ertugliflozin).

G-protein-coupled receptor 40 (GPR40) is a cell-surface GPCR that is highly expressed in human (and rodent) islets as well as in insulin-secreting cell lines. The human G-protein-coupled receptor hGPR40 is primarily localized in pancreatic R cells and intestinal enteroendocrine cells. Other organs expressing GPR40 include brain (hippocampus and hypothalamus) and liver. Medium- to long-chain fatty acids (FFAs) are endogenous ligands of GPR40. Upon binding to GPR40, FFAs trigger a signaling cascade that results in increased levels of [Ca²] in β-cells and subsequent stimulation of insulin secretion. In the gut, FFAs also stimulate secretion of incretins, including glucagon-like peptide-1 (GLP-1) and glucose-dependent insulinotropic polypeptide (GIP), cholocystokinine (CCK), and peptide YY (PYY). The recent recognition of the function of GPR40 in modulating insulin secretion has provided insights into regulation of carbohydrate and lipid metabolism in vertebrates, and further provided targets for the development of therapeutic agents for metabolic disorders such as obesity, diabetes, cardiovascular disease and dyslipidemia.

Agonists of G-protein-coupled receptor 40 (GPR40) have been shown to be useful in treating type 2 diabetes mellitus, obesity, hypertension, dyslipidemia, cancer, and metabolic syndrome, as well as cardiovascular diseases, such as myocardial infarction and stroke. New GPR40 agonists that have pharmacokinetic and pharmacodynamic properties suitable for use as human pharmaceuticals are needed.

### BRIEF SUMMARY

Provided herein are compounds, pharmaceutical compositions, and methods of using related to GPR40. The compounds herein are typically GPR40 agonists, which can be used for treating a disorder, condition or disease such as Type 2 diabetes, obesity, hyperglycemia, glucose intolerance, insulin resistance, hyperinsulinemia, hypercholesterolemia, hypertension, hyperlipoproteinemia, hyperlipidemia, myocardial infarction, stroke, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer, edema, nonalcoholic steatohepatitis (NASH), lipodystrophy, Prader Willi syndrome, and/or neurodegenerative diseases including but not limited to Alzheimer's disease, Parkinson's disease, amyotrophic lateral sclerosis.

Some embodiments of the present disclosure are directed to compounds of Formula I, or pharmaceutically acceptable salts or esters thereof:

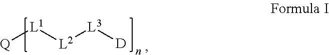

wherein the variables Q, L¹, L², L³, D, and n are defined herein. For example, Q is typically a hydrophilic carrier, D is a residue of a GPR40 agonist, and L¹, L², and L³ are together a linker that connects D with Q. In some embodiments, the compounds of Formula I can have a subformulae I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3, as defined herein.

Some embodiments of the present disclosure are directed to compounds of Formula II, or pharmaceutically acceptable salts or esters thereof:

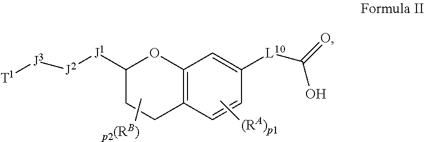

wherein the variables L¹⁰, R^{A}, R^{B}, J¹, J², J³, T¹, p1, and p2 are defined herein. In some embodiments, the compounds of Formula II can have a subformulae II-1, II-1-A, II-1-A-1, II-1-A-2, II-1-A-3, II-1-A-4, or II-1-A-5, as defined herein.

In some embodiments, the present disclosure provides a compound of Formula III-1, III-2, III-3, III-4, III-5, III-6, III-7, III-8, or III-9, as defined herein, or a pharmaceutically acceptable salt or ester thereof. In some embodiments, the present disclosure also provides a compound selected from Compound Nos. 1-237, or a pharmaceutically acceptable salt or ester thereof.

In some embodiments, the present disclosure provides a pharmaceutical composition comprising one or more compounds of the present disclosure and optionally a pharmaceutically acceptable excipient. In some embodiments, the pharmaceutical composition comprises a compound of Formula I (e.g., I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237, or a pharmaceutically acceptable salt or ester thereof, and a pharmaceutically acceptable excipient. The pharmaceutical composition can be typically formulated for oral administration. In some embodiments, the pharmaceutical composition is administered to a subject in need to deliver an effective amount of GPR40 agonist in the gastrointestinal tract with minimal or no absorption of GPR40 agonist in systemic circulation.

In some embodiments, the present disclosure provides a method of treating or preventing a disorder, condition or disease that may be responsive to the activation of the GPR40 in a subject in need thereof. In some embodiments, the method comprises administering to the subject an effective amount of one or more compounds of the present disclosure or the pharmaceutical composition herein. In some embodiments, the method comprises administering to the subject an effective amount of a compound of Formula I (e.g., I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237, or a pharmaceutically acceptable salt or ester thereof, or a pharmaceutical composition comprising the same. In some embodiments, the administering is an oral administration.

In some embodiments, the present disclosure provides a method of treating type 2 diabetes mellitus in a subject in need thereof. In some embodiments, the method comprises administering to the subject a therapeutically effective amount of one or more compounds of the present disclosure or the pharmaceutical composition herein. In some embodiments, the method comprises administering to the subject a therapeutically effective amount of a compound of Formula I (e.g., I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237, or a pharmaceutically acceptable salt or ester thereof, or a pharmaceutical composition comprising the same. In some embodiments, the administering is an oral administration.

In some embodiments, the method herein further comprises administering to the subject an additional therapeutic agent. In some embodiments, the additional therapeutic agent can be PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; α-glucosidase inhibitors; agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (vi) acyl CoA:cholesterol acyltransferase (ACAT) inhibitors, (vii) CETP inhibitors, (viii) PCSK9 inhibitor or antibodies; (ix) apolipoproteins inhibitors; and (x) phenolic anti-oxidants; PPARα/γ dual agonists; PPARS agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists; GLP-1/GIP receptor dual agonists; GLP-1/GIP/insulin receptor triple agonists; GLP-1/GIP/glucagon receptor triple agonists; HSD-1 inhibitors; HSD-17 inhibitors; SGLT-2 inhibitors; SGLT-1/SGLT-2 inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin.

It is to be understood that both the foregoing summary and the following detailed description are exemplary and explanatory only, and are not restrictive of the invention herein.

### DETAILED DESCRIPTION

In various embodiments, the present disclosure provides compounds that are useful for modulating GPR40. The compounds herein typically have no or reduced systemic exposure and therefore are expected to have reduced side effects due to such systemic exposure. In some embodiments, the present disclosure also provides pharmaceutical compositions comprising the compound(s) and methods of using the same, such as in treating type 2 diabetes.

### Compounds

In various embodiments, the present disclosure provides a conjugate of a GPR40 agonist covalently linked to a carrier, see e.g., Formula I described herein. Typically, the GPR40 agonist is covalently linked to the carrier through a linker containing an aliphatic group with the longest chain length of at least 10 carbons (such as 10-50 carbons). The aliphatic group can be fully saturated or partially unsaturated. The GPR40 agonist and carrier are not particularly limited and are exemplified herein. As apparent from the present disclosure, a carrier for conjugation with the GPR40 agonist herein generally means any molecule/moiety that can form a covalent link with the GPR40 agonist herein (e.g., through L¹-L²-L³ shown in Formula I). Typically, the carrier has a hydrophilic moiety such as an alcohol, e.g., a diol (e.g., glycol) or a polyol (e.g., glycerol, sugar alcohol, etc.), a sugar, a monosaccharide, disaccharide, or polysaccharide, an amine, an amide, an amino alcohol, an amino ether, water soluble ether, polyethylene glycol (PEG) chain, a carboxylic acid, an amino acid, a peptide, a charged group, or a group that can become charged at pH 7, or any combinations thereof. In some embodiments, the carrier can be a dendrimer, oligomer, or polymer, which has one or more hydrophilic moiety described herein. Each carrier molecule can have one or more available attaching points (typically functional groups, e.g., those derived from OH, NH₂, or carbonyl moieties) suitable for covalently linking to one or more molecules of the GPR40 agonist. Although not prohibited, it should be understood that all of the one or more available attaching points of the carrier are not required to form covalent links with the GPR40 agonist. For example, in some embodiments, the carrier can have five terminal primary amine functional groups, each of the five amine functional groups can independently form a covalent link to the GPR40 agonist or not, so long as one GPR40 agonist is covalently linked to the carrier. The conjugate (e.g., compounds of Formula I herein) can exist as a substantially pure single compound or a mixture of compounds, e.g., a mixture of related compounds having the same carrier core structure and same GPR40 agonist, but with different molar ratios of GPR40 agonist to the carrier.

Without wishing to be bound by theories, it is believed that when administered, the conjugate can be retained in the gastrointestinal tract without being absorbed in the systemic circulation in any significant amount to cause side effects, due to the hydrophilicity and/or size of the carrier molecule. Additionally, as the aliphatic linker of the conjugate is of sufficient length, the conjugate can docket the GPR40 agonist in the transmembrane domains of the GPR40 protein and thus can continue to act as an agonist. Among other advantages, the conjugate of the present disclosure is expected to be useful for modulating GPR40 without side effects or with reduced side effects due to systemic exposure.

Formula I

In some embodiments, the present disclosure provides a compound of Formula I, or a pharmaceutically acceptable salt or ester thereof:

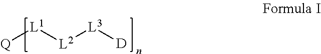

wherein:
Q is a carrier covalently bonded to L¹;
n is an integer of 1-500; L¹ at each occurrence is independently a structure of Formula L-1:

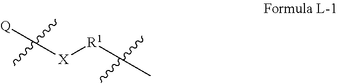

wherein: X is a bond, —CR¹⁰³R¹⁰⁴—, N(R¹⁰⁰)—, —O—, —S—, —SO₂—, —C(═O)—, —C(═O)—N(R¹⁰⁰)—, —S(═O)₂—N(R¹⁰⁰)—, —P(═O)(OR¹⁰²)—N(R¹⁰⁰)—, —C(═O)—O—, —S(═O)₂—O—, or —P(═O)(OR¹⁰²)—O—; and R¹ is a saturated or partially unsaturated aliphatic group, or aromatic group, wherein the longest chain length of the aliphatic group is at least 10 carbons, such as a C₁₀₋₅₀ alkyl (e.g., straight or branched chain C₁₀₋₃₀ alkyl), C₁₀₋₅₀ alkenyl, or C₁₀₋₅₀ alkynyl;
L² at each occurrence is independently —N(R¹⁰⁰)—, —O—, —S—, —SO₂—, —C(═O)—, or a moiety selected from:

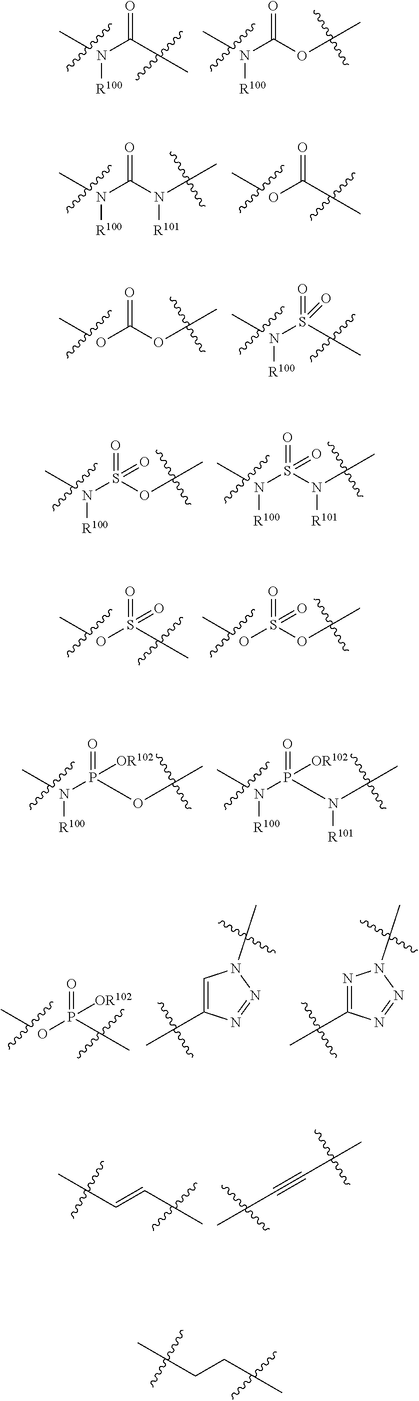

L³ at each occurrence is independently a bond, optionally substituted alkylene, optionally substituted heteroalkylene, optionally substituted carbocyclylene, optionally substituted heterocyclylene, optionally substituted arylene, or optionally substituted heteroarylene, and D is a residue of a GPR40 agonist;
wherein R¹⁰⁰, R¹⁰¹ and R¹⁰² at each occurrence is independently hydrogen, optionally substituted alkyl, or optionally substituted cycloalkyl, wherein R¹⁰³ and R¹⁰⁴ are independently hydrogen, halogen, optionally substituted alkyl, or optionally substituted cycloalkyl; or R¹⁰³ and R¹⁰⁴ are joined to form a C(═O) or an optionally substituted cyclic structure.
   As used herein, the divalent structures can link to the remainder of the compound in either direction, left to right, or right to left, unless otherwise specified. For example, as shown in Formula L-1, “Q” is added at the left end beyond the wiggly line, which means that the “X” in Formula L-1 should form a bond with “Q”, and R¹ would then necessarily connect to L² (although not showing in Formula L-1). On the other hand, the direction of divalent structure of —C(═O)—N(R¹⁰⁰)— as one definition of “X” is not specifically limited. As such, when X is —C(═O)—N(R¹⁰⁰)—, it can connect to the remainder of the molecule in either direction to yield either Q-C(═O)—N(R¹⁰⁰)—R¹ or R¹—C(═O)—N(R¹⁰⁰)-Q. Other divalent structures should be understood similarly.

As discussed herein, the carrier Q is not particularly limited. Without wishing to be bound by theories, one function of Q, by itself or with other features of the compound, is to prevent the compound of Formula I or a relevant GPR40 agonist (e.g., from degradation or hydrolysis) from entering systemic circulation, or to reduce the extent of the compound of Formula I or a relevant GPR40 agonist being absorbed in the systemic circulation. Thus, in some embodiments, the carrier Q can be characterized as a carrier capable of achieving such function. It is believed that carriers that are hydrophilic are better suited for the purposes herein.

In some preferred embodiments, Q is a hydrophilic carrier. As used herein, a hydrophilic carrier refers to a carrier that has one or more hydrophilic moieties, such as an alcohol, such as a diol (e.g., glycol) or a polyol (e.g., glycerol, sugar alcohol, etc.), a sugar, a monosaccharide, disaccharide, or polysaccharide, an amine, an amino alcohol, an amino ether, water soluble ether, a carboxylic acid, an amino acid, a peptide, a charged group, or a group that can become charged at pH 7, or any combinations thereof.

Q typically has one or more available attaching points (typically derived from functional groups, such as OH, NH₂, COOH, etc.) suitable for forming one or more covalent linkage with one or more D, the residue of GPR40 agonist. The number of such available attaching points in Q are not particularly limited, but it obviously needs to be equal to or more than the integer “n” in Formula I. In some embodiments, the number of available attaching points in Q is equal to the integer “n”, wherein each of the available attaching points forms a covalent linkage with D (through L¹-L²-L³). In some embodiments, the number of available attaching points in Q is greater than the integer “n”, wherein some of the available attaching points form a covalent linkage with D and some do not. It would be apparent to those skilled in the art that in certain cases, the compound of Formula I and its subformulae herein can have regioisomers and/or stereoisomers. For example, even for a symmetrical Q, due to partial attachments of D, regioisomers and/or stereoisomers can exist. While not precluded, separating such regioisomers and/or stereoisomers is not necessary for the present disclosure. And the present disclosure is not limited to a particular regioisomer and/or stereoisomer. In any of the embodiments described herein, unless otherwise specified or contrary from context, compounds of the present disclosure (e.g., a compound of Formula I (e.g., I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237) can include any of the potential regioisomers and/or stereoisomers, which include individual isomers or a mixture of isomers in any ratio, for example, as a racemic mixture (as applicable).

Each unit of L¹-L²-L³-D in Formula I can be the same or different. However, in some preferred embodiments, each unit of L¹-L²-L³-D is typically the same.

In some embodiments, Q comprises a diol or polyol unit. For example, in some embodiments, Q can have the following formula:

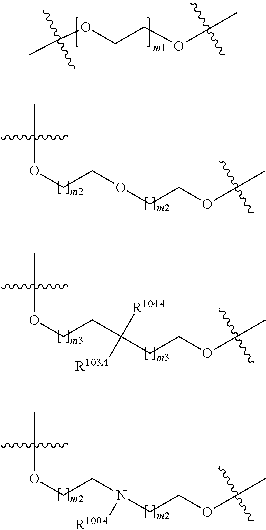

wherein one or both terminal oxygen atom are linked to a unit of L¹-L²-L³-D,
wherein R^{100A} at each occurrence is independently hydrogen, optionally substituted alkyl, or optionally substituted cycloalkyl;
R^{103A} and R^{104A} are independently hydrogen, halogen, optionally substituted alkyl, or optionally substituted cycloalkyl; or R^{103A} and R^{104A} are joined to form a C(═O) or an optionally substituted cyclic structure;
m1 is an integer of 0-100, such as 0-10, e.g., 1-5 (e.g., 1, 2, 3, 4, or 5), 10-50, or 50-100, etc.;
each m2 is independently an integer of 0-10, e.g., 1-5 (e.g., 1, 2, 3, 4, or 5); and
each m3 is independently an integer of 0-10, e.g., 0-5 (e.g., 0, 1, 2, 3, 4, or 5).

For example, in some embodiments, the compound of Formula I can have a Formula I-1, I-2, I-3, I-4, I-5, I-6, I-7, or I-8,

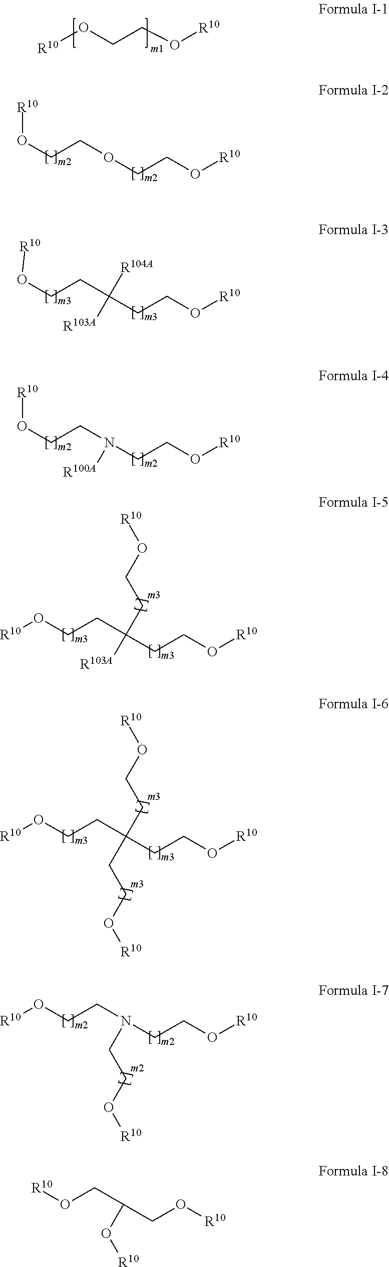

wherein
R¹⁰ at each occurrence is independently hydrogen, optionally substituted alkyl, optionally substituted cycloalkyl, optionally substituted heteroalkyl, optionally substituted heterocyclyl, an oxygen protecting group, or -L¹-L²-L³-D, provided that at least one of the R¹⁰ comprises -L¹-L²-L³-D,
L¹, L², L³, and D are defined herein,
R^{100A} at each occurrence is independently hydrogen, optionally substituted alkyl, or optionally substituted cycloalkyl;
R^{103A} and R^{104A} are independently hydrogen, halogen, optionally substituted alkyl, or optionally substituted cycloalkyl; or R¹⁰³ and R¹⁰⁴ are joined to form a C(═O) or an optionally substituted cyclic structure;
m1 is an integer of 0-100, such as 0-10, e.g., 1-5 (e.g., 1, 2, 3, 4, or 5), 10-50, or 50-100, etc.;
each m2 is independently an integer of 0-10, e.g., 1-5 (e.g., 1, 2, 3, 4, or 5); and
each m3 is independently an integer of 0-10, e.g., 0-5 (e.g., 0, 1, 2, 3, 4, or 5).
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.

In some embodiments, the compound of Formula I has a Formula I-1, wherein one R¹⁰ is hydrogen and the other R¹⁰ is -L¹-L²-L³-D. In some embodiments, the compound has a Formula I-1, wherein both R¹⁰ are -L¹-L²-L³-D. In some embodiments, m1 in formula I-1 is 1, 2, 3, 4, or 5. In some embodiments, m1 in formula I-1 is greater than 5, such as 6-8.

In some embodiments, the compound of Formula I has a Formula I-2, wherein one R¹⁰ is hydrogen and the other R¹⁰ is -L¹-L²-L³-D. In some embodiments, the compound has a Formula I-2, wherein both R¹⁰ are -L¹-L²-L³-D. In some embodiments, each m2 in Formula I-2 is 1, 2, 3, 4, or 5. In some embodiments, both m2 in Formula I-2 are the same.

In some embodiments, the compound of Formula I has a Formula I-3, wherein one R¹⁰ is hydrogen and the other R¹⁰ is -L¹-L²-L³-D. In some embodiments, the compound has a Formula I-3, wherein both R¹⁰ are -L¹-L²-L³-D. In some embodiments, each m3 in formula I-3 is 0, 1, 2, 3, 4, or 5. In some embodiments, both m3 in Formula I-3 are the same. In some embodiments, R^{103A} and R^{104A} are both hydrogen. In some embodiments, R^{103A} and R^{104A} are independently hydrogen or a C₁₋₄ alkyl optionally substituted with one or more (e.g., 1, 2, or 3) substituents independently selected from hydroxyl, amino, C₁₋₆ heteroalkyl, -L¹-L²-L³-D, —O-L¹-L²-L³-D, —N(H)-L¹-L²-L³-D, and —N(-L¹-L²-L³-D)₂. In some embodiments, R^{103A} and R^{104A} are joined to form a C(═O) or a C₃₋₆ cycloalkyl or a 3-7 membered heterocyclic structure having 1 or 2 ring heteroatoms independently selected from N, O, and S, such as

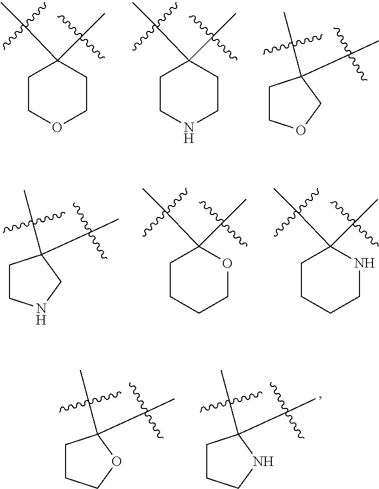

wherein the cycloalkyl or heterocyclic structure can be optionally substituted with one or more (e.g., 1, 2, or 3) substituents independently selected from a C₁₋₄ alkyl, hydroxyl, amino, C₁₋₆ heteroalkyl, -L¹-L²-L³-D, —O-L¹-L²-L³-D, —N(H)-L¹-L²-L³-D, and —N(-L¹-L²-L³-D)₂, wherein the C₁₋₄ alkyl is optionally substituted with one or more (e.g., 1, 2, or 3) substituents independently selected from hydroxyl, amino, C₁₋₆ heteroalkyl, -L¹-L²-L³-D, —O-L¹-L²-L³-D, —N(H)-L¹-L²-L³-D, and —N(-L¹-L²-L³-D)₂.

In some embodiments, the compound of Formula I has a Formula I-4, wherein one R¹⁰ is hydrogen and the other R¹⁰ is -L¹-L²-L³-D. In some embodiments, the compound has a Formula I-4, wherein both R¹⁰ are -L¹-L²-L³-D. In some embodiments, each m2 in Formula I-4 is 1, 2, 3, 4, or 5. In some embodiments, both m2 in Formula I-4 are the same. In some embodiments, R^{100A} is hydrogen. In some embodiments, R^{100A} is a C₁₋₄ alkyl optionally substituted with one or more (e.g., 1, 2, or 3) substituents independently selected from hydroxyl, amino, C₁₋₆ heteroalkyl, -L¹-L²-L³-D, —O-L¹-L²-L³-D, —N(H)-L¹-L²-L³-D, and —N(-L¹-L²-L³-D)₂.

In some embodiments, the compound of Formula I has a Formula I-5, wherein one R¹⁰ is hydrogen and the other two R¹⁰ are -L¹-L²-L³-D. In some embodiments, the compound has a Formula I-5, wherein all three R¹⁰ are -L¹-L²-L³-D. In some embodiments, each m3 in Formula I-5 is 0, 1, 2, 3, 4, or 5. In some embodiments, all m3 in Formula I-5 are the same. In some embodiments, R^{103A} is hydrogen. In some embodiments, R^{103A} is a C₁₋₄ alkyl optionally substituted with one or more (e.g., 1, 2, or 3) substituents independently selected from hydroxyl, amino, C₁₋₆ heteroalkyl, -L¹-L²-L³-D, —O-L¹-L²-L³-D, —N(H)-L¹-L²-L³-D, and —N(-L¹-L²-L³-D)₂.

In some embodiments, the compound of Formula I has a Formula I-6, wherein one or two of R¹⁰ are hydrogen and the other R¹⁰ are -L¹-L²-L³-D. In some embodiments, the compound has a Formula I-6, wherein all four R¹⁰ are -L¹-L²-L³-D. In some embodiments, each m3 in Formula I-6 is 0, 1, 2, 3, 4, or 5. In some embodiments, all m3 in Formula I-6 are the same.

In some embodiments, the compound of Formula I has a Formula I-7, wherein one or two of R¹⁰ are hydrogen and the other R¹⁰ are -L¹-L²-L³-D. In some embodiments, the compound has a Formula I-7, wherein all three R¹⁰ are -L¹-L²-L³-D. In some embodiments, each m2 in Formula I-7 is 1, 2, 3, 4, or 5. In some embodiments, all m2 in Formula I-2 are the same.

In some embodiments, the compound of Formula I has a Formula I-8, wherein one or two of R¹⁰ are hydrogen and the other R¹⁰ are (is) -L¹-L²-L³-D, such as Formula I-8-A, I-8-B, I-8-C, I-8-D. In some embodiments, the compound has a Formula I-8, wherein all three R are -L¹-L²-L³-D.

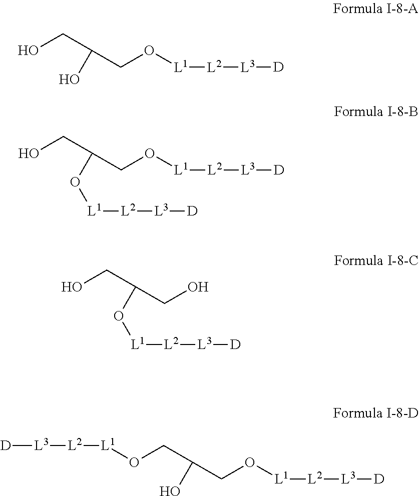

In some embodiments, Q can be a residue of a dendrimer. As understood by those skilled in the art, a dendrimer typically has a core, a number of branches or repeating units, and terminal groups or end groups. Shown below is an example of a G-1 poly(amido amine) dendrimer, which has an ethylene diamine core, with branches that can be formed through Michael addition to methyl acrylate followed by aminolysis with ethylene diamine, and the terminus comprises primary NH₂ groups. The shown G-1 dendrimer has 8 primary NH₂ end groups.

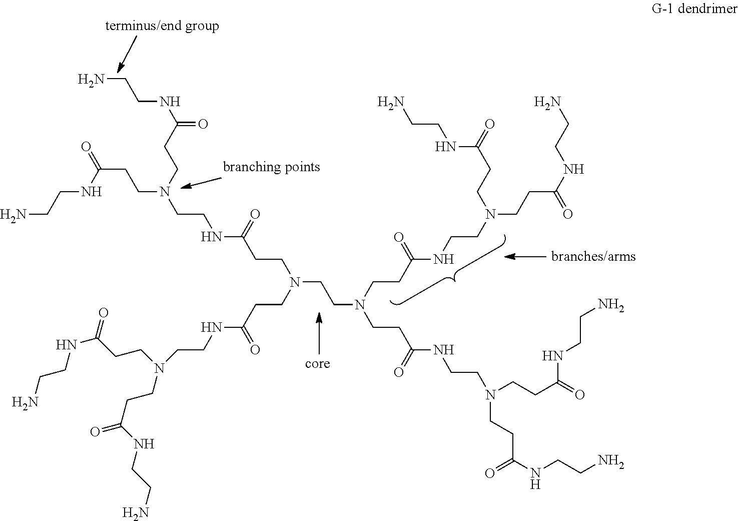 [SMILES: NCCNC(=O)CCN(CCNC(=O)CCN(CCC(=O)NCCN(CCC(=O)NCCN)CCC(=O)NCCN)CCN(CCC(=O)NCCN(CCC(=O)NCCN)CCC(=O)NCCN)CCC(=O)NCCN(CCC(=O)NCCN)CCC(=O)NCCN)CCC(=O)NCCN]

Useful dendrimers for compounds of Formula I are not particularly limited, which include any of those known in the art that have one or more end groups that can form a covalent bond with L¹. For example, in some embodiments, Q is the residue of a poly(amide amine) dendrimer, a poly(propylene amine) dendrimer, or a poly (amide amine)-poly(propylene amine) dendrimer. In some embodiments, Q is the residue of a dendrimer which has a hydroxyl, amine, or carbonyl moiety at each terminus. Such dendrimer can form covalent bonds with -L¹-L²-L³-D at one or more of its termini via various chemical couplings, such as ether formation, ester formation, amide formation, carbonate formation, urea formation, carbamate formation, imine formation, amine formation, etc.

Dendrimers having various core structures are useful for compounds of Formula I. For example, in some embodiments, Q can be a residue of a dendrimer which has a core of a diamine or polyamine (e.g., triamine, tetraamine, etc.), such as a C₂₋₈ alkylene diamine, C₂₋₈ heteroalkylene diamine, C₃₋₆ cycloalkylene diamine, 3-8 membered heterocyclylene diamine, C₂₋₈ alkylene-C₃₋₆ cycloalkylene diamine, C₂₋₈ alkylene-3-8 membered heterocyclylene diamine, C₂₋₈ heteroalkylene-C₃₋₆ cycloalkylene diamine, C₂₋₈ heteroalkylene-3-8 membered heterocyclylene diamine, C₂₋₈ alkylene-C₃₋₆ cycloalkylene-C₂₋₈ alkylene diamine, C₂₋₈ alkylene-3-8 membered heterocyclylene-C₂₋₈ alkylene diamine, C₂₋₈ heteroalkylene-C₃₋₆ cycloalkylene-C₂₋₈ heteroalkylene diamine, C₂₋₈ heteroalkylene-3-8 membered heterocyclylene-C₂₋₈ heteroalkylene diamine, etc., wherein each of the alkylene, heteroalkylene, cycloalkylene, cycloalkylene, heterocyclylene is optionally substituted, for example, with C₁₋₄ alkyl, hydroxyl, and/or amine groups. In some embodiments, Q can be a residue of a dendrimer which has a core of ethylene diamine, propylene diamine, butylene diamine, pentylene diamine, cyclohexylene diamine, cyclobutylene diamine, NH₂—CH₂CH₂-piperizine-CH₂CH₂—NH₂, etc.

In some embodiments, Q can be a residue of a dendrimer which has a core of Formula Q:

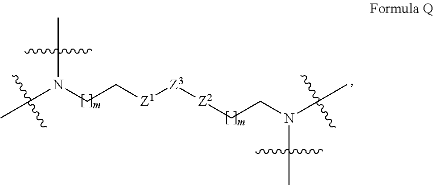

wherein:
Z¹ is a bond, NR^{100B}, or O;
Z² is a bond, NR^{100B}, or O;
Z³ is a bond, —C(═O)—, —SO₂—, —C(═O)—R²—C(═O)—, —C(═O)—R²—, —S(O)₂—R²—S(O)₂—, —S(O)₂—R²—, a polyethylene glycol chain, optionally substituted alkylene, optionally substituted heteroalkylene, optionally substituted carbocyclylene, optionally substituted heterocyclylene, optionally substituted arylene, or optionally substituted heteroarylene, wherein R² is optionally substituted alkylene, optionally substituted heteroalkylene, optionally substituted carbocyclylene, optionally substituted heterocyclylene, optionally substituted arylene, or optionally substituted heteroarylene, provided that when neither of Z¹ and Z² is a bond, then Z³ is not a bond, and when Z³ is a polyethylene glycol (PEG) chain, then Z¹ and Z² are both a bond;
each m is independently an integer of 0-10, e.g., 1-5; and
   R^{100B} at each occurrence is independently hydrogen, optionally substituted alkyl, or optionally substituted cycloalkyl.

In some embodiments, in Formula Q, one of Z¹ and Z² is a bond. In some embodiments, in Formula Q, both Z¹ and Z² is a bond. In some embodiments, in Formula Q, neither of Z¹ and Z² is a bond. It should be clear to those skilled in the art that when a variable is said to be a bond, the two immediately groups/atoms connected to the variable are directly connected to each other, as if the variable does not exist. For example, in the unit of Z¹—Z³—Z² in Formula Q, when Z³ is a bond, Z¹ and Z² are not a bond, then the unit of Z¹—Z³—Z² in Formula Q would be the same as Z¹—Z² because Z³ does not exist. Other expressions should be understood similarly.

For example, in some embodiments, in Formula Q, Z¹ and Z² are both a bond, and Z³ is —C(═O)—, —SO₂—, —C(═O)—R²—C(═O)—, —C(═O)—R²—, —S(O)₂—R²—S(O)₂—, or —S(O)₂—R²—, wherein R² is C₁₋₁₀ alkylene, C₁₋₁₀ heteroalkylene, C₃₋₁₀ carbocyclylene, 3-10 membered heterocyclylene, phenylene, or 5-10 membered heteroarylene, wherein the heteroalkylene has 1-5 heteroatoms independently selected from O, N, and S, wherein the heterocyclylene or heteroarylene has 1-3 ring heteroatoms independently selected from O, N, and S. For example, in some embodiments, Z³ can be —C(═O)—. In some embodiments, Z³ can be —C(═O)—(C₁₋₁₀ alkylene)- or —C(═O)—(C₁₋₁₀ heteroalkylene)-.

In some embodiments, in Formula Q, Z¹ and Z² are both a bond, and Z³ is C₁₋₁₀ alkylene, C₁₋₁₀ heteroalkylene, C₃₋₁₀ carbocyclylene, 3-10 membered heterocyclylene, phenylene, or 5-10 membered heteroarylene, wherein the heteroalkylene has 1-5 heteroatoms independently selected from O, N, and S, wherein the heterocyclylene or heteroarylene has 1-3 ring heteroatoms independently selected from O, N, and S. In some embodiments, Z³ can be C₁₋₁₀ alkylene or C₁₋₁₀ heteroalkylene.

In some embodiments, in Formula Q, Z¹ and Z² are both a bond, and Z³ is a PEG chain, with various suitable molecular weights. For example, in some embodiments, the PEG can be a low molecular weight PEG having a number average molecular weight (Mₙ) or a weight average molecular weight (M_{w}) of about 200 to about 5000 g/mol. In some embodiments, the PEG can have a Mₙ or M_{w} of about 5000 to about 20000 g/mol. In some embodiments, the PEG can have a Mₙ or M_{w} of about 20000 to about 100,000 g/mol. In some embodiments, the PEG can also have a Mₙ or M_{w} of about 100,000 to about 500,000 g/mol. As will be understood by those skilled in the art, a PEG chain typically have two end hydroxyl groups available for conjugation. In Formula Q, when Z³ is a PEG chain, the two oxygen atoms of the end hydroxyl groups of the PEG chain can be covalently linked to the remainder of the molecule.

For example, in some embodiments, in Formula Q, Z¹ is NR^{100B} or O; Z² is a bond; and Z³ is —C(═O)—, —SO₂—, —C(═O)—R²—C(═O)—, —C(═O)—R²—, —S(O)₂—R²—S(O)₂—, or —S(O)₂—R²—, wherein R² is C₁₋₁₀ alkylene, C₁₋₁₀ heteroalkylene, C₃₋₁₀ carbocyclylene, 3-10 membered heterocyclylene, phenylene, or 5-10 membered heteroarylene, wherein the heteroalkylene has 1-5 heteroatoms independently selected from O, N, and S, wherein the heterocyclylene or heteroarylene has 1-3 ring heteroatoms independently selected from O, N, and S. For example, in some embodiments, Z³ can be —C(═O)—. In some embodiments, Z³ can be —C(═O)—(C₁₋₁₀ alkylene)- or —C(═O)—(C₁₋₁₀ heteroalkylene)-, wherein the carbonyl group is connected to Z¹. In some embodiments, R^{100B} at each occurrence is independently hydrogen, a C₁₋₄ alkyl optionally substituted with one or more (e.g., 1, 2, or 3) substituents independently selected from hydroxyl, amino, and C₁₋₆ heteroalkyl, or a dendron of the dendrimer. In some embodiments, R^{100B} at each occurrence is independently hydrogen or a C₁₋₄ alkyl (e.g., methyl, ethyl, or isopropyl).

In some embodiments, in Formula Q, Z¹ is NR^{100B} or O; Z² is a bond; and Z³ is C₂₋₁₀ alkylene, C₂₋₁₀ heteroalkylene, C₃₋₁₀ carbocyclylene, 3-10 membered heterocyclylene, phenylene, or 5-10 membered heteroarylene, wherein the heteroalkylene has 1-5 heteroatoms independently selected from O, N, and S, wherein the heterocyclylene or heteroarylene has 1-3 ring heteroatoms independently selected from O, N, and S. In some embodiments, Z³ can be C₂₋₁₀ alkylene or C₂₋₁₀ heteroalkylene. In some embodiments, R^{100B} at each occurrence is independently hydrogen, a C₁₋₄ alkyl optionally substituted with one or more (e.g., 1, 2, or 3) substituents independently selected from hydroxyl, amino, and C₁₋₆ heteroalkyl, or a dendron of the dendrimer. In some embodiments, R^{100B} at each occurrence is independently hydrogen or a C₁₋₄ alkyl (e.g., methyl, ethyl, or isopropyl).

In some embodiments, in Formula Q, Z¹ is NR^{100B} or O; Z² is NR^{100B} or O; and Z³ is —C(═O)—, —SO₂—, —C(═O)—R²—C(═O)—, —C(═O)—R²—, —S(O)₂—R²—S(O)₂—, or —S(O)₂—R²—, wherein R² is C₁₋₁₀ alkylene, C₁₋₁₀ heteroalkylene, C₃₋₁₀ carbocyclylene, 3-10 membered heterocyclylene, phenylene, or 5-10 membered heteroarylene, wherein the heteroalkylene has 1-5 heteroatoms independently selected from O, N, and S, wherein the heterocyclylene or heteroarylene has 1-3 ring heteroatoms independently selected from O, N, and S. For example, in some embodiments, Z³ can be —C(═O)—. In some embodiments, Z³ can be —C(═O)—(C₁₋₁₀ alkylene)- or —C(═O)—(C₁₋₁₀ heteroalkylene)-. In some embodiments, Z³ can be —C(═O)—(C₁₋₁₀ alkylene)-C(═O)— or —C(═O)—(C₁₋₁₀ heteroalkylene)-C(═O)—. In some embodiments, R^{100B} at each occurrence is independently hydrogen, a C₁₋₄ alkyl optionally substituted with one or more (e.g., 1, 2, or 3) substituents independently selected from hydroxyl, amino, and C₁₋₆ heteroalkyl, or a dendron of the dendrimer. In some embodiments, R^{100B} at each occurrence is independently hydrogen or a C₁₋₄ alkyl (e.g., methyl, ethyl, or isopropyl).

In some embodiments, in Formula Q, Z¹ is NR^{100B} or O; Z² is NR^{100B} or O; and Z³ is C₂₋₁₀ alkylene, C₂₋₁₀ heteroalkylene, C₃₋₁₀ carbocyclylene, 3-10 membered heterocyclylene, phenylene, or 5-10 membered heteroarylene, wherein the heteroalkylene has 1-5 heteroatoms independently selected from O, N, and S, wherein the heterocyclylene or heteroarylene has 1-3 ring heteroatoms independently selected from O, N, and S. In some embodiments, Z³ can be C₁₋₁₀ alkylene or C₁₋₁₀ heteroalkylene. In some embodiments, R^{100B} at each occurrence is independently hydrogen, a C₁₋₄ alkyl optionally substituted with one or more (e.g., 1, 2, or 3) substituents independently selected from hydroxyl, amino, and C₁₋₆ heteroalkyl, or a dendron of the dendrimer. In some embodiments, R^{100B} at each occurrence is independently hydrogen or a C₁₋₄ alkyl (e.g., methyl, ethyl, or isopropyl).

In any of the embodiments herein, unless specified or contrary from context, m in Formula Q at each occurrence can be independently 0, 1, 2, 3, 4, or 5.

In some embodiments, Q can be a residue of a dendrimer which has a core selected from:

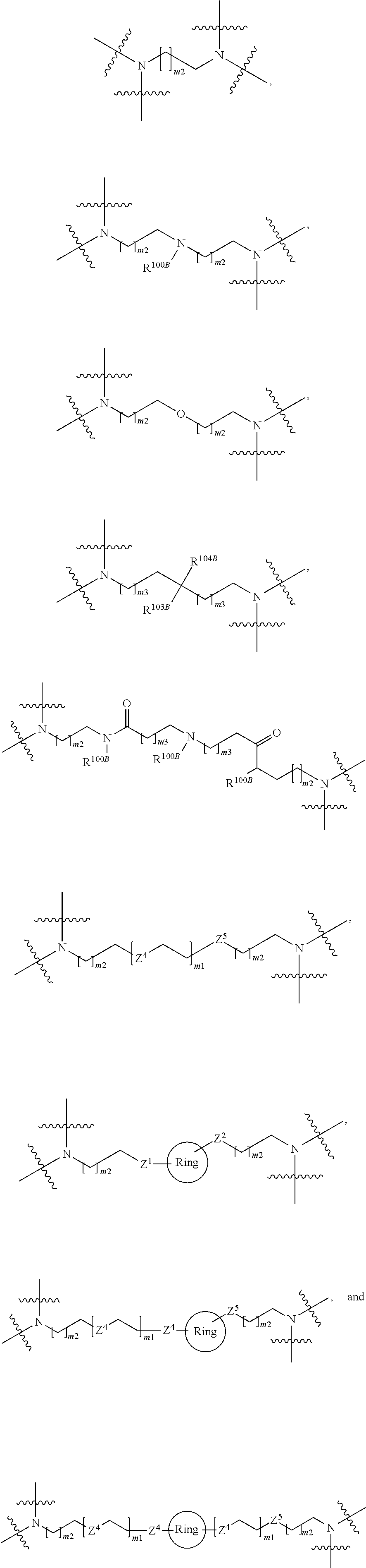

wherein:
Z¹ at each occurrence is independently a bond, NR^{100B} or O;
Z² is a bond, NR^{100B} or O;
Z⁴ at each occurrence is independently a bond, NR^{100B} or O;
Z⁵ is a bond, NR^{100B} or O;
R^{100B} at each occurrence is independently hydrogen, optionally substituted alkyl, or optionally substituted cycloalkyl;
R^{103B} and R^{104B} are independently hydrogen, halogen, optionally substituted alkyl, or optionally substituted cycloalkyl; or R^{103B} and R^{104B} are joined to form a C(═O) or an optionally substituted cyclic structure;
Ring is an optionally substituted ring structure optionally having ring heteroatoms, wherein the ring structure is aromatic or non-aromatic;
m1 is an integer of 0-100, such as 1-10, e.g., 1-5;
each m2 is independently an integer of 0-10, e.g., 1-5; and
each m3 is independently an integer of 0-10, e.g., 0-5.

In some embodiments, Q can be a residue of a dendrimer which has a core of

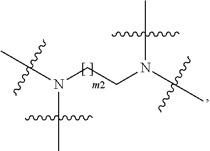

wherein m2 is 1-10, such as 1, 2, 3, 4, or 5. For example, the core can be an ethylene diamine or propylene diamine core, i.e., m2 is 1 or 2 respectively.

In some embodiments, Q can be a residue of a dendrimer which has a core of

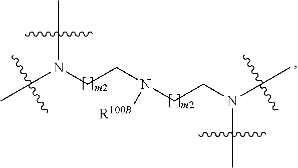

wherein m2 is 1-10, such as 1, 2, 3, 4, or 5, and R^{100B} is hydrogen, a C₁₋₄ alkyl optionally substituted with one or more (e.g., 1, 2, or 3) substituents independently selected from hydroxyl, amino, and C₁₋₆ heteroalkyl, or a dendron of the dendrimer. In some embodiments, R^{100B} is hydrogen or a C₁₋₄ alkyl (e.g., methyl, ethyl, or isopropyl).

In some embodiments, Q can be a residue of a dendrimer which has a core of

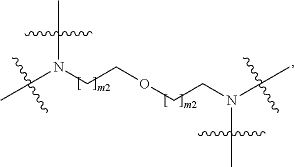

wherein m2 is 1-10, such as 1, 2, 3, 4, or 5. In some embodiments, m2 is 1 or 2.

In some embodiments, Q can be a residue of a dendrimer which has a core of

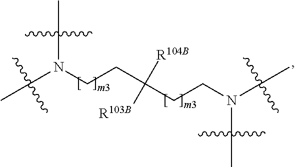

wherein each m3 is 0-10, such as 0, 1, 2, 3, 4, or 5. In some embodiments, each m3 is 0, 1 or 2. In some embodiments, R^{103B} and R^{104B} are both hydrogen. In some embodiments, R^{103B} and R^{104B} are independently hydrogen or a C₁₋₄ alkyl optionally substituted with one or more (e.g., 1, 2, or 3) substituents independently selected from hydroxyl, amino, and C₁₋₆ heteroalkyl. In some embodiments, R^{103A} and R^{104A} are joined to form a C(═O) or a C₃₋₆ cycloalkyl or a 3-7 membered heterocyclic structure having 1 or 2 ring heteroatoms independently selected from N, O, and S, such as

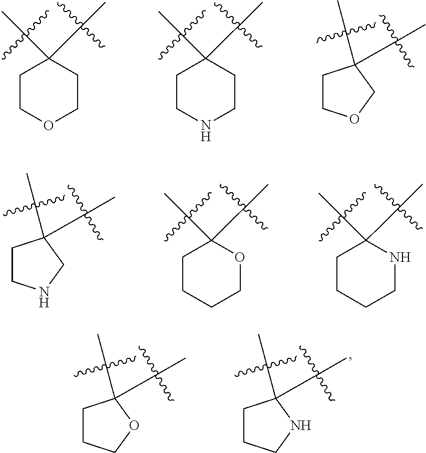

wherein the cycloalkyl or heterocyclic structure can be optionally substituted with one or more (e.g., 1, 2, or 3) substituents independently selected from C₁₋₄ alkyl, hydroxyl, amino and C₁₋₆ heteroalkyl, wherein the C₁₋₄ alkyl is optionally substituted with one or more (e.g., 1, 2, or 3) substituents independently selected from hydroxyl, amino, and C₁₋₆ heteroalkyl.

In some embodiments, Q can be a residue of a dendrimer which has a core of

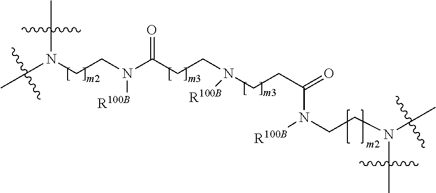

wherein each m2 is independently 1-10, such as 1, 2, 3, 4, or 5, m3 is 0-10, such as 0, 1, 2, 3, 4, or 5, and R^{100B} at each occurrence is independently hydrogen, a C₁₋₄ alkyl optionally substituted with one or more (e.g., 1, 2, or 3) substituents independently selected from hydroxyl, amino, and C₁₋₆ heteroalkyl. In some embodiments, R^{100B} at each occurrence is independently hydrogen or a C₁₋₄ alkyl (e.g., methyl, ethyl, or isopropyl). In some embodiments, R^{100B} at each occurrence is hydrogen. In some embodiments, the center R^{100B} is a C₁₋₄ alkyl (e.g., methyl, ethyl, or isopropyl) and the other R^{100B} is hydrogen. In some embodiments, each m2 is 1. In some embodiments, each m2 is 2. In some embodiments, each m3 is 0. In some embodiments, each m3 is 1. In some embodiments, each m3 is 2.

In some embodiments, Q can be a residue of a dendrimer which has a core of a nitrogen atom or

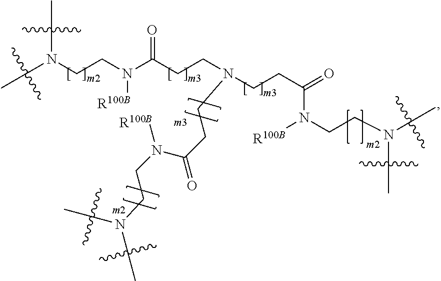

wherein each m2 is independently 1-10, such as 1, 2, 3, 4, or 5, m3 is 0-10 such as 0, 1, 2, 3, 4, or 5, and R^{100B} at each occurrence is independently hydrogen, a C₁₋₄ alkyl optionally substituted with one or more (e.g., 1, 2, or 3) substituents independently selected from hydroxyl, amino, and C₁₋₆ heteroalkyl. In some embodiments, R_{100B} at each occurrence is independently hydrogen or a C₁₋₄ alkyl (e.g., methyl, ethyl, or isopropyl). In some embodiments, R^{100B} at each occurrence is hydrogen. In some embodiments, each m2 is 1. In some embodiments, each m2 is 2. In some embodiments, each m3 is 0. In some embodiments, each m3 is 1. In some embodiments, each m3 is 2.

In some embodiments, Q can be a residue of a dendrimer which has a core of

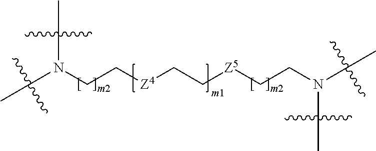

wherein m1 is 0-100, e.g., 0-10, such as 0, 1, 2, 3, 4, 5, 6, 7, or 8, each m2 is 0-10, such as 1, 2, 3, 4, or 5, Z⁴ at each occurrence is independently NR^{100B} or O, and Z⁵ is NR^{100B} or O. In some embodiments, Z⁴ at each occurrence is O, and Z⁵ is NR^{100B} or O. In some embodiments, Z⁴ at each occurrence is O, and Z⁵ is O. In some embodiments, R^{100B} at each occurrence is independently hydrogen or a C₁₋₄ alkyl (e.g., methyl, ethyl, or isopropyl). In some embodiments, R^{100B} at each occurrence is hydrogen. In some embodiments, each m2 is 1. In some embodiments, each m2 is 2. In some embodiments, m1 is 3, 4, 5, or 6. In some embodiments, m1 is 0, 1 or 2.

In some embodiments, Q can be a residue of a dendrimer which has a core of

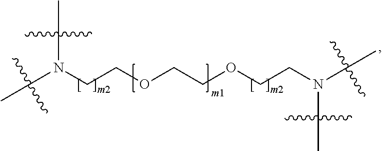

wherein: m1 is an integer of 0-100, such as 0-10 (e.g., 1, 2, 3, 4, 5, 6, 7, or 8); and each m2 is independently an integer of 0-5 (e.g., 1, 2, or 3). For example, in some embodiments, each m2 is 1. In some embodiments, each m2 is 2. In some embodiments, m1 is 3, 4, 5, or 6. In some embodiments, m1 is 1 or 2.

In some embodiments, Q can be a residue of a dendrimer which has a core of

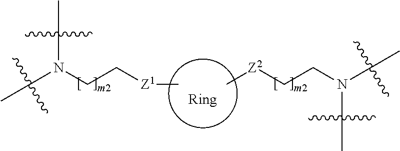

wherein each m2 is 1, 2, 3, 4, or 5, Z¹ is a bond, NR^{100B} or O, and Z² is a bond, NR^{100B} or O. In some embodiments, Ring is a C₃₋₆ cycloalkyl, for example, 1,4-cyclohexylene. In some embodiments, Ring is a 3-10 membered heterocyclic ring having 1-3 ring heteroatoms independently selected from N, O, and S. For example, in some embodiments, both Z¹ and Z² are a bond, and Ring can be selected from

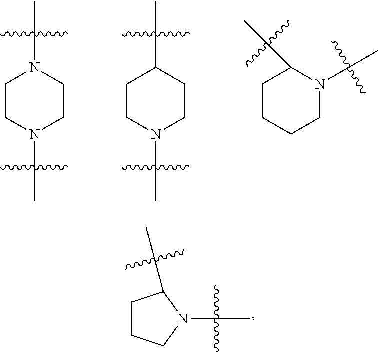 [SMILES: *N1CCN(*)CC1 ; *C1CCN(*)CC1 ; *C1CCCCN1* ; *C1CCCN1*]

e.g., the core can be

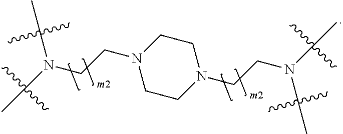

In some embodiments, R^{100B} at each occurrence is independently hydrogen or a C₁₋₄ alkyl (e.g., methyl, ethyl, or isopropyl). In some embodiments, R^{100B} at each occurrence is hydrogen.

In some embodiments, Q can be a residue of a dendrimer which has a core of

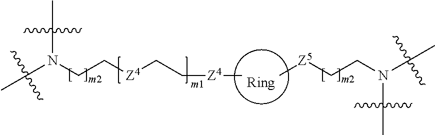

wherein m1 is 0, 1, 2, 3, 4, 5, 6, 7, or 8, each m2 is 1, 2, 3, 4, or 5, Z⁴ at each occurrence is independently a bond, NR^{100B} or O, and Z⁵ is a bond, NR^{100B} or O. In some embodiments, Z⁴ at each occurrence is O, and Z⁵ is NR^{100B} or O. In some embodiments, Z⁴ at each occurrence is O, and Z⁵ is O. In some embodiments, Ring is a C₃₋₆ cycloalkyl, for example, 1,4-cyclohexylene. In some embodiments, Ring is a 3-10 membered heterocyclic ring having 1-3 ring heteroatoms independently selected from N, O, and S. For example, in some embodiments, both Z⁴ and Z⁵ that are immediately connected to the Ring are a bond, and Ring can be selected from

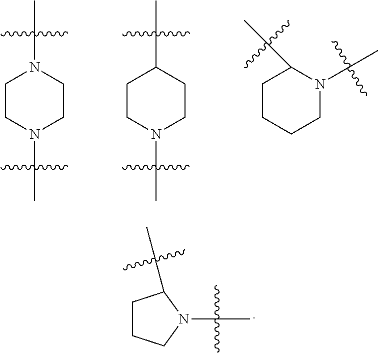 [SMILES: *N1CCN(*)CC1 ; *C1CCN(*)CC1 ; *C1CCCCN1* ; *C1CCCN1*]

In some embodiments, R^{100B} at each occurrence is independently hydrogen or a C₁₋₄ alkyl (e.g., methyl, ethyl, or isopropyl). In some embodiments, R^{100B} at each occurrence is hydrogen. In some embodiments, each m2 is 1. In some embodiments, each m2 is 2. In some embodiments, m1 is 3, 4, 5, or 6. In some embodiments, m1 is 0, 1 or 2.

In some embodiments, Q can be a residue of a dendrimer which has a core of

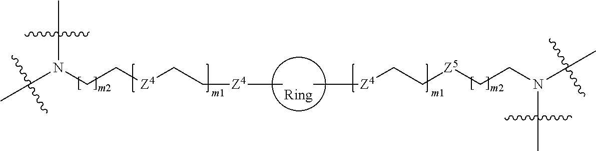

wherein each m1 is 0, 1, 2, 3, 4, 5, 6, 7, or 8, each m2 is 1, 2, 3, 4, or 5, Z⁴ at each occurrence is independently a bond, NR^{100B} or O, and Z⁵ is a bond, NR^{100B} or O. In some embodiments, Z⁴ at each occurrence is O, and Z⁵ is NR^{100B} or O. In some embodiments, Z⁴ at each occurrence is O, and Z⁵ is O. In some embodiments, Ring is a C₃₋₆ cycloalkyl, for example, 1,4-cyclohexylene. In some embodiments, Ring is a 3-10 membered heterocyclic ring having 1-3 ring heteroatoms independently selected from N, O, and S. For example, in some embodiments, both Z⁴ that are immediately connected to the Ring are a bond, and Ring can be selected from

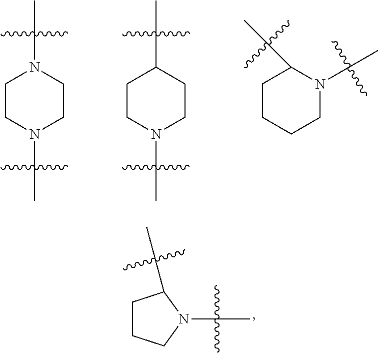 [SMILES: *N1CCN(*)CC1 ; *C1CCN(*)CC1 ; *C1CCCCN1* ; *C1CCCN1*]

In some embodiments, R^{100B} at each occurrence is independently hydrogen or a C₁₋₄ alkyl (e.g., methyl, ethyl, or isopropyl). In some embodiments, R^{100B} at each occurrence is hydrogen. In some embodiments, each m2 is 1. In some embodiments, each m2 is 2. In some embodiments, each m1 is 1. In some embodiments, each m1 is 2. In some embodiments, each m1 is 3. In some embodiments, each m1 is 4.

In some embodiments, Q in Formula I can have any of the cores described herein, with any suitable branches and termini (e.g., described herein). For example, in some embodiments, Q has one or more terminus, with each terminus capable of forming a covalent bond with L¹, e.g., each terminus has a hydroxyl, amine, or carbonyl moiety, such as OH, NH₂, or COOH. In some embodiments, the Q can have one or more branches (inclusive of the branching point), characterized by having a structural moiety of

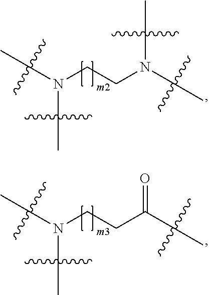

or a combination thereof, wherein m2 is an integer of 0-10, e.g., 1, 2 or 3, and m3 is an integer of 0-10, e.g., 0, 1, 2, or 3. In some embodiments, the Q can have one or more branches having a structural moiety of

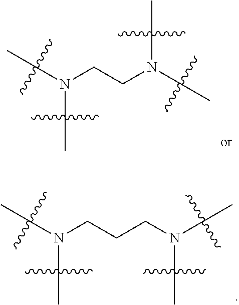 [SMILES: *N(*)CCN(*)* ; *N(*)CCCN(*)*]

In some embodiments, the Q can have one or more branches having a structural moiety of

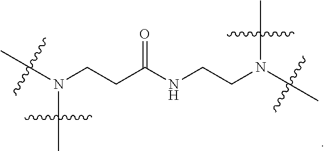 [SMILES: *N(*)CCNC(=O)CCN(*)*]

In some embodiments, the Q in Formula I can have a Formula Q-1:

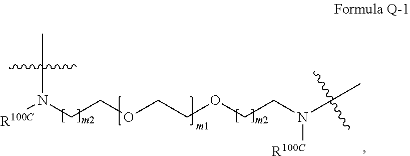

wherein: m1 is an integer of 0-100, such as 0-10, 10-50, 50-100, etc., and each m2 is independently an integer of 0-5, and each R^{100C} is independently hydrogen, optionally substituted alkyl, or optionally substituted cycloalkyl,
wherein at least one of the terminal NR^{100C} of Formula Q-1 forms a covalent bond with an L¹.

For example, in some embodiments, the compound of Formula I can have a Formula I-9A or I-9B:

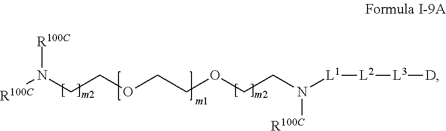

or

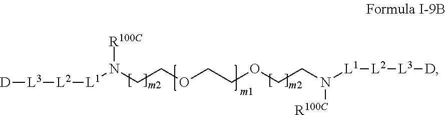

wherein:
L¹, L², L³, and D are defined herein;
m1 is an integer of 0-100, such as 0-10 (e.g., 1, 2, 3, 4, 5, 6, 7, 8, 9, or 10) or 10-50, 50-100, etc.;
each m2 is independently 0-5 (e.g., 1, 2, or 3); and
each R^{100C} is independently hydrogen, optionally substituted alkyl, or optionally substituted cycloalkyl.
   In some embodiments, m1 in Formula I-9A or I-9B is 1, 2, 3, 4, 5, or 6. In some embodiments, each m2 in Formula I-9A or I-9B is 1. In some embodiments, each m2 in Formula I-9A or I-9B is 2. In some embodiments, each R^{100C} in Formula I-9A or I-9B is independently hydrogen or a C₁₋₄ alkyl (e.g., methyl, ethyl or isopropyl). In some embodiments, each R^{100C} in Formula I-9B is hydrogen.

In some embodiments, the Q in Formula I can have a Formula Q-1-P:

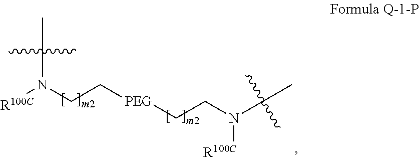

wherein: PEG is a residue of a polyethylene glycol chain or PEG chain, and each m2 is independently an integer of 0-5, and each R^{100C} is independently hydrogen, optionally substituted alkyl, or optionally substituted cycloalkyl,
wherein at least one of the terminal NR^{100C} of Formula Q-1-P forms a covalent bond with an L¹. In some embodiments, the PEG can be a low molecular weight PEG having a number average molecular weight (Mₙ) or a weight average molecular weight (M_{w}) of about 200 to about 5000 g/mol. In some embodiments, the PEG can also have a Mₙ or M_{w} of about 5000 to about 20000 g/mol. In some embodiments, the PEG can also have a Mₙ or M_{w} of about 20000 to about 100,000 g/mol. In some embodiments, the PEG can have a Mₙ or M_{w} of about 100,000 to about 500,000 g/mol.

For example, in some embodiments, the compound of Formula I can have a Formula I-9A-P or I-9B-P:

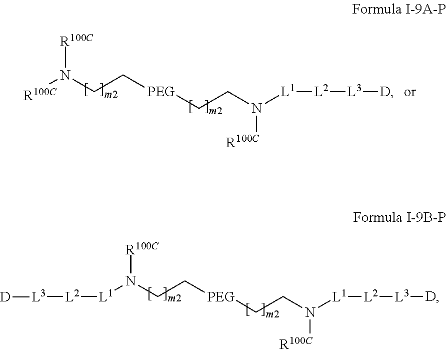

wherein:
L¹, L², L³, and D are defined herein;
PEG is a residue of a polyethylene glycol chain or PEG chain;
each m2 is independently 0-5 (e.g., 1, 2, or 3); and
each R^{100C} is independently hydrogen, optionally substituted alkyl, or optionally substituted cycloalkyl.
In some embodiments, the PEG in Formula I-9A-P or I-9B-P can be a low molecular weight PEG having a number average molecular weight (Mₙ) or a weight average molecular weight (M_{w}) of about 200 to about 5000 g/mol. In some embodiments, the PEG in Formula I-9A-P or I-9B-P can have a Mₙ or M_{w} of about 5000 to about 20000 g/mol. In some embodiments, the PEG in Formula I-9A-P or I-9B-P can have a Mₙ or M_{w} of about 20000 to about 100,000 g/mol. In some embodiments, the PEG in Formula I-9A-P or I-9B-P can have a Mₙ or M_{w} of about 100,000 to about 500,000 g/mol. In some embodiments, each m2 in Formula I-9A-P or I-9B-P is 1. In some embodiments, each m2 in Formula I-9A-P or I-9B-P is 2. In some embodiments, each R^{100C} in Formula I-9A-P or I-9B-P is independently hydrogen or a C₁₋₄ alkyl (e.g., methyl, ethyl or isopropyl). In some embodiments, each R^{100C} in Formula I-9B-P is hydrogen.

In some embodiments, the Q in Formula I can have a Formula Q-2:

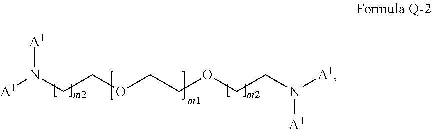

wherein each A¹ is independently F-1, F-2, or F-3,

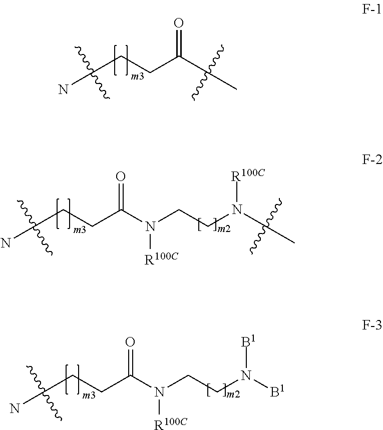

wherein each B¹ group in F-3 is independently F-1 or F-2, or a moiety having at least one repeating units of

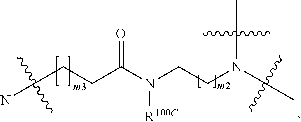

wherein the moiety terminates with a structure comprising

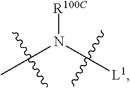

(L¹ is showing to show connectivity if L¹-L²-L³-D is bond at the terminal);
wherein:
m1 is an integer of 0-100, such as 0-10 (e.g., 1, 2, 3, 4, 5, 6, 7, 8, 9, or 10), 10-50, or 50-100 etc.,
each m2 is an integer of 0-5 (e.g., 1, 2, or 3),
each m3 is independently an integer of 0-5 (e.g., 0, 1, 2, or 3),
R^{100C} at each occurrence is independently hydrogen, optionally substituted alkyl, or optionally substituted cycloalkyl; and
wherein at least one of the A¹ forms a covalent bond with an L¹ through a terminal carbonyl group or —N—R^{100C} group.

For example, in some embodiments, the compound of Formula I can have a Formula I-10A:

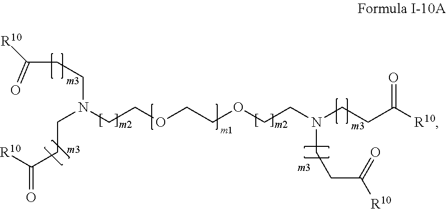

wherein:
R¹⁰ at each occurrence is independently —OH, alkoxy (e.g., C₁₋₄ alkoxy), NH₂, monoalkyl amine (e.g., NH(C₁₋₄ alkyl)), dialkyl amine (e.g., N(C₁₋₄ alkyl)(C₁₋₄ alkyl)), optionally substituted alkyl, optionally substituted cycloalkyl, optionally substituted heteroalkyl, optionally substituted heterocyclyl, or -L¹-L²-L³-D, provided that at least one of the R¹⁰ comprises -L¹-L²-L³-D,
L¹, L², L³, and D are defined herein,
m1 is 0-100, such as 0-10 (e.g., 0, 1, 2, 3, 4, 5, 6, 7, or 8), 10-50, 50-100, etc.,
each m2 is an integer of 0-5 (e.g., 1, 2, or 3),
each m3 is independently an integer of 0-5 (e.g., 0, 1, 2, or 3),
In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
   In some embodiments, one, two, or three R¹⁰ in Formula I-10A is -L¹-L²-L³-D, and the remaining R¹⁰ are (is) —OH, alkoxy (e.g., C₁₋₄ alkoxy), NH₂, monoalkyl amine (e.g., NH(C¹⁻⁴ alkyl)), or dialkyl amine (e.g., N(C₁₋₄ alkyl)(C₁₋₄ alkyl)). In some embodiments, all four R¹⁰ in Formula I-10A are -L¹-L²-L³-D.

In some embodiments, the compound of Formula I can have a Formula I-10B:

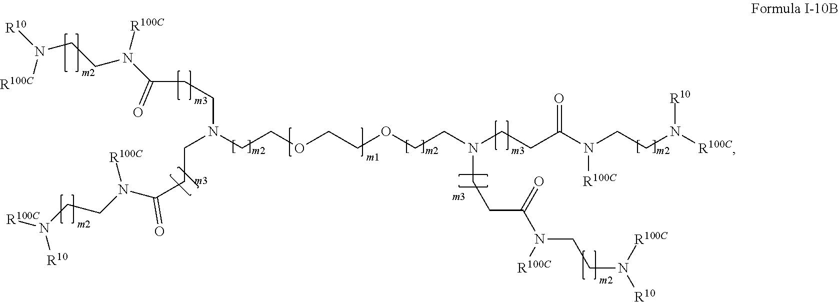

wherein:
R¹⁰ at each occurrence is independently hydrogen, optionally substituted alkyl, optionally substituted cycloalkyl, optionally substituted heteroalkyl, optionally substituted heterocyclyl, or -L¹-L²-L³-D, provided that at least one of the R¹⁰ comprises -L¹-L²-L³-D,
L¹, L², L³, and D are defined herein,
m1 is 0-100 such as 0-10 (e.g., 0, 1, 2, 3, 4, 5, 6, 7, or 8), 10-50, 50-100, etc.,
each m2 is an integer of 0-5 (e.g., 1, 2, or 3),
each m3 is independently an integer of 0-5 (e.g., 0, 1, 2, or 3),
each R^{100C} is independently hydrogen, optionally substituted alkyl, or optionally substituted cycloalkyl.
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
   In some embodiments, each R^{100C} in Formula I-10B is hydrogen or a C₁₋₄ alkyl (e.g., methyl, ethyl, or isopropyl). In some embodiments, each R^{100C} in Formula I-10B is hydrogen. In some embodiments, m1 in Formula I-10B is 0, 1, 2, 3, 4, 5, 6, 7, 8, 9, or 10. In some embodiments, each m2 in Formula I-10B is 1. In some embodiments, each m2 in Formula I-10B is 2. In some embodiments, each m3 in Formula I-10B is 0, 1, or 2. In some embodiments, each m3 in Formula I-10B is 1. In some embodiments, each m3 in Formula I-10B is 2. In some embodiments, one R¹⁰ in Formula I-10B is -L¹-L²-L³-D, and the remaining R¹⁰ are hydrogen. In some embodiments, two R¹⁰ in Formula I-10B are -L¹-L²-L³-D, and the remaining R¹⁰ are hydrogen. In some embodiments, three R¹⁰ in Formula I-10B are -L¹-L²-L³-D, and the remaining R¹⁰ is hydrogen. In some embodiments, all four R¹⁰ in Formula I-10B are -L¹-L²-L³-D.

In some embodiments, the compound of Formula I can have a Formula I-10C:

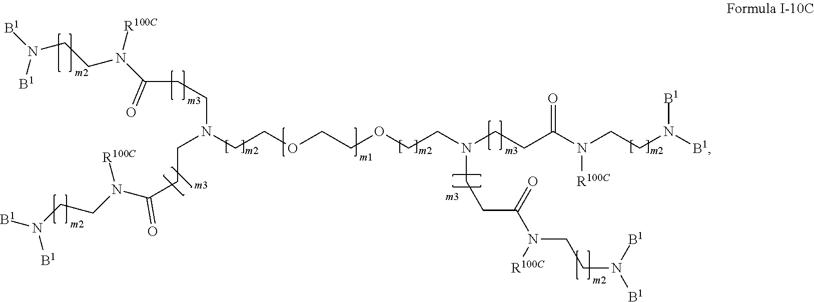

wherein:
each B¹ group is independently

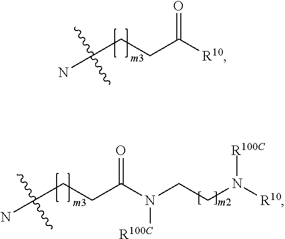

or a moiety having at least one repeating units of

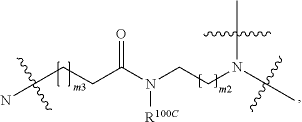

wherein the moiety terminates with a structure comprising

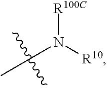

wherein
m1 is 0-100 such as 0-10 (e.g., 0, 1, 2, 3, 4, 5, 6, 7, or 8), 10-50, 50-100, etc.
m2 at each occurrence is independently an integer of 0-5 (e.g., 1, 2, or 3),
m3 at each occurrence is independently an integer of 0-5 (e.g., 0, 1, 2, or 3),
R¹⁰ at each occurrence is independently, hydrogen, —OH, alkoxy (e.g., C₁₋₄ alkoxy), NH₂, monoalkyl amine (e.g., NH(C₁₋₄ alkyl)), dialkyl amine (e.g., N(C₁₋₄ alkyl)(C₁₋₄ alkyl)), optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D, and
R^{100C} at each occurrence is independently hydrogen, optionally substituted alkyl, or optionally substituted cycloalkyl.
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.

In some embodiments, each B¹ group in Formula I-10C is

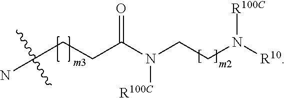

In some embodiments, R^{100C} at each occurrence in Formula I-10C (inclusive of B¹) is independently hydrogen or a C₁₋₄ alkyl (e.g., methyl, ethyl, or isopropyl). In some embodiments, R^{100C} at each occurrence in Formula I-10C (inclusive of B¹) is hydrogen. In some embodiments, m1 in Formula I-10C is 0, 1, 2, 3, 4, 5, 6, 7, 8, 9, or 10. In some embodiments, each m2 in Formula I-10C (inclusive of B¹) is 1. In some embodiments, each m2 in Formula I-10C (inclusive of B¹) is 2. In some embodiments, each m3 in Formula I-10C (inclusive of B¹) is 0, 1, or 2. In some embodiments, each m3 in Formula I-10C (inclusive of B¹) is 1. In some embodiments, each m3 in Formula I-10C (inclusive of B¹) is 2. In some embodiments, 1, 2, 3, 4, 5, 6, or 7 of R¹⁰ in Formula I-10C (inclusive of B¹) is -L¹-L²-L³-D, and the remaining R¹⁰ are (is) hydrogen. In some embodiments, all 8 of R¹⁰ in Formula I-10C are -L¹-L²-L³-D.

In some embodiments, each B¹ group in Formula I-10C is a moiety having at least one (e.g., 2, 3, 4, 5, 6, 7, or 8) repeating units of

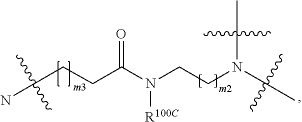

wherein the moiety terminates with a structure comprising

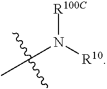

For example, a moiety having 3 repeating units and ends with

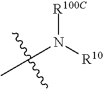

can be represented by the structure below:

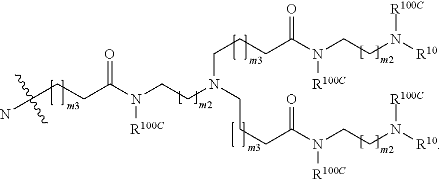

In some embodiments, R^{100C} at each occurrence in Formula I-10C (inclusive of B¹) is independently hydrogen or a C₁₋₄ alkyl (e.g., methyl, ethyl, or isopropyl). In some embodiments, R^{100C} at each occurrence in Formula I-10C (inclusive of B¹) is hydrogen. In some embodiments, m1 in Formula I-10C is 0, 1, 2, 3, 4, 5, 6, 7, 8, 9, or 10. In some embodiments, each m2 in Formula I-10C (inclusive of B¹) is 1. In some embodiments, each m2 in Formula I-10C (inclusive of B¹) is 2. In some embodiments, each m3 in Formula I-10C (inclusive of B¹) is 0, 1, or 2. In some embodiments, each m3 in Formula I-10C (inclusive of B¹) is 1. In some embodiments, each m3 in Formula I-10C (inclusive of B¹) is 2. As would be understood by those skilled in the art, when the moiety has three (3) repeating units shown above, the compound of Formula I-10C can have up to 16 R¹⁰ groups, and with seven (7) repeating units, can have up to 32 R¹⁰ groups, etc. In some embodiments, one or more of R¹⁰ in Formula I-10C (inclusive of B¹) can be -L¹-L²-L³-D, and the remaining R¹⁰ are (is) hydrogen. In some embodiments, all of R¹⁰ in Formula I-10C are -L¹-L²-L³-D.

In some embodiments, the Q in Formula I can have a Formula Q-3:

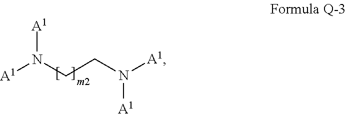

wherein each A¹ is independently F-1, F-2, or F-3,

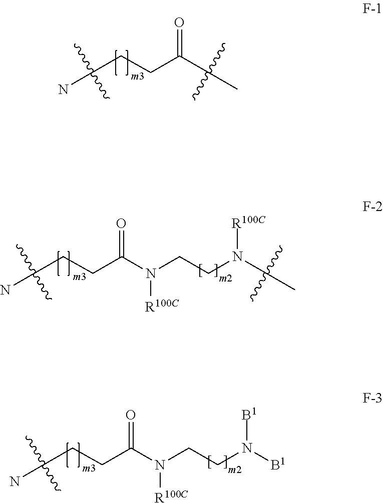

wherein each B¹ group in F-3 is independently F-1 or F-2, or a moiety having at least one repeating units of

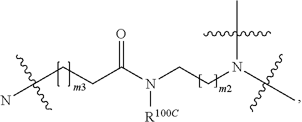

wherein the moiety terminates with a structure
comprising

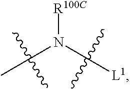

(L¹ is showing to show connectivity if L¹-L²-L³-D is bond at the terminal);
wherein:
each m2 is an integer of 0-5 (e.g., 1, 2, or 3),
each m3 is independently an integer of 0-5 (e.g., 0, 1, 2, or 3),
R^{100C} at each occurrence is independently hydrogen, optionally substituted alkyl, or optionally substituted cycloalkyl; and
wherein at least one of the A¹ forms a covalent bond with an L¹ through a terminal carbonyl group or —N—R^{100C} group.

For example, in some embodiments, the compound of Formula I can have a Formula I-11A:

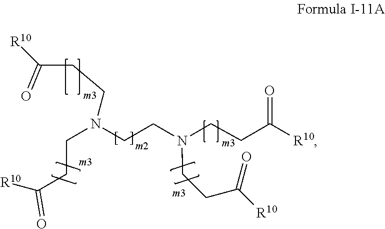

wherein:
R¹⁰ at each occurrence is independently —OH, alkoxy (e.g., C₁₋₄ alkoxy), NH₂, monoalkyl amine (e.g., NH(C₁₋₄ alkyl)), dialkyl amine (e.g., N(C₁₋₄ alkyl)(C₁₋₄ alkyl)), optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D,
L¹, L², L³, and D are defined herein,
each m2 is an integer of 0-5 (e.g., 1, 2, or 3), and
each m3 is independently an integer of 0-5 (e.g., 0, 1, 2, or 3).
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
   In some embodiments, one, two, or three R in Formula I-11A is -L¹-L²-L³-D, and the remaining R¹⁰ are (is) —OH, alkoxy (e.g., C₁₋₄ alkoxy), NH₂, monoalkyl amine (e.g., NH(C₁₋₄ alkyl)), or dialkyl amine (e.g., N(C₁₋₄ alkyl)(C₁₋₄ alkyl)). In some embodiments, all four R¹⁰ in Formula I-11A are -L¹-L²-L³-D.

In some embodiments, the compound of Formula I can have a Formula I-11B:

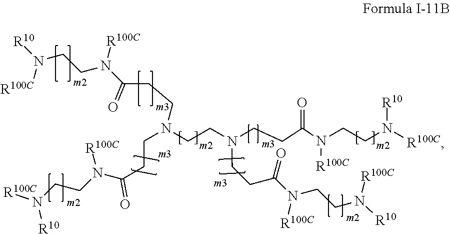

wherein:
R¹⁰ at each occurrence is independently hydrogen, optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D,
L¹, L², L³, and D are defined herein,
each m2 is an integer of 0-5 (e.g., 1, 2, or 3),
each m3 is independently an integer of 0-5 (e.g., 0, 1, 2, or 3), and
each R^{100C} is independently hydrogen, optionally substituted alkyl, or optionally substituted cycloalkyl.
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
   In some embodiments, each R^{100C} in Formula I-11B is hydrogen or a C₁₋₄ alkyl (e.g., methyl, ethyl, or isopropyl). In some embodiments, each R^{100C} in Formula I-11B is hydrogen. In some embodiments, each m2 in Formula I-11B is 1. In some embodiments, each m2 in Formula I-11B is 2. In some embodiments, each m3 in Formula I-11B is 0, 1, or 2. In some embodiments, each m3 in Formula I-11B is 1. In some embodiments, each m3 in Formula I-11B is 2. In some embodiments, one R¹⁰ in Formula I-11B is -L¹-L²-L³-D, and the remaining R¹⁰ are hydrogen. In some embodiments, two R¹⁰ in Formula I-11B are -L¹-L²-L³-D, and the remaining R¹⁰ are hydrogen. In some embodiments, three R¹⁰ in Formula I-11B are -L¹-L²-L³-D, and the remaining R¹⁰ is hydrogen. In some embodiments, all four R¹⁰ in Formula I-11B are -L¹-L²-L³-D.

In some embodiments, the compound of Formula I can have a Formula I-11C:

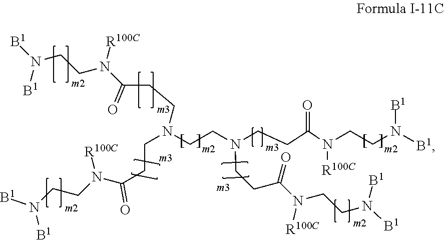

wherein:
each B¹ group is independently

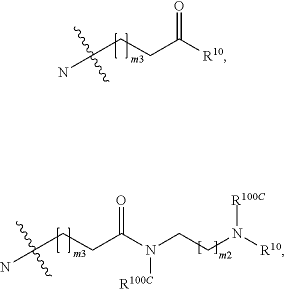

or a moiety having at least one repeating units of

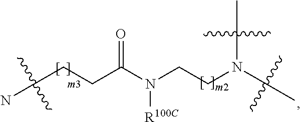

wherein the moiety terminates with a structure comprising

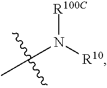

wherein
m2 at each occurrence is independently an integer of 0-5 (e.g., 1, 2, or 3),
m3 at each occurrence is independently an integer of 0-5 (e.g., 0, 1, 2, or 3),
R¹⁰ at each occurrence is independently, hydrogen, —OH, alkoxy (e.g., C₁₋₄ alkoxy), NH₂, monoalkyl amine (e.g., NH(C₁₋₄ alkyl)), dialkyl amine (e.g., N(C₁₋₄ alkyl)(C₁₋₄ alkyl)), optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D, and
R^{100C} at each occurrence is independently hydrogen, optionally substituted alkyl, or optionally substituted cycloalkyl.
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.

In some embodiments, each B¹ group in Formula I-11C is

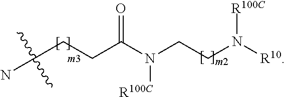

In some embodiments, R^{100C} at each occurrence in Formula I-11C (inclusive of B¹) is independently hydrogen or a C₁₋₄ alkyl (e.g., methyl, ethyl, or isopropyl). In some embodiments, R^{100C} at each occurrence in Formula I-11C (inclusive of B¹) is hydrogen. In some embodiments, each m2 in Formula I-11C (inclusive of B¹) is 1. In some embodiments, each m2 in Formula I-11C (inclusive of B¹) is 2. In some embodiments, each m3 in Formula I-11C (inclusive of B¹) is 0, 1, or 2. In some embodiments, each m3 in Formula I-11C (inclusive of B¹) is 1. In some embodiments, each m3 in Formula I-11C (inclusive of B¹) is 2. In some embodiments, 1, 2, 3, 4, 5, 6, or 7 of R¹⁰ in Formula I-11C (inclusive of B¹) is -L¹-L²-L³-D, and the remaining R¹⁰ are (is) hydrogen. In some embodiments, all 8 of R¹⁰ in Formula I-11C are -L¹-L²-L³-D.

In some embodiments, each B¹ group in Formula I-11C is a moiety having at least one (e.g., 2, 3, 4, 5, 6, 7, or 8) repeating units of

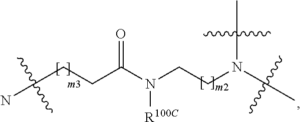

wherein the moiety terminates with a structure comprising

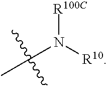

In some embodiments, R^{100C} at each occurrence in Formula I-11C (inclusive of B¹) is independently hydrogen or a C₁₋₄ alkyl (e.g., methyl, ethyl, or isopropyl). In some embodiments, R^{100C} at each occurrence in Formula I-11C (inclusive of B¹) is hydrogen. In some embodiments, each m2 in Formula I-11C (inclusive of B¹) is 1. In some embodiments, each m2 in Formula I-11C (inclusive of B¹) is 2. In some embodiments, each m3 in Formula I-11C (inclusive of B¹) is 0, 1, or 2. In some embodiments, each m3 in Formula I-11C (inclusive of B¹) is 1. In some embodiments, each m3 in Formula I-11C (inclusive of B¹) is 2. As would be understood by those skilled in the art, when the moiety has three (3) repeating units shown above, the compound of Formula I-11C can have up to 16 R¹⁰ groups, and with seven (7) repeating units, can have up to 32 R¹⁰ groups, etc. In some embodiments, one or more of R¹⁰ in Formula I-11C (inclusive of B¹) can be -L¹-L²-L³-D, and the remaining R¹⁰ are (is) hydrogen. In some embodiments, all of R¹⁰ in Formula I-11C are -L¹-L²-L³-D.

In some embodiments, the Q in Formula I can have a Formula Q-4:

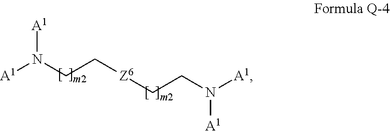

wherein:
Z⁶ is O, NR^{100D}, a polyethylene glycol (PEG) chain, optionally substituted alkylene, optionally substituted heteroalkylene, optionally substituted carbocyclylene, optionally substituted heterocyclylene, optionally substituted arylene, or optionally substituted heteroarylene,
each A¹ is independently F-1, F-2, or F-3,

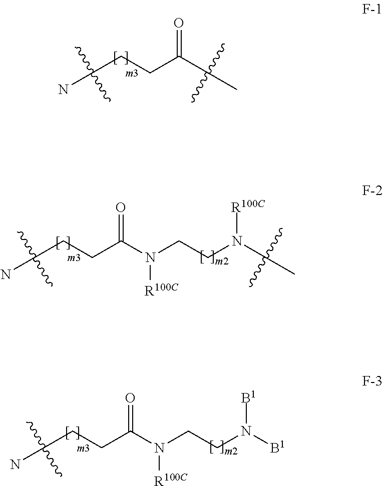

wherein each B¹ group in F-3 is independently F-1 or F-2, or a moiety having at least one repeating units of

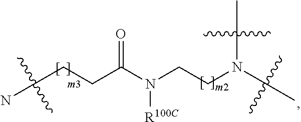

wherein the moiety terminates with a structure comprising

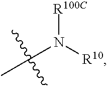

(L¹ is showing to show connectivity if L¹-L²-L³-D is bond at the terminal);
each m2 and m3 is independently an integer of 0-5 (e.g., 1, 2, or 3);
R^{100C} at each occurrence is independently hydrogen, optionally substituted alkyl, or optionally substituted cycloalkyl;
R^{100D} is hydrogen, optionally substituted alkyl, or optionally substituted cycloalkyl; and wherein at least one of the A¹ forms a covalent bond with an L¹ through a terminal carbonyl group or —N—R^{100C} group.
   In some embodiments, Z⁶ is O. In some embodiments, Z⁶ is NR^{100D}, wherein R^{100D} is hydrogen or a C₁₋₄ alkyl. In some embodiments, Z⁶ is C₁₋₁₀ alkylene, C₁₋₁₀ heteroalkylene having 1-5 heteroatoms independently selected from O and N, C₃₋₈ carbocyclylene, 3-10 membered heterocyclylene having 1-3 ring heteroatoms independently selected from O, N and S, phenylene, or 5-10 membered heteroarylene having 1-3 ring heteroatoms independently selected from O, N and S, each of which is optionally substituted with one or more (e.g., 1, 2, or 3) substituents independently selected from F, C₁₋₄ alkyl, and C₁₋₄ alkoxy. In some embodiments, Z⁶ is a PEG chain (any of those described herein).

For example, in some embodiments, the compound of Formula I can have a Formula I-12A:

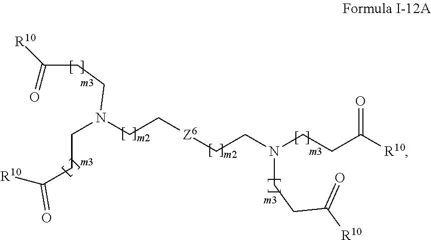

wherein:
Z⁶ is O, NR^{100D}, a PEG chain (e.g., any of those described herein), optionally substituted alkylene, optionally substituted heteroalkylene, optionally substituted carbocyclylene, optionally substituted heterocyclylene, optionally substituted arylene, or optionally substituted heteroarylene,
R¹⁰ at each occurrence is independently —OH, alkoxy (e.g., C₁₋₄ alkoxy), NH₂, monoalkyl amine (e.g., NH(C₁₋₄ alkyl)), dialkyl amine (e.g., N(C₁₋₄ alkyl)(C₁₋₄ alkyl)), optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D,
L¹, L², L³, and D are defined herein,
each m2 is an integer of 0-5 (e.g., 1, 2, or 3), and
each m3 is independently an integer of 0-5 (e.g., 0, 1, 2, or 3).
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
   In some embodiments, Z⁶ is O. In some embodiments, Z⁶ is NR^{100D}, wherein R^{100D} is hydrogen or a C₁₋₄ alkyl. In some embodiments, Z⁶ is C₁₋₁₀ alkylene, C₁₋₁₀ heteroalkylene having 1-5 heteroatoms independently selected from O and N, C₃₋₈ carbocyclylene, 3-10 membered heterocyclylene having 1-3 ring heteroatoms independently selected from O, N and S, phenylene, or 5-10 membered heteroarylene having 1-3 ring heteroatoms independently selected from O, N and S, each of which is optionally substituted with one or more (e.g., 1, 2, or 3) substituents independently selected from F, C₁₋₄ alkyl, and C₁₋₄ alkoxy. In some embodiments, Z⁶ is a PEG chain. In some embodiments, Z⁶ is a low molecular weight PEG having a number average molecular weight (Mₙ) or a weight average molecular weight (M_{w}) of about 200 to about 5000 g/mol. In some embodiments, Z⁶ is a PEG chain having a Mₙ or M_{w} of about 5000 to about 20000 g/mol. In some embodiments, Z⁶ is a PEG chain having a Mₙ or M_{w} of about 20000 to about 100,000 g/mol. In some embodiments, Z⁶ is a PEG chain having a Mₙ or M_{w} of about 100,000 to about 500,000 g/mol. In some embodiments, one, two, or three R in Formula I-12A is -L¹-L²-L³-D, and the remaining R¹⁰ are (is) —OH, alkoxy (e.g., C₁₋₄ alkoxy), NH₂, monoalkyl amine (e.g., NH(C₁₋₄ alkyl)), or dialkyl amine (e.g., N(C₁₋₄ alkyl)(C₁₋₄ alkyl)). In some embodiments, all four R¹⁰ in Formula I-12A are -L¹-L²-L³-D.

In some embodiments, the compound of Formula I can have a Formula I-12B:

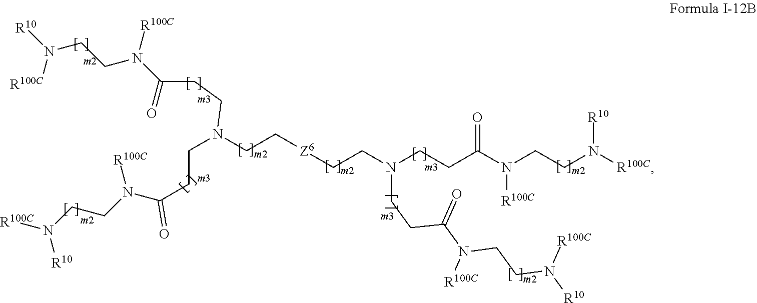

wherein:
Z⁶ is O, NR^{100D}, a PEG chain (e.g., any of those described herein), optionally substituted alkylene, optionally substituted heteroalkylene, optionally substituted carbocyclylene, optionally substituted heterocyclylene, optionally substituted arylene, or optionally substituted heteroarylene,
R¹⁰ at each occurrence is independently hydrogen, optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D,
L¹, L², L³, and D are defined herein,
each m2 is an integer of 0-5 (e.g., 1, 2, or 3),
each m3 is independently an integer of 0-5 (e.g., 0, 1, 2, or 3), and
each R^{100C} is independently hydrogen, optionally substituted alkyl, or optionally substituted cycloalkyl.
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
   In some embodiments, each R^{100C} in Formula I-12B is hydrogen or a C₁₋₄ alkyl (e.g., methyl, ethyl, or isopropyl). In some embodiments, each R^{100C} in Formula I-12B is hydrogen. In some embodiments, each m2 in Formula I-12B is 1. In some embodiments, each m2 in Formula I-12B is 2. In some embodiments, each m3 in Formula I-12B is 0, 1, or 2. In some embodiments, each m3 in Formula I-12B is 1. In some embodiments, each m3 in Formula I-12B is 2. In some embodiments, one R¹⁰ in Formula I-12B is -L¹-L²-L³-D, and the remaining R¹⁰ are hydrogen. In some embodiments, two R¹⁰ in Formula I-12B are -L¹-L²-L³-D, and the remaining R¹⁰ are hydrogen. In some embodiments, three R¹⁰ in Formula I-12B are -L¹-L²-L³-D, and the remaining R¹⁰ is hydrogen. In some embodiments, all four R¹⁰ in Formula I-12B are -L¹-L²-L³-D. In some embodiments, Z⁶ in Formula I-12B is O. In some embodiments, Z⁶ in Formula I-12B is NR^{100D} wherein R^{100D} is hydrogen or a C₁₋₄ alkyl. In some embodiments, Z⁶ in Formula I-12B is C₁₋₁₀ alkylene, C₁₋₁₀ heteroalkylene having 1-5 heteroatoms independently selected from O and N, C₃₋₈ carbocyclylene, 3-10 membered heterocyclylene having 1-3 ring heteroatoms independently selected from O, N and S, phenylene, or 5-10 membered heteroarylene having 1-3 ring heteroatoms independently selected from O, N and S, each of which is optionally substituted with one or more (e.g., 1, 2, or 3) substituents independently selected from F, C₁₋₄ alkyl, and C₁₋₄ alkoxy. In some embodiments, Z⁶ is a PEG chain. In some embodiments, Z⁶ is a low molecular weight PEG having a number average molecular weight (Mₙ) or a weight average molecular weight (M_{w}) of about 200 to about 5000 g/mol. In some embodiments, Z⁶ is a PEG chain having a Mₙ or M_{w} of about 5000 to about 20000 g/mol. In some embodiments, Z⁶ is a PEG chain having a Mₙ or M_{w} of about 20000 to about 100,000 g/mol. In some embodiments, Z⁶ is a PEG chain having a Mₙ or M_{w} of about 100,000 to about 500,000 g/mol.

In some embodiments, the compound of Formula I can have a Formula I-12C:

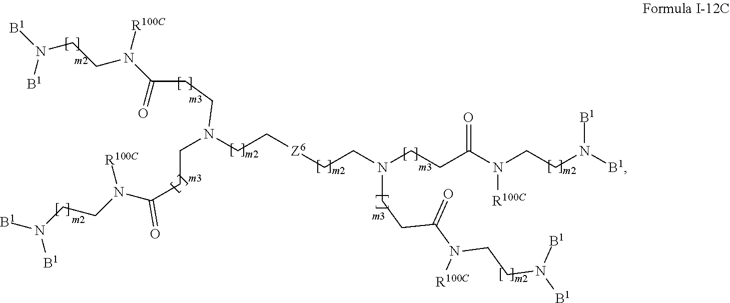

wherein:
Z⁶ is O, NR^{100D}, a PEG chain (e.g., any of those described herein), optionally substituted alkylene, optionally substituted heteroalkylene, optionally substituted carbocyclylene, optionally substituted heterocyclylene, optionally substituted arylene, or optionally substituted heteroarylene,
each B¹ group is independently

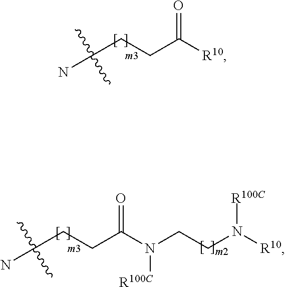

or a moiety having at least one repeating units of

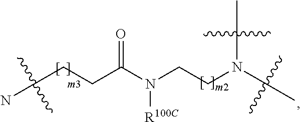

wherein the moiety terminates with a structure comprising

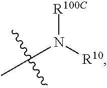

wherein
m2 at each occurrence is independently an integer of 0-5 (e.g., 1, 2, or 3),
m3 at each occurrence is independently an integer of 0-5 (e.g., 0, 1, 2, or 3),
R¹⁰ at each occurrence is independently, hydrogen, —OH, alkoxy (e.g., C₁₋₄ alkoxy), NH₂, monoalkyl amine (e.g., NH(C₁₋₄ alkyl)), dialkyl amine (e.g., N(C₁₋₄ alkyl)(C₁₋₄ alkyl)), optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D, and
R^{100C} at each occurrence is independently hydrogen, optionally substituted alkyl, or optionally substituted cycloalkyl.
   In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
   In some embodiments, Z⁶ in Formula I-12C is O. In some embodiments, Z⁶ in Formula I-12C is NR^{100D}, wherein R^{100D} is hydrogen or a C₁₋₄ alkyl. In some embodiments, Z in Formula I-12C is C₁₋₁₀ alkylene, C₁₋₁₀ heteroalkylene having 1-5 heteroatoms independently selected from O and N, C₃₋₈ carbocyclylene, 3-10 membered heterocyclylene having 1-3 ring heteroatoms independently selected from O, N and S, phenylene, or 5-10 membered heteroarylene having 1-3 ring heteroatoms independently selected from O, N and S, each of which is optionally substituted with one or more (e.g., 1, 2, or 3) substituents independently selected from F, C₁₋₄ alkyl, and C₁₋₄ alkoxy. In some embodiments, Z⁶ is a PEG chain. In some embodiments, Z⁶ is a low molecular weight PEG having a number average molecular weight (Mₙ) or a weight average molecular weight (M_{w}) of about 200 to about 5000 g/mol. In some embodiments, Z⁶ is a PEG chain having a Mₙ or M_{w} of about 5000 to about 20000 g/mol. In some embodiments, Z⁶ is a PEG chain having a Mₙ or M_{w} of about 20000 to about 100,000 g/mol. In some embodiments, Z⁶ is a PEG chain having a Mₙ or M_{w} of about 100,000 to about 500,000 g/mol.

In some embodiments, each B¹ group in Formula I-12C is

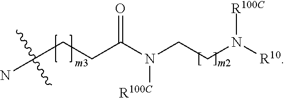

In some embodiments, R^{100C} at each occurrence in Formula I-12C (inclusive of B¹) is independently hydrogen or a C₁₋₄ alkyl (e.g., methyl, ethyl, or isopropyl). In some embodiments, R^{100C} at each occurrence in Formula I-12C (inclusive of B¹) is hydrogen. In some embodiments, each m2 in Formula I-12C (inclusive of B¹) is 1. In some embodiments, each m2 in Formula I-12C (inclusive of B¹) is 2. In some embodiments, each m3 in Formula I-12C (inclusive of B¹) is 0, 1, or 2. In some embodiments, each m3 in Formula I-12C (inclusive of B¹) is 1. In some embodiments, each m3 in Formula I-12C (inclusive of B¹) is 2. In some embodiments, 1, 2, 3, 4, 5, 6, or 7 of R¹⁰ in Formula I-12C (inclusive of B¹) is -L¹-L²-L³-D, and the remaining R¹⁰ are (is) hydrogen. In some embodiments, all 8 of R¹⁰ in Formula I-12C are -L¹-L²-L³-D.

In some embodiments, each B¹ group in Formula I-12C is a moiety having at least one (e.g., 2, 3, 4, 5, 6, 7, or 8) repeating units of

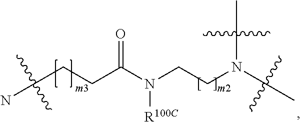

wherein the moiety terminates with a structure comprising

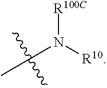

In some embodiments, R^{100C} at each occurrence in Formula I-12C (inclusive of B¹) is independently hydrogen or a C₁₋₄ alkyl (e.g., methyl, ethyl, or isopropyl). In some embodiments, R^{100C} at each occurrence in Formula I-12C (inclusive of B¹) is hydrogen. In some embodiments, each m2 in Formula I-12C (inclusive of B¹) is 1. In some embodiments, each m2 in Formula I-12C (inclusive of B¹) is 2. In some embodiments, each m3 in Formula I-12C (inclusive of B¹) is 0, 1, or 2. In some embodiments, each m3 in Formula I-12C (inclusive of B¹) is 1. In some embodiments, each m3 in Formula I-12C (inclusive of B¹) is 2. As would be understood by those skilled in the art, when the moiety has three (3) repeating units shown above, the compound of Formula I-10C can have up to 16 R¹⁰ groups, and with seven (7) repeating units, can have up to 32 R¹⁰ groups, etc. In some embodiments, one or more of R¹⁰ in Formula I-12C (inclusive of B¹) can be -L¹-L²-L³-D, and the remaining R¹⁰ are (is) hydrogen. In some embodiments, all of R¹⁰ in Formula I-12C are -L¹-L²-L³-D.

In some embodiments, the Q in Formula I can have a Formula Q-5A:

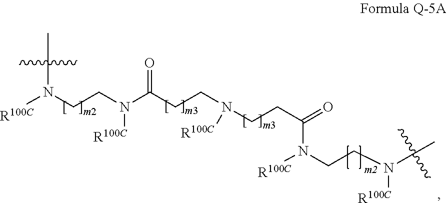

wherein: each m2 is independently an integer of 0-10 (e.g., 1, 2, 3, 4, or 5), and each m3 is independently an integer of 0-10 (e.g., 0, 1, or 2), and each R^{100C} is independently hydrogen, optionally substituted alkyl, or optionally substituted cycloalkyl,
wherein at least one of the terminal NR^{100C} of Formula Q-5A forms a covalent bond with an L¹.

For example, in some embodiments, the compound of Formula I can have a Formula I-13A or I-13B:

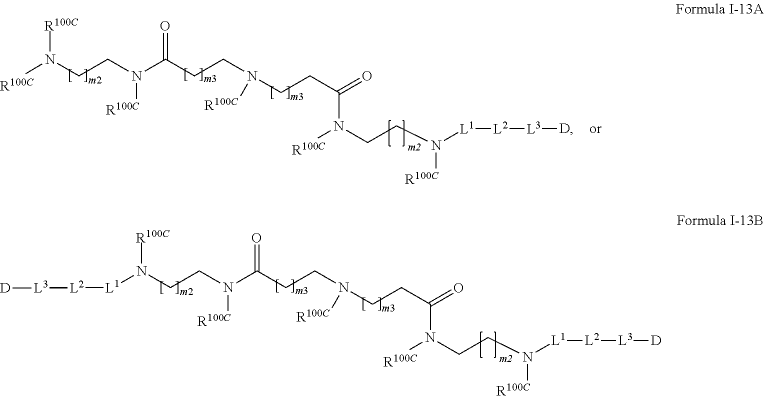

wherein
L¹, L², L³, and D are defined herein;
each m2 is independently 0-5 (e.g., 1, 2, or 3);
each m3 is independently 0-5 (e.g., 0, 1, 2, or 3); and
each R^{100C} is independently hydrogen, optionally substituted alkyl, or optionally substituted cycloalkyl.
In some embodiments, each m2 in Formula I-13A or I-13B is 1. In some embodiments, each m2 in Formula I-13A or I-13B is 2. In some embodiments, each m3 in Formula I-13A or I-13B is 0. In some embodiments, each m2 in Formula I-13A or I-13B is 1. In some embodiments, each m2 in Formula I-13A or I-13B is 2. In some embodiments, each R^{100C} in Formula I-13A or I-13B is independently hydrogen or a C₁₋₄ alkyl (e.g., methyl, ethyl or isopropyl). In some embodiments, each R^{100C} in Formula I-13B is hydrogen.

In some embodiments, the Q in Formula I can have a Formula Q-5B:

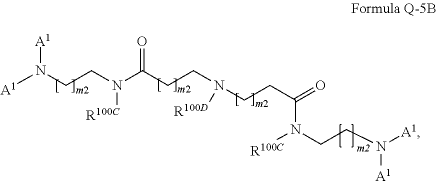

wherein each A¹ is independently F-1, F-2, or F-3,

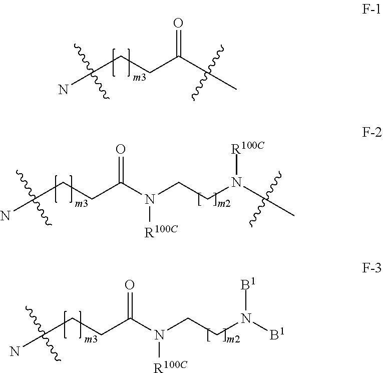

wherein each B¹ group in F-3 is independently F-1 or F-2, or a moiety having at least one repeating units of

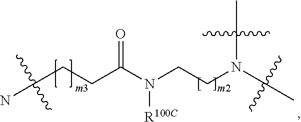

wherein the moiety terminates with a structure comprising

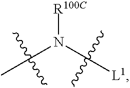

(L¹ is showing to show connectivity if L¹-L²-L³-D is bond at the terminal);
each m2 is independently an integer of 0-5 (e.g., 1, 2, or 3);
each m3 is independently an integer of 0-5 (e.g., 0, 1, 2, or 3);
R^{100C} at each occurrence is independently hydrogen, optionally substituted alkyl, or optionally substituted cycloalkyl;
R^{100D} is hydrogen, optionally substituted alkyl, or optionally substituted cycloalkyl; and wherein at least one of the A¹ forms a covalent bond with an L¹ through a terminal carbonyl group or —N—R^{100C} group.

For example, in some embodiments, the compound of Formula I can have a Formula I-14A:

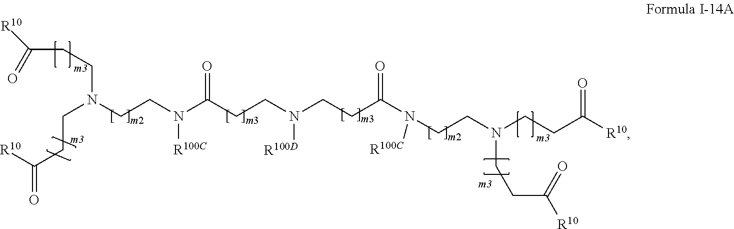

wherein:
R¹⁰ at each occurrence is independently —OH, alkoxy (e.g., C₁₋₄ alkoxy), NH₂, monoalkyl amine (e.g., NH(C₁₋₄ alkyl)), dialkyl amine (e.g., N(C₁₋₄ alkyl)(C₁₋₄ alkyl)), optionally substituted alkyl, optionally substituted cycloalkyl, optionally substituted heteroalkyl, optionally substituted heterocyclyl or -L¹-La-L³-D, provided that at least one of the R¹⁰ comprises -L¹-L²-L³-D,
L¹, L², L³, and D are defined herein,
each m2 is an integer of 0-5 (e.g., 1, 2, or 3),
each m3 is independently an integer of 0-5 (e.g., 0, 1, 2, or 3),
R^{100C} at each occurrence is independently hydrogen, optionally substituted alkyl, or optionally substituted cycloalkyl, and
R^{100D} is hydrogen, optionally substituted alkyl, or optionally substituted cycloalkyl.
In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
In some embodiments, each R^{100C} in Formula I-14A is hydrogen or a C₁₋₄ alkyl (e.g., methyl, ethyl, or isopropyl). In some embodiments, each R^{100C} in Formula I-14A is hydrogen. In some embodiments, R^{100D} in Formula I-14A is hydrogen or a C₁₋₄ alkyl (e.g., methyl, ethyl, or isopropyl). In some embodiments, each m2 in Formula I-14A is 1. In some embodiments, each m2 in Formula I-14A is 2. In some embodiments, each m3 in Formula I-14A is 0, 1, or 2. In some embodiments, each m3 in Formula I-14A is 1. In some embodiments, one, two, or three R¹⁰ in Formula I-14A is -L¹-L²-L³-D, and the remaining R¹⁰ are (is) —OH, alkoxy (e.g., C₁₋₄ alkoxy), NH₂, monoalkyl amine (e.g., NH(C₁₋₄ alkyl)), or dialkyl amine (e.g., N(C₁₋₄ alkyl)(C₁₋₄ alkyl)). In some embodiments, all four R¹⁰ in Formula I-14A are -L¹-L²-L³-D.

In some embodiments, the compound of Formula I can have a Formula I-14B:

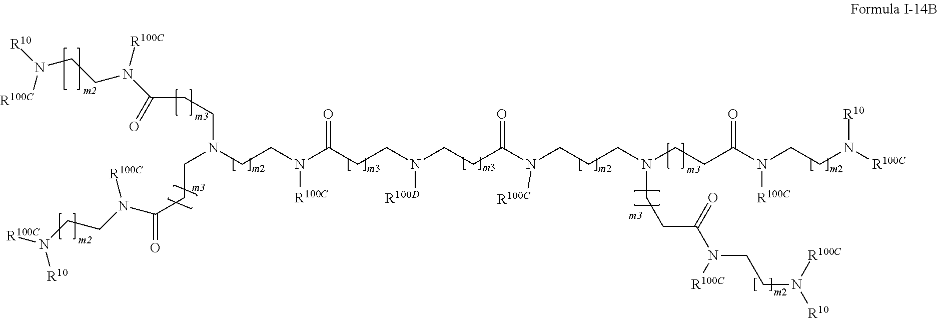

wherein:
R¹⁰ at each occurrence is independently hydrogen, optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D,
L¹, L², L³, and D are defined herein,
each m2 is an integer of 0-5 (e.g., 1, 2, or 3),
each m3 is independently an integer of 0-5 (e.g., 0, 1, 2, or 3),
each R^{100C} is independently hydrogen, optionally substituted alkyl, or optionally substituted cycloalkyl, and
R^{100D} is independently hydrogen, optionally substituted alkyl, or optionally substituted cycloalkyl.
In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
In some embodiments, each R^{100C} in Formula I-14B is hydrogen or a C₁₋₄ alkyl (e.g., methyl, ethyl, or isopropyl). In some embodiments, each R^{100C} in Formula I-14B is hydrogen. In some embodiments, R^{100D} in Formula I-14B is hydrogen or a C₁₋₄ alkyl (e.g., methyl, ethyl, or isopropyl). In some embodiments, each m2 in Formula I-14B is 1. In some embodiments, each m2 in Formula I-14B is 2. In some embodiments, each m3 in Formula I-14B is 0, 1, or 2. In some embodiments, each m3 in Formula I-14B is 1. In some embodiments, each m3 in Formula I-14B is 2. In some embodiments, one R¹⁰ in Formula I-14B is -L¹-L²-L³-D, and the remaining R¹⁰ are hydrogen. In some embodiments, two R¹⁰ in Formula I-14B are -L¹-L²-L³-D, and the remaining R¹⁰ are hydrogen. In some embodiments, three R¹⁰ in Formula I-14B are -L¹-L²-L³-D, and the remaining R¹⁰ is hydrogen. In some embodiments, all four R¹⁰ in Formula I-14B are -L¹-L²-L³-D.

In some embodiments, the compound of Formula I can have a Formula I-14C:

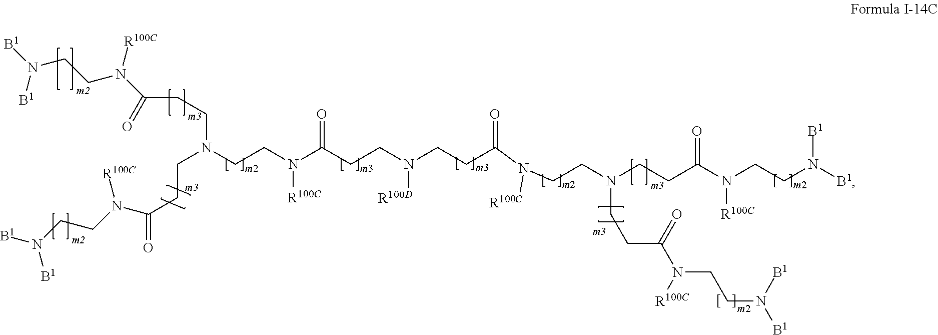

wherein:
each B¹ group is independently

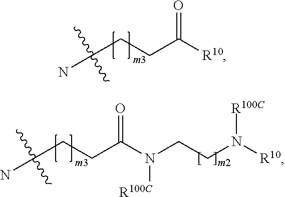

or a moiety having at least one repeating units of

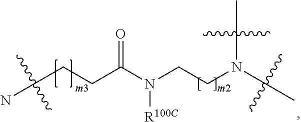

wherein the moiety terminates with a structure comprising

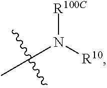

wherein
m2 at each occurrence is independently an integer of 0-5 (e.g., 1, 2, or 3),
m3 at each occurrence is independently an integer of 0-5 (e.g., 0, 1, 2, or 3),
R¹⁰ at each occurrence is independently, hydrogen, —OH, alkoxy (e.g., C₁₋₄ alkoxy), NH₂, monoalkyl amine (e.g., NH(C₁₋₄ alkyl)), dialkyl amine (e.g., N(C₁₋₄ alkyl)(C₁₋₄ alkyl)), optionally substituted alkyl, optionally substituted cycloalkyl, optionally substituted heteroalkyl, optionally substituted heterocyclyl or -L¹-L²-L³-D, provided that at least one of the R¹⁰ comprises -L¹-L²-L³-D,
R^{100C} at each occurrence is independently hydrogen, optionally substituted alkyl, or optionally substituted cycloalkyl, and
R^{100D} is hydrogen, optionally substituted alkyl, or optionally substituted cycloalkyl. In some embodiments, the optionally substituted alkyl, optionally substituted cycloalkyl, optionally substituted heteroalkyl, or optionally substituted heterocyclyl is independently optionally substituted with one or more (e.g., 1, 2, or 3) substituents independently selected from OH, amine, halogen, C₁₋₆ alkoxy, C₁₋₆ heteroalkyl, -L¹-L²-L³-D, —O-L¹-L²-L³-D, —N(H)-L¹-L²-L³-D, or —N(-L¹-L²-L³-D)₂.
In some embodiments, each R^{100C} in Formula I-14C is hydrogen or a C₁₋₄ alkyl (e.g., methyl, ethyl, or isopropyl). In some embodiments, each R^{100C} in Formula I-14C is hydrogen. In some embodiments, ROOD in Formula I-14C is hydrogen or a C₁₋₄ alkyl (e.g., methyl, ethyl, or isopropyl). In some embodiments, each m2 in Formula I-14C is 1. In some embodiments, each m2 in Formula I-14C is 2. In some embodiments, each m3 in Formula I-14C is 0, 1, or 2. In some embodiments, each m3 in Formula I-14C is 1. In some embodiments, each m3 in Formula I-14C is 2.

In some embodiments, each B¹ group in Formula I-14C is

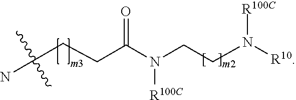

In some embodiments, R^{100C} at each occurrence in Formula I-14C (inclusive of B¹) is independently hydrogen or a C₁₋₄ alkyl (e.g., methyl, ethyl, or isopropyl). In some embodiments, R^{100C} at each occurrence in Formula I-14C (inclusive of B¹) is hydrogen. In some embodiments, R^{100D} in Formula I-14C is hydrogen or a C₁₋₄ alkyl (e.g., methyl, ethyl, or isopropyl). In some embodiments, each m2 in Formula I-14C (inclusive of B¹) is 1. In some embodiments, each m2 in Formula I-14C (inclusive of B¹) is 2. In some embodiments, each m3 in Formula I-14C (inclusive of B¹) is 0, 1, or 2. In some embodiments, each m3 in Formula I-14C (inclusive of B¹) is 1. In some embodiments, each m3 in Formula I-14C (inclusive of B¹) is 2. In some embodiments, 1, 2, 3, 4, 5, 6, or 7 of R¹⁰ in Formula I-14C (inclusive of B¹) is -L¹-L²-L³-D, and the remaining R¹⁰ are (is) hydrogen. In some embodiments, all 8 of R¹⁰ in Formula I-14C are -L¹-L²-L³-D.

In some embodiments, each B¹ group in Formula I-14C is a moiety having at least one (e.g., 2, 3, 4, 5, 6, 7, or 8) repeating units of

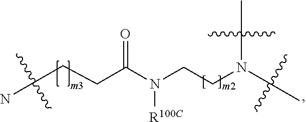

wherein the moiety terminates with a structure comprising

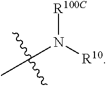

In some embodiments, R^{100C} at each occurrence in Formula I-14C (inclusive of B¹) is independently hydrogen or a C₁₋₄ alkyl (e.g., methyl, ethyl, or isopropyl). In some embodiments, R^{100C} at each occurrence in Formula I-14C (inclusive of B¹) is hydrogen. In some embodiments, R^{100D} in Formula I-14C is hydrogen or a C₁₋₄ alkyl (e.g., methyl, ethyl, or isopropyl). In some embodiments, each m2 in Formula I-14C (inclusive of B¹) is 1. In some embodiments, each m2 in Formula I-14C (inclusive of B¹) is 2. In some embodiments, each m3 in Formula I-14C (inclusive of B¹) is 0, 1, or 2. In some embodiments, each m3 in Formula I-14C (inclusive of B¹) is 1. In some embodiments, each m3 in Formula I-14C (inclusive of B¹) is 2. As would be understood by those skilled in the art, when the moiety has three (3) repeating units shown above, the compound of Formula I-14C can have up to 16 R¹⁰ groups, and with seven (7) repeating units, can have up to 32 R¹⁰ groups, etc. In some embodiments, one or more of R¹⁰ in Formula I-14C (inclusive of B¹) can be -L¹-L²-L³-D, and the remaining R¹⁰ are (is) hydrogen. In some embodiments, all of R¹⁰ in Formula I-14C are -L¹-L²-L³-D.

The linkage of Q to D in Formula I can be typically categorized in three parts, L¹, L², and L³. It should be understood that this categorization is for the ease of discussion. It should be understood that the precursors prior to conjugation with Q containing the residue of -L¹-L²-L³-D, -L²-L³-D, or -L³-D are typically also an agonist of GPR40. Such precursors are also novel compositions of the present disclosure.

In some embodiments, L¹ in Formula I (e.g., any of the subformula I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3) can have a structure of Formula L-1,

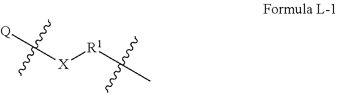

wherein: X is a bond, —CR¹⁰³R¹⁰⁴—, N(R¹⁰⁰)—, —O—, —C(═O)—, —C(═O)—N(R¹⁰⁰)—, —SO₂—, or —C(═O)—O—; and R¹ is a C₁₀₋₅₀ alkyl (e.g., C₁₀₋₃₀ alkyl) or C₁₀₋₅₀ alkenyl (e.g., C₁₀₋₃₀ alkenyl). The identity of X can depend on the respective attaching points at Q. For example, in some embodiments, Q forms a covalent bond with X through a —C(═O)— group, with the carbon of the carbonyl group of Q being the attaching point, then X is typically N(R¹⁰⁰)— or —O— such that an amide or ester bond is formed, wherein R¹⁰⁰ can be for example hydrogen or a C₁₋₄ alkyl. In some embodiments, Q forms a covalent bond with X through a —NR¹⁰⁰— group, with the N atom being the attaching point, then X can be —C(═O)—, —C(═O)—N(R¹⁰⁰)—, —SO₂— or —C(═O)—O—, preferably, X is —C(═O)—, such that an amide, urea, sulfonamide, or carbamate bond is formed, wherein R¹⁰⁰ at each occurrence can be for example hydrogen or a C₁₋₄ alkyl. In some embodiments, Q forms a covalent bond with X through an —NR¹⁰⁰— group, with the N atom being the attaching point, X can also be a bond or —CR¹⁰³R¹⁰⁴—, wherein R¹⁰⁰, R¹⁰³ and R¹⁰⁴ at each occurrence can be independently for example hydrogen or a C₁₋₄ alkyl. In some embodiments, Q forms a covalent bond with X through an oxygen atom, i.e., the oxygen atom is the attaching point, then X can be a bond, —CR¹⁰³R¹⁰⁴—, —C(═O)—, —C(═O)—N(R¹⁰⁰)—, —SO₂— or —C(═O)—O—, preferably, X is —C(═O)—, such that an ether, ester, carbamate, sulfonate, or carbonate bond is formed, wherein R¹⁰⁰, R¹⁰³ and R¹⁰⁴ at each occurrence can be independently for example hydrogen or a C₁₋₄ alkyl. In some embodiments, L¹ can be selected from the following (L² and the attaching point/group of Q (the left end) are included to show connectivity):

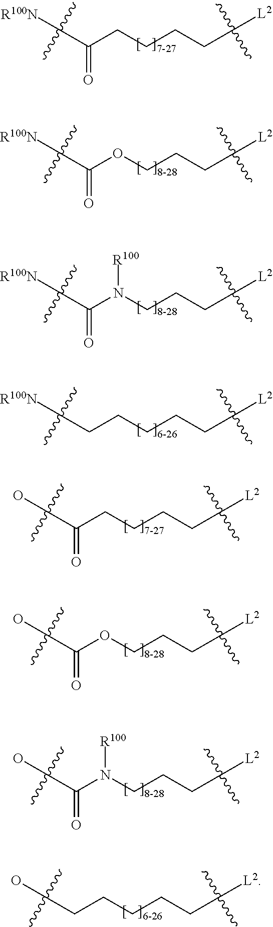

In some embodiments, L² in Formula I (e.g., any of the subformula I-1, I-2, I-3, I-4, I-5, I-6, I-7, I-8, I-8-A, I-8-B, I-8-C, I-8-D, I-9, I-10A, I-10B, I-10C, I-11A, I-11B, I-11C, I-12A, I-12B, I-12C, I-13A, I-13B, I-14A, I-14B, I-14C, I-S-1, I-S-2, or I-S-3) at each occurrence can be independently —N(R¹⁰⁰)—, —O—, or a moiety selected from:

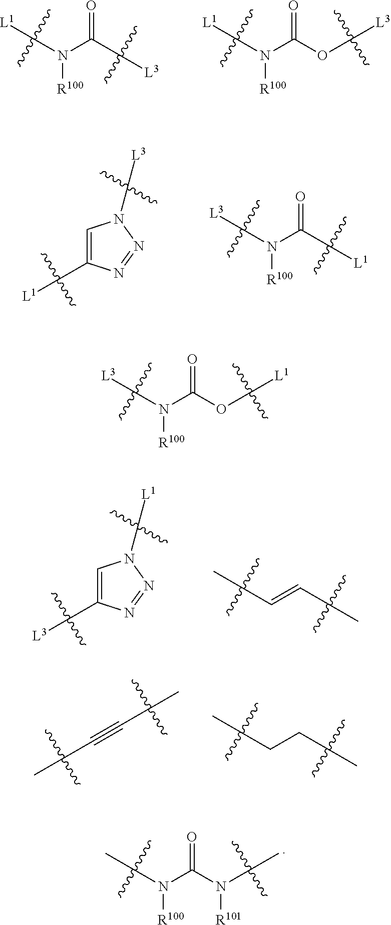

In some embodiments, R¹⁰⁰ and R¹⁰¹ is independently hydrogen or a C₁₋₄ alkyl. In some embodiments, L² at each occurrence can be independently

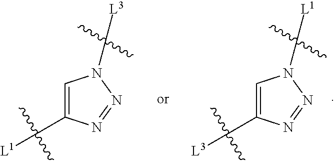

In some embodiments, L³ in Formula I (e.g., any of the subformula I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3) at each occurrence can be independently a bond, optionally substituted C₁₋₁₀ alkylene, or optionally substituted C₁₋₁₀ heteroalkylene having 1-5 heteroatoms independently selected from O and N. In some embodiments, L³ at each occurrence can be independently selected from a bond, or a moiety selected from:

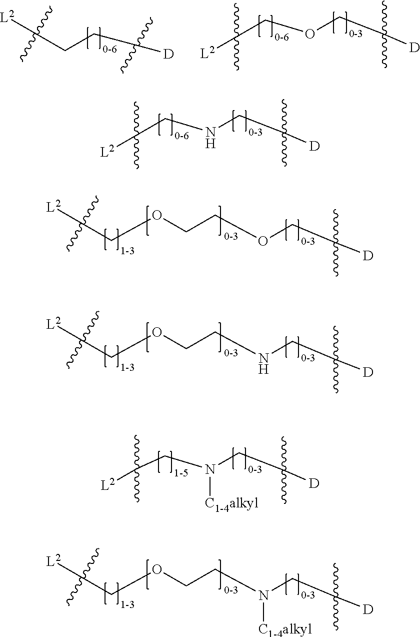

Useful GPR40 agonists for the compound of Formula I (e.g., any of the subformula I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3) are not particularly limited. For example, GPR40 agonists that can be used to link to Q via L¹-L²-L³ include those described in U.S. Pat. Nos. 7,442,808, 7,456,218, 7,465,804, 7,517,910, 7,553,867, 7,572,934, 7,582,803, 7,585,880, 7,649,110, 7,687,526, 7,714,008, 7,759,493, 7,786,165, 7,820,837, 8,030,354, 8,039,484, 8,153,694, 8,399,507, 8,450,522, 8,575,166, 8,748,462, 9,181,186, 9,278,965, 9,382,188, 9,527,875, 9,776,962, 9,834,563, 9,840,512, 9,932,311, 10,000,454, 10,059,667, 10,100,042, 10,131,651, and U.S. Published Application No. 20190367495, the content of each of which is herein incorporated by reference.

In some embodiments, D at each occurrence is independently a residue of a GPR40 full agonist.

In some embodiments, D at each occurrence is independently selected from:

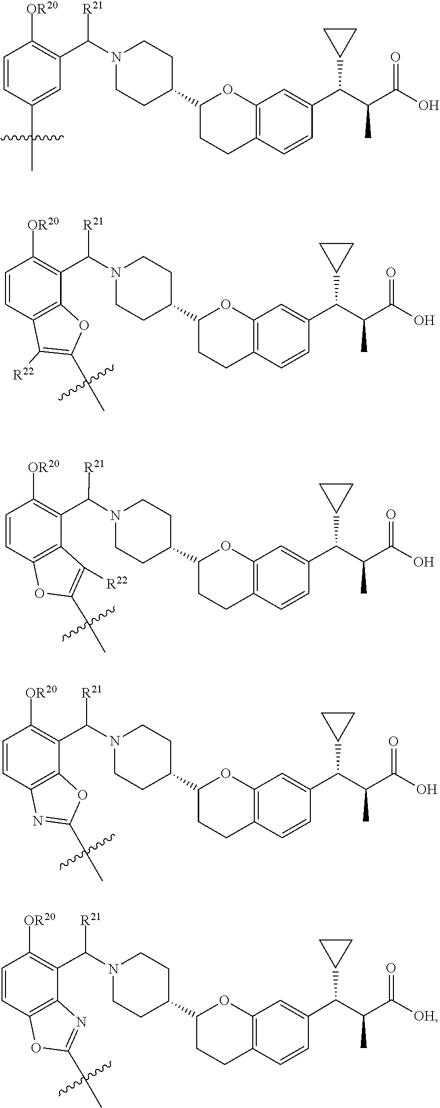

wherein: R²⁰ is C₁₋₆ alkyl or fluorine substituted C₁₋₆ alkyl; R²¹ is hydrogen or a C₁₋₆ alkyl; and R²² is hydrogen, halogen, CN, C₁₋₆ alkyl or fluorine substituted C₁₋₆ alkyl or a C₃₋₆ cycloalkyl. In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

In some embodiments, D can be a residue of a GPR40 agonist selected from:

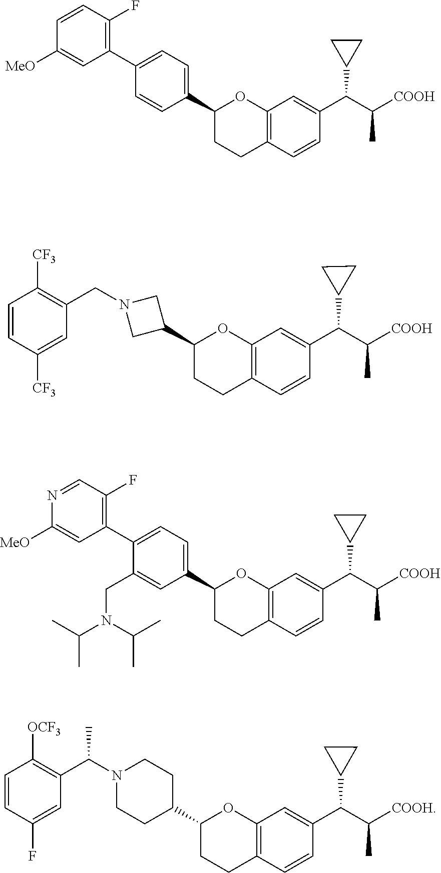 [SMILES: COc1ccc(F)c(-c2ccc([C@@H]3CCc4ccc([C@H](C5CC5)[C@H](C)C(=O)O)cc4O3)cc2)c1 ; C[C@H](C(=O)O)[C@H](c1ccc2c(c1)O[C@H](C1CN(Cc3cc(C(F)(F)F)ccc3C(F)(F)F)C1)CC2)C1CC1 ; COc1cc(-c2ccc([C@@H]3CCc4ccc([C@H](C5CC5)[C@H](C)C(=O)O)cc4O3)cc2CN(C(C)C)C(C)C)c(F)cn1 ; C[C@H](C(=O)O)[C@H](c1ccc2c(c1)O[C@@H](C1CCN([C@@H](C)c3cc(F)ccc3OC(F)(F)F)CC1)CC2)C1CC1]

In some embodiments, D can be a residue of:

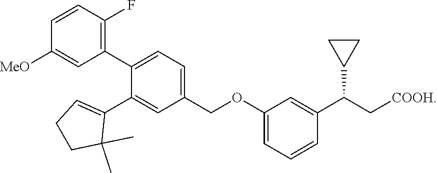 [SMILES: COc1ccc(F)c(-c2ccc(COc3cccc([C@@H](CC(=O)O)C4CC4)c3)cc2C2=CCCC2(C)C)c1]

In some embodiments, D can be selected from:

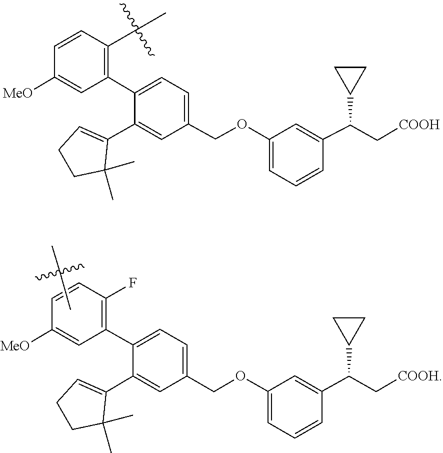

In some embodiments, D can be selected from:

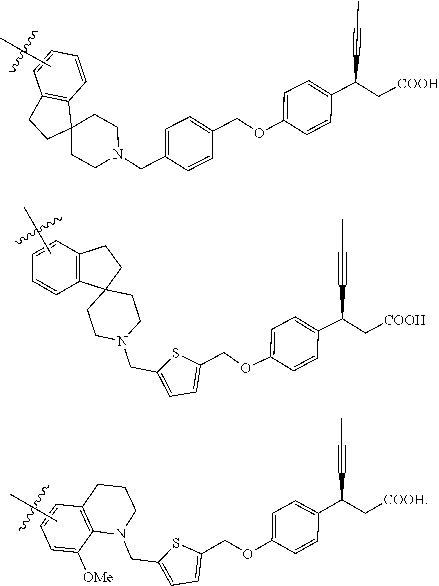

In some embodiments, D can be selected from:

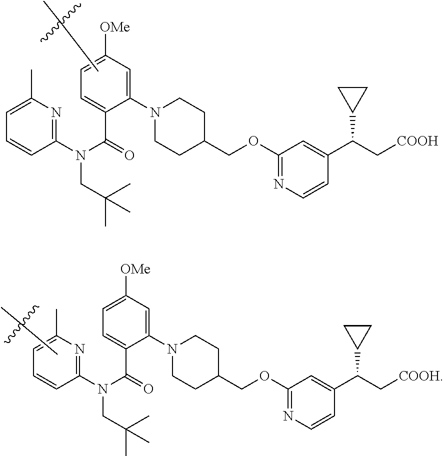

The integer “n” in Formula I (e.g., any of the subformula I-1, I-2, I-3, I-4, I-5, I-6, I-7, I-8, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3) is typically 1-64, for example, 2, 3, 4, 5, 6, 7, 8, or more, such as 1-64, e.g., 1-4, 2-8, 4-16, etc. When “n” is greater than 1, each unit of L¹-L²-L³-D is typically the same. The integer “n” in Formula I typically depends on the number of available attaching points in Q. As discussed hereinabove, in some embodiments, Q can be a residue of a dendrimer which can have multiple numbers of attaching points depending on the generation of the dendrimer. For example, a typical G-0 to G-4 poly (amide amine) dendrimer can have 4, 8, 16, 32, 64, end groups (e.g., primary amine, carboxylic acid, etc.) suitable for forming covalent bonds with L¹-L²-L³-D. In some embodiments, “n” is 1-4. In some embodiments, “n” is 2-8. In some embodiments, “n” is 4-16.

In some embodiments, each unit of L¹-L²-L³-D in Formula I (e.g., any of the subformula I-1, I-2, I-3, I-4, I-5, I-6, I-7, I-8, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3) can be the same. For example, in some embodiments, when the attaching point in Q is NR¹⁰⁰, each unit of L¹-L²-L³-D can for example, be selected from:

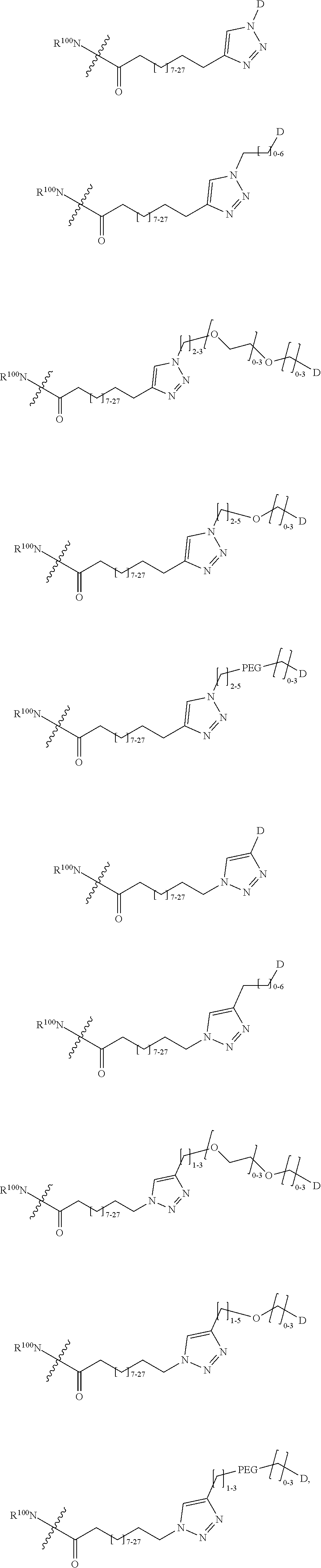

wherein D is defined herein, and PEG can be any of the PEG chain described herein. It should be understood that when two or more ranges are recited in the formulae herein, each range includes any of the integers or subranges within the range, and each of the integers or subranges of one range can be combined with each of the integers or subranges of another range. For example, the range “7-27” shown in the formulae can be any individual integer within 7-27, i.e., 7, 8, 9, 10, 11, 12, 13, 14, 15, 16, 17, 18, 19, 20, 21, 22, 23, 24, 25, 26, or 27, the range “0-3” can be 0, 1, 2, or 3, etc. When the formula include both of the ranges “7-27” and “0-3”, the formula can have each and every combination of any of the individual integer within 7-27 with the individual value of 0, 1, 2, or 3. Other formulae with such range(s) should be understood similarly. In some embodiments, D is selected from:

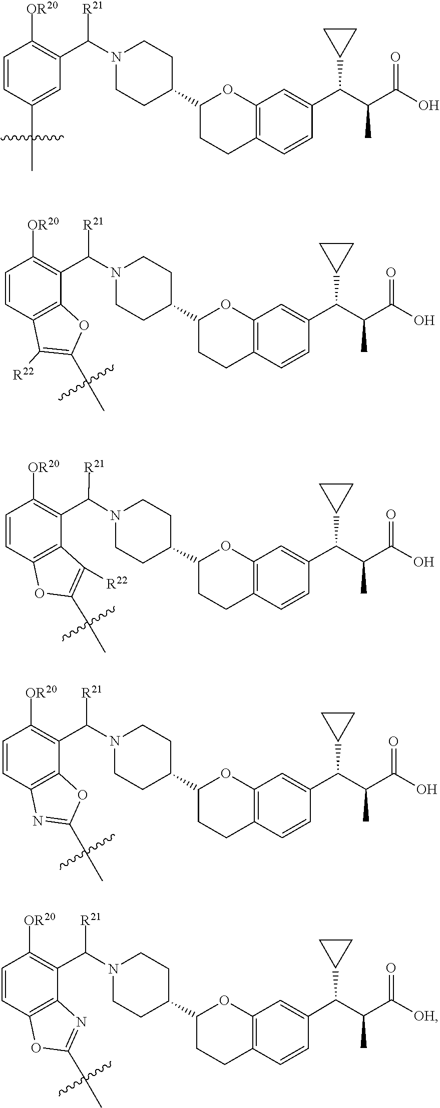

wherein: R²⁰ is C₁₋₆ alkyl or fluorine substituted C₁₋₆ alkyl; R²¹ is hydrogen or a C₁₋₆ alkyl; and R²² is hydrogen, halogen, CN, C₁₋₆ alkyl or fluorine substituted C₁₋₆ alkyl or a C₃₋₆ cycloalkyl. In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

In some embodiments, when the attaching point in Q is O, e.g., in any of the subformula I-1, I-2, I-3, I-4, I-5, I-6, I-7, I-8, I-8-A, I-8-B, I-8-C, I-8-D, each unit of L¹-L²-L³-D can be the same and selected from:

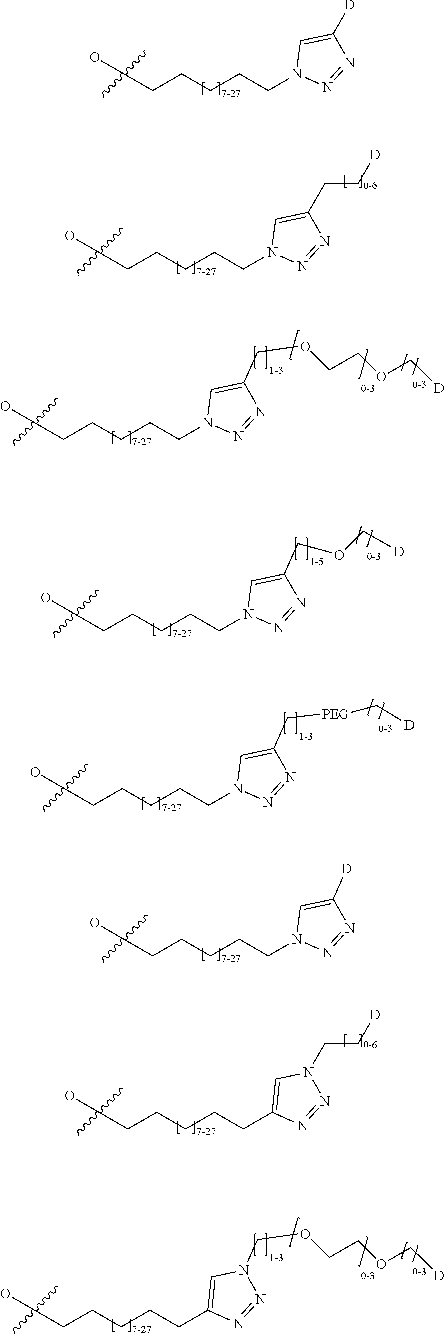

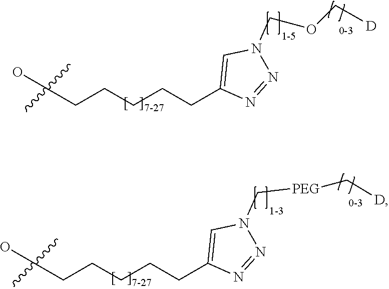

wherein D is defined herein, and PEG can be any of the PEG chain described herein. For example, in some embodiments, D is selected from:

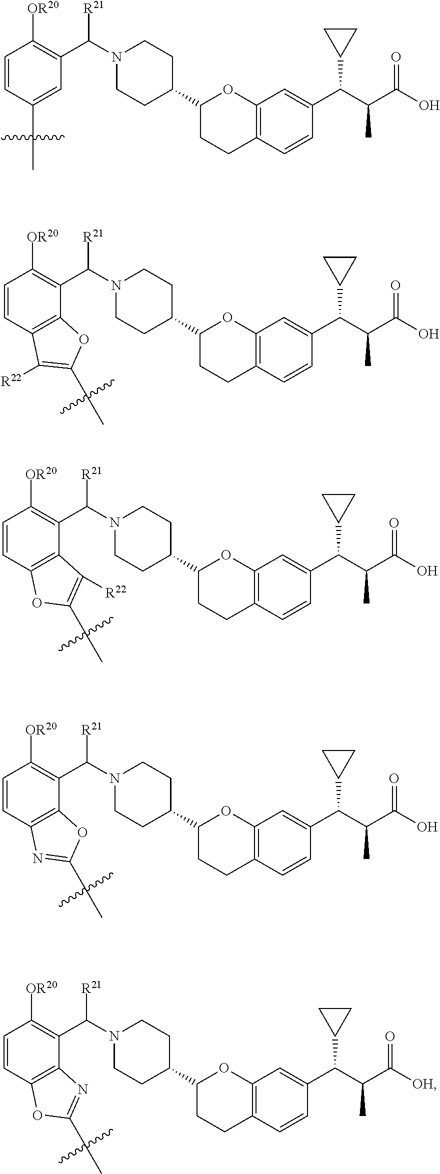

wherein: R²⁰ is C₁₋₆ alkyl or fluorine substituted C₁₋₆ alkyl; R²¹ is hydrogen or a C₁₋₆ alkyl; and R²² is hydrogen, halogen, CN, C₁₋₆ alkyl or fluorine substituted C₁₋₆ alkyl or a C₃₋₆ cycloalkyl. In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

In some embodiments, the compound of Formula I can have a Formula I-S-1, I-S-2, or I-S-3, in which each of the R¹⁰ is hydrogen or -L¹-L²-L³-D, typically the O-L¹ bond is an ether bond. The stereochemistry of the sugar alcohol or saccharide in Formula I-S-1, I-S-2, or I-S-3 can include any of those known. For example, in some embodiments, the sugar alcohol in Formula I-S-1 can be based on inositol.

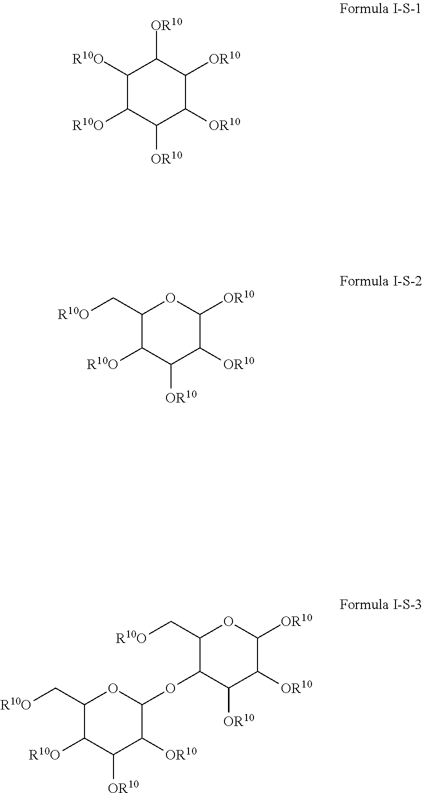

In some embodiments, each unit of L¹-L²-L³-D in Formula I-S-1, I-S-2, or I-S-3 can be the same and can be selected from:

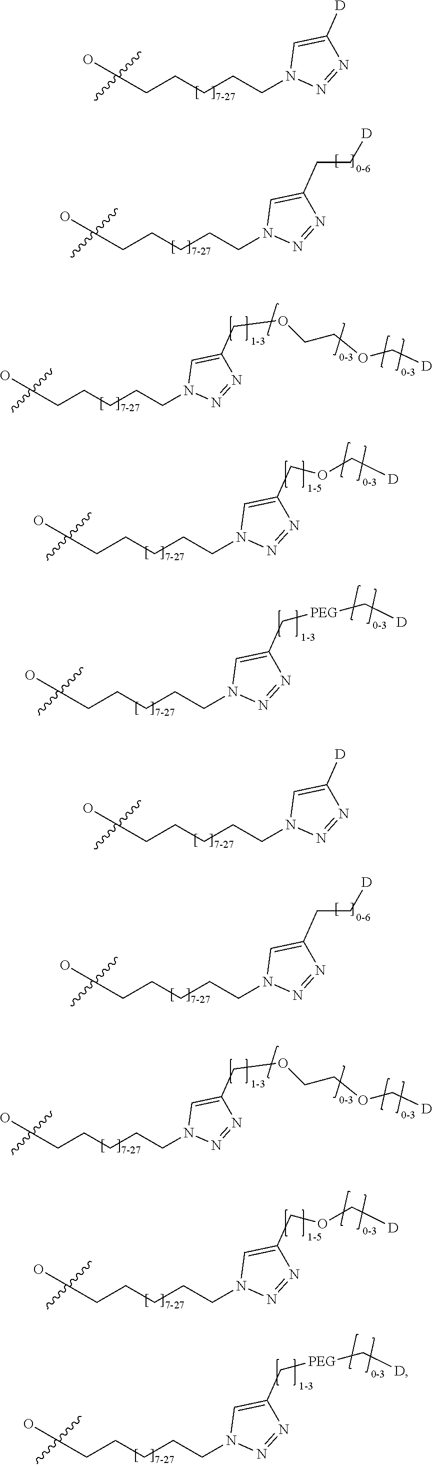

wherein D is defined herein and PEG can be any of the PEG chain described herein. As discussed herein, any of the potential regioisomers and/or stereoisomers are encompassed by the present disclosure, either as individual isomers or a mixture of isomers in any ratio. In some embodiments, D is selected from:

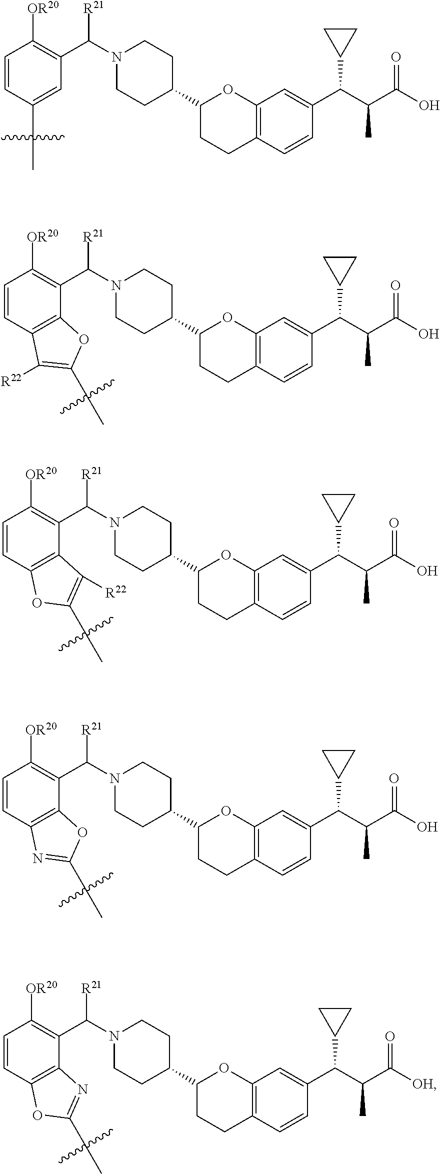

wherein: R²⁰ is C₁₋₆ alkyl or fluorine substituted C₁₋₆ alkyl; R²¹ is hydrogen or a C₁₋₆ alkyl; and R²² is hydrogen, halogen, CN, C₁₋₆ alkyl or fluorine substituted C₁₋₆ alkyl or a C₃₋₆ cycloalkyl. In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

### Formula II

In some embodiments, the present disclosure provides a compound of Formula II, or a pharmaceutically acceptable salt or ester thereof:

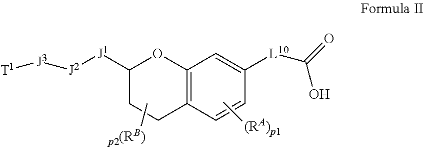

wherein:
L¹⁰ is an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{B} at each occurrence is independently halogen, hydroxyl, amino, substituted amino, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{B} are joined to form an optionally substituted ring structure; p2 is 0, 1, 2, 3, or 4;
J¹ is a bond, an optionally substituted aryl or heteroaryl ring, —C₁₋₆alkylene-N(R¹⁰⁰)—, 3-14 membered optionally substituted heterocyclylene containing at least one ring nitrogen atom, or —C₁₋₆alkylene-(3-14 membered optionally substituted heterocyclylene containing at least one ring nitrogen atom)-;
J² is a bond or an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, or two substituents are joined to form an optionally substituted ring structure;
J³ is an optionally substituted cycloalkyl, heterocyclyl, aryl or heteroaryl ring,
T¹ is selected from:
   1) —C₅₋₅₀ alkylene-T^{A}, wherein T^{A} is hydrogen or a structure having a hydrophilic moiety, e.g., a moiety having one or more ethylene glycol unit, one or more ethylene diamine unit, one or more ethylene amino ether or alcohol unit, one or more groups that are charged or can become charged at pH about 7, etc., or T^{A} is a moiety that includes one or more functional groups suitable for a coupling reaction, such as a coupling reaction for forming a carbon-carbon bond, carbon-heteroatom bond, or heteroatom-heteroatom bond, such as those forming an amide, ether, thioether, carbamate, carbonate, ester, phosphonate, sulfonate, sulfonamide, or urea linkage, for example, T^{A} is OH, SH, SO₃H, NH₂, NHR¹⁰⁰, COOH, COOR¹⁰², CONR¹⁰⁰R¹⁰¹, or a leaving group;
   2) -T^{B}-C₅₋₅₀ alkylene-T^{A}; wherein T^{A} is defined above, T^{B} is —N(R¹⁰⁰)—, —O—, —S—, —SO₂—, —C(═O)—, or a moiety selected from:

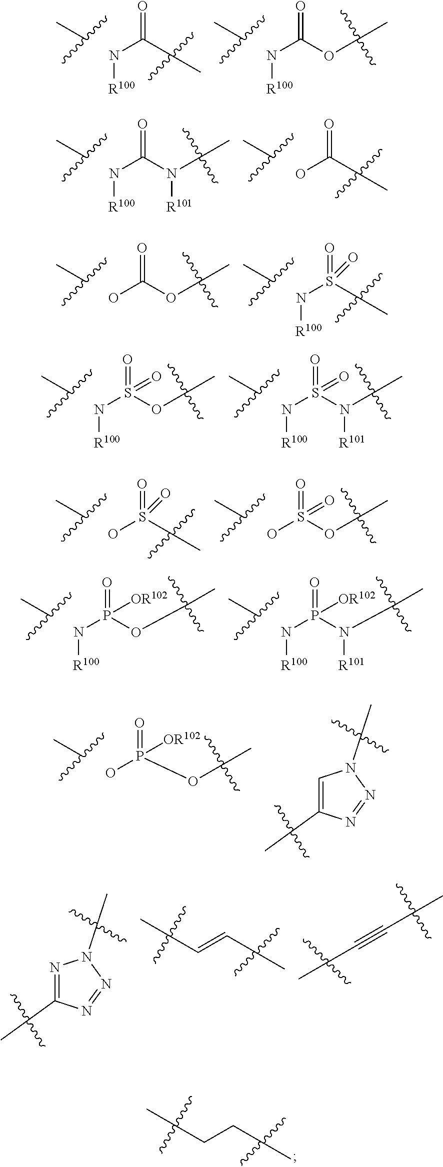

3) a moiety having a formula of -T^{C}-T^{B}-T_{D}-C₅₋₅₀ alkylene-T^{A}, wherein T^{C} and T^{D} are independently a bond, optionally substituted alkylene, optionally substituted heteroalkylene, optionally substituted carbocyclylene, optionally substituted heterocyclylene, optionally substituted arylene, or optionally substituted heteroarylene, and T^{A} and T^{B} are defined above, or
4) a moiety having a formula of -T^{C}-G, wherein T^{C} is defined above, and G is hydrogen, OH, N₃ or acetylene.
wherein R¹⁰⁰, R¹⁰¹ and R¹⁰² at each occurrence is independently hydrogen, optionally substituted alkyl, or optionally substituted cycloalkyl.

In some embodiments, the compound of Formula II can have a formula II-1:

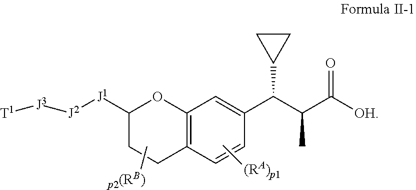

The phenyl ring in Formula II or II-1 is typically not further substituted, i.e., p1 is O. However, in some embodiments, p1 in Formula II or II-1 can also be 1, and in such embodiments, R^{A} can be for example, F, Cl, CN, C₁₋₄ alkyl optionally substituted with 1-3 fluorine, or C₁₋₄ alkoxy optionally substituted with 1-3 fluorine.

In some embodiments, p2 in Formula II or II-1 is 0. In some embodiments, p2 in Formula II or II-1 can also be 1 or 2, and in such embodiments, R^{B} at each occurrence can be independently F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine. As used herein, the two C₁₋₄ alkyl in N(C₁₋₄ alkyl)(C₁₋₄ alkyl) can be the same or different.

In some specific embodiments, the compound of Formula II can have a formula II-1-A:

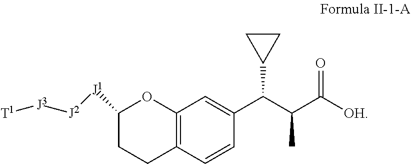

In some embodiments, J¹ in Formula II (e.g., Formula II-1 or II-1-A) is —C₁₋₆alkylene-N(R¹⁰⁰) such as CH₂—N(C₁₋₄ alkyl)-. In some embodiments, J¹ in Formula II (e.g., Formula II-1 or II-1-A) is a 4-12 membered optionally substituted heterocyclic ring having one or two ring nitrogen atoms. For example, in some embodiments, J¹ is a 4-8 (e.g., 4, 5, 6, or 7) membered monocyclic optionally substituted saturated heterocyclic ring having one or two ring heteroatoms independently selected from S, O, and N, provided at least one of the ring heteroatom is nitrogen. In some embodiments, J¹ is selected from the following (J² is included to show direction of connections):

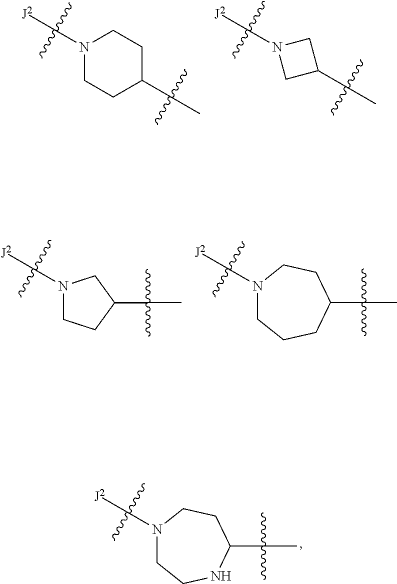

each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, and C₁₋₄ alkoxy optionally substituted with 1-3 fluorine.

In some embodiments, J¹ in Formula II (e.g., Formula II-1 or II-1-A) can also be a bicyclic or polycyclic 6-12 membered optionally substituted saturated heterocyclic ring having one or two ring heteroatoms independently selected from S, O, and N, provided at least one of the ring heteroatom is nitrogen. For example, in some embodiments, J¹ is selected from the following (J² is included to show direction of connections):

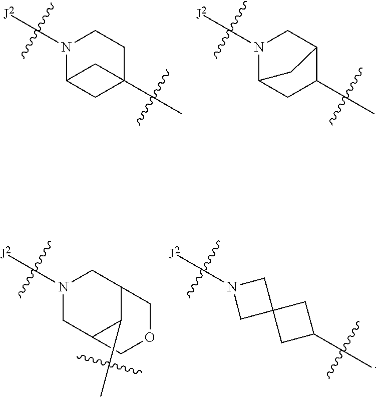

In some embodiments, J² in Formula II (e.g., Formula II-1 or II-1-A) is a straight chain or branched C₁₋₄ alkylene, optionally substituted with 1-3 fluorine. For example, in some embodiments, J² is CH₂ or —CH(CH₃)—.

J³ in Formula II (e.g., Formula II-1 or II-1-A) is typically an aryl (e.g., phenyl) or heteroaryl ring (e.g., pyridyl), each of which is unsubstituted or substituted with one or more (e.g., 1, 2, or 3) substituents independently selected from 1) halogen, CN, —CF₃, OH, amino, substituted amino, ester, amide, carbonate, or carbamate; and 2) C₁₋₆ alkyl, C₂₋₆ alkenyl, C₂₋₆ alkynyl, C₁₋₆ heteroalkyl, C₃₋₆ cycloalkyl, C₁₋₆ alkoxy, C₃₋₆ cycloalkoxy, aryl, heteroaryl, 3-8 membered heterocycloalkyl having one or two ring heteroatoms independently selected from N, O, and S, wherein each of which is optionally substituted with one or more (e.g., 1, 2, or 3) substituents independently selected from F, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy.

In some embodiments, J³ in Formula II (e.g., Formula II-1 or II-1-A) is a phenyl ring, which is substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F. For example, in some embodiments, the phenyl ring can be substituted with one or two substituents independently selected from C₁₋₄ alkyl optionally substituted with fluorine, e.g., CF₃, and C₁₋₆ alkoxy optionally substituted with fluorine, such as methoxy, ethoxy, isopropoxy, or O—CF₃.

In some embodiments, J³ in Formula II (e.g., Formula II-1 or II-1-A) is a 5-10 membered monocyclic or bicyclic heteroaryl ring, which is substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

For example, in some embodiments, J³ in Formula II (e.g., Formula II-1 or II-1-A) is selected from:

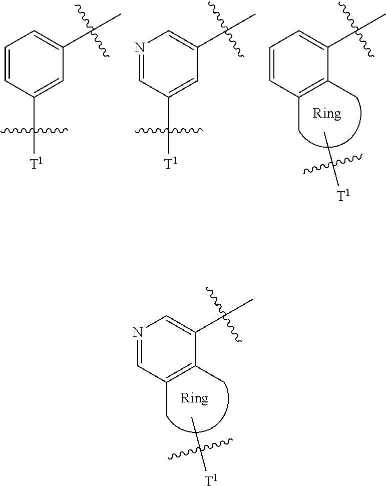

wherein: Ring represents an aromatic or non-aromatic ring structure,
wherein each of the phenyl, pyridyl, or fused ring structure is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F. For example, in some embodiments, the phenyl, pyridyl, or fused ring structure can be substituted with one or two substituents independently selected from C₁₋₄ alkyl optionally substituted with fluorine, e.g., CF₃, and C₁₋₆ alkoxy optionally substituted with fluorine, such as methoxy, ethoxy, isopropoxy, or O—CF₃.

In some embodiments, J³ in Formula II (e.g., Formula II-1 or II-1-A) is selected from:

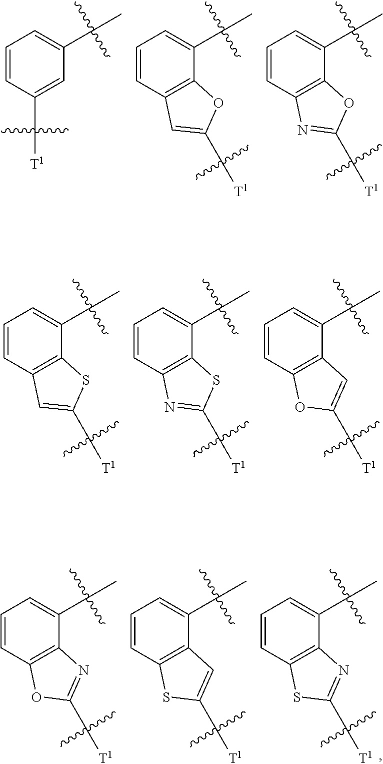

wherein each of which is optionally substituted with 1-3 substituents independently selected from F, Cl, CN, OH, C₁₋₆ alkyl, C₁₋₆ heteroalkyl, C₃₋₆ cycloalkyl, C₁₋₆ alkoxy, or C₃₋₆ cycloalkoxy, wherein the alkyl, heteroalkyl, cycloalkyl, alkoxy or cycloalkoxy is optionally substituted with one or more (e.g., 1, 2, or 3) substituents independently selected from F, —OH, C₁₋₄ alkoxy optionally substituted with F, oxo (as applicable), NH₂, NH(C₁₋₄ alkyl), N(C₁₋₄ alkyl((C₁₋₄ alkyl), C₁₋₄ alkyl optionally substituted with F.

In some embodiments, J³ in Formula II (e.g., Formula II-1 or II-1-A) is selected from:

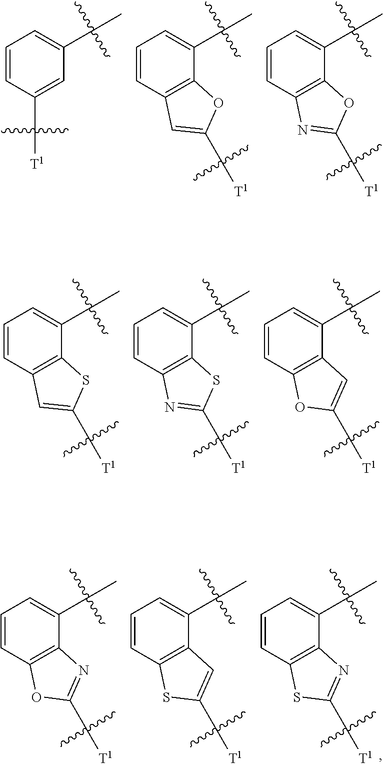

wherein each of which is optionally substituted with 1-3 substituents independently selected from F, Cl, CN, OH, C₁₋₆ alkyl optionally substituted with F (e.g., CF₃), cyclopropyl, cyclobutyl, C₁₋₆ alkoxy optionally substituted with F (e.g., —O—CF₃), or C₃₋₆ cycloalkoxy. For example, in some embodiments, the phenyl, benzofuran, benzothiophene, benzoxazol, or benzothiazol ring can be substituted with one or two substituents independently selected from C1-4 alkyl optionally substituted with fluorine, e.g., CF₃, C₁₋₆ alkoxy optionally substituted with fluorine, such as methoxy, ethoxy, isopropoxy, or O—CF₃. Preferably, the one substituent is ortho to J².

In some embodiments, the compound of Formula II is characterized as having a Formula II-1-A-1, II-1-A-2, II-1-A-3, II-1-A-4, or II-1-A-5:

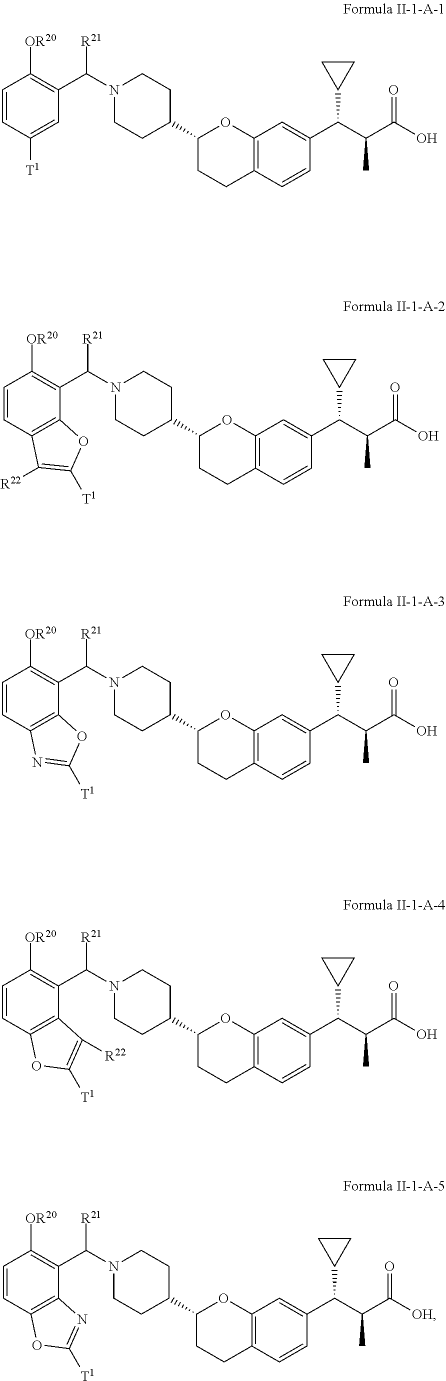

wherein:
T¹ is defined herein,
R²⁰ is C₁₋₆ alkyl or fluorine substituted C₁₋₆ alkyl,
R²¹ is hydrogen or C₁₋₆ alkyl, and
R²² is hydrogen, halogen, CN, C₁₋₆ alkyl or fluorine substituted C₁₋₆ alkyl or a C₃₋₆ cycloalkyl.
In some embodiments, R²⁰ is methyl, ethyl, n-propyl, isopropyl, or CF₃. In some embodiments, R²¹ is hydrogen, methyl, ethyl, n-propyl, or isopropyl. In some embodiments, R²⁰ is CF₃ and R²¹ is hydrogen or methyl. In some embodiments, R²⁰ is CH₃ and R²¹ is hydrogen or methyl. In some embodiments, R²² is hydrogen. In some embodiments, R²² is methyl. In some embodiments, R²² is cyclopropyl.

In some embodiments, T¹ in Formula II (e.g., Formula II-1, II-1-A, II-1-A-1, II-1-A-2, II-1-A-3, II-1-A-4, or II-1-A-5) can be —C₁₀₋₃₀ alkylene-T^{A}, wherein T^{A} is defined herein. In some embodiments, the C₁₀₋₃₀ alkylene can be a straight chain C₁₂₋₂₄ alkylene, such as a straight chain C₁₄, C₁₆, C₁₈, C₂₀, C₂₂, or C₂₄ alkylene. In some embodiments, T^{A} is hydrogen. In some embodiments, T^{A} comprises a polyethylene glycol (PEG) chain, e.g., any of those described herein. In some embodiments, T^{A} is —OH, amine, amidine, guanidine, phosphate, sulfate, carboxylic acid, a polyol (e.g., sugar alcohol), amino alcohol, a short peptide, monosaccharide, disaccharide, polysaccharide, or a basic heterocycle or heteroaryl. For example, in some embodiments, T^{A} is —OH, NH₂, or COOH. In some embodiments, T^{A} is COOH. In some embodiments, T^{A} is a polyol residue selected from:

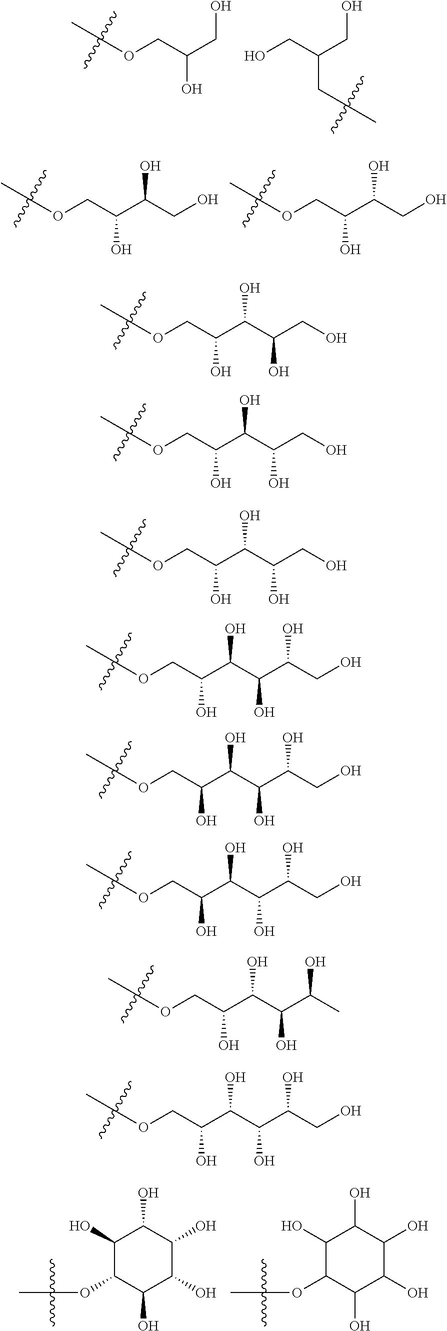

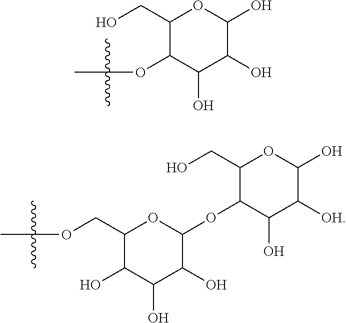 [SMILES: *OC1C(CO)OC(O)C(O)C1O ; *OCC1OC(OC2C(CO)OC(O)C(O)C2O)C(O)C(O)C1O]

While the polyols shown above show connection to the remainder of the molecule via one of the hydroxyl groups, this disclosure also contemplates all other possible connections through a different hydroxyl group. As shown above, for glycerol, the attaching point can be through the primary hydroxyl group or the secondary hydroxyl group. Similarly, for the other polyols shown above, the connection can be through any of the available hydroxyl groups. In some embodiments, T^{A} is a covalently bonded carrier having a hydrophilic moiety. For example, in some embodiments, T^{A} can also be —X-Q, wherein X and Q are as defined hereinabove for Formula I. In some embodiments, T^{A} is a covalently bonded carrier having one or more ethylene glycol unit, one or more ethylene diamine unit, one or more ethylene amino ether or alcohol unit, and/or one or more groups that are charged or can become charged at pH about 7. In some embodiments, T^{A} comprises a residue of a dendrimer (e.g., any of those described herein). In some embodiments, T^{A} comprises a residue of a poly(amide amine) dendrimer, a poly(propylene amine) dendrimer, or a poly (amide amine)-poly(propylene amine) dendrimer.

In some embodiments, T¹ in Formula II (e.g., Formula II-1, II-1-A, II-1-A-1, II-1-A-2, II-1-A-3, II-1-A-4, or II-1-A-5) can be -T^{B}-C₁₀₋₃₀ alkylene-T^{A}, wherein T^{A} and T^{B} are defined herein. In some embodiments, T^{B} is N(R¹⁰⁰)—, —O—, —C(═O)—, or a moiety selected from:

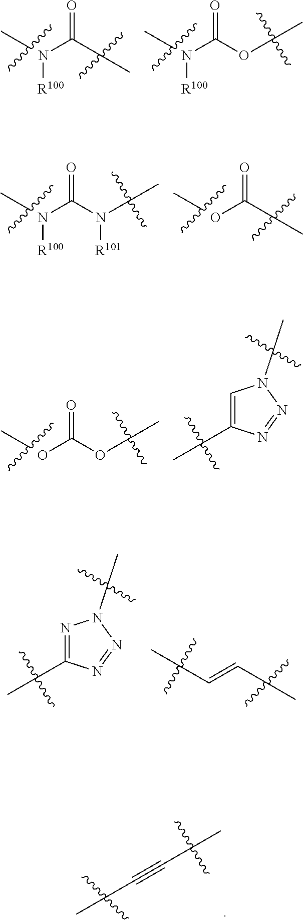

In some embodiments, R¹⁰⁰ and R¹⁰¹ at each occurrence is independently hydrogen or a C₁₋₄ alkyl (e.g., methyl, ethyl, isopropyl, etc.). In some embodiments, T^{B} is N(R¹⁰⁰)—, —O—, or a moiety selected from:

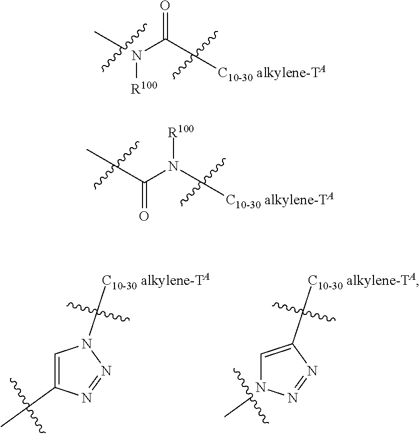

wherein R¹⁰⁰ is hydrogen or a C₁₋₄ alkyl (e.g., methyl, ethyl, isopropyl, etc.). In some embodiments, the C₁₀₋₃₀ alkylene can be a straight chain C₁₂₋₂₄ alkylene, such as a straight chain C₁₄, C₁₆, C₁₈, C₂₀, C₂₂, or C₂₄ alkylene. In some embodiments, T^{A} is hydrogen. In some embodiments, T^{A} comprises a polyethylene glycol (PEG) chain, e.g., any of those described herein. In some embodiments, T^{A} is —OH, amine, amidine, guanidine, phosphate, sulfate, carboxylic acid, a polyol (e.g., sugar alcohol), amino alcohol, a short peptide, monosaccharide, disaccharide, polysaccharide, or a basic heterocycle or heteroaryl. For example, in some embodiments, T^{A} is —OH, NH₂, or COOH. In some embodiments, T^{A} is COOH. In some embodiments, T^{A} is a polyol selected from:

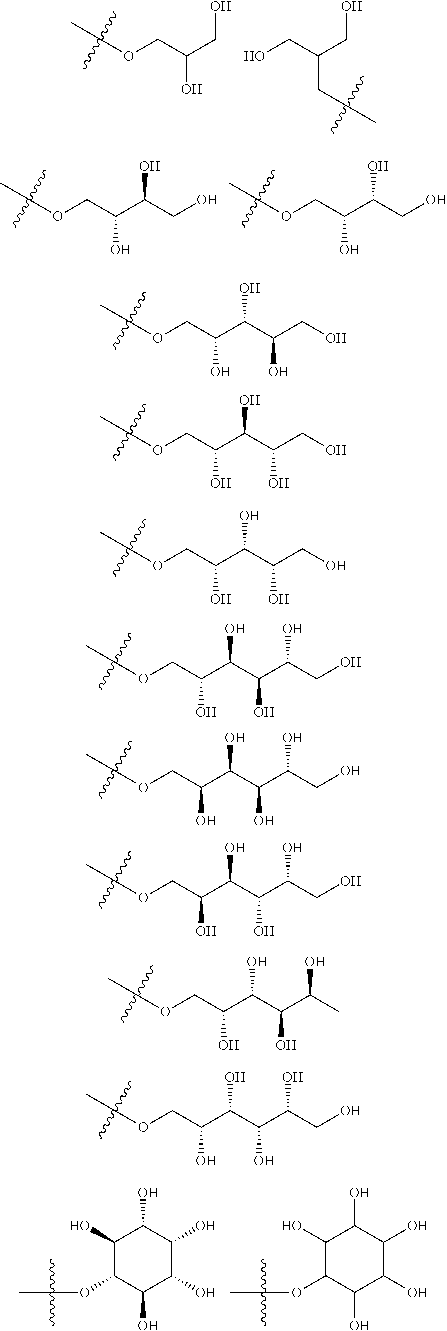

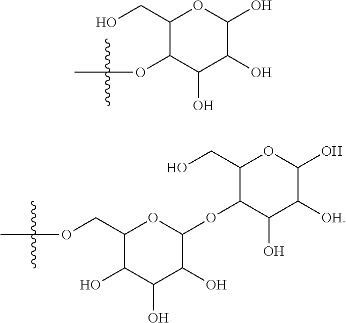 [SMILES: *OC1C(CO)OC(O)C(O)C1O ; *OCC1OC(OC2C(CO)OC(O)C(O)C2O)C(O)C(O)C1O]

While the polyols shown above show connection to the remainder of the molecule via one of the hydroxyl groups, this disclosure also contemplates all other possible connections through a different hydroxyl group. As shown above, for glycerol, the attaching point can be through the primary hydroxyl group or the secondary hydroxyl group. Similarly, for the other polyols shown above, the connection can be through any of the available hydroxyl groups. In some embodiments, T^{A} is a covalently bonded carrier having a hydrophilic moiety. For example, in some embodiments, T^{A} can also be —X-Q, wherein X and Q are as defined hereinabove for Formula I. In some embodiments, T^{A} is a covalently bonded carrier having one or more ethylene glycol unit, one or more ethylene diamine unit, one or more ethylene amino ether or alcohol unit, and/or one or more groups that are charged or can become charged at pH about 7. In some embodiments, T^{A} comprises a residue of a dendrimer (e.g., any of those described herein). In some embodiments, T^{A} comprises a residue of a poly(amide amine) dendrimer, a poly(propylene amine) dendrimer, or a poly (amide amine)-poly(propylene amine) dendrimer.

In some embodiments, T¹ in Formula II (e.g., Formula II-1, II-1-A, II-1-A-1, II-1-A-2, II-1-A-3, II-1-A-4, or II-1-A-5) can be -T^{C}-T^{B}-T^{D}-C₁₀₋₃₀ alkylene-T^{A}, wherein T^{A}, T^{B}, T^{C}, and T^{D} are defined herein. In some embodiments, T^{B} is N(R¹⁰⁰)—, —O—, —C(═O)—, or a moiety selected from:

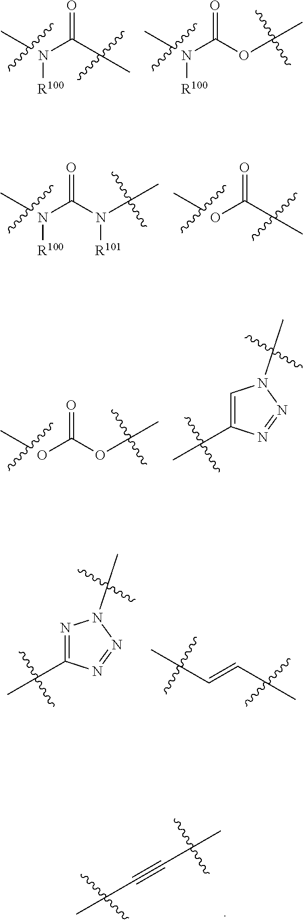

In some embodiments, R¹⁰⁰ and R¹⁰¹ at each occurrence is independently hydrogen or a C₁₋₄ alkyl (e.g., methyl, ethyl, isopropyl, etc.). In some embodiments, T^{B} is N(R¹⁰⁰)—, —O—, or a moiety selected from:

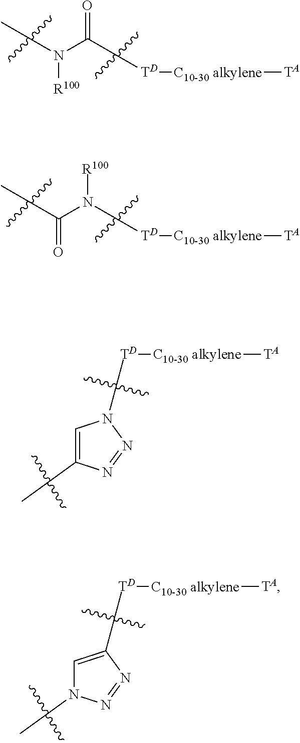

wherein R¹⁰⁰ is hydrogen or a C₁₋₄ alkyl (e.g., methyl, ethyl, isopropyl, etc.). In some embodiments, the C₁₀₋₃₀ alkylene can be a straight chain C₁₂₋₂₄ alkylene, such as a straight chain C₁₄, C₁₆, C₁₈, C₂₀, C₂₂, or C₂₄ alkylene. In some embodiments, T^{C} and T^{D} are independently a bond, optionally substituted C₁₋₁₀ alkylene, optionally substituted C₁₋₁₀ heteroalkylene having 1-5 heteroatoms independently selected from 0 and N, e.g., —CH₂—O—CH₂—. For example, in some embodiments, T^{C} is a bond. In some embodiments, T^{D} is a bond. In some embodiments, T^{C} is a bond and T^{D} is a C₁₋₁₀ alkylene or a C₁-10 heteroalkylene having 1-5 heteroatoms independently selected from 0 and N, e.g., —CH₂—O—CH₂—. In some embodiments, T^{D} is a bond and T^{C} is a C₁₋₁₀ alkylene or a C₁₋₁₀ heteroalkylene having 1-5 heteroatoms independently selected from O and N, e.g., —CH₂—O—CH₂—. In some embodiments, T^{C} and T^{D} are independently a C₁₋₁₀ alkylene or a C₁₋₁₀ heteroalkylene having 1-5 heteroatoms independently selected from 0 and N, e.g., —CH₂—O—CH₂—. In some embodiments, T^{A} is hydrogen. In some embodiments, T^{A} comprises a polyethylene glycol (PEG) chain, e.g., any of those described herein. In some embodiments, T^{A} is —OH, amine, amidine, guanidine, phosphate, sulfate, carboxylic acid, a polyol (e.g., sugar alcohol), amino alcohol, a short peptide, monosaccharide, disaccharide, polysaccharide, or a basic heterocycle or heteroaryl. For example, in some embodiments, T^{A} is —OH, NH₂, or COOH. In some embodiments, T^{A} is COOH. In some embodiments, T^{A} is a polyol selected from:

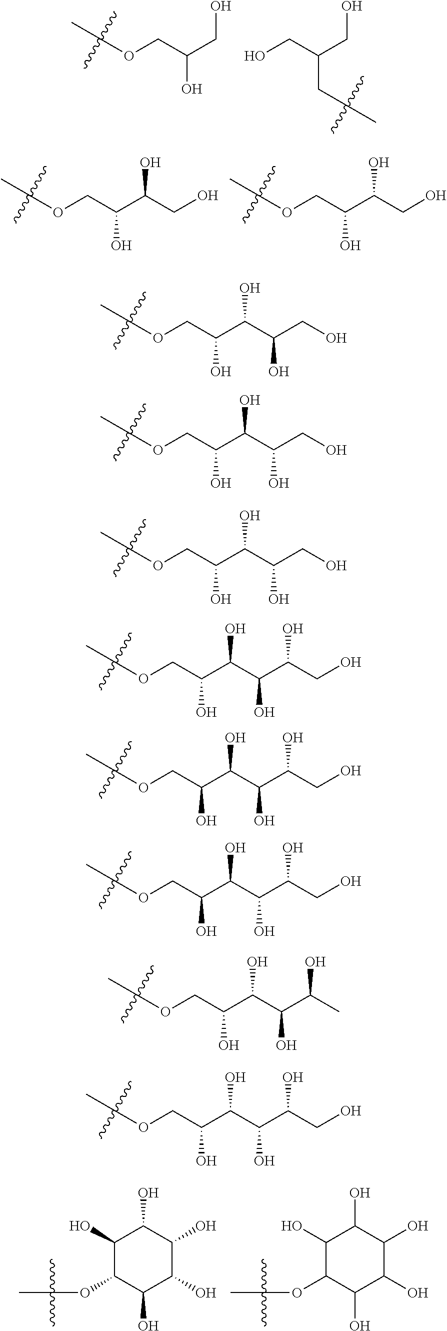

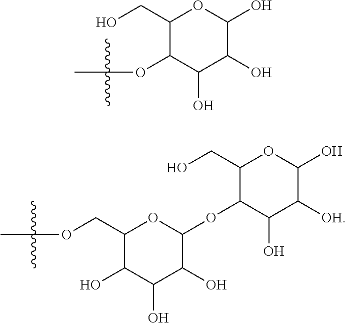 [SMILES: *OC1C(CO)OC(O)C(O)C1O ; *OCC1OC(OC2C(CO)OC(O)C(O)C2O)C(O)C(O)C1O]

While the polyols shown above show connection to the remainder of the molecule via one of the hydroxyl groups, this disclosure also contemplates all other possible connections through a different hydroxyl group. As shown above, for glycerol, the attaching point can be through the primary hydroxyl group or the secondary hydroxyl group. Similarly, for the other polyols shown above, the connection can be through any of the available hydroxyl groups. In some embodiments, T^{A} is a covalently bonded carrier having a hydrophilic moiety. For example, in some embodiments, T^{A} can also be —X-Q, wherein X and Q are as defined hereinabove for Formula I. In some embodiments, T^{A} is a covalently bonded carrier having one or more ethylene glycol unit, one or more ethylene diamine unit, one or more ethylene amino ether or alcohol unit, and/or one or more groups that are charged or can become charged at pH about 7. In some embodiments, T^{A} comprises a residue of a dendrimer (e.g., any of those described herein). In some embodiments, T^{A} comprises a residue of a poly(amide amine) dendrimer, a poly(propylene amine) dendrimer, or a poly (amide amine)-poly(propylene amine) dendrimer.

In some embodiments, T¹ in Formula II (e.g., Formula II-1, II-1-A, II-1-A-1, II-1-A-2, II-1-A-3, II-1-A-4, or II-1-A-5) can be -T^{C}-G, wherein T^{C}, and G are defined herein. In some embodiments, G is OH. In some embodiments, G is hydrogen. In some embodiments, G is N₃. In some embodiments, G is

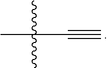 [SMILES: *C#C]

In some embodiments, T^{C} is a bond, optionally substituted C₁₋₁₀ alkylene, optionally substituted C₁₋₁₀ heteroalkylene having 1-5 heteroatoms independently selected from O and N, e.g., —CH₂—O—CH₂—. For example, in some embodiments, T^{C} is a bond. In some embodiments, T^{C} is a C₁₋₁₀ alkylene or a C₁₋₁₀ heteroalkylene having 1-5 heteroatoms independently selected from O and N, e.g., —CH₂—O—, or —CH₂—O—CH₂—.

In some embodiments, the present disclosure also provides compounds of the following formulae, or pharmaceutically acceptable salts or ester thereof.

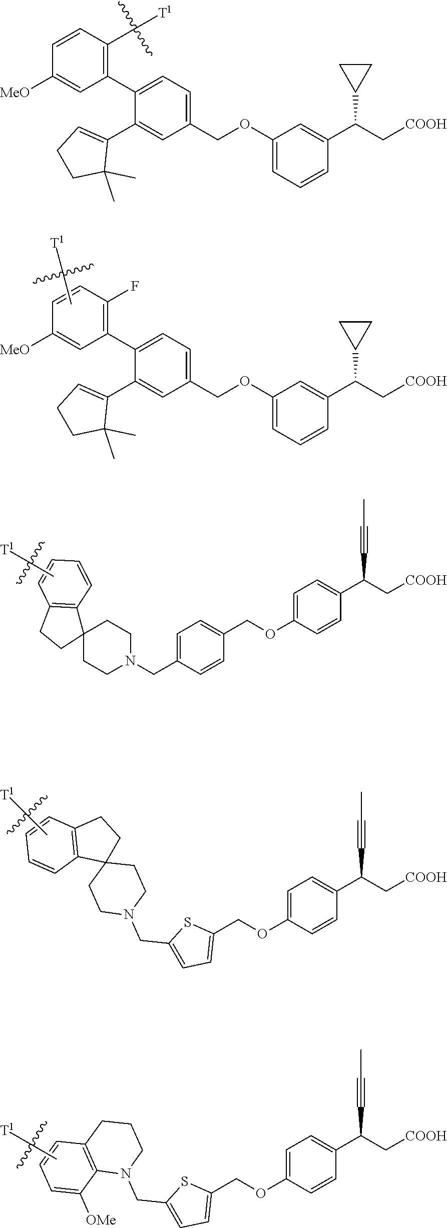

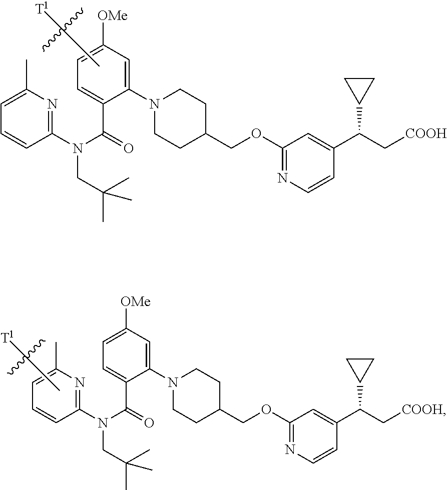

wherein T¹ is any of those defined herein in connection with Formula II or its subformulae.

In some embodiments, the present disclosure also provides compounds of the following formulae III-1, III-2, III-3, III-4, III-5, III-6, III-7, III-8, or III-9, or pharmaceutically acceptable salts or ester thereof:

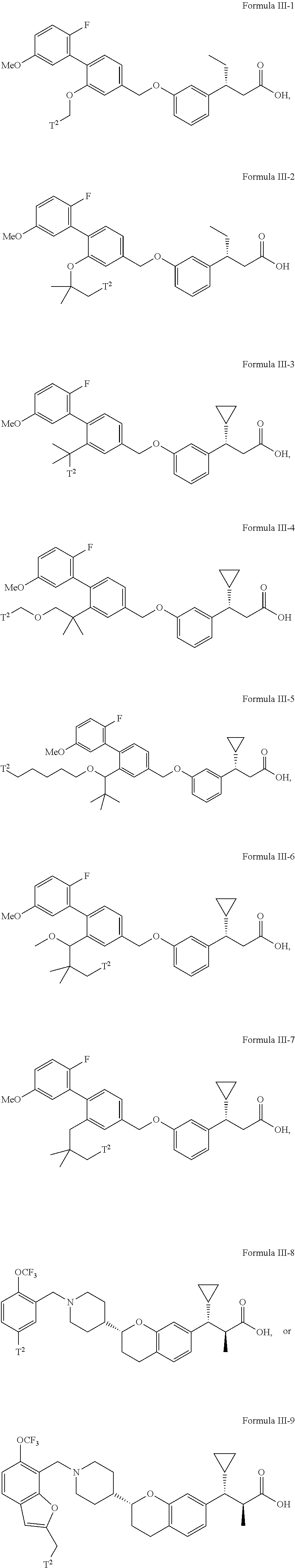

wherein T² is selected from:
   1) —C₅₋₅₀ alkylene-T^{A1}, wherein T^{A1} is hydrogen or a moiety that includes one or more functional groups suitable for a coupling reaction, such as a coupling reaction for forming a carbon-carbon bond, carbon-heteroatom bond, or heteroatom-heteroatom bond, such as those forming an amide, ether, thioether, carbamate, carbonate, ester, phosphonate, sulfonate, sulfonamide, or urea linkage, for example, T^{A1} is OH, SH, SO₃H, NH₂, NHR¹⁰⁰, COOH, COOR¹⁰², CONR¹⁰⁰R¹⁰¹, or a leaving group;
   2) -T^{B}-C₅₋₅₀ alkylene-T^{A1}; wherein T^{A1} is defined above, T^{B} is —N(R¹⁰⁰)—, —O—, —S—, —SO₂—, —C(═O)—, or a moiety selected from:

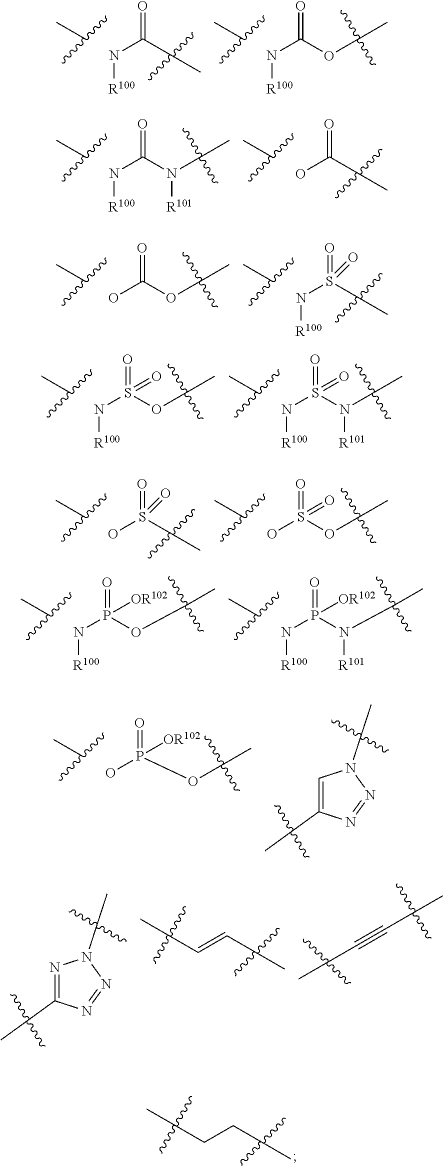

3) a moiety having a formula of -T^{C}-T^{B}-T^{D}-C₅₋₅₀ alkylene-T^{A}, wherein T^{C} and T^{D} are independently a bond, optionally substituted alkylene, optionally substituted heteroalkylene, optionally substituted carbocyclylene, optionally substituted heterocyclylene, optionally substituted arylene, or optionally substituted heteroarylene, and T^{A} and T^{B} are defined above, or
4) a moiety having a formula of -T^{C}-G, wherein T^{C} is defined above, and G is hydrogen, OH, N₃ or acetylene, wherein R¹⁰⁰, R¹⁰¹ and R¹⁰² at each occurrence is independently hydrogen, optionally substituted alkyl, or optionally substituted cycloalkyl. In some preferred embodiments, T^{A1} is OH, NH₂, or COOH. In some specific embodiments, T^{A1} is COOH.

In some embodiments, T² in Formula III-1 to III-9 can be —C₁₀₋₃₀ alkylene-T^{A1}, wherein T^{A1} is defined herein. In some embodiments, the C₁₀₋₃₀ alkylene can be a straight chain C₁₂₋₂₄ alkylene, such as a straight chain C₁₄, C₁₆, C₁₈, C₂₀, C₂₂, or C₂₄ alkylene. In some embodiments, T^{A1} is hydrogen. In some preferred embodiments, T^{A1} is OH, NH₂, or COOH. In some specific embodiments, T^{A1} is COOH.

In some embodiments, T² in Formula III-1 to III-9 can be -T^{B}-C₁₀₋₃₀ alkylene-T^{A1} wherein T^{A1} and T^{B} are defined herein. In some embodiments, T^{B} is N(R¹⁰⁰)—, —O—, —C(═O)—, or a moiety selected from:

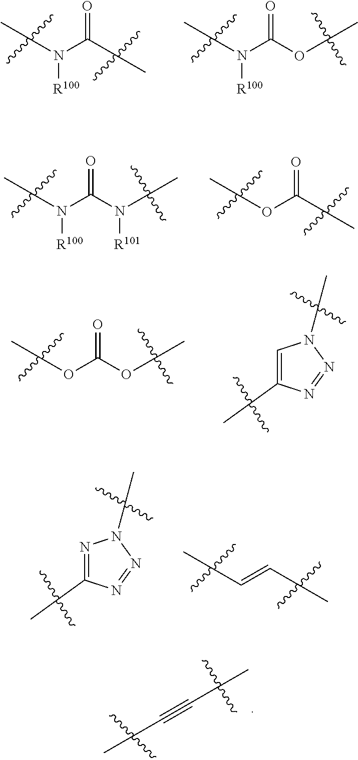

In some embodiments, R¹⁰⁰ and R¹⁰¹ at each occurrence is independently hydrogen or a C₁₋₄ alkyl (e.g., methyl, ethyl, isopropyl, etc.). In some embodiments, T^{B} is N(R¹⁰⁰)—, —O—, or a moiety selected from:

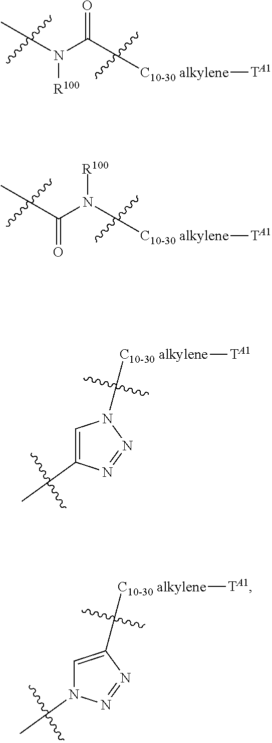

wherein R¹⁰⁰ is hydrogen or a C₁₋₄ alkyl (e.g., methyl, ethyl, isopropyl, etc.). In some embodiments, the C₁₀₋₃₀ alkylene can be a straight chain C₁₂₋₂₄ alkylene, such as a straight chain C₁₄, C₁₆, C₁₈, C₂₀, C₂₂, or C₂₄ alkylene. In some embodiments, T^{A1} is hydrogen. In some preferred embodiments, T^{A1} is OH, NH₂, or COOH. In some specific embodiments, T^{A1} is COOH.

In some embodiments, T² in Formula III-1 to III-9 can be -T^{C}-T^{B}-T^{D}-C₁₀₋₃₀ alkylene-T^{A1}, wherein T^{A1}, T^{B}, T^{C}, and T^{D} are defined herein. In some embodiments, T^{B} is —N(R¹⁰⁰)—. —O—, —C(═O)—, or a moiety selected from:

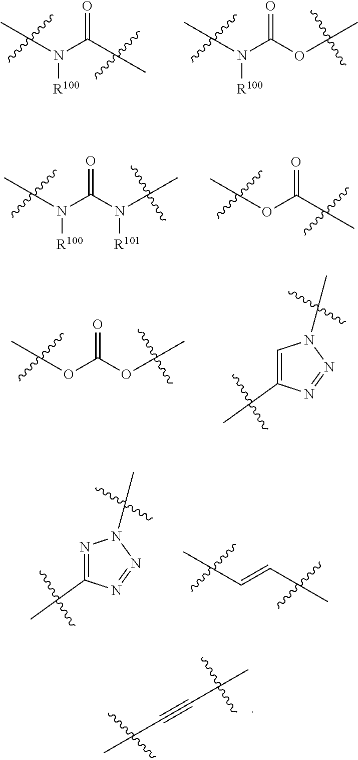

In some embodiments, R¹⁰⁰ and R¹⁰¹ at each occurrence is independently hydrogen or a C₁₋₄ alkyl (e.g., methyl, ethyl, isopropyl, etc.). In some embodiments, T^{B} is N(R¹⁰⁰)—, —O—, or a moiety selected from:

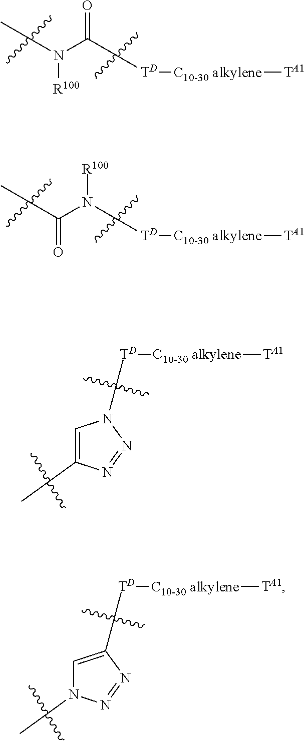

wherein R¹⁰⁰ is hydrogen or a C₁₋₄ alkyl (e.g., methyl, ethyl, isopropyl, etc.). In some embodiments, the C₁₀₋₃₀ alkylene can be a straight chain C₁₂₋₂₄ alkylene, such as a straight chain C₁₄, C₁₆, C₁₈, C₂₀, C₂₂, or C₂₄ alkylene. In some embodiments, T^{C} and T^{D} are independently a bond, optionally substituted C₁₋₁₀ alkylene, optionally substituted C₁₋₁₀ heteroalkylene having 1-5 heteroatoms independently selected from 0 and N, e.g., —CH₂—O—CH₂—. For example, in some embodiments, T^{C} is a bond. In some embodiments, T^{D} is a bond. In some embodiments, T^{C} is a bond and T^{D} is a C₁₋₁₀ alkylene or a C₁₋₁₀ heteroalkylene having 1-5 heteroatoms independently selected from O and N, e.g., —CH₂—O—CH₂—. In some embodiments, T^{D} is a bond and T^{C} is a C₁₋₁₀ alkylene or a C₁₋₁₀ heteroalkylene having 1-5 heteroatoms independently selected from O and N, e.g., —CH₂—O—CH₂—. In some embodiments, T^{C} and T^{D} are independently a C₁₋₁₀ alkylene or a C₁₋₁₀ heteroalkylene having 1-5 heteroatoms independently selected from O and N, e.g., —CH₂—O—CH₂—. In some embodiments, T^{A1} is hydrogen. In some preferred embodiments, T^{A1} is OH, NH₂, or COOH. In some specific embodiments, T^{A1} is COOH.

In some embodiments, T² in Formula III-1 to III-9 can be -T^{C}-G, wherein T^{C}, and G are defined herein. In some embodiments, G is OH. In some embodiments, G is hydrogen. In some embodiments, G is N₃. In some embodiments, G is

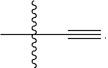 [SMILES: *C#C]

In some embodiments, T^{C} is a bond, optionally substituted C₁₋₁₀ alkylene, optionally substituted C₁₋₁₀ heteroalkylene having 1-5 heteroatoms independently selected from O and N, e.g., —CH₂—O—CH₂—. For example, in some embodiments, T^{C} is a bond. In some embodiments, T^{C} is a C₁₋₁₀ alkylene or a C₁₋₁₀ heteroalkylene having 1-5 heteroatoms independently selected from O and N, e.g., —CH₂—O—, or —CH₂—O—CH₂—.

In some embodiments, compounds of Formula III-1 to III-9 or pharmaceutically acceptable salts or esters thereof are useful as GPR40 agonists.

In some embodiments, the present disclosure also provides Compound Nos. 1-7 with the general formula below:

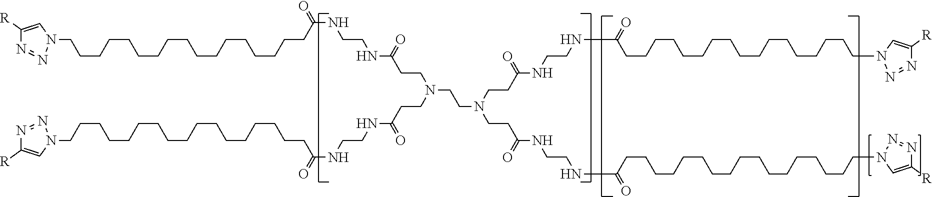

| Compound No. | R |
|---|---|
| 1 | 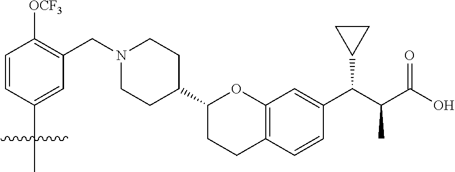 [SMILES: *c1ccc(OC(F)(F)F)c(CN2CCC([C@H]3CCc4ccc([C@H](C5CC5)[C@H](C)C(=O)O)cc4O3)CC2)c1] |
| 2 | 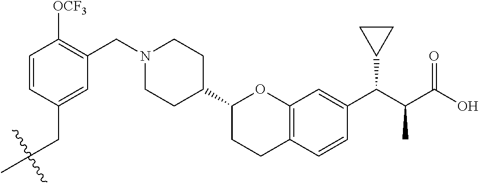 [SMILES: *Cc1ccc(OC(F)(F)F)c(CN2CCC([C@H]3CCc4ccc([C@H](C5CC5)[C@H](C)C(=O)O)cc4O3)CC2)c1] |
| 3 | 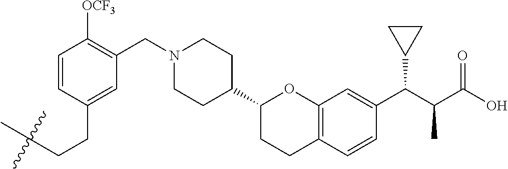 [SMILES: *CCc1ccc(OC(F)(F)F)c(CN2CCC([C@H]3CCc4ccc([C@H](C5CC5)[C@H](C)C(=O)O)cc4O3)CC2)c1] |
| 4 | 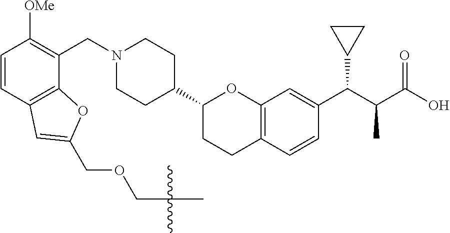 [SMILES: *COCc1cc2ccc(OC)c(CN3CCC([C@H]4CCc5ccc([C@H](C6CC6)[C@H](C)C(=O)O)cc5O4)CC3)c2o1] |
| 5 | 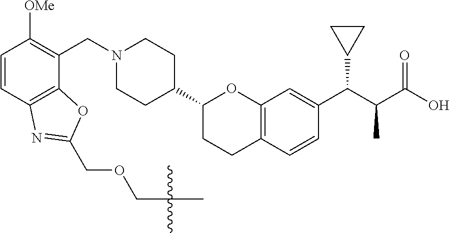 [SMILES: *COCc1nc2ccc(OC)c(CN3CCC([C@H]4CCc5ccc([C@H](C6CC6)[C@H](C)C(=O)O)cc5O4)CC3)c2o1] |
| 6 | 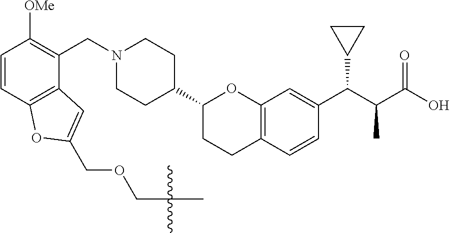 [SMILES: *COCc1cc2c(CN3CCC([C@H]4CCc5ccc([C@H](C6CC6)[C@H](C)C(=O)O)cc5O4)CC3)c(OC)ccc2o1] |
| 7 | 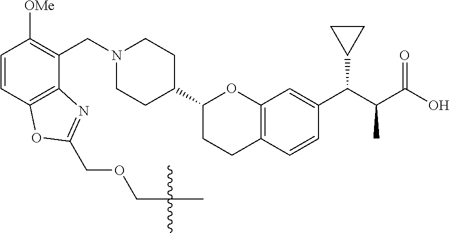 [SMILES: *COCc1nc2c(CN3CCC([C@H]4CCc5ccc([C@H](C6CC6)[C@H](C)C(=O)O)cc5O4)CC3)c(OC)ccc2o1] |

In some embodiments, the present disclosure also provides Compound Nos. 8-14 with the general formula below:

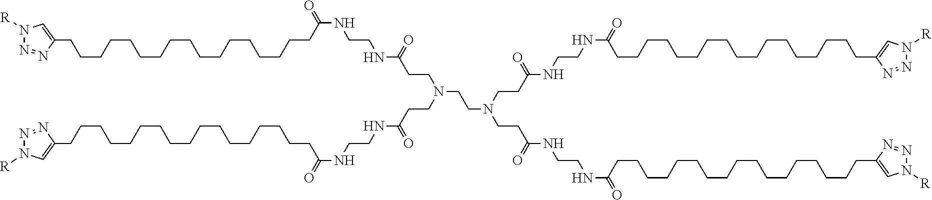

| Compound No. | R |
|---|---|
| 8 | 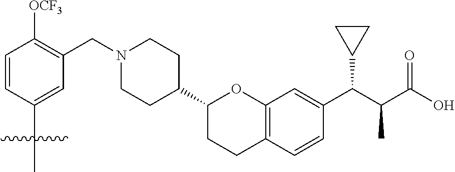 [SMILES: *c1ccc(OC(F)(F)F)c(CN2CCC([C@H]3CCc4ccc([C@H](C5CC5)[C@H](C)C(=O)O)cc4O3)CC2)c1] |
| 9 | 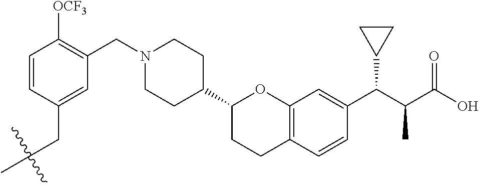 [SMILES: *Cc1ccc(OC(F)(F)F)c(CN2CCC([C@H]3CCc4ccc([C@H](C5CC5)[C@H](C)C(=O)O)cc4O3)CC2)c1] |
| 10 | 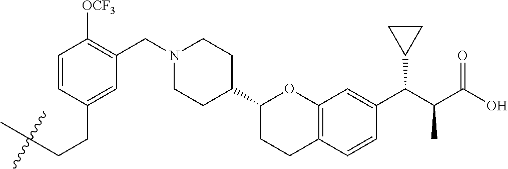 [SMILES: *CCc1ccc(OC(F)(F)F)c(CN2CCC([C@H]3CCc4ccc([C@H](C5CC5)[C@H](C)C(=O)O)cc4O3)CC2)c1] |
| 11 | 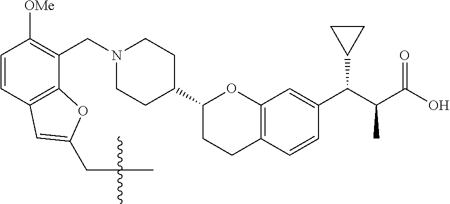 [SMILES: *Cc1cc2ccc(OC)c(CN3CCC([C@H]4CCc5ccc([C@H](C6CC6)[C@H](C)C(=O)O)cc5O4)CC3)c2o1] |
| 12 | 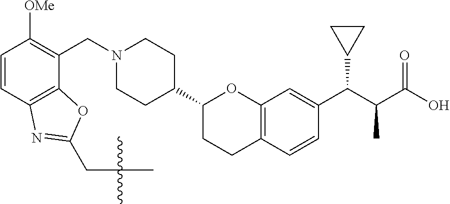 [SMILES: *Cc1nc2ccc(OC)c(CN3CCC([C@H]4CCc5ccc([C@H](C6CC6)[C@H](C)C(=O)O)cc5O4)CC3)c2o1] |
| 13 | 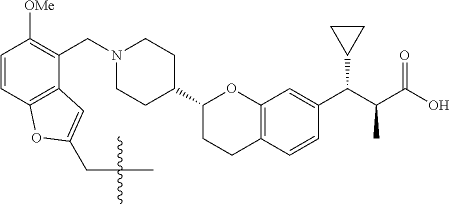 [SMILES: *Cc1cc2c(CN3CCC([C@H]4CCc5ccc([C@H](C6CC6)[C@H](C)C(=O)O)cc5O4)CC3)c(OC)ccc2o1] |
| 14 | 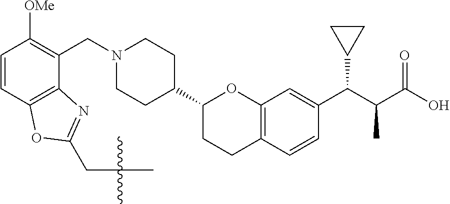 [SMILES: *Cc1nc2c(CN3CCC([C@H]4CCc5ccc([C@H](C6CC6)[C@H](C)C(=O)O)cc5O4)CC3)c(OC)ccc2o1] |

In some embodiments, the present disclosure also provides Compound Nos. 15-42 with the general formula below:

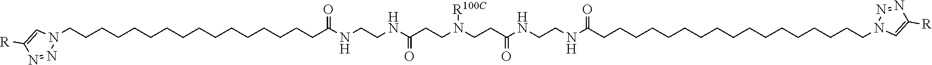

| Compound No. | R^{100C} | R |
|---|---|---|
| 15 16 17 18 | methyl ethyl n-propyl n-butyl | 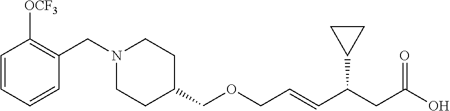 |
| 19 20 21 22 | methyl ethyl n-propyl n-butyl | 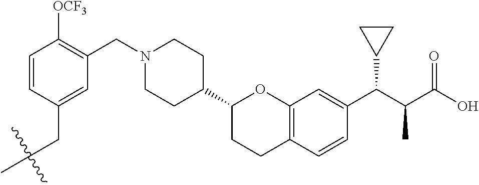 [SMILES: *Cc1ccc(OC(F)(F)F)c(CN2CCC([C@H]3CCc4ccc([C@H](C5CC5)[C@H](C)C(=O)O)cc4O3)CC2)c1] |
| 23 24 25 26 | methyl ethyl n-propyl n-butyl | 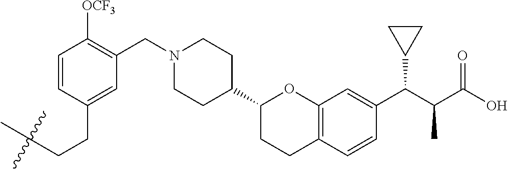 [SMILES: *CCc1ccc(OC(F)(F)F)c(CN2CCC([C@H]3CCc4ccc([C@H](C5CC5)[C@H](C)C(=O)O)cc4O3)CC2)c1] |
| 27 28 29 30 | methyl ethyl n-propyl n-butyl | 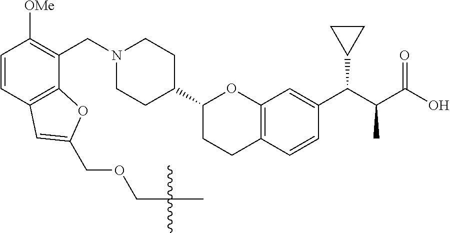 [SMILES: *COCc1cc2ccc(OC)c(CN3CCC([C@H]4CCc5ccc([C@H](C6CC6)[C@H](C)C(=O)O)cc5O4)CC3)c2o1] |
| 31 32 33 34 | methyl ethyl n-propyl n-butyl | 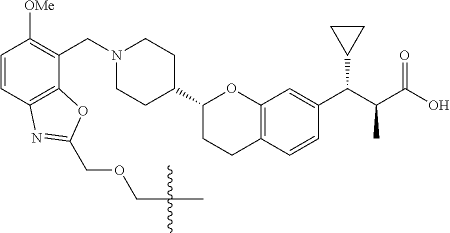 [SMILES: *COCc1nc2ccc(OC)c(CN3CCC([C@H]4CCc5ccc([C@H](C6CC6)[C@H](C)C(=O)O)cc5O4)CC3)c2o1] |
| 35 36 37 38 | methyl ethyl n-propyl n-butyl | 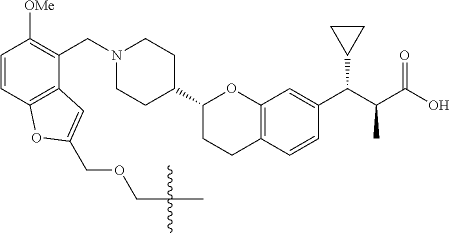 [SMILES: *COCc1cc2c(CN3CCC([C@H]4CCc5ccc([C@H](C6CC6)[C@H](C)C(=O)O)cc5O4)CC3)c(OC)ccc2o1] |
| 39 40 41 42 | methyl ethyl n-propyl n-butyl | 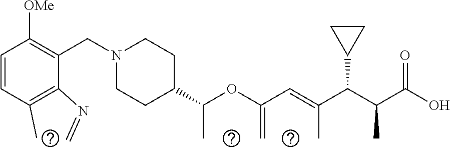 |

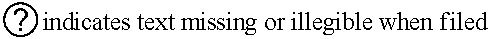

In some embodiments, the present disclosure also provides Compound Nos. 43-70 with the general formula below:

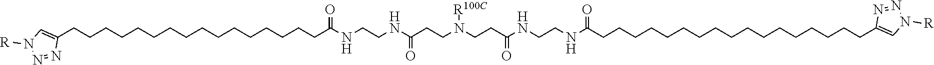

| Compound No. | R^{100C} | R |
|---|---|---|
| 43 44 45 46 | methyl ethyl n-propyl n-butyl | 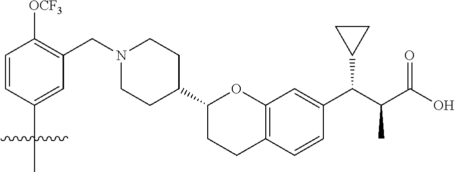 [SMILES: *c1ccc(OC(F)(F)F)c(CN2CCC([C@H]3CCc4ccc([C@H](C5CC5)[C@H](C)C(=O)O)cc4O3)CC2)c1] |
| 47 48 49 50 | methyl ethyl n-propyl n-butyl | 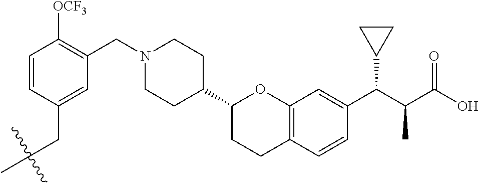 [SMILES: *Cc1ccc(OC(F)(F)F)c(CN2CCC([C@H]3CCc4ccc([C@H](C5CC5)[C@H](C)C(=O)O)cc4O3)CC2)c1] |
| 51 52 53 54 | methyl ethyl n-propyl n-butyl | 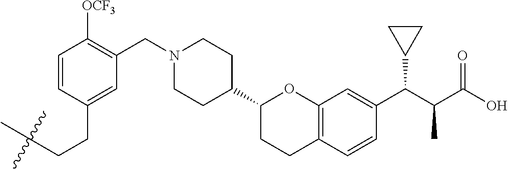 [SMILES: *CCc1ccc(OC(F)(F)F)c(CN2CCC([C@H]3CCc4ccc([C@H](C5CC5)[C@H](C)C(=O)O)cc4O3)CC2)c1] |
| 55 56 57 58 | methyl ethyl n-propyl n-butyl | 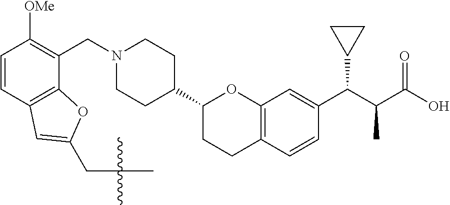 [SMILES: *Cc1cc2ccc(OC)c(CN3CCC([C@H]4CCc5ccc([C@H](C6CC6)[C@H](C)C(=O)O)cc5O4)CC3)c2o1] |
| 59 60 61 62 | methyl ethyl n-propyl n-butyl | 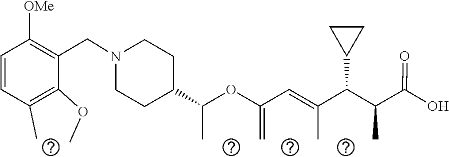 |
| 63 64 65 66 | methyl ethyl n-propyl n-butyl | 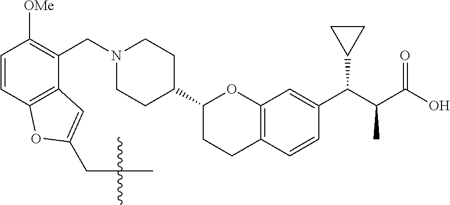 [SMILES: *Cc1cc2c(CN3CCC([C@H]4CCc5ccc([C@H](C6CC6)[C@H](C)C(=O)O)cc5O4)CC3)c(OC)ccc2o1] |
| 67 68 69 70 | methyl ethyl n-propyl n-butyl | 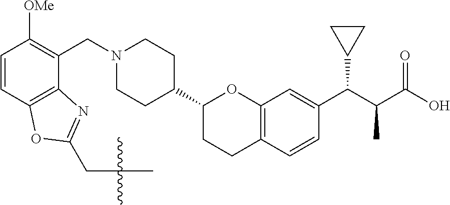 [SMILES: *Cc1nc2c(CN3CCC([C@H]4CCc5ccc([C@H](C6CC6)[C@H](C)C(=O)O)cc5O4)CC3)c(OC)ccc2o1] |

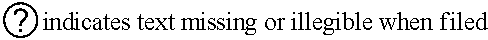

In some embodiments, the present disclosure also provides Compound Nos. 71-98 with the general formula below:

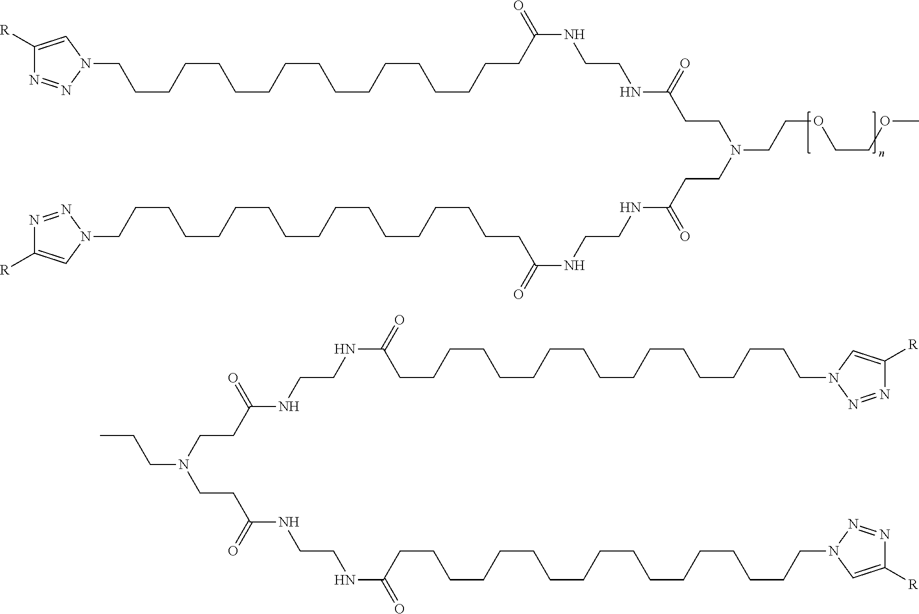

| Compound No. | n | R |
|---|---|---|
| 71 72 73 74 | 1 2 3 4 | 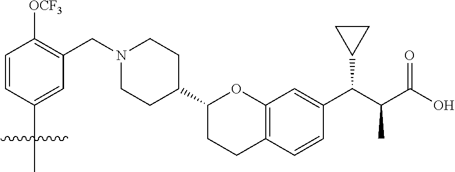 [SMILES: *c1ccc(OC(F)(F)F)c(CN2CCC([C@H]3CCc4ccc([C@H](C5CC5)[C@H](C)C(=O)O)cc4O3)CC2)c1] |
| 75 76 77 78 | 1 2 3 4 | 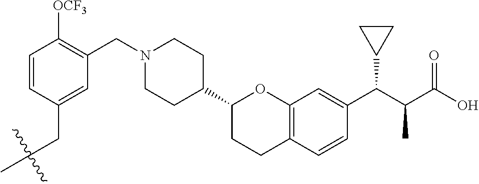 [SMILES: *Cc1ccc(OC(F)(F)F)c(CN2CCC([C@H]3CCc4ccc([C@H](C5CC5)[C@H](C)C(=O)O)cc4O3)CC2)c1] |
| 79 80 81 82 | 1 2 3 4 | 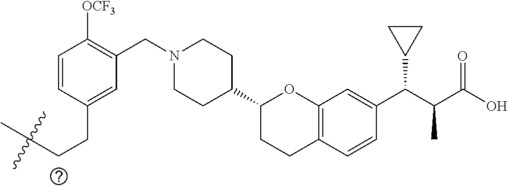 [SMILES: *CCc1ccc(OC(F)(F)F)c(CN2CCC([C@H]3CCc4ccc([C@H](C5CC5)[C@H](C)C(=O)O)cc4O3)CC2)c1] |
| 83 84 85 86 | 1 2 3 4 | 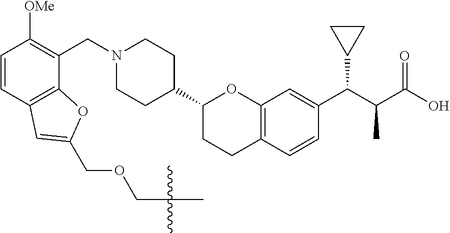 [SMILES: *COCc1cc2ccc(OC)c(CN3CCC([C@H]4CCc5ccc([C@H](C6CC6)[C@H](C)C(=O)O)cc5O4)CC3)c2o1] |
| 87 88 89 90 | 1 2 3 4 | 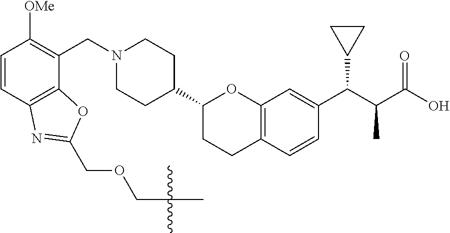 [SMILES: *COCc1nc2ccc(OC)c(CN3CCC([C@H]4CCc5ccc([C@H](C6CC6)[C@H](C)C(=O)O)cc5O4)CC3)c2o1] |
| 91 92 93 94 | 1 2 3 4 | 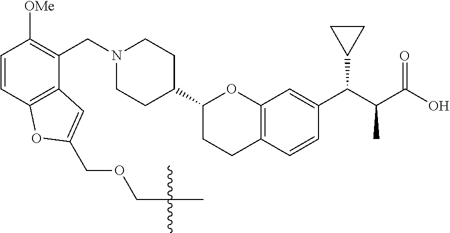 [SMILES: *COCc1cc2c(CN3CCC([C@H]4CCc5ccc([C@H](C6CC6)[C@H](C)C(=O)O)cc5O4)CC3)c(OC)ccc2o1] |
| 95 96 97 98 | 1 2 3 4 | 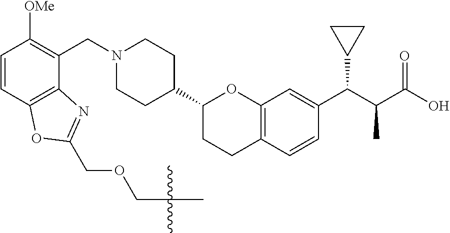 [SMILES: *COCc1nc2c(CN3CCC([C@H]4CCc5ccc([C@H](C6CC6)[C@H](C)C(=O)O)cc5O4)CC3)c(OC)ccc2o1] |

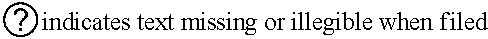

In some embodiments, the present disclosure also provides Compound Nos. 99-126 with the general formula below:

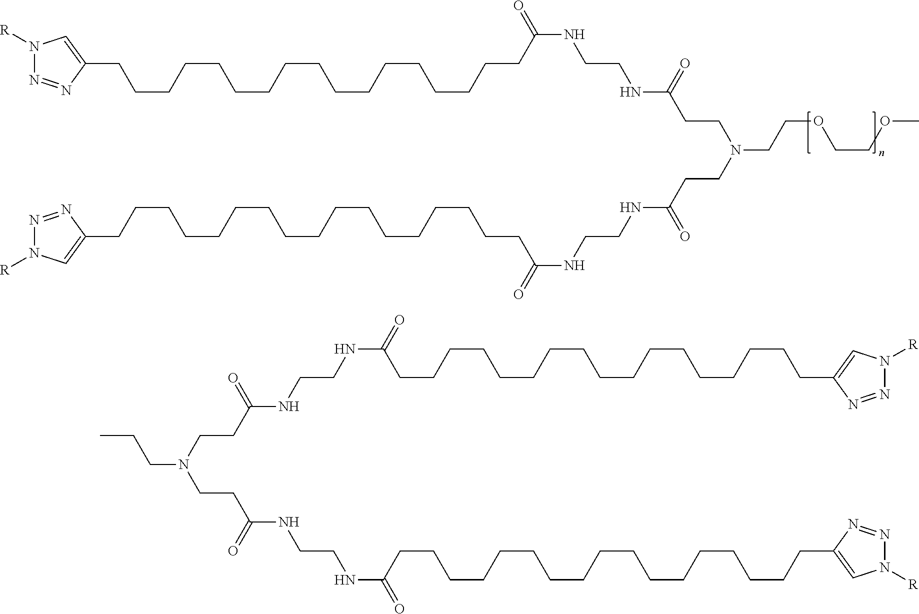

| Compound No. | n | R |
|---|---|---|
| 99 100 101 102 | 1 2 3 4 | 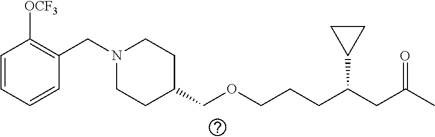 |
| 103 104 105 106 | 1 2 3 4 | 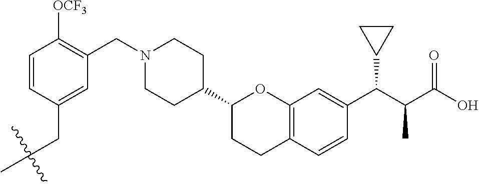 [SMILES: *Cc1ccc(OC(F)(F)F)c(CN2CCC([C@H]3CCc4ccc([C@H](C5CC5)[C@H](C)C(=O)O)cc4O3)CC2)c1] |
| 107 108 109 110 | 1 2 3 4 | 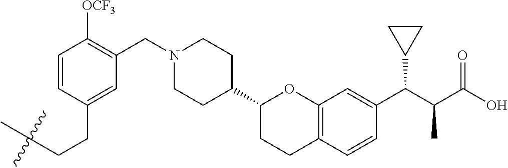 [SMILES: *CCc1ccc(OC(F)(F)F)c(CN2CCC([C@H]3CCc4ccc([C@H](C5CC5)[C@H](C)C(=O)O)cc4O3)CC2)c1] |
| 111 112 113 114 | 1 2 3 4 | 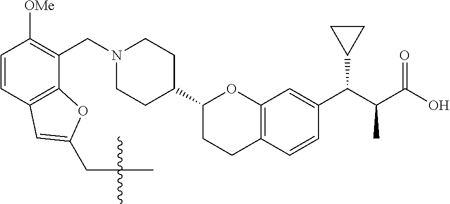 [SMILES: *Cc1cc2ccc(OC)c(CN3CCC([C@H]4CCc5ccc([C@H](C6CC6)[C@H](C)C(=O)O)cc5O4)CC3)c2o1] |
| 115 116 117 118 | 1 2 3 4 | 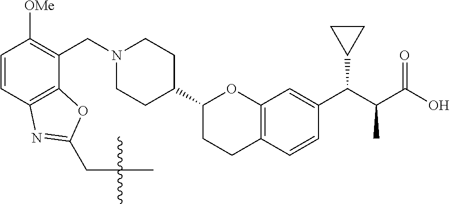 [SMILES: *Cc1nc2ccc(OC)c(CN3CCC([C@H]4CCc5ccc([C@H](C6CC6)[C@H](C)C(=O)O)cc5O4)CC3)c2o1] |
| 119 120 121 122 | 1 2 3 4 | 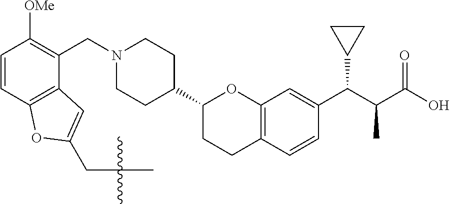 [SMILES: *Cc1cc2c(CN3CCC([C@H]4CCc5ccc([C@H](C6CC6)[C@H](C)C(=O)O)cc5O4)CC3)c(OC)ccc2o1] |
| 123 124 125 126 | 1 2 3 4 | 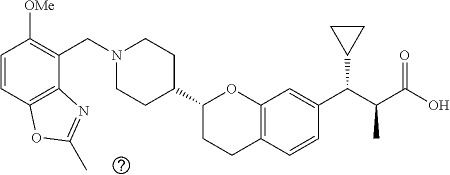 |

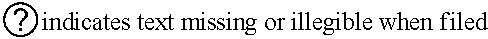

In some embodiments, the present disclosure also provides Compound Nos. 127-154 with the general formula below:

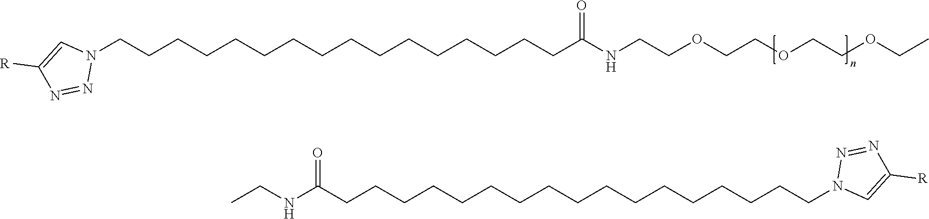

| Compound No. | n | R |
|---|---|---|
| 127 128 129 130 | 1 2 3 4 | 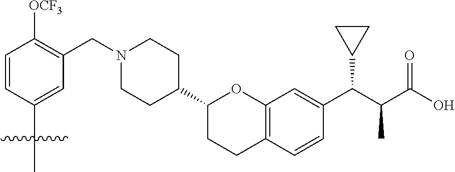 [SMILES: *c1ccc(OC(F)(F)F)c(CN2CCC([C@H]3CCc4ccc([C@H](C5CC5)[C@H](C)C(=O)O)cc4O3)CC2)c1] |
| 131 132 133 134 | 1 2 3 4 | 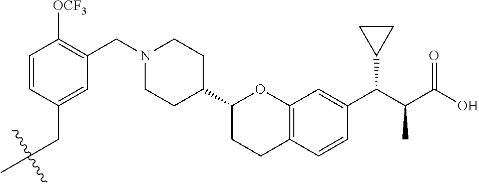 [SMILES: *Cc1ccc(OC(F)(F)F)c(CN2CCC([C@H]3CCc4ccc([C@H](C5CC5)[C@H](C)C(=O)O)cc4O3)CC2)c1] |
| 135 136 137 138 | 1 2 3 4 | 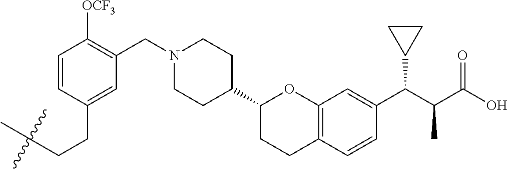 [SMILES: *CCc1ccc(OC(F)(F)F)c(CN2CCC([C@H]3CCc4ccc([C@H](C5CC5)[C@H](C)C(=O)O)cc4O3)CC2)c1] |
| 139 140 141 142 | 1 2 3 4 | 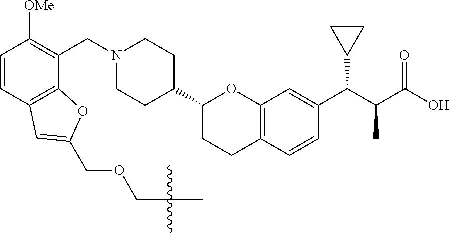 [SMILES: *COCc1cc2ccc(OC)c(CN3CCC([C@H]4CCc5ccc([C@H](C6CC6)[C@H](C)C(=O)O)cc5O4)CC3)c2o1] |
| 143 144 145 146 | 1 2 3 4 | 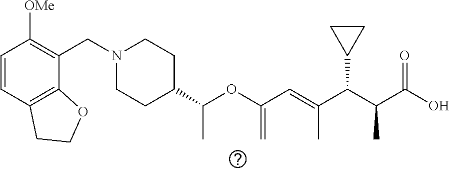 |
| 147 148 149 150 | 1 2 3 4 | 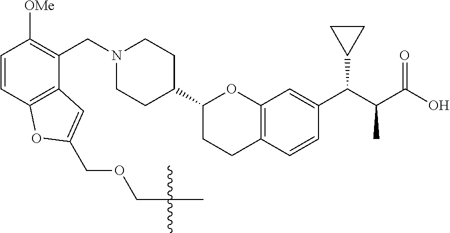 [SMILES: *COCc1cc2c(CN3CCC([C@H]4CCc5ccc([C@H](C6CC6)[C@H](C)C(=O)O)cc5O4)CC3)c(OC)ccc2o1] |
| 151 152 153 154 | 1 2 3 4 | 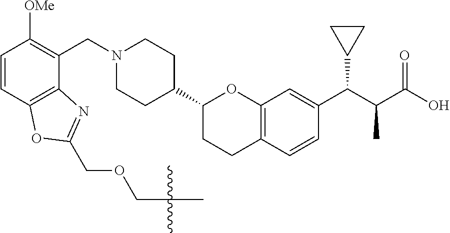 [SMILES: *COCc1nc2c(CN3CCC([C@H]4CCc5ccc([C@H](C6CC6)[C@H](C)C(=O)O)cc5O4)CC3)c(OC)ccc2o1] |

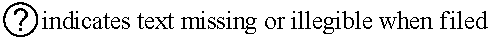

In some embodiments, the present disclosure also provides Compound Nos. 155-182 with the general formula below:

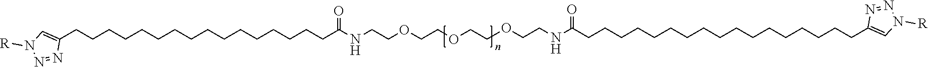

| Compound No. | n | R |
|---|---|---|
| 155 156 157 158 | 1 2 3 4 | 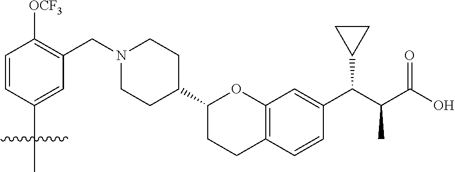 [SMILES: *c1ccc(OC(F)(F)F)c(CN2CCC([C@H]3CCc4ccc([C@H](C5CC5)[C@H](C)C(=O)O)cc4O3)CC2)c1] |
| 159 160 161 162 | 1 2 3 4 | 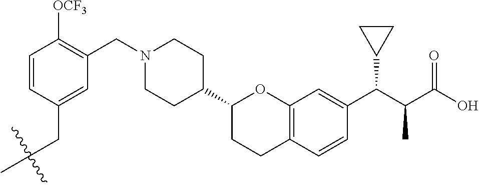 [SMILES: *Cc1ccc(OC(F)(F)F)c(CN2CCC([C@H]3CCc4ccc([C@H](C5CC5)[C@H](C)C(=O)O)cc4O3)CC2)c1] |
| 163 164 165 166 | 1 2 3 4 | 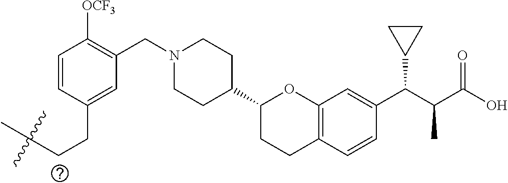 [SMILES: *CCc1ccc(OC(F)(F)F)c(CN2CCC([C@H]3CCc4ccc([C@H](C5CC5)[C@H](C)C(=O)O)cc4O3)CC2)c1] |
| 167 168 169 170 | 1 2 3 4 | 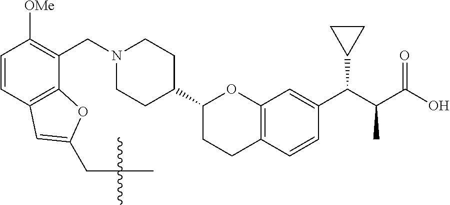 [SMILES: *Cc1cc2ccc(OC)c(CN3CCC([C@H]4CCc5ccc([C@H](C6CC6)[C@H](C)C(=O)O)cc5O4)CC3)c2o1] |
| 171 172 173 174 | 1 2 3 4 | 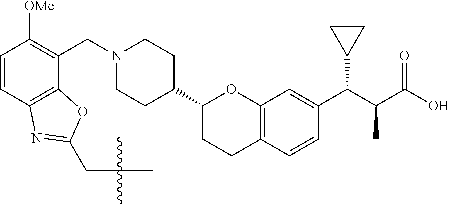 [SMILES: *Cc1nc2ccc(OC)c(CN3CCC([C@H]4CCc5ccc([C@H](C6CC6)[C@H](C)C(=O)O)cc5O4)CC3)c2o1] |
| 175 176 177 178 | 1 2 3 4 | 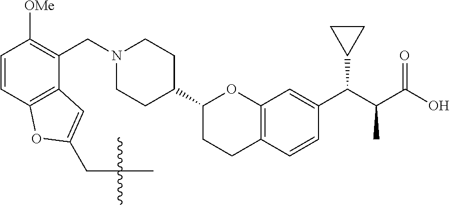 [SMILES: *Cc1cc2c(CN3CCC([C@H]4CCc5ccc([C@H](C6CC6)[C@H](C)C(=O)O)cc5O4)CC3)c(OC)ccc2o1] |
| 179 180 181 182 | 1 2 3 4 | 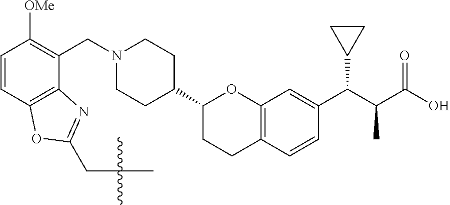 [SMILES: *Cc1nc2c(CN3CCC([C@H]4CCc5ccc([C@H](C6CC6)[C@H](C)C(=O)O)cc5O4)CC3)c(OC)ccc2o1] |

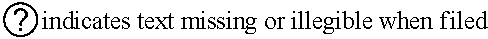

In some embodiments, the present disclosure also provides Compounds 183-237:

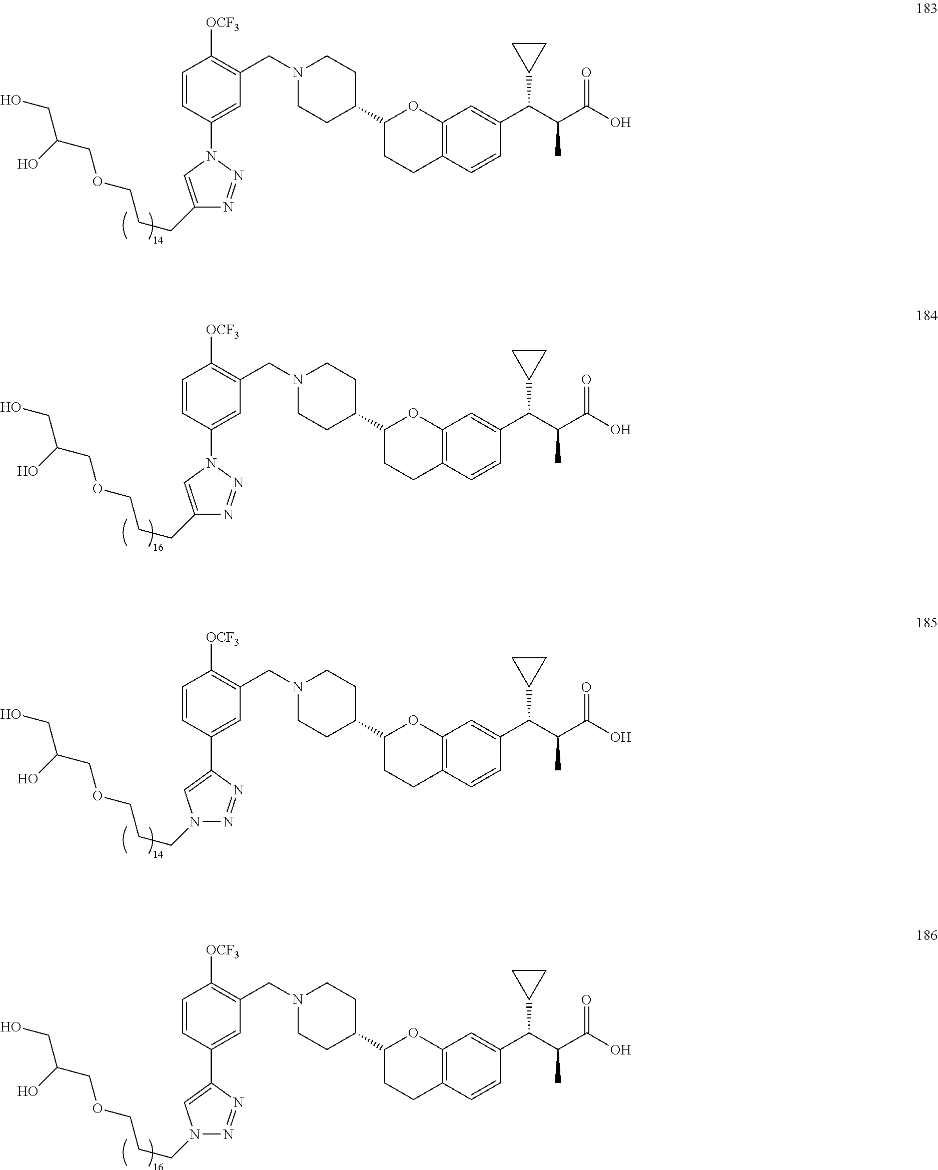

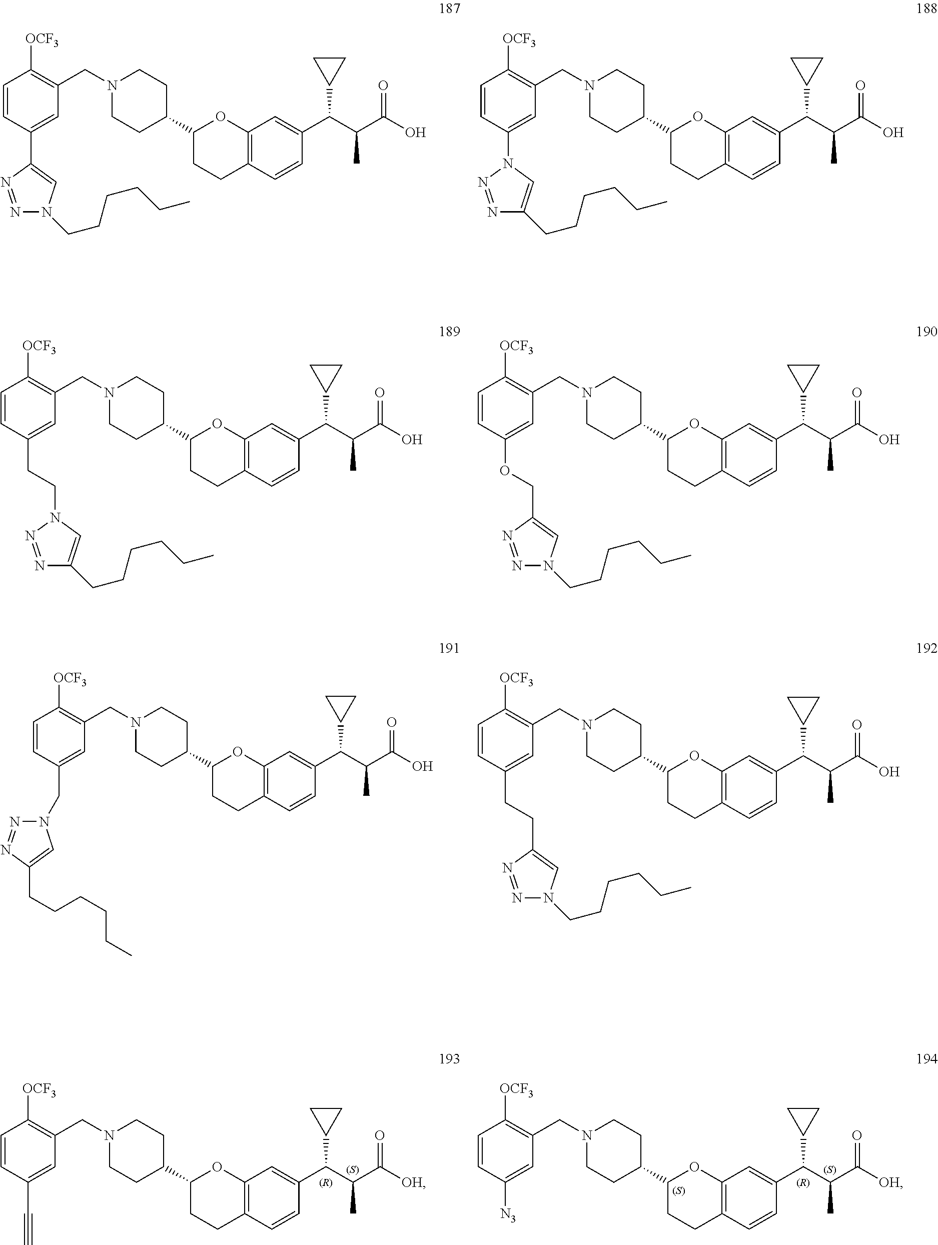

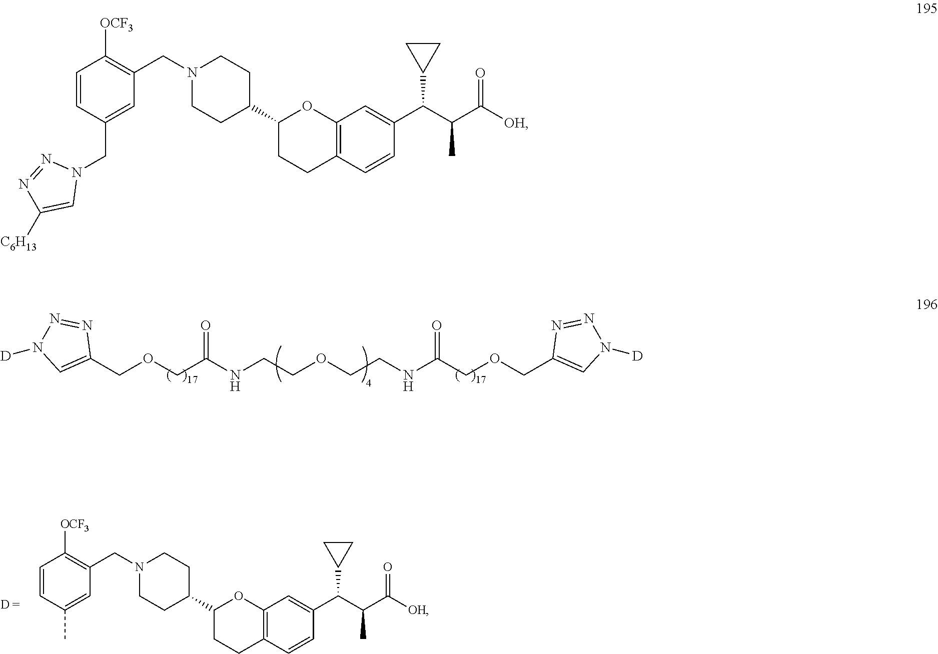

D indicates the attaching point/bond, same for compound Nos. 197-200),

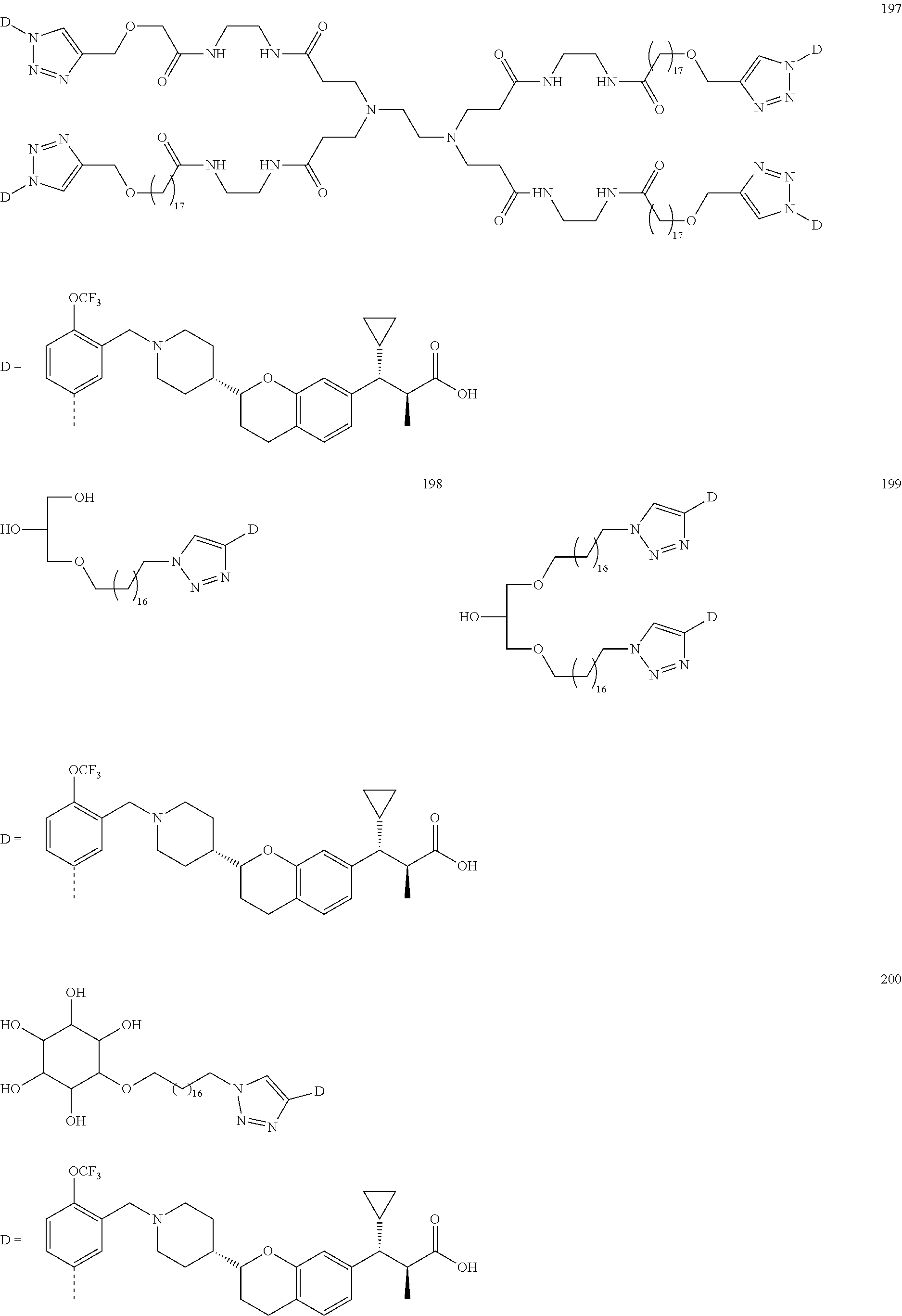

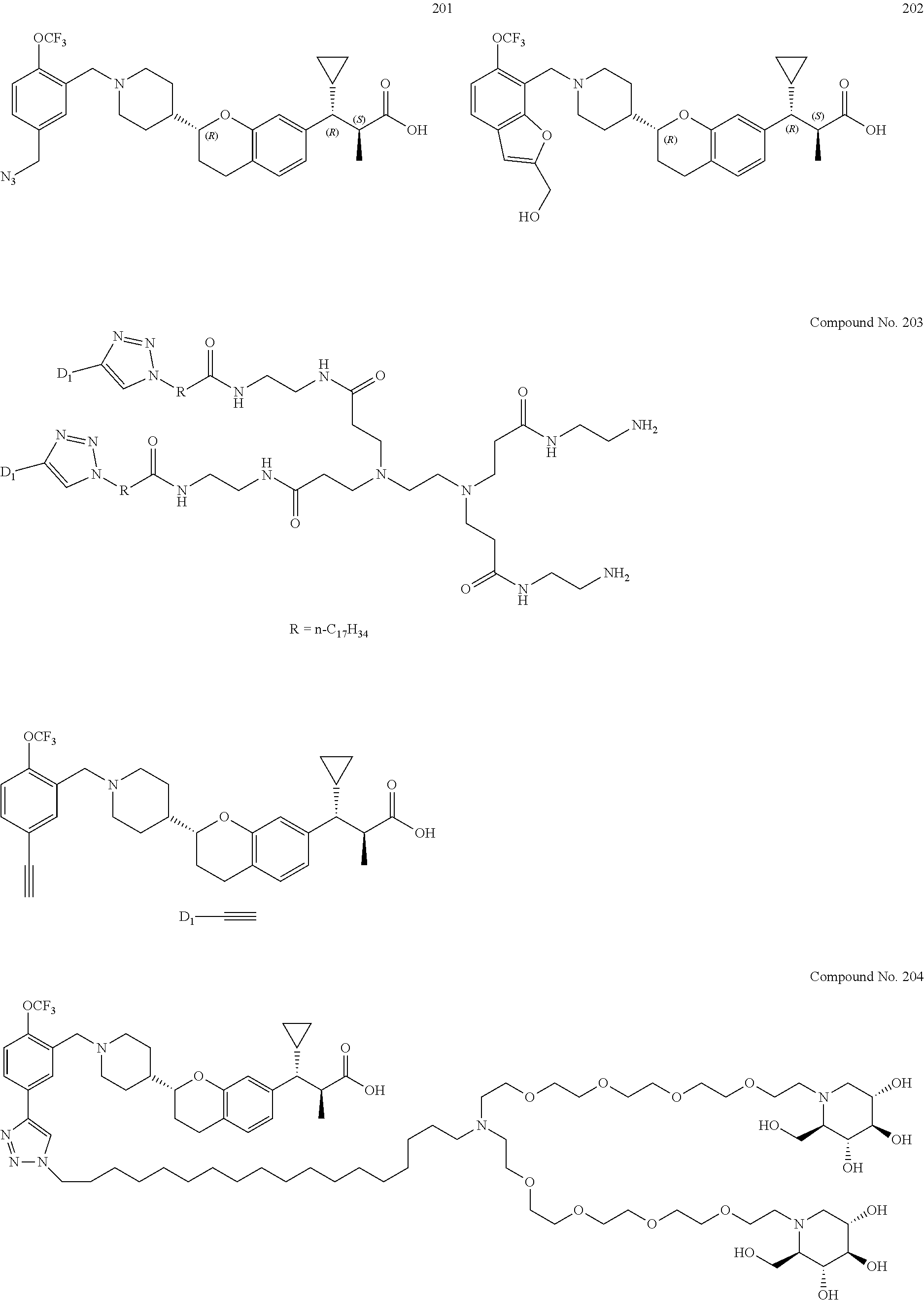

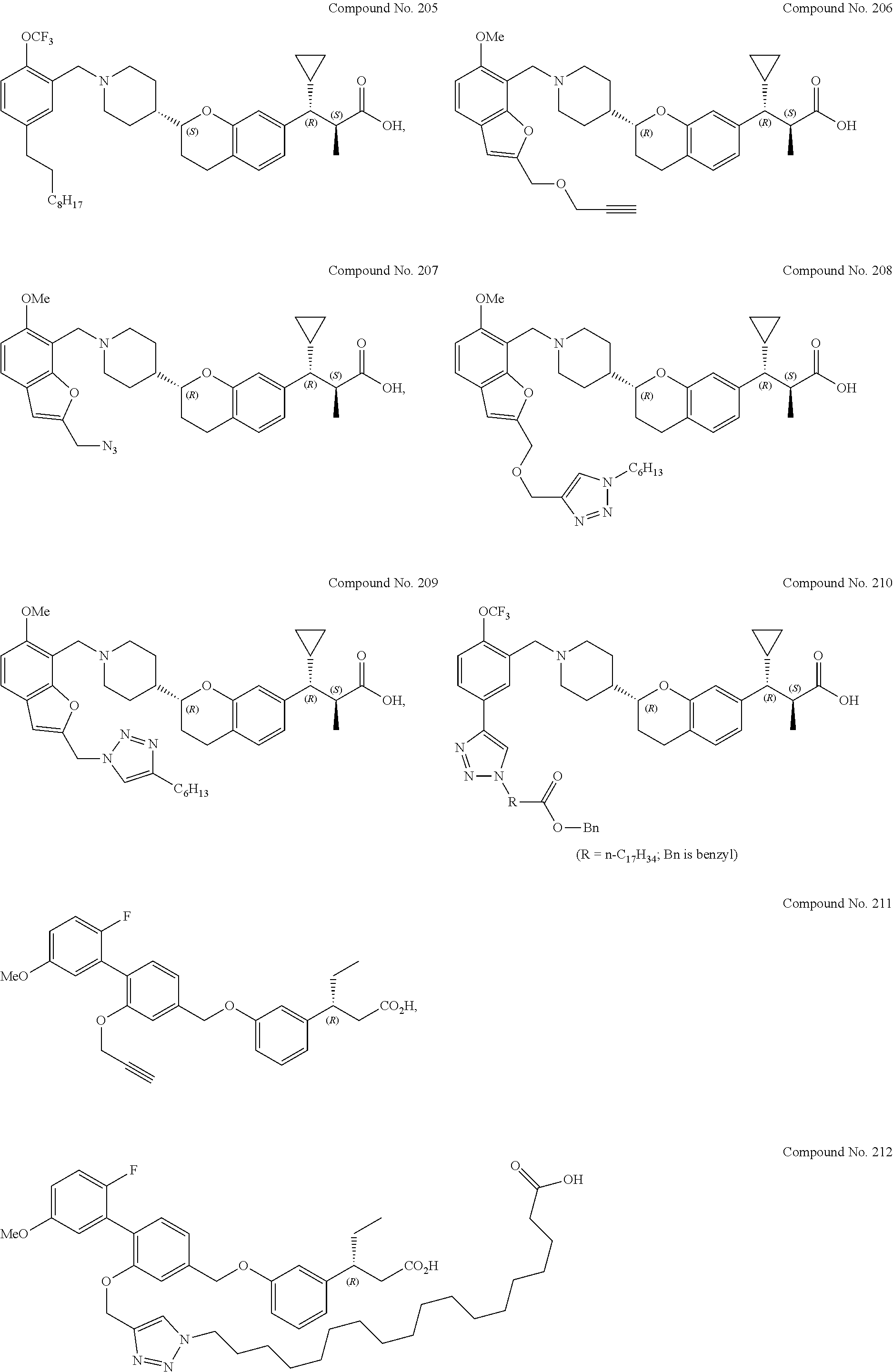

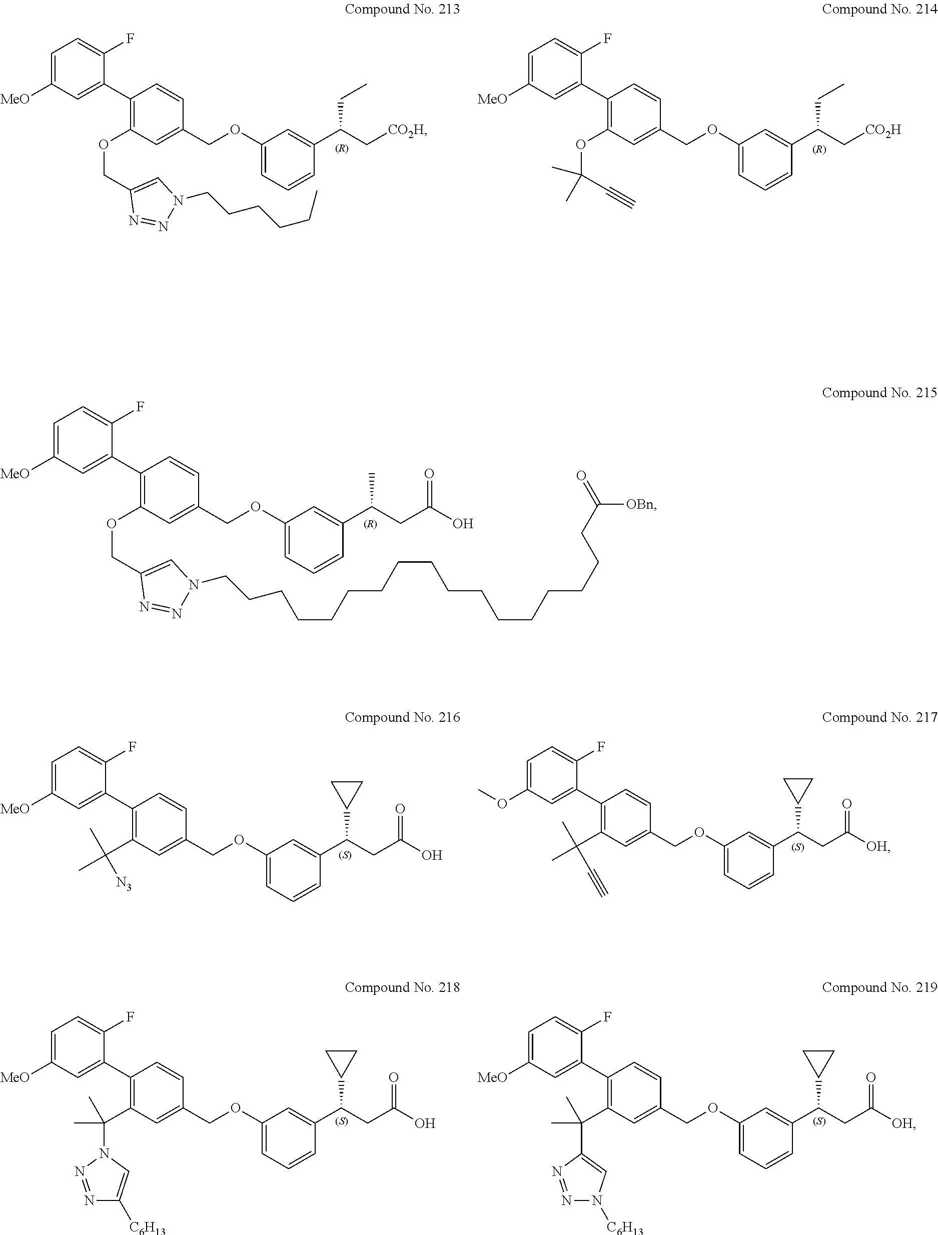

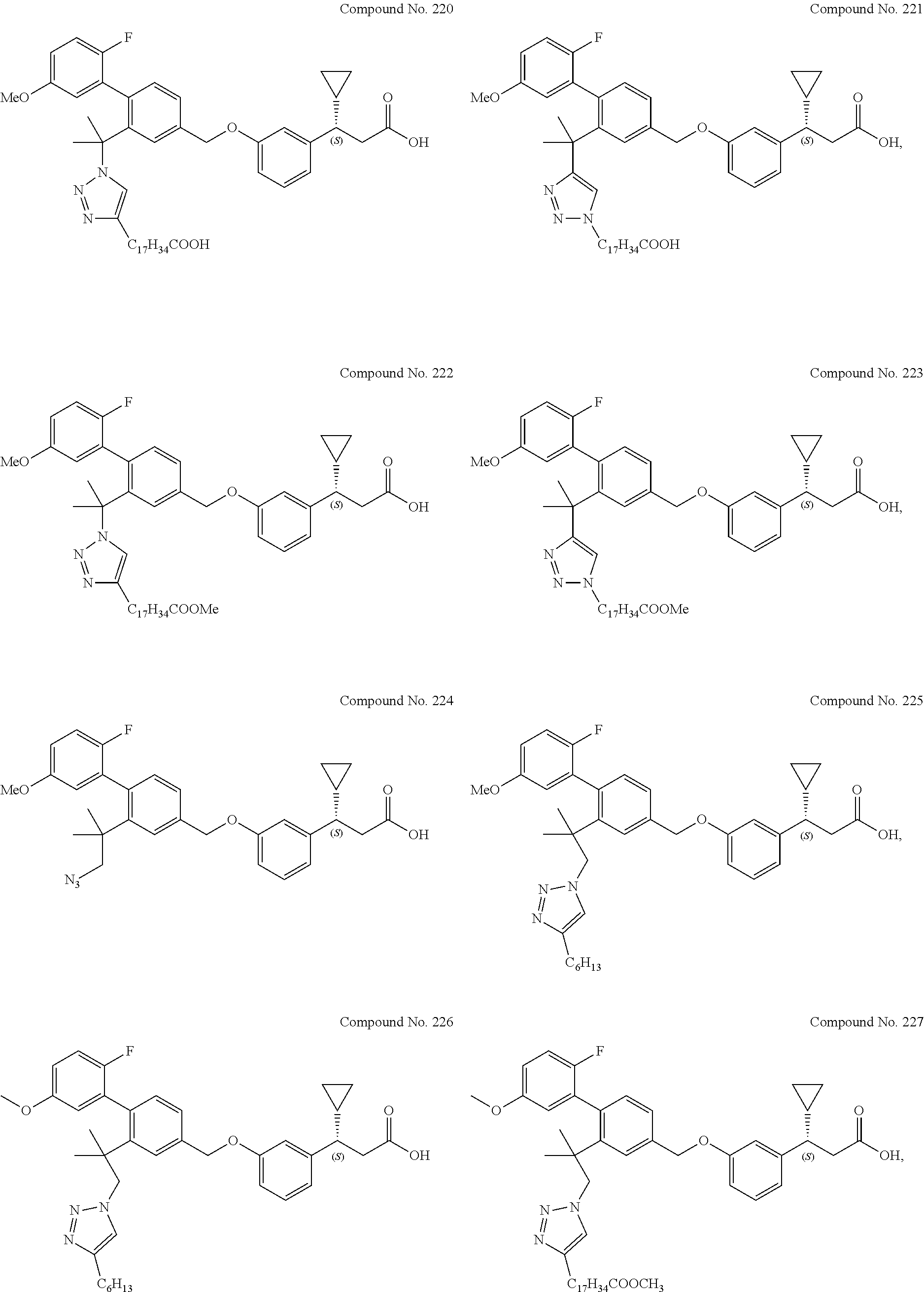

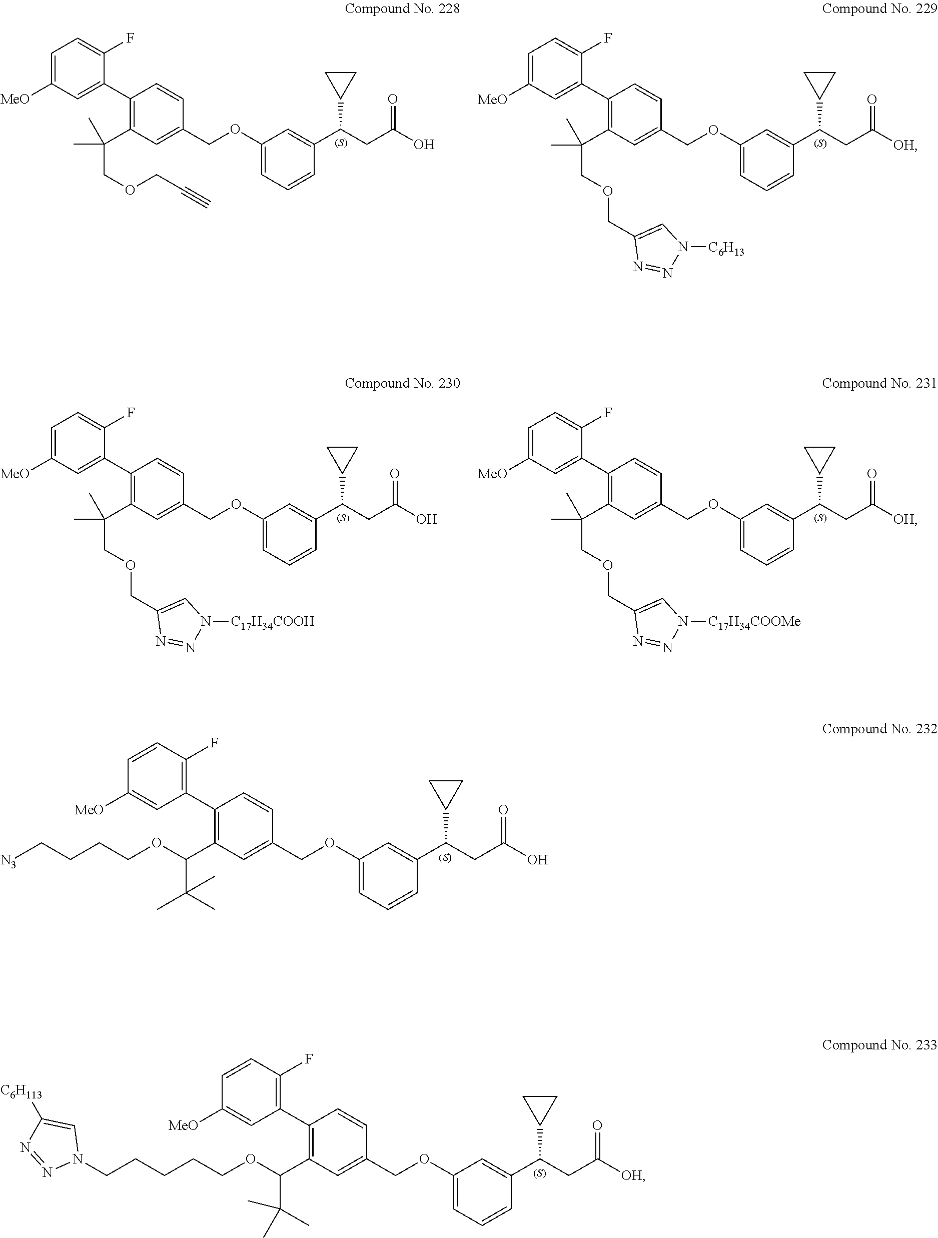

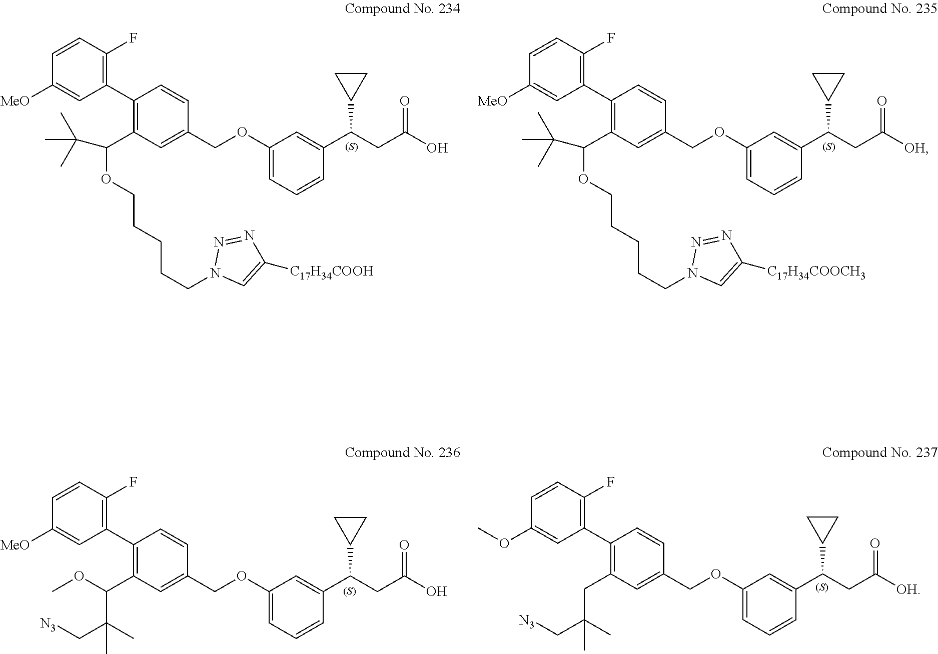

In some embodiments, the compound of any one of Compound Nos. 1-237 can be present in a form of a pharmaceutically acceptable salt or ester.

The compounds herein can be prepared by those skilled in the art in view of the present disclosure. Generally, for the synthesis of compounds of Formula I, the GPR40 agonist residues can be linked to a carrier through a variety of different chemical coupling reactions, such as amide formation, click chemistry, etc. The following scheme (Scheme 1) shows an exemplary synthetic process, which can be adapted for the preparation of other compounds described herein. For example, in some embodiments, the compounds of Formula I can be prepared from S-1 and S-2 to form the L² linkage. Suitable G¹ and G² for forming the L² linkage are not particularly limited. For example, in some embodiments, G¹ in S-1 can be an acetylene,

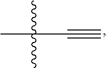 [SMILES: *C#C]

and G² in S-2 can be an azide (—N₃), or G² in S-2 can be an acetylene,

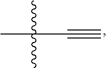 [SMILES: *C#C]

and G¹ in S-1 can be an azide (—N₃), and S-1 and S-2 can react under click chemistry conditions to yield a L² of

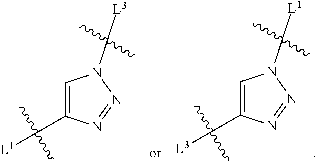

Other coupling partners, such as those forming an amide, an ether, an amine, etc., can also be used to yield different L² linkage in Formula I. Compounds of S-1 and S-2 can be readily prepared by those skilled in the art in view of the present disclosure, which shows some examples of the compounds of S-1 and/or S-2. An example of compound of S-1 is shown in Example 3, see compound No. 201. While Scheme 1 shows the formation of the L² linkage in Formula I, those skilled in the art would know that similar strategies can also be used for the synthesis of compounds of Formula I by forming the L¹ or L³ linkage. The variables D, L¹, L², L³, Q, and n in Scheme 1 include any of those described herein in any combination. It should be noted that the compound of S-1 or S-2 is also novel compounds/intermediates. For example, in some embodiments, the present disclosure also provide a compound of S-1, or a salt or ester thereof, wherein D, L³ and G¹ include any of those described herein in any combination. Typically, L³ can be a bond, optionally substituted C₁₋₁₀ alkylene, optionally substituted C₁₋₁₀ heteroalkylene having 1-5 heteroatoms independently selected from O and N, e.g., —CH₂—O—, —CH₂—O—CH₂—. G¹ typically can be

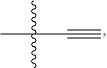 [SMILES: *C#C]

or an azide (—N₃). In some embodiments, G¹ can also be OH. D can be any of those described herein.

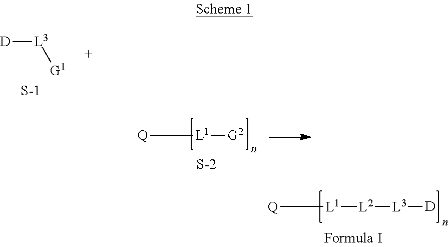

Additional synthetic examples are shown in the Examples section, see also Schemes 2-50.

In some embodiments, the present disclosure provides a method of preparing a conjugate of a GPR40 agonist. The method typically comprises reacting a suitably derivatized/functionalized GPR40 agonist with a hydrophilic molecule to form one or more covalent bonds to form the conjugate. For example, in some embodiments, the method comprises providing a compound according to any one of Formula III-1 to III-9 herein, and reacting the compound with a hydrophilic molecule having one or more functional groups that are suitable to form one or more covalent bonds with the compound of any one of Formula III-1 to III-9 to form the conjugate. For example, in some embodiments, the compound according to any one of Formula III-1 to III-9 can have one or more azido (N₃) groups, for example, when T² is -T^{C}-G, and G is N₃, and the hydrophilic molecule can have one or more acetylene groups, and the method can comprise coupling the compound according to any one of Formula III-1 to III-9 with the hydrophilic molecule to form one or more triazole rings under click-chemistry conditions. Alternatively, in some embodiments, the compound according to any one of Formula III-1 to III-9 can have one or more acetylene groups, for example, when T² is -T^{C}-G, and G is acetylene, and the hydrophilic molecule can have one or more azido (N₃) groups, and the method can comprise coupling the compound according to any one of Formula III-1 to III-9 with the hydrophilic molecule to form one or more triazole rings under click-chemistry conditions. Click-chemistry is well known in the art and suitable reaction conditions include those known in the art and those exemplified herein. In some embodiments, the compound according to any one of Formula III-1 to III-9 can have one or more carboxylic acid groups, for example, T² is a moiety having T^{A1}, wherein T^{A1} is COOH, and the hydrophilic molecule can have one or more functional groups that can react with T^{A1} to form an amide bond to form the conjugate. In some embodiments, the compound according to any one of Formula III-1 to III-9 can have one or more amino groups, for example, T² is a moiety having T^{A1}, wherein T^{A1} is NH₂, and the hydrophilic molecule can have one or more functional groups that can react with T^{A1} to form an amide bond to form the conjugate. Suitable hydrohylic molecules for the method are not particularly limited. Typically, in addition to the reacting functional group(s), the hydrophilic molecules also include one or more hydrophilic moieties such as an alcohol, e.g., a diol (e.g., glycol) or a polyol (e.g., glycerol, sugar alcohol, etc.), a sugar, a monosaccharide, disaccharide, or polysaccharide, an amine, an amide, an amino alcohol, an amino ether, water soluble ether, polyethylene glycol (PEG) chain, a carboxylic acid, an amino acid, a peptide, a charged group, or a group that can become charged at pH 7, or any combinations thereof.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, or I-14C), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237, or a pharmaceutically acceptable salt or ester thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., I-1, I-2, I-3, I-4, I-5, I-6, I-7, I-8, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237, or a pharmaceutically acceptable salt or ester thereof, e.g., in a therapeutically effective amount. In any of the embodiments described herein, the pharmaceutical composition can comprise a therapeutically effective amount of a compound selected from Compound Nos. 1-237, or a pharmaceutically acceptable salt or ester thereof.

In some embodiments, the pharmaceutical composition can be formulated for oral administration. Typically, the pharmaceutical composition is administered to a subject in need to deliver an effective amount of GPR40 agonist in the gastrointestinal tract with minimal or no absorption of GPR40 agonist in systemic circulation. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

Compounds of the present disclosure can be used alone, in combination with each other, or in combination with one or more additional therapeutic agents, e.g., PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; α-glucosidase inhibitors; agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (vi) acyl CoA:cholesterol acyltransferase (ACAT) inhibitors, (vii) CETP inhibitors, (viii) PCSK9 inhibitor or antibodies; (ix) apolipoproteins inhibitors; and (x) phenolic anti-oxidants; PPARα/γ dual agonists; PPARS agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists; GLP-1/GIP receptor dual agonists; GLP-1/GIP/insulin receptor triple agonists; GLP-1/GIP/glucagon receptor triple agonists; HSD-1 inhibitors; HSD-17 inhibitors; SGLT-2 inhibitors; SGLT-1/SGLT-2 inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin. These additional therapeutic agents are known in the art, some of which are exemplified in the background section. Additional example can be found in various patent literatures, for example, as described in U.S. Published Application No. 20190367495, the content of which is herein incorporated by reference.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disorder, condition or disease as described herein, such as type 2 diabetes, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of disorders, conditions or diseases that are associated with G-protein-coupled receptor 40 (“GPR40”). Accordingly, some embodiments of the present disclosure are also directed to methods of using one or more compounds of the present disclosure or pharmaceutical compositions herein for treating or preventing a disorder, condition or disease that may be responsive to the agonism of the G-protein-coupled receptor 40 (“GPR40”) in a subject in need thereof, such as for treating type 2 diabetes mellitus in a subject in need thereof.

In some embodiments, the present disclosure provides a method of treating or preventing a disorder, condition or disease that may be responsive to the agonism of the G-protein-coupled receptor 40 (“GPR40”) in a subject in need thereof. In some embodiments, the method comprises administering an effective amount of a compound of the present disclosure (e.g., a compound of Formula I (e.g., I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237, or a pharmaceutically acceptable salt or ester thereof) or an effective amount of a pharmaceutical composition described herein. In some embodiments, the disorder, condition or disease that may be responsive to agonism of GPR40 is Type 2 diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer, edema, nonalcoholic steatohepatitis (NASH), lipodystrophy, Prader Willi syndrome, and/or neurodegenerative diseases including but not limited to Alzheimer's disease, Parkinson's disease, amyotrophic lateral sclerosis.

In some embodiments, the present disclosure also provides a method of treating type 2 diabetes mellitus in a subject in need thereof. In some embodiments, the method comprises administering an effective amount of a compound of the present disclosure (e.g., a compound of Formula I (e.g., I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237, or a pharmaceutically acceptable salt or ester thereof) or an effective amount of a pharmaceutical composition described herein.

The administering in the methods herein is not limited. In some embodiments, the administering is orally.

As discussed herein, compounds of the present disclosure can be used as a monotherapy or in a combination therapy. In some embodiments according to the methods described herein, compounds of the present disclosure can be administered as the only active ingredient(s).

In some embodiments according to the methods described herein, compounds of the present disclosure can also be co-administered with an additional therapeutic agent, either concurrently or sequentially in any order, to the subject in need thereof. In some embodiments, the additional therapeutic agent can be PPAR gamma agonists and partial agonists; biguanides; protein tyrosine phosphatase-1B (PTP-1B) inhibitors; dipeptidyl peptidase IV (DPP-IV) inhibitors; insulin or an insulin mimetic; sulfonylureas; α-glucosidase inhibitors; agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (vi) acyl CoA:cholesterol acyltransferase (ACAT) inhibitors, (vii) CETP inhibitors, (viii) PCSK9 inhibitor or antibodies; (ix) apolipoproteins inhibitors; and (x) phenolic anti-oxidants; PPARα/γ dual agonists; PPARS agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists; GLP-1/GIP receptor dual agonists; GLP-1/GIP/insulin receptor triple agonists; GLP-1/GIP/glucagon receptor triple agonists; HSD-1 inhibitors; HSD-17 inhibitors; SGLT-2 inhibitors; SGLT-1/SGLT-2 inhibitors; FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable groups for in compounds of Formula I, II, III-1 to III-9, or subformula thereof, as applicable, are independently selected. The described embodiments of the present disclosure can be combined. Such combination is contemplated and within the scope of the present disclosure. For example, it is contemplated that the definition(s) of any one or more of Q, D, L¹, L², L³, and n of Formula I can be combined with the definition of any one or more of the other(s) of Q, D, L¹, L², L³, and n, as applicable, and the resulted compounds from the combination are within the scope of the present disclosure. Combinations of other variables for other Formulae should be understood similarly.

The symbol, [symbol] , whether utilized as a bond or displayed perpendicular to (or otherwise crossing) a bond, indicates the point at which the displayed moiety is attached to the remainder of the molecule. It should be noted that in some chemical drawings herein, the immediately connected group or groups are shown beyond the symbol, [symbol] to indicate connectivity, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC area, or both, or with a non-detectable amount of the other stereoisomer(s). The presence and/or amounts of stereoisomers can be determined by those skilled in the art in view of the present disclosure, including through the use of chiral HPLC.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula I (e.g., I-1, I-2, I-3, I-4, I-5, I-6, I-7, I-8, I-8-A, I-8-B, I-8-C, I-8-D, I-9A, I-9B, I-9A-P, I-9B-P, I-10A, I-10B, I-10C, I-11A, I-11B, I-11C, I-12A, I-12B, I-12C, I-13A, I-13B, I-14A, I-14B, I-14C, I-S-1, I-S-2, or I-S-3), Formula II (e.g., Formula II-1, II-1-A, II-1-A-1, II-1-A-2, II-1-A-3, II-1-A-4, or II-1-A-5), Formula III-1, III-2, III-3, III-4, III-5, III-6, III-7, III-8, or III-9, or any of Compound Nos. 1-237, isotopically labeled compound(s) thereof (such as a deuterated analog wherein at least one of the hydrogen atoms is substituted with a deuterium atom with an abundance above its natural abundance), possible regioisomers, possible stereoisomers thereof (including diastereoisomers, enantiomers, and racemic mixtures), tautomers thereof, conformational isomers thereof, pharmaceutically acceptable esters thereof, and/or possible pharmaceutically acceptable salts thereof (e.g., acid addition salt such as HCl salt or base addition salt such as Na salt). For the avoidance of doubt, Compound Nos. 1-237 or Compounds 1-237 refer to the compounds described herein labeled as integers 1, 2, 3, . . . , 237, which are shown under the section Compounds. For ease of description, synthetic starting materials or intermediates may be labeled with an integer (compound number) followed by a “-” and additional numeric values, such as 193-1, 193-2, etc., see examples for details. The labeling of such synthetic starting materials or intermediates should not be confused with the compounds labeled with an integer only. Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl which can include one to twelve carbon atoms (i.e., C₁₋₁₂ alkyl) or the number of carbon atoms designated. In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of which has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. For example, C₁₋₄ heteroalkyl include but not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, and —CH₂—CH₂—S(O)₂—CH₃, C₂ heteroalkyl such as —O—CH₂—CH₃ and C₁ heteroalkyl such as O—CH₃, etc. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring. As used herein, the term “cycloalkylene” as used by itself or as part of another group refers to a divalent radical derived from a cycloalkyl group, for example,

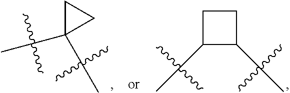 [SMILES: *C1(*)CC1 ; *C1CCC1*]

etc.

“Heterocyclyl” or “heterocyclic” as used by itself or as part of another group refers to a radical of a 3-membered or greater, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is on the heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. As used herein, the term “heterocyclylene” as used by itself or as part of another group refers to a divalent radical derived from the heterocyclyl group defined herein. For example, a piperidinylene group includes two attaching points from the piperidine ring:

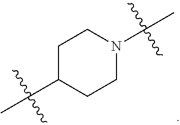 [SMILES: *C1CCN(*)CC1]

The heterocyclyl or heterocylylene can be optionally linked to the rest of the molecule through a carbon or nitrogen atom.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. As used herein, the term “arylene” as used by itself or as part of another group refers to a divalent radical derived from the aryl group defined herein. For example, a phenylene group includes two attaching points from the benzene ring, for example, 1,3-phenylene, 1,4-phenylene:

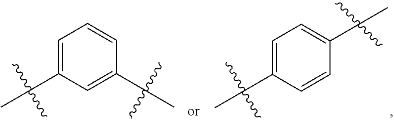 [SMILES: *c1cccc(*)c1 ; *c1ccc(*)cc1]

etc.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-14 membered monocyclic, bicyclic, or tricyclic 4n+2 aromatic ring system (e.g., having 6 or 10 pi electrons shared in a cyclic array) having ring carbon atoms and at least one, preferably, 1-4, ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein. For example, a pyridinylene group includes two attaching points from the pyridine ring, for example, 2,4-pyridinylene, 2,5-pyridinylene:

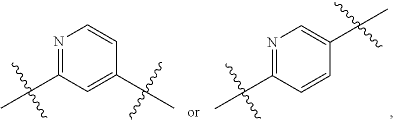 [SMILES: *c1ccnc(*)c1 ; *c1ccc(*)nc1]

etc.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N (except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(N—CN)—NH₂, —C(NH)—NH₂, —C(NH)—NH(alkyl), —N(Y₁)(Y₂), -alkylene-N(Y₁)(Y₂), C(O)N(Y₁)(Y₂) and S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O)(C₁-C₈)alkyl groups, —NHC(O)(C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₅)alkyl)₂ groups, —N((C₁-C₈)alkyl)C(O)NH((C₁-C₅)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₅)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “leaving group” is given its ordinary meaning in the art of synthetic organic chemistry and refers to an atom or a group capable of being displaced by a nucleophile. See, for example, Smith, March Advanced Organic Chemistry 6th ed. (501-502). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “pharmaceutically acceptable ester” refers to those esters which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable esters are well known in the art, for example, a C₁₋₄ alkyl ester, such as ethyl ester.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of the preferred embodiments can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra.

The abbreviations used in the Examples section should be understood as having their ordinary meanings in the art unless specifically indicated otherwise or obviously contrary from context. The following shows a list of some of the abbreviations used in the Examples section and their ordinary meanings in the art:
AIBN azobisisobutyronitrile
ACN acetonitrile
Bn benzyl
DBU 1,8-Diazabicyclo[5.4.0]undec-7-ene
DCM dichloromethane
DEAD Diethyl azodicarboxylate
DHP 3,4-dihydropyran
DIBAL-H Diisobutylaluminium hydride
DMF dimethylformamide
DMP Dess-Martin periodinane
DMSO Dimethyl sulfoxide
DPPA Diphenylphosphoryl azide
Dppf 1,1′-Bis(diphenylphosphino)ferrocene
EA or EtOAc ethyl acetate
EDCI N-(3-Dimethylaminopropyl)-N′-ethylcarbodiimide
HMDS Hexamethyldisilazane
IPA isopropyl alcohol
LAH Lithium Aliminium hydride
LDA Lithium diisopropylamide
MTBE Methyl tertiary-butyl ether
NMP N-methylpyrrolidinone
NBS N-Bromosuccinimide
NIS N-Iodosuccinimide
O/N overnight
PCC pyridinium chlorochromate
PE petroleum ether
PPTS Pyridinium p-toluenesulfonate
Rt retention time (e.g., when describing HPLC peaks)
RT room temperature (describing reaction conditions)
TBAF tetra-n-butylammonium fluoride
TBS tert-butyldimethylsilyl (or TBDMS)
TBDPS tert-butyldiphenylsilyl
TEA triethyl amine
TFA trifluoroacetic acid
THE tetrahydrofuran
THP tetrahydropyran
TMS Trimethylsilyl
TPP triphenyl phosphine
TLC thin-layer chromatography
TsOH p-Toluenesulfonic acid (or PTSA)
Z benzyloxycarbonyl (benzyl chloroformate (Z—Cl))

### Example A. Synthesis of (2S,3R)-3-cyclopropyl-2-methyl-3-((R)-2-(piperidin-4-yl)chroman-7-yl)-propanoic acid (Intermediate A)

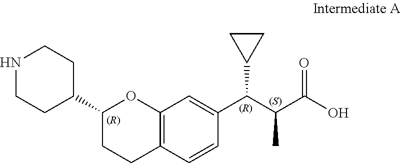

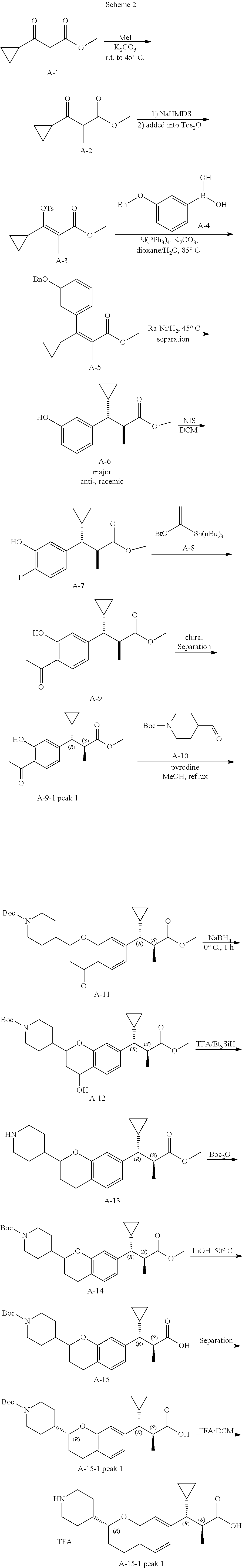

Step 1. To a mixture of K₂CO₃ (35 g, 0.253 mol) in THE (300 mL) at room temperature was added a solution of A-1 (18 g, 0.127 mol) in THE (30 mL) dropwise in 15 min. The resulting mixture was stirred for 30 min at room temperature, followed by dropwise addition of a solution of Mel (8.8 mL, 0.139 mol) in THE (20 mL) in 15 min. The resulting mixture was stirred at 40° C. for 48 hrs. The reaction mixture was filtered, and the cake was washed with EA (200 mL×2). The organic phase was combined and concentrated. The residue was dissolved with EA (250 mL) and washed with brine (100 mL×2), dried and concentrated to give crude methyl 3-cyclopropyl-2-methyl-3-oxopropanoate (A-2) as a pale-yellow oil. ¹H NMR (400 MHz, CDCl₃): δ=3.75 (s, 3H), 3.68 (q, J=7.2 Hz, 1H), 2.08-2.03 (m, 1H), 1.42 (d, J=7.2 Hz, 3H), 1.12-1.05 (m, 2H), 0.98-0.92 (m, 2H).

Step 2. To a solution of crude A-2 (10 g, 0.064 mol) in THE (100 mL) at room temperature (20° C.) was added NaHMDS (2 M, 40 mL) dropwise in 20 min. The resulting mixture, after stirring for 30 min at room temperature, was added dropwise to a solution of Tos₂O (23 g, 0.07 mol) in THE (200 mL) at room temperature in 20 min. The resulting mixture was stirred for additional 16 h at 30° C. The reaction mixture was cooled with ice-water and quenched with aq. NH₄Cl (200 mL). The water phase was extracted with EA (100 mL×2). The combined organic phase was dried over Na₂SO₄ and concentrated to give a yellow residue, which was treated with IPA (40 mL) and cooled to 4° C. (in a refrigerator). White solid was collected as methyl (Z)-3-cyclopropyl-2-methyl-3-(tosyloxy)acrylate (A-3) by filtration. MS Calcd.: 310.1; MS Found: 311.1 [M+H]⁺.

Step 3. A flask charged with A-3 (5 g, 0.0161 moL), A-4 (4.76 g, 0.0209 moL), Pd(PPh₃)₄ (920 mg, 0.0008 moL) and K₂CO₃ (4.48 g, 0.0322 moL) in dioxane (85 mL) and water (12 mL) was degassed and filled with N₂. The reaction mixture was heated at 85° C. for 16 hrs. Solvent was removed and the residue was purified by silica gel column chromatography (PE/EA=100:0 to 95:5) to give methyl (Z)-3-(3-(benzyloxy)phenyl)-3-cyclopropyl-2-methylacrylate (A-5) as a white solid. MS Calcd.: 322.2; MS Found: 323.3 [M+H]⁺. ¹H NMR (400 MHz, CDCl₃): δ=7.43-7.31 (m, 5H), 7.16 (t, J=8.0 Hz, 1H), 6.85-6.83 (m, 1H), 6.59-6.55 (m, 2H), 5.03 (s, 2H), 3.34 (s, 3H), 2.13 (s, 3H), 1.84-1.80 (m, 1H), 0.73-0.69 (m, 2H), 0.32-0.28 (m, 2H).

Step 4. A mixture of A-5 (1 g, 3.105 mmol) and Raney-Ni (˜500 mg, 50% w.t.) in MeOH (50 mL) was hydrogenation under H₂ (using a balloon) at 45° C. for 20 hrs. The mixture was filtered and the residue was purified by silica gel column chromatography (5% EA in PE) to give methyl (2S,3R)-3-cyclopropyl-3-(3-hydroxyphenyl)-2-methylpropanoate (A-6) as a white gum. MS Calcd.: 234.1; MS Found: 235.0 [M+H]⁺. ¹H NMR (400 MHz, CDCl₃): δ=7.16 (t, J=8.0 Hz, 1H), 6.73-6.63 (m, 3H), 4.82 (brs, 1H), 3.72 (s, 3H), 2.81-2.77 (m, 1H), 1.88-1.57 (m, 1H), 1.05-1.00 (m, 1H), 0.94 (d, J=7.2 Hz, 3H), 0.57-0.51 (m, 2H), 0.31-0.18 (m, 1H), 0.001-−0.003 (m, 1H).

Step 5. To a mixture of A-6 (1.45 g, 6.2 mmol) in DCM (100 mL) at room temperature was added NIS (1.68 g, 7.45 mmoL) in portions. After the addition, the resulting mixture was stirred for 14 hr at room temperature. Solvent was removed and the residue was purified by silica gel column chromatography (PE/EA=100:0 to 90:10) to give methyl (2S,3R)-3-cyclopropyl-3-(3-hydroxy-4-iodophenyl)-2-methylpropanoate (A-7) as a pale-yellow solid. MS Calcd.: 360.0; MS Found: 360.8 [M+H]⁺. H NMR (400 MHz, CDCl₃): δ=7.56 (d, J=8.0 Hz, 1H), 6.82 (s, 1H), 6.50-6.48 (m, 1H), 5.35 (brs, 1H), 3.72 (s, 3H), 2.79-2.75 (m, 1H), 1.87 (t, J=10.0 Hz, 1H), 1.02-0.99 (m, 1H), 0.94 (d, J=6.8 Hz, 3H), 0.57-0.54 (m, 1H), 0.32-0.22 (m, 2H), −0.001-−0.005 (m, 1H).

Step 6. A flask charged with A-7 (1.55 g, 4.305 mmoL), A-8 (2.02 g, 5.597 mmoL) and Pd(PPh₃)₄ (301 mg, 0.43 mmoL) in toluene (100 mL) was degassed and filled with N₂. The reaction mixture was heated at 100° C. for 16 hrs. Solvent was removed and the residue was purified by silica gel column chromatography (PE/EA=100:0 to 90:10) to give methyl (2S,3R)-3-(4-acetyl-3-hydroxyphenyl)-3-cyclopropyl-2-methylpropanoate (A-9) as a brown gum. MS Calcd.: 276.1; MS Found: 277.1 [M+H]⁺. H NMR (400 MHz, CDCl₃): δ=12.29 (s, 1H), 7.68 (d, J=8.4 Hz, 1H), 6.79 (s, 1H), 6.72-6.70 (m, 1H), 3.73 (s, 3H), 2.85-2.80 (m, 1H), 2.60 (s, 3H), 1.94 (t, J=9.6 Hz, 1H), 1.05-1.02 (m, 1H), 0.96 (d, J=6.8 Hz, 3H), 0.60-0.58 (m, 1H), 0.35-0.24 (m, 2H), 0.01-−0.006 (m, 1H).

Step 7. Product A-9-1 was obtained by chiral separation (Column: ChiralPak IA from Daicel, mobile phase: ACN:IPA, 90%:10%) as peak 1, Rt=3.7 min; The other isomer 9-2 was obtained as peak 2, Rt=7.6 min.

Step 8. To a stirred mixture of A-9-1 (250 mg, 0.9 mmol) in MeOH (15 mL) was added A-10 (230 mg, 1.08 mmol) and pyrrolidine (96 mg, 1.35 mmol). The resulting mixture was then heated at 65° C. for 12 hrs. Solvent was removed and the residue was purified by silica gel column chromatography (PE/EA=100:0 to 80:20) to give tert-butyl 4-(7-((1R,2S)-1-cyclopropyl-3-methoxy-2-methyl-3-oxopropyl)-4-oxochroman-2-yl)piperidine-1-carboxylate (A-11) as a white gum. MS Calcd.: 471.2; MS Found: 494.2 [M+Na]⁺.

Step 9. To a stirred mixture of A-11 (400 mg, 0.85 mmol) in MeOH (10 mL) at 0° C. was added NaBH₄ (48 mg, 1.27 mmol) in small portions. The resulting mixture was stirred for 1 hr, allowing the temperature to slowly warm to room temperature. Solvent was removed and the residue was treated with EA (50 mL), washed with brine, dried and concentrated to give tert-butyl 4-(7-((1R,2S)-1-cyclopropyl-3-methoxy-2-methyl-3-oxopropyl)-4-hydroxychroman-2-yl)piperidine-1-carboxylate (A-12) as a yellow gum. MS Calcd.: 473.3; MS Found: 496.3 [M+Na]⁺.

Step 10. To a stirred mixture of crude A-12 (400 mg, 0.85 mmol) in DCM (8 mL) was added TFA (2 mL) and stirred for 20 min at room temperature. Et₃SiH (0.6 mL, 4.25 mmol) was added dropwise. The resulting mixture was stirred for additional 12 hrs at room temperature. Solvent was removed and the residue was basified with aq. NaHCO₃ until pH reached 8 to 9, extracted with EA (20 mL×3), dried and concentrated to give methyl (2S,3R)-3-cyclopropyl-2-methyl-3-(2-(piperidin-4-yl)chroman-7-yl)propanoate (A-13) as a yellow gum. MS Calcd.: 357.2; MS Found: 358.2 [M+H]⁺.

Step 11. To a stirred mixture of crude A-13 (400 mg, 0.85 mmol) in DCM (20 mL) and MeOH (5 mL) was added TEA (260 mg, 2.55 mmol)) and Boc₂O (280 mg, 1.27 mmol) at room temperature. The resulting mixture was stirred at room temperature for 6 hrs. Solvent was removed and the residue was purified by silica gel column chromatography (PE/EA=100:0 to 80:20) to give tert-butyl 4-(7-((1R,2S)-1-cyclopropyl-3-methoxy-2-methyl-3-oxopropyl)chroman-2-yl)piperidine-1-carboxylate (A-14) as a colorless gum. MS Calcd.: 457.3; MS Found: 480.2 [M+Na]⁺.

Step 12. A mixture of A-14 (500 mg, 1.09 mmol) and LiOH·H₂O (470 mg, 10.9 mmol) in MeOH (15 mL)/THF (15 mL)/water (15 mL) was heated at 50° C. for 48 hrs. Volatiles were removed and the aqueous layer was acidified with 1M HCl until pH reached 3 to 4, extracted with EA (30 mL×4), dried and concentrated. The residue was purified by chromatography to give (2S,3R)-3-(2-(1-(tert-butoxycarbonyl)piperidin-4-yl)chroman-7-yl)-3-cyclopropyl-2-methylpropanoic acid (A-15) as a white solid. MS Calcd.: 443.3; MS Found: 466.2 [M+Na]⁺.

Step 13. Product A-15-1 was obtained by chiral separation (Method Info: Column: ChiralpakAD-H, Mobile phase:Hex:EtOH:TFA=90:10:0.2) as peak 1, Rt=7.9 min. The other isomer A-15-2 was obtained as peak 2, Rt=9.4 min.

Step 14. To a mixture of A-15-1 (150 mg, 0.337 mmol) in DCM (6 mL) at room temperature was added TFA (1.5 mL) dropwise. The resulting mixture was stirred for 2 hrs at room temperature. Volatiles were removed in vacuum to give (2S,3R)-3-cyclopropyl-2-methyl-3-((R)-2-(piperidin-4-yl)chroman-7-yl)propanoic acid as a TFA salt (Intermediate A). MS Calcd.: 343.2; MS Found: 344.1 [M+H]⁺.]⁺. ¹H NMR (400 MHz, DMSO-d6): δ 12.17 (br, 1H); 8.76 (br, 1H); 8.40 (br, 1H); 6.97 (d, J=8.0 Hz, 1H); 6.66 (d, J=8.0 Hz, 1H); 6.53 (s, 1H); 3.83-3.79 (m, 1H); 3.38-3.33 (m, 2H); 2.92-2.89 (m, 2H); 2.78-2.62 (m, 3H); 2.06-1.95 (m, 2H); 1.88-1.83 (m, 3H); 1.66-1.24 (m, 3H); 1.09-1.03 (m, 1H); 0.82 (d, J=6.8 Hz, 3H); 0.55-0.45 (m, 1H); 0.30-0.20 (m, 2H); −0.05-0.15 (m 1H).

### Example B. Synthesis of (S)-3-cyclopropyl-3-(3-hydroxyphenyl)propanoic acid (Intermediate B)

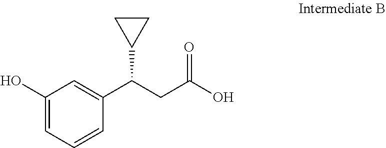 [SMILES: O=C(O)C[C@H](c1cccc(O)c1)C1CC1]

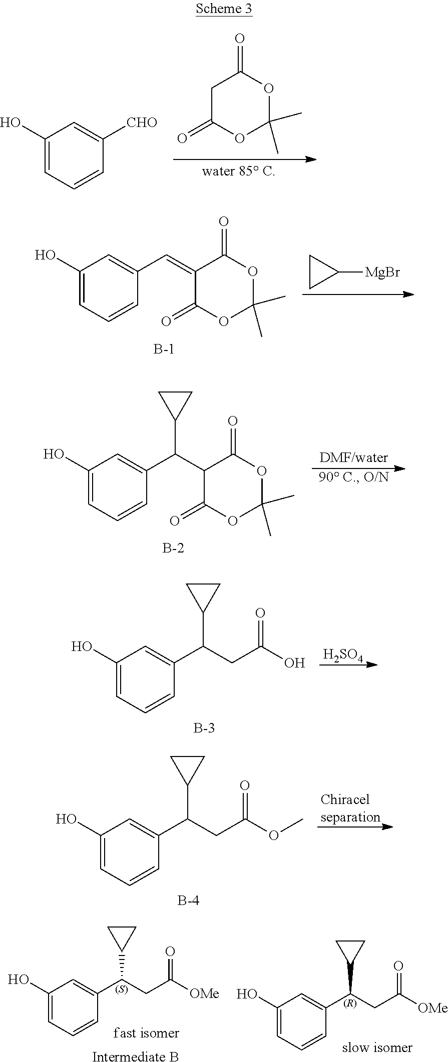

Step 1. A mixture of 3-hydroxybenzaldehyde (15.0 g, 122.95 mmol) in water (120 mL) was heated at 85° C. for 10 min until the mixture became clear. Then 2,2-dimethyl-1,3-dioxane-4,6-dione (17.7 g, 122.95 mmol) was added in 3 portions. After addition the resulting mixture was stirred at 85° C. for 1.5 h. Heating was stopped and the reaction mixture was cooled naturally with stirring. 5-(3-hydroxybenzylidene)-2,2-dimethyl-1,3-dioxane-4,6-dione, B-1, (26.3 g, 86.3%) was collected by filtration as yellow solid. ¹H NMR (400 MHz, CDCl₃) δ 9.79 (s, 1H); 8.37 (s, 1H); 7.78 (t, J=1.8 Hz, 1H); 7.48 (d, J=8.0 Hz, 1H); 7.38 (t, J=8.0 Hz, 1H); 7.09-7.07 (m, 1H); 5.73 (s, 1H); 1.80 (s, 6H).

Step 2. To a solution of B-1 (4.0 g, 16.13 mmol) in THE (60 mL) under Nitrogen was added dropwise cyclopropylmagnesium bromide (1.0 M, 80 mL, 80.65 mmol) at 0° C. The reaction mixture was warmed to RT stirred for 1.5 h. The reaction mixture was cooled to 0° C. and quenched by 1 N HCl until pH reached 5 to 6. The mixture was separated and the water phase was extracted with EA (50 ml×3). The organic layer was combined, dried by anhydrous Sodium sulfate and evaporated. The residue was purified by silica-gel column chromatography (PE/EA=5/1) to give 5-(cyclopropyl(3-hydroxyphenyl)methyl)-2,2-dimethyl-1,3-dioxane-4,6-dione, B-2, (2.0 g, 42.7%) as yellow oil. ¹H NMR (400 MHz, CDCl₃) δ: 9.30 (s, 1H); 7.18 (t, J=7.8 Hz, 1H); 6.75 (s, 1H); 6.69 (d, 1H); 6.70 (m, 1H); 4.56 (s, 1H); 2.69 (m, 1H); 1.75 (s, 3H); 1.74 (m, 1H); 1.44 (s, 3H); 0.60 (m, 2H); 0.37 (m, 1H); 0.13 (m, 1H).

Step 3. A mixture of B-2 (2.0 g, 6.90 mmol) in DMF/water (30 mL/3 mL) was heated at 90° C. for overnight. The reaction mixture was diluted with water and extracted with ethyl acetate. The organic layer was washed with brine, dried by anhydrous sodium sulfate and evaporated. The residue was purified by silica-gel column chromatography (Petroleum ether/Ethyl acetate=2/1) to give 3-cyclopropyl-3-(3-hydroxyphenyl)propanoic acid, B-3, as a yellow oil. MS (ESI) m/z=207.0 [M+H]⁺

Step 4. To a stirred solution of B-3 (1.0 g, 4.85 mmol) in MeOH (30 mL) was added H₂SO₄ (conc., 0.5 mL, 9.2 mmol) dropwise at 0° C. The resulting mixture was stirred at 70° C. for 12 h. After the completion of reaction, MeOH was removed and the mixture was diluted with water (50 mL), extracted with EA (30 mL×3) and concentrated. The residue was purified by silica-gel column chromatography (Petroleum ether/Ethyl acetate=10/1) to give methyl 3-cyclopropyl-3-(3-hydroxyphenyl)propanoate, B-4, (680 mg, 63.7%) as white solid. MS (ESI) m/z=221.0 [M+H]⁺. ¹H NMR (400 MHz, DMSO) δ 9.24 (s, 1H); 7.06 (t, J=7.8 Hz, 1H); 6.81 (d, J=7.6 Hz, 1H), 6.72-6.71 (m, 1H), 6.67-6.64 (d, 1H); 6.63 (s, 1H); 3.51 (s, 3H); 2.73-2.62 (m, 2H); 2.21-2.15 (m, 1H); 0.99-0.95 (m, 1H); 0.51-0.45 (m, 1H); 0.40-0.36 (m, 1H); 0.19-0.14 (m, 1H); 0.12-0.08 (m, 1H).

Step 5. Intermediate B was obtained by chiral separation (Column: ChiralpakOD-H, Mobile phase: Hex:EtOH=95:5, peak 1, Rt=9.5); The other enantiomer eluted out as peak 2 at 11.2 min.

### Example 1. Synthesis of Compound No. 193

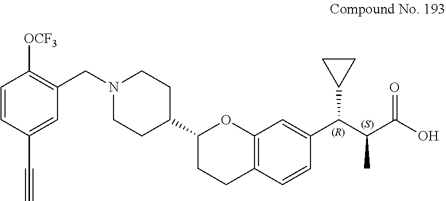

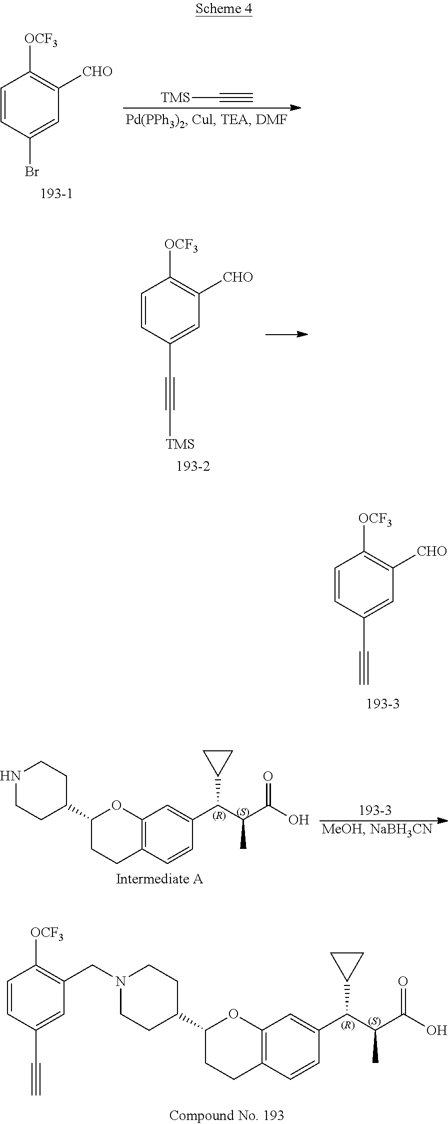

Step 1. To a solution of 5-bromo-2-(trifluoromethoxy)benzaldehyde (193-1) (1.0 g, 3.717 mmol) in DMF (20 mL) was added ethynyltrimethylsilane (730.0 mg, 7.434 mmol), CuI (141.0 mg, 0.744 mmol), TEA (1.88 g, 18.585 mmol) and Pd(PPh₃)Cl₂ (261.0 mg, 0.372 mmol) under nitrogen atmosphere. The mixture was stirred at room temperature for 16 hours. The reaction mixture was quenched with water and extracted with EA (50 mL×3). The combined organic layers were washed with water (50 mL×2) and brine (50 mL), dried over Na₂SO₄, and concentrated in vacuum (35° C.). The residue was purified by flash chromatography (PE) to give 2-(trifluoro-methoxy)-5-((trimethylsilyl)ethynyl)benzaldehyde (193-2) as a yellow oil. ¹H NMR (400 MHz, CDCl₃): δ=10.33 (s, 1H), 8.03 (d, J=2 Hz, 1H), 7.70 (dd, J=2, 9 Hz, 1H), 7.29 (d, J=9 Hz, 1H), 0.26 (s, 9H).

Step 2. To a solution of 193-2 (1.0 g, 3.493 mmol) in MeOH/H₂O (15/5 mL) was added KOH (587.0 mg, 10.479 mmol) at room temperature. The mixture was stirred at room temperature overnight. The reaction mixture was quenched with water and extracted with EA (50 mL×3). The combined organic layers were washed with water (50 mL×2), brine (50 mL), dried over Na₂SO₄ and concentrated in vacuum (35° C.). The residue was purified by flash chromatography (PE) to give 5-ethynyl-2-(trifluoromethoxy)benzaldehyde (193-3) as a yellow oil. ¹H NMR (400 MHz, CDCl₃): δ=10.34 (s, 1H), 8.06 (d, J=2 Hz, 1H), 7.74 (dd, J=2, 9 Hz, 1H), 7.29 (d, J=9 Hz, 1H), 3.17 (s, 1H).

Step 3. To a solution of Intermediate A (20.0 mg, 0.044 mmol) in MeOH (5 mL) was added compound 193-3 (28.0 mg, 0.131 mmol). The mixture was stirred at 35° C. for 6 hrs, followed by the addition of NaBH₃CN (8.3 mg, 0.131 mmol). The resulting mixture was stirred overnight. After concentration in vacuum the residue was purified by prep-HPLC (0.1% NH₄OAc as additive) to give (2S,3R)-3-cyclopropyl-3-((R)-2-(1-(5-ethynyl-2-(trifluoromethoxy)benzyl) piperidin-4-yl)-chroman-7-yl)-2-methylpropanoic acid, Compound No. 193, as a white solid. ¹H NMR (400 MHz, CDCl₃): δ=7.71 (d, J=1.6 Hz, 1H), 7.41 (dd, J=8.8, 2.0 Hz, 1H), 7.19 (dd, J=8.8, 1.2 Hz, 1H), 6.92 (d, J=7.2 Hz, 1H), 6.63-6.59 (m, 2H), 3.81-3.78 (m, 1H), 3.71-3.59 (q, 2H), 3.12 (d, 1H), 3.07 (s, 1H), 2.99 (d, 1H), 2.84-2.69 (m, 3H), 2.15 (m, 2H), 1.9 (m, 3H), 1.85-1.57 (m, 5H), 1.1 (m, 1H), 0.98 (d, J=7.2 Hz, 3H), 0.60-0.58 (m, 1H), 0.38-0.35 (m, 1H), 0.27-0.23 (m, 1H), −0.03-−0.05 (m, 1H). MS: m/z 542.3 (M+H+).

### Example 2. Synthesis of Compound No. 194

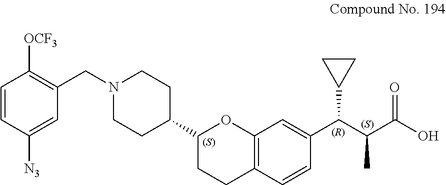

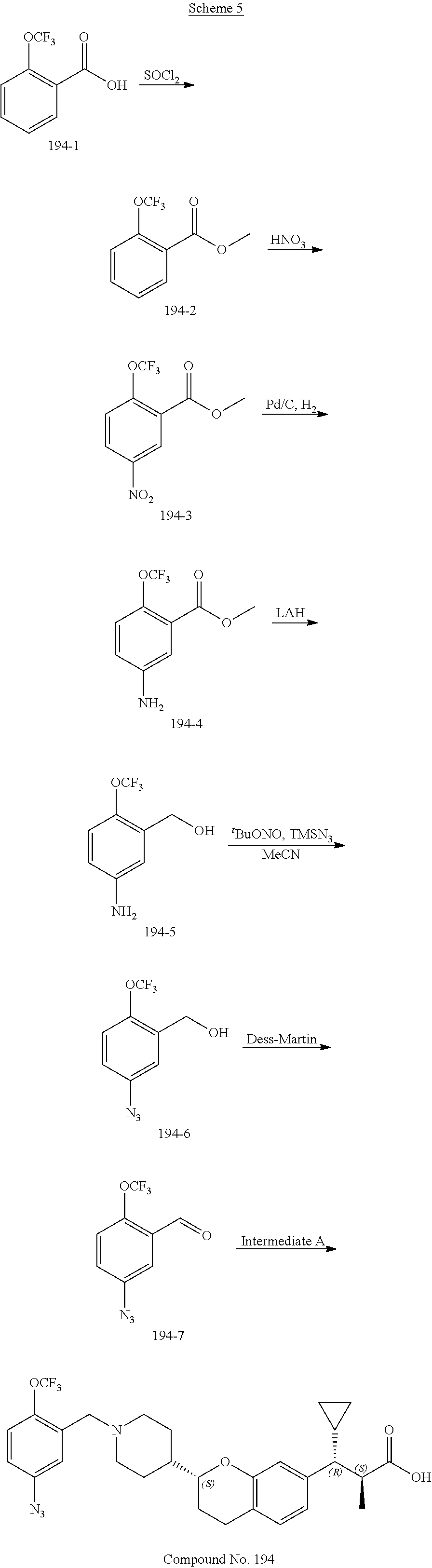

Step 1. To a solution of 2-(trifluoromethoxy)benzoic acid (194-1) (2.55 g, 12.4 mmol) in MeOH (120 mL) was added SOCl₂ (1.32 mL, 18.6 mmol) at 0° C. under nitrogen atmosphere. The mixture was heated at 65° C. overnight. The reaction mixture was evaporated to dryness (residual SOCl₂ was azeotropically removed under reduced pressure with toluene). The residue was diluted with EA (50 mL), washed with aq. NaHCO₃ (30 mL×2) and brine, dried over Na₂SO₄ and concentrated to give crude methyl 2-(trifluoromethoxy)benzoate (194-2) as a pale yellow liquid. ¹H NMR (400 MHz, CDCl₃): δ=7.95 (dd, J=7.6, 1.6 Hz, 1H), 7.55 (dt, J=8.0, 1.6 Hz, 1H), 7.37 (dt, J=7.6, 0.8 Hz, 1H), 7.33 (d, J=8.0 Hz, 1H), 3.93 (s, 3H). MS: m/z 221.0 (M+H+).

Step 2. A solution of 194-2 (2.04 g, 9.3 mmol) in concentrated H₂SO₄ (32 mL) was stirred at 0° C. for 15 min, followed with addition of HNO₃ (4.2 mL)/H₂SO₄ (15.8 mL) dropwise at 0° C. The mixture was stirred for 2 hrs. The reaction mixture was poured into 100 mL of ice water and the aqueous phase was extracted with EA (50 mL×3). The combined organic layers were washed with aq. NaOH (1 g dissolved in 50 mL of water) and brine (50 mL), dried over Na₂SO₄ and concentrated to give methyl 5-nitro-2-(trifluoromethoxy)benzoate (194-3) as an orange liquid. The crude was used for next step without further purification. ¹H NMR (400 MHz, CDCl₃): δ=8.83 (d, J=3 Hz, 1H), 8.44 (dd, J=9, 3 Hz, 1H), 7.54 (d, J=9 Hz, 1H), 4.00 (s, 3H).

Step 3. A mixture of 194-3 (3.53 g, 9.25 mmol) and Pd/C (530 mg) in MeOH (100 mL) was stirred under hydrogen balloon atmosphere at room temperature for 16 hrs. The reaction mixture was filtered over Celite and concentrated. The residue was purified by silica gel column chromatography (PE:EA=3:1) to give methyl 5-amino-2-(trifluoromethoxy)benzoate (194-4) as a pale yellow liquid. ¹H NMR (400 MHz, CDCl₃): δ=7.19 (d, J=3 Hz, 1H), 7.09 (dd, J=9, 3 Hz, 1H), 6.79 (d, J=9 Hz, 1H), 3.90 (s, 3H). MS: m/z 236.0 (M+H⁺).

Step 4. To a mixture of 194-4 (490 mg, 2.085 mmol) in dry THE (12 mL) was added LiAlH₄ (150 mg, 4.17 mmol) in small portions at 0° C. under N₂ atmosphere. After addition, the resulting mixture was stirred for 4 hrs at room temperature. The reaction mixture was quenched with water (0.15 mL), aq. NaOH (15%, 0.15 mL) and water (0.45 mL) successively. EA (50 mL) and Na₂SO₄ (˜5 g) was added into the mixture and stirred for 15 min. The mixture was then filtered and the filter cake was washed with EA (20 mL×2). The organic phase was combined and concentrated. The residue was purified by silica gel column chromatography (PE:EA=70:30) to give (5-amino-2-trifluoromethoxy-phenyl)-methanol (194-5) as a yellow oil. MS Calcd.: 207.0; MS Found: 207.9 [M+H]⁺. ¹H NMR (400 MHz, DMSO-d6): δ=6.90 (dd, J=8.8, 1.2 Hz, 1H), 6.74 (d, J=2.8 Hz, 1H), 6.45 (dd, J=8.4, 2.8 Hz, 1H), 5.26 (brs, 2H), 5.17 (t, J=5.6 Hz, 1H), 4.41 (d, J=5.6 Hz, 2H).

Step 5. To a solution of 194-5 (300 mg, 1.45 mmol) in acetonitrile (20 mL) was added tert-butyl nitrite (300 mg, 2.9 mmol) and TMSN₃ (250 mg, 2.175 mmol) successively at 0° C. under nitrogen atmosphere. After addition, the resulting mixture was allowed to warm to room temperature and stirred for 3 hrs. Volatiles were evaporated and the residue was treated with EA (50 mL), washed with water (20 mL×2), dried and concentrated to give crude (5-azido-2-trifluoromethoxy-phenyl)-methanol (194-6) as yellow solid. ¹H NMR (400 MHz, CDCl₃): δ=7.26 (s, overlap, 1H), 7.22-7.20 (m, 1H), 6.96-6.93 (m, 1H), 4.77 (s, 2H).

Step 6. To a mixture of crude 194-6 (100 mg, 0.43 mmol) in DCM (8 mL) was added DMP (273 mg, 0.64 mmol) in portions. After addition, the resulting mixture was stirred for 6 hrs at room temperature. Solvent was removed and the residue was purified by flash chromatography (PE:EA=95:5) to give 5-azido-2-trifluoromethoxy-benzaldehyde (194-7) as a yellow oil. ¹H NMR (400 MHz, CDCl₃): δ=10.33 (s, 1H), 7.62 (d, J=2.8 Hz, 1H), 7.37-7.35 (m, 1H), 7.27-7.25 (m, 1H).

Step 7. (2S,3R)-3-((S)-2-(1-(5-azido-2-(trifluoromethoxy)benzyl) piperidin-4-yl)chroman-7-yl)-3-cyclopropyl-2-methylpropanoic acid, Compound No. 194, was prepared from 194-7 and Intermediate A in the same way as Compound No. 193. MS Calcd.: 558.2; MS Found: 559.3 [M+H]⁺. H NMR (400 MHz, CDCl₃): δ=7.28 (d, 1H), 7.20 (d, 1H), 6.94-6.92 (m, 2H), 6.62-6.60 (m, 2H), 3.79-3.76 (m, 1H), 3.64-3.55 (q, 2H), 3.04-2.94 (m, 2H), 2.80-2.73 (m, 3H), 2.26-1.88 (m, 5H), 1.80-1.60 (m, 5H), 1.10-1.04 (m, 1H), 0.98 (d, J=6.4 Hz, 3H), 0.62-0.55 (m, 1H), 0.37-0.26 (m, 2H), −0.01-−0.03 (m, 1H).

### Example 3. Synthesis of Compound 201

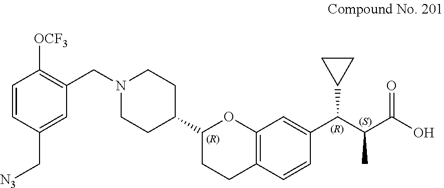

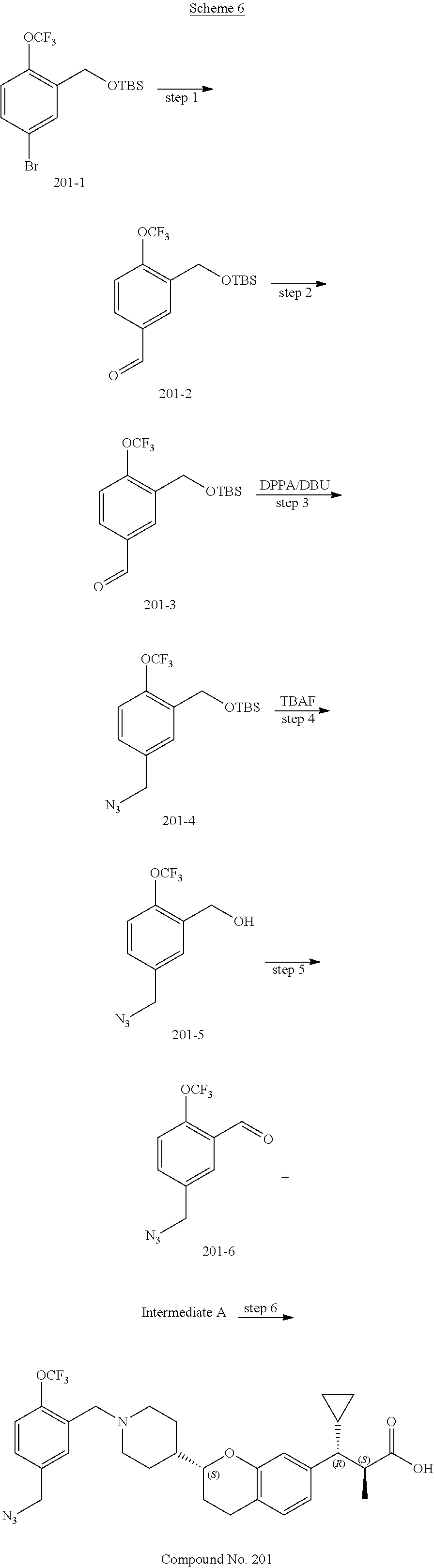

Step 1. To a solution of ((5-bromo-2-(trifluoromethoxy)benzyl)oxy)(tert-butyl)dimethylsilane (12.5 g, 0.032 mol) in THF (100 mL) was added n-BuLi (2.5M, 16 mL, 0.039 mmol) at −78° C. for 1.5 hrs, followed by the addition of DMF (2.6 g, 0.036 mmol) at −78° C. The mixture was stirred at −78° C. for 2 hrs, and quenched with saturated aqueous NH₄Cl (100 mL), and extracted with EA (100 mL×2). The organic layer was washed with brine (100 mL), dried over Na₂SO₄ and filtered. The filtrate was concentrated, and the residue was purified by silica gel column chromatography (PE/EA=30/1, v/v) to afford 3-(((tert-butyldimethylsilyl)oxy)methyl)-4-(trifluoromethoxy)benzaldehyde (201-2) as a yellow oil. ¹H NMR (400 MHz, DMSO-d₆): δ=9.93 (s, 1H), 7.99-7.98 (m, 1H), 7.89-7.87 (m, 1H), 7.49-7.47 (m, 1H), 4.70 (s, 2H), 0.80 (s, 9H), 0.01 (s, 6H).

Step 2. To a solution of 201-2 (4 g, 11.9 mmol) in MeOH (50 mL) at 0° C. was added NaBH₄ (906 mg, 23.9 mmol), the resulting mixture was stirred for 2 hrs. The reaction was quenched with saturated aqueous NH₄Cl (50 mL) and extracted with EA (50 mL×2). The organic layer was dried over Na₂SO₄, filtered and concentrated and the residue was purified by silica gel column chromatography (PE/EA=5/1, v/v) to afford (3-(((tert-butyldimethylsilyl)oxy)methyl)-4-(trifluoromethoxy)phenyl)methanol (201-3) as a colorless oil. ¹H NMR (400 MHz, DMSO-d₆): δ=7.43 (d, J=1.6 Hz, 1H), 7.26-7.23 (m, 1H), 7.20-7.18 (m, 1H), 5.20 (s, 1H), 4.64 (s, 2H), 4.43 (s, 2H), 0.81 (s, 9H), 0.01 (s, 6H).

Step 3. To a solution of 201-3 (500 mg, 1.49 mmol) in DMF (10 mL) was added DPPA (491 mg, 1.79 mmol) and DBU (270 mg, 1.79 mmol) at RT. The mixture was stirred at 90° C. for overnight. The residue was poured into water (10 mL), extracted with EA (20 mL×2). The organic layer was washed with brine (100 mL×2), dried over Na₂SO₄ and filtered. The filtrate was concentrated, and the residue was purified by silica gel column chromatography (PE) to afford ((5-(azidomethyl)-2-(trifluoromethoxy)benzyl)oxy)(tert-butyl)dimethylsilane (201-4) as a colorless oil.

Step 4. To a solution of 201-4 (170 mg, 0.47 mmol) in THE (100 mL) was added TBAF (1.0M, 1 mL, 0.94 mmol) at room temperature. The mixture was stirred for 2 h. The mixture was concentrated in vacuo to afford (5-(azidomethyl)-2-(trifluoromethoxy) phenyl)methanol (201-5) as a yellow oil.

Step 5. To a solution of 201-5 (120 mg, 0.49 mmol) in DCM (6 mL) was added DMP (412 mg, 0.97 mmol) at ice-bath and the mixture was stirred at room temperature for 2 hrs. The reaction was concentrated in vacuo. The residue was purified by silica gel column chromatography (PE/EA=20/1, v/v) to afford 5-(azidomethyl)-2-(trifluoromethoxy) benzaldehyde (201-6) as a colorless oil. ¹H NMR (400 MHz, CDCl₃): δ=10.38 (s, 1H), 7.92 (d, J=2.4 Hz, 1H), 7.65-7.62 (m, 1H), 7.41-7.38 (m, 1H), 4.45 (s, 2H).

Step 6. (2S,3R)-3-((R)-2-(1-(5-(azidomethyl)-2-(trifluoromethoxy)benzyl) piperidin-4-yl)chroman-7-yl)-3-cyclopropyl-2-methylpropanoic acid, Compound 201, was prepared from 201-6 and intermediate A in the same way as Compound No, 193. ¹H NMR (400 MHz, CD₃OD): δ=7.68 (s, 1H), 7.48 (d, J=9.2 Hz, 1H), 7.41 (d, J=8.8 Hz, 1H), 7.00 (d, J=8.0 Hz, 1H), 6.67 (d, J=7.6 Hz, 1H), 6.60 (s, 1H), 4.49 (s, 2H), 3.80 (m, 3H), 3.14 (m, 2H), 2.85-2.72 (m, 3H), 2.33-2.22 (m, 2H), 2.08-2.05 (m, 3H), 1.83-1.60 (m, 5H), 1.11-1.09 (m, 1H), 0.96-0.93 (d, 3H), 0.62-0.60 (m, 1H), 0.39-0.30 (m, 2H), 0.01-−0.02 (m, 1H).

### Example 4. Synthesis of Compound 202

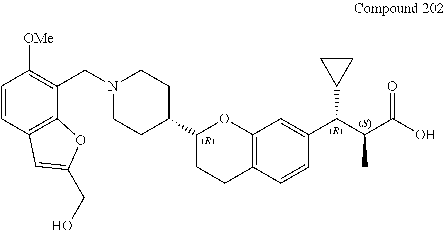

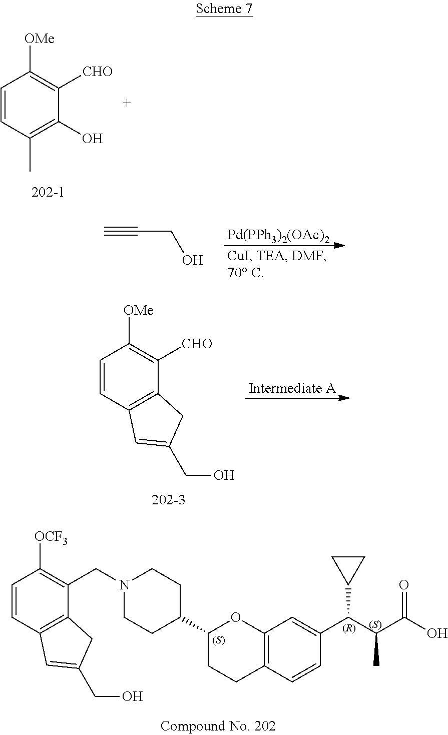

Step 1. To a solution of 2-hydroxy-3-iodo-6-methoxybenzaldehyde (300 mg, 1.07 mmol), prop-2-yn-1-ol (90.6 mg, 1.62 mmol), TEA (324 mg, 3.21 mmol) in DMF (5 mL) was added CuI (20 mg, 0.107 mmol) and Pd(PPh3)2Cl2(75 mg, 0.107 mmol) under N2. The mixture was stirred at 75° C. overnight in a sealed tube. The mixture was added H2O (30 mL) and was extracted with EA (20 mL×3). The oil layer was dried with Na2SO4 and concentrated. The crude product was purified with chromatography (PE/EA=1/1) to give 2-(hydroxymethyl)-6-methoxybenzofuran-7-carbaldehyde as a yellow solid. 1H NMR (400 MHz, DMSO-d6): δ=10.49 (s, 1H); 7.87 (d, 1H); 7.14 (d, 1H); 6.75 (s, 1H); 5.49 (s, 1H); 4.56 (s, 2H); 3.96 (s, 3H). MS: m/z 207.1 (MH+).

Step 2. Compound 202 was made from 202-3 and Intermediate A in the same way as Compound 193. MS: m/z 534.3 (MH+).

### Example 5. Synthesis of Compound No. 187

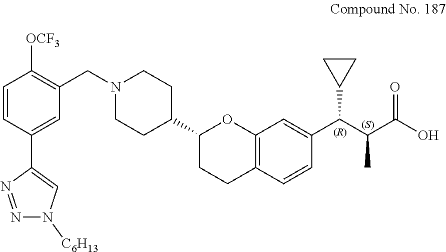

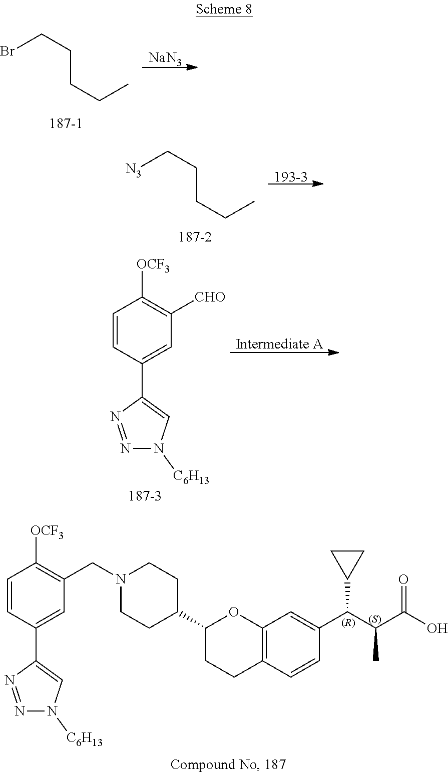

Step 1. To a solution of 1-bromohexane (100.0 mg, 0.606 mmol) in EtOH/H₂O (5/5 mL) was added NaN₃ (43.0 mg, 0.666 mmol) at room temperature. The mixture was heated to 60° C. overnight to give 1-azidohexane (187-2) as a solution of -0.06 mmol/mL. The reaction mixture was used directly for next step without further purification.

Step 2. To a solution of 193-3 (300.0 mg, 1.401 mmol) in EtOH/H₂O (10/10 mL) was added 187-2 (1.75 mL, 1.401 mmol, 0.8 M in EtOH/H₂O), L-Ascorbic acid sodium salt (or “L-AASS”, 111.0 mg, 0.560 mmol) and copper(II) sulfate pentahydrate (70.0 mg, 0.280 mmol). The mixture was stirred at room temperature overnight. The reaction mixture was quenched with water and extracted with EA (50 mL×3). The combined organic layers were washed with water (50 mL×2) and brine (50 mL), dried over Na₂SO₄ and concentrated. The residue was purified by flash (EA/PE=0-5%) to give 5-(1-hexyl-1H-1,2,3-triazol-4-yl)-2-(trifluoromethoxy)benzaldehyde (187-3) as a white solid. H NMR (400 MHz, CDCl₃): δ=10.40 (s, 1H), 8.31 (dd, J=2.8, 8.8 Hz, 1H), 8.24 (d, J=2.4 Hz, 1H), 7.86 (s, 1H), 7.44 (dd, J=1.6, 8.4 Hz, 1H), 4.42 (t, J=7.2 Hz, 2H), 1.96 (m, 2H), 1.39-1.30 (m, 6H), 0.89 (t, J=7.2 Hz, 3H). MS: m/z 342.1 (M+H⁺).

Step 3. (2S,3R)-3-cyclopropyl-3-((R)-2-(1-(5-(1-hexyl-1H-1,2,3-triazol-4-yl)-2-(trifluoro-methoxy)benzyl)piperidin-4-yl)chroman-7-yl)-2-methylpropanoic acid, Compound No. 187, was prepared from Intermediate A and 187-3 in the same way as Compound No. 193. ¹H NMR (400 MHz, CDCl₃): δ=7.99 (d, J=1.6 Hz, 1H), 7.85 (dd, 1H), 7.79 (s, 1H), 7.30 (dd, 1H), 6.91 (d, 1H), 6.61 (s, 1H), 6.60 (d, 1H), 4.38 (t, 2H), 3.79-3.68 (m, 3H), 3.17 (d, 1H), 3.05 (d, 1H), 2.81-2.72 (m, 3H), 2.24-1.59 (m, 12H), 1.36-1.20 (m, 6H), 1.10-1.03 (m, 1H), 0.95 (d, 3H), 0.89-0.85 (m, 3H), 0.59-0.54 (m, 1H), 0.38-0.32 (m, 1H), 0.28-0.21 (m, 1H), −0.02-−0.06 (m, 1H). MS: m/z 669.4 (M+H⁺).

### Example 6. Synthesis of Compound No. 188

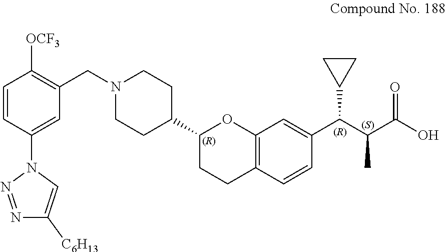

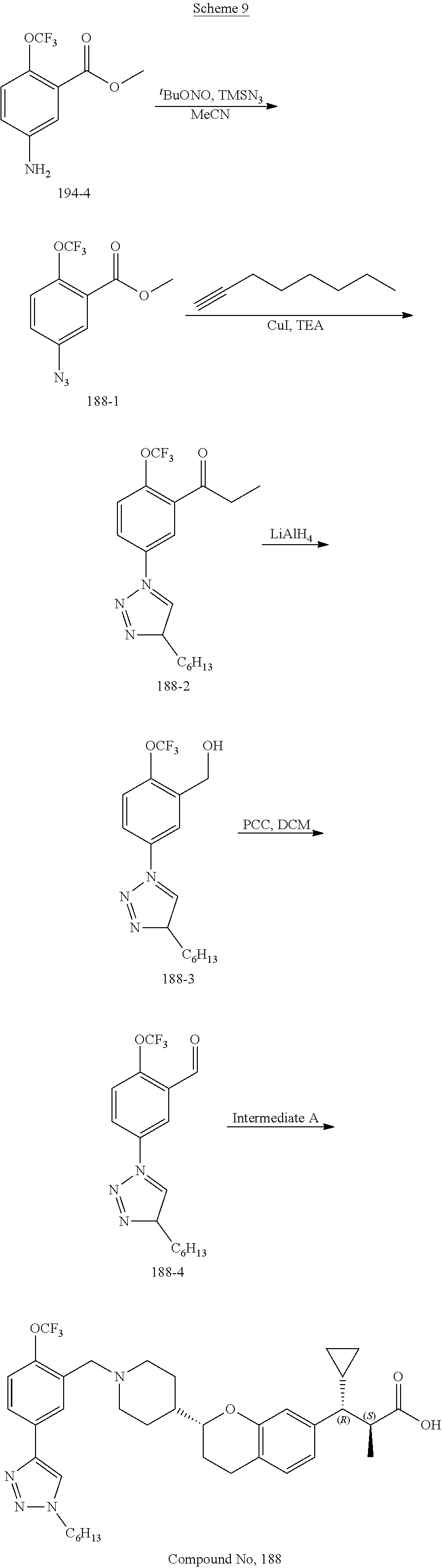

Step 1. To a solution of methyl 5-amino-2-(trifluoromethoxy)benzoate (1.94 g, 7.1 mmol) in acetonitrile (100 mL) was added tert-butyl nitrite (1100 mg, 10.7 mmol) and TMSN₃ (990 mg, 8.6 mmol) successively at 0° C. under nitrogen atmosphere. After addition, the resulting mixture was allowed to warm to room temperature and stirred for 1 hr. The crude methyl 5-azido-2-(trifluoromethoxy)benzoate (188-1) solution (0.07 M in MeCN) was obtained which was directly used for the next step.

Step 2. To a solution of crude 188-1 (1.85 g, 7.1 mmol) in acetonitrile (100 mL) was added oct-1-yne (780 mg, 7.1 mmol), CuI (270 mg, 1.42 mmol) and Et₃N (0.72 mL, 8.52 mmol) at room temperature under nitrogen atmosphere and the mixture was stirred overnight. The reaction mixture was evaporated to dryness. The residue was purified by flash chromatography (PE:EA=5:1) to give methyl 5-(4-hexyl-1H-1,2,3-triazol-1-yl)-2-(trifluoromethoxy)benzoate (188-2) as a tan solid. ¹H NMR (400 MHz, CDCl₃): δ=8.27 (d, J=2.8 Hz, 1H), 8.04 (dd, J=8.8, 2.8 Hz, 1H), 7.77 (s, 1H), 7.50 (dd, J=8.8, 0.8 Hz, 1H), 3.98 (s, 3H), 2.81 (t, J=8.0 Hz, 2H), 1.78-1.70 (m, 2H), 1.43-1.26 (m, 6H), 0.90 (t, J=7.2 Hz, 3H). MS: m/z 372.0 (M+H⁺).

Step 3. To a solution of 188-2 (480 mg, 1.2 mmol) in THE (40 mL) was added LiAlH₄ (256 mg, 6.7 mmol) at 0° C. under nitrogen atmosphere. The cooling bath was removed and the reaction mixture was stirred overnight. The reaction mixture was quenched by slow addition of 0.256 mL of 15% NaOH(aq) and 0.768 mL of H₂O. The resulting white solid was filtered, and the filtrate was dried over Na₂SO₄. Solvent was removed by rotavap, and the residue was purified by silica gel column chromatography (PE:EA=5:1) to give (5-(4-hexyl-1H-1,2,3-triazol-1-yl)-2-(trifluoromethoxy) phenyl)methanol (188-3) as a white solid. ¹H NMR (400 MHz, CDCl₃): δ=7.98 (d, J=2.8 Hz, 1H), 7.74 (s, 1H), 7.69 (dd, J=8.6, 2.6 Hz, 1H), 7.36 (dd, J=8.8, 1.2 Hz, 1H), 4.88 (s, 2H), 2.89 (br, 1H), 2.78 (t, J=8.0 Hz, 2H), 1.75-1.68 (m, 2H), 1.43-1.26 (m, 6H), 0.89 (t, J=7.0 Hz, 3H). MS: m/z 344.0 (M+H⁺).

Step 4. To a solution of 188-3 (100.0 mg, 0.28 mmol) in DCM (15 mL) was added pyridinium chlorochromate (121 mg, 0.56 mmol) at 0° C. under nitrogen atmosphere. The mixture was stirred overnight. The reaction mixture was quenched with water and extracted with DCM (20 mL×3). The combined organic layers were dried over Na₂SO₄ and concentrated to dryness. The residue was purified by silica gel column chromatography (PE:EA=10:1) to give 5-(4-hexyl-1H-1,2,3-triazol-1-yl)-2-(trifluoro-methoxy)benzaldehyde (188-4) as a white solid. ¹H NMR (400 MHz, CDCl₃): δ=10.41 (s, 1H), 8.24 (dd, J=8.8, 2.8 Hz, 1H), 8.17 (d, J=2.8 Hz, 1H), 7.80 (s, 1H), 7.55 (dd, J=9.0, 1.4 Hz, 1H), 2.81 (t, J=7.6, 2H), 1.78-1.70 (m, 2H), 1.44-1.25 (m, 6H), 0.90 (t, J=7.0 Hz, 3H). MS: m/z 342.0 (M+H⁺).

Step 5. (2S,3R)-3-cyclopropyl-3-((R)-2-(1-(5-(4-hexyl-1H-1,2,3-triazol-1-yl)-2-(trifluoro-methoxy)benzyl)piperidin-4-yl)chroman-7-yl)-2-methylpropanoic acid, Compound No. 188, was prepared from Intermediate A and 188-4 in the same way as Compound No. 193. ¹H NMR (400 MHz, CDCl₃): δ=7.99 (s, 1H); 7.75-7.71 (m, 2H); 7.38 (d, 1H); 6.93 (d, 1H); 6.62-6.60 (m, 2H); 3.78 (m, 1H); 3.72-3.67 (q, 2H); 3.09 (d, 1H); 3.01 (d, 1H); 2.83-2.74 (m, 5H); 2.24-2.14 (m, 2H); 1.94-1.89 (m, 3H); 1.79-1.62 (m, 7H); 1.4 (m, 2H); 1.32-1.27 (m, 4H); 1.12-1.03 (m, 1H); 0.96 (d, 3H); 0.88 (t, 3H); 0.59-0.54 (m, 1H); 0.38-0.21 (m, 2H); −0.02-−0.06 (m, 1H). MS: m/z 669.4 (M+H⁺).

### Example 7. Synthesis of Compound No. 195

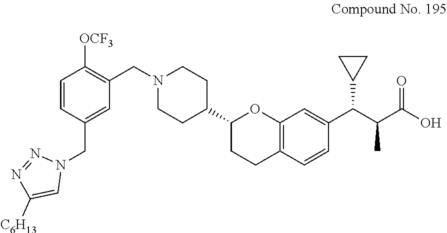

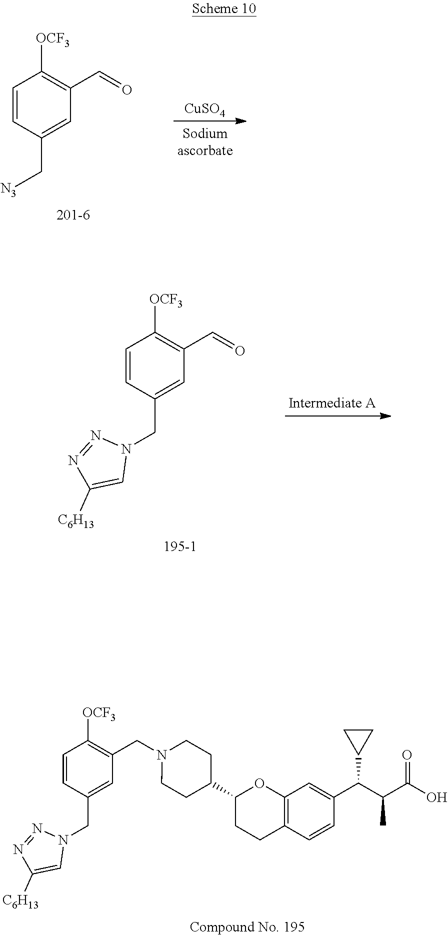

Step 1. A mixture of 5-(azidomethyl)-2-(trifluoromethoxy)benzaldehyde (201-6) (240 mg, 0.98 mmol), 1-octyne (540 mg, 4.90 mmol), Sodium ascorbate (388 mg, 1.96 mmol) and CuSO₄·5H₂O (245 mg, 0.98 mmol) in EtOH (5 mL) and water (5 mL) was stirred at room temperature overnight. The residue was poured into water (10 mL) and the aqueous phase was extracted with EA (20 mL×2). The organic layer was washed with brine (20 mL), dried over Na₂SO₄ and filtered. The filtrate was concentrated, and the residue was purified by silica gel column chromatography (PE/EA=5/1, v/v) to afford 5-((4-hexyl-1H-1,2,3-triazol-1-yl)methyl)-2-(trifluoromethoxy)benzaldehyde (195-1) as a white solid. H NMR (400 MHz, CDCl₃): δ=10.36 (s, 1H), 7.88 (d, J=2.0 Hz, 1H), 7.54-7.52 (m, 1H), 7.38-7.35 (m, 1H), 7.27 (s, 1H), 5.55 (s, 2H), 2.72-2.68 (m, 2H), 1.67-1.62 (m, 2H), 1.36-1.26 (m, 6H), 0.89-0.85 (m, 3H).

Step 2. (2S,3R)-3-cyclopropyl-3-((R)-2-(1-(5-((4-hexyl-1H-1,2,3-triazol-1-yl)methyl)-2-(trifluoro-methoxy)benzyl)piperidin-4-yl)chroman-7-yl)-2-methylpropanoic acid, Compound No. 195, was prepared from Intermediate A and 195-1 in the same way as Compound No. 193. ¹H NMR (400 MHz, CDCl₃): δ=7.47 (s, 1H); 7. (m, 2H); 7.09 (d, 1H); 6.88 (d, 1H); 6.58 (m, 2H); 5.43 (s, 2H); 3.73-23.71 (m, 1H); 3.61-3.55 (m, 2H); 2.93-2.84 (m, 2H); 2.78-2.64 (m, 3H); 2.65-2.60 (m, 2H); 2.13-1.80 (m, 5H); 1.65-1.50 (m, 7H); 1.30-1.15 (m, 6H); 1.10-1.02 (m, 1H); 0.94 (d, 3H); 0.83-0.76 (m, 3H); 0.54-0.52 (m, 1H); 0.29-0.24 (m, 2H); −0.01-0.11 (m, 1H).

### Example 8. Synthesis of Compound No. 189

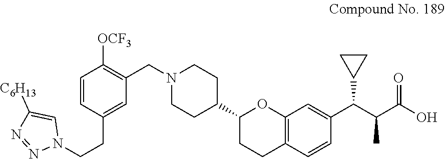

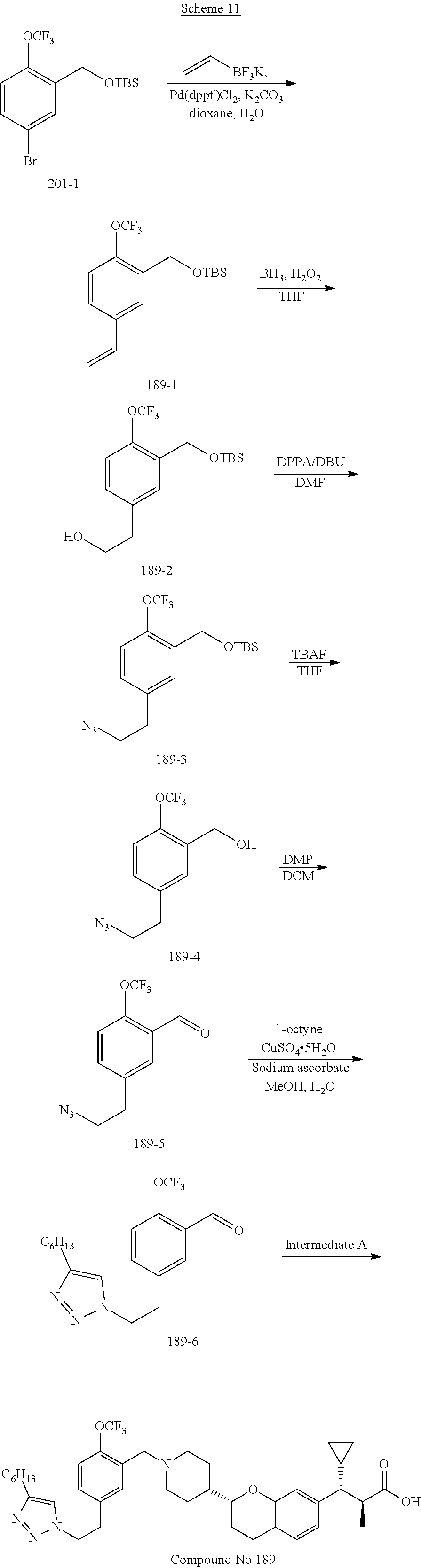

Step 1. To a mixture of ((5-bromo-2-(trifluoromethoxy)benzyl)oxy)(tert-butyl)dimethylsilane (3 g, 7.81 mmol) in 1,4-dioxane (100 mL) and H₂O (5 mL) was added potassium trifluoro(vinyl)borate (1.57 g, 11.72 mmol), K₂CO₃ (2.17 g, 15.62 mmol) and Pd(dppf)Cl₂ (0.57 g, 0.781 mmol). The mixture was stirred under N₂ atmosphere at 95° C. overnight. The reaction mixture was concentrated and the residue was purified by silica gel column chromatography (PE/EA=100/1) to give product tert-butyldimethyl((2-(trifluoromethoxy)-5-vinylbenzyl)oxy)-silane (189-1) (2.1 g, yield: 80%) as colorless oil. ¹H NMR (400 MHz, CDCl₃): δ=7.64 (s, 1H), 7.30-7.27 (m, 1H), 7.15-7.12 (m, 1H), 6.70 (dd, J=17.6, 11.2 Hz, 1H), 5.73 (d, J=17.6 Hz, 1H), 5.27 (d, J=11.2 Hz, 1H), 4.71 (s, 2H), 0.95 (s, 12H), 0.11 (s, 6H).

Step 2. To a solution of 189-1 (1 g, 3.01 mmol) in THE (20 mL) was added BH₃·THF (4.5 ml, 4.52 mmol) dropwise at 0° C. The mixture was stirred for 2 hrs. Then NaOH aqueous solution (2M, 3 mL) and H₂O₂ (0.68 g, 6.02 mmol, 30% in wt.) was added dropwise at 0° C. The resulting mixture was stirred at room temperature overnight. The reaction mixture was diluted with water (40 mL) and extracted with EA (40 mL). The organic phase was washed with brine, dried over Na₂SO₄, filtered and concentrated. The residue was purified by silica gel column chromatography (PE/EA=20/1) to give product 2-(3-(((tert-butyldimethylsilyl)oxy)methyl)-4-(trifluoromethoxy)phenyl)ethan-1-ol (189-2) as a colorless oil. ¹H NMR (400 MHz, DMSO-d6): δ=7.31 (s, 1H), 7.16 (d, J=2.0 Hz, 2H), 4.63 (s, 2H), 4.58 (t, J=5.0 Hz, 1H), 3.55-3.50 (m, 2H), 2.66 (t, J=7.0 Hz, 2H), 0.82 (s, 9H), 0.01 (s, 6H).

Step 3. To a mixture of 189-2 (0.7 g, 2.00 mmol) in DMF (10 mL) was added DPPA (0.66 g, 2.40 mmol) and DBU (0.4 g, 2.60 mmol). The mixture was stirred at 90° C. overnight. The reaction mixture was diluted with water (50 mL) and extracted with PE (50 mL). The organic phase was washed with brine, dried over Na₂SO₄, filtered and concentrated. The residue was purified by silica gel column chromatography (PE/EA=100/1) to give ((5-(2-azidoethyl)-2-(trifluoro-methoxy)benzyl)oxy)(tert-butyl)dimethylsilane (189-3) as a colorless oil.

Step 4. A mixture of 189-3 (140 mg, 0.373 mmol) and TBAF (1 M, 0.75 mL) in THF (5 mL) was stirred for 3 hrs at RT. The reaction mixture was diluted with water (10 mL) and extracted with EA (20 mL). The organic phase was washed with brine, dried over Na₂SO₄, filtered and concentrated to provide product (5-(2-azidoethyl)-2-(trifluoromethoxy)phenyl)methanol (189-4).

Step 5. To a mixture of 189-4 (50 mg, 0.192 mmol) in DCM (5 mL) was added Dess-Martin periodinane (162.5 mg, 0.383 mmol). The mixture under was stirred at room temperature for 3 hrs. The reaction mixture was concentrated and the residue was purified by silica gel column chromatography (PE/EA=10/1) to give product 5-(2-azidoethyl)-2-(trifluoromethoxy)benzaldehyde (189-5) as colorless oil. ¹H NMR (400 MHz, DMSO-d6): δ=10.23 (s, 1H), 7.87 (d, J=2.4 Hz, 1H), 7.78-7.75 (dd, J=8.4 Hz, J=2.0 Hz, 1H), 7.55-7.52 (dd, J=8.0 Hz, J=1.2 Hz, 1H), 3.65-3.60 (m, 2H), 2.97 (t, J=6.8 Hz, 2H).

Step 6. A mixture of 189-5 (100 mg, 0.386 mmol) in MeOH/H₂O (10 mL/2 mL), oct-1-yne (42.5 mg, 0.386 mmol), CuSO₄·5H₂O (19.3 mg, 0.077 mmol) and sodium ascorbate (30.6 mg, 0.154 mmol) was stirred under N₂ atmosphere at room temperature overnight. The reaction mixture was diluted with water (20 mL) and extracted with EA (20 mL). The organic phase was washed with brine, dried over Na₂SO₄, filtered and concentrated. The residue was purified by silica gel column chromatography (PE/EA=4/1 to 2/1) to give product 5-(2-(4-hexyl-1H-1,2,3-triazol-1-yl)ethyl)-2-(trifluoromethoxy)benzaldehyde (189-6) as a white solid. ¹H NMR (400 MHz, DMSO-d6): δ=10.20 (s, 1H), 7.74 (s, s, 2H), 7.62-7.59 (dd, J=8.4 Hz, J=2.0 Hz, 1H), 7.48-7.45 (dd, J=8.0 Hz, J=1.2 Hz, 1H), 4.60 (t, J=7.0 Hz, 2H), 3.27-3.22 (m, 2H), 2.54 (t, J=7.4 Hz, 2H), 1.51 (t, J=7.0 Hz, 2H), 1.24 (s, 6H), 0.85 (t, J=7.8 Hz, 3H). MS Calcd.: 669; MS Found: 670 [M+H]⁺.

Step 7. (2S,3R)-3-cyclopropyl-3-((R)-2-(1-(5-(2-(4-hexyl-1H-1,2,3-triazol-1-yl)ethyl)-2-(trifluoro-methoxy)benzyl)piperidin-4-yl)chroman-7-yl)-2-methylpropanoic acid, Compound No. 189, was prepared from 189-6 and intermediate A in the same way as Compound No. 193. ¹H NMR (400 MHz, CDCl₃): δ=7.39 (s, 1H); 7.15 (d, 1H); 7.04 (s, 1H); 6.99 (d, 1H); 6.92 (d, 1H); 6.65 (s, 1H); 6.63 (d, 1H); 4.55 (t, 2H); 3.79 (d, 1H); 3.58-3.54 (m, 2H); 3.20 (t, 2H); 2.98-2.85 (m, 2H); 2.85-70 (m, 3H); 2.63 (t, 2H); 2.1-1.4 (m, 12H, overlapped with H2O peak); 1.27 (m, 6H); 1.12-1.06 (m, 1H); 0.97 (d, 3H); 0.86 (t, 3H); 0.58 (s, 1H); 0.37-0.27 (m, 2H); −0.01-0.03 (m, 1H). MS Calcd.: 696; MS Found: 697 [M+H]⁺.

### Example 9. Synthesis of Compound No. 190

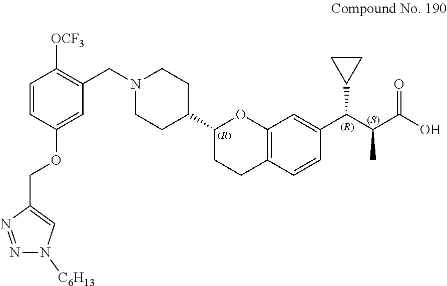

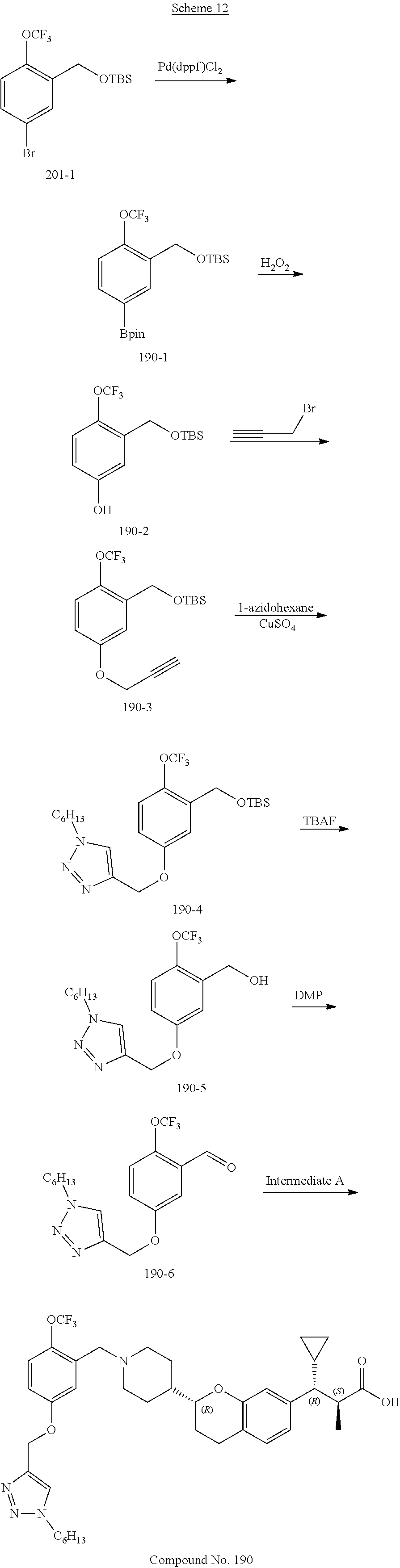

Step 1. A flask charged with ((5-bromo-2-(trifluoromethoxy)benzyl)oxy)(tert-butyl)dimethylsilane (201-1) (2.5 g, 6.51 moL), 4,4,4′,4′,5,5,5′,5′-octamethyl-2,2′-bi(1,3,2-dioxaborolane) (2.48 g, 9.76 mmoL), Pd(dppf)Cl₂ (476 mg, 0.651 mmoL) and KOAc (1.275 g, 13.02 mmoL) in dioxane (100 mL) was degassed and filled with N₂. The reaction mixture was heated at 95° C. for 16 hrs. Solvent was removed and the residue was purified by silica gel column chromatography (PE/EA=100:0 to 90:10) to give product tert-butyldimethyl((5-(4,4,5,5-tetramethyl-1,3,2-dioxaborolan-2-yl)-2-(trifluoromethoxy)benzyl)oxy)silane (190-1) as pale-yellow oil. ¹H NMR (400 MHz, CDCl₃): δ=7.89 (s, 1H), 7.62-7.60 (m, 1H), 7.09-7.06 (m, 1H), 4.66 (s, 2H), 1.23 (s, 12H), 0.99 (s, 9H), 0.02 (s, 6H).

Step 2. To a stirred solution of 190-1 (500 mg, 1.16 mmol) in THE (20 mL) at 0° C. was added 1N aqueous NaOH solution (5.8 mL), followed by slow addition of 30% aqueous H₂O₂ (1.2 mL, 11.6 mmol). After addition, the reaction mixture was stirred for 12 hrs, allowing the temperature to slowly warm to room temperature. The mixture was quenched with aq, NH₄Cl (20 mL) and separated. The water phase was extract with EA (20 mL×2). The organic phase was combined, dried and concentrated. The residue was purified by flash chromatography (PE/EA=100:0 to 80:10) to give 3-(((tert-butyldimethylsilyl)oxy)methyl)-4-(trifluoromethoxy)phenol (190-2) as colorless oil. ¹H NMR (400 MHz, DMSO-d6): δ=9.74 (s, 1H), 7.11-7.08 (m, 1H), 6.93 (s, 1H), 6.72-6.69 (m, 1H), 4.65 (s, 2H), 0.89 (s, 9H), 0.07 (s, 6H).

Step 3. To a mixture of 190-2 (490 mg, 1.52 mmol) and K₂CO₃ (422 mg, 3.04 mmol) in MeCN (20 mL) at room temperature was added 3-bromo-propyne (272 mg, 2.28 mmol) dropwise. After addition, the resulting mixture was stirred at 80° C. for 14 hrs. Solvent was removed and the residue was purified by flash chromatography (PE/EA=100:0 to 98:2) to give tert-butyldimethyl((5-(prop-2-yn-1-yloxy)-2-(trifluoromethoxy)benzyl)oxy)silane (190-3) as a white oil. ¹H NMR (400 MHz, CDCl₃): δ=7.23 (d, J=3.2 Hz, 1H), 7.12-7.10 (m, 1H), 6.85-6.82 (m, 1H), 4.76 (s, 2H), 4.68 (d, J=2.4 Hz, 2H), 2.51 (t, J=2.4 Hz, 1H), 0.95 (s, 9H), 0.11 (s, 6H).

Step 4. A mixture of 190-3 (450 mg, 1.25 mmol) in EtOH (2 mL), 1-azidohexane (˜2 eq., 9 mL 1:1 EtOH:H2O), CuSO₄·5H₂O (31 mg, 0.125 mmol) and AscNa (99 mg, 0.5 mmol) was stirred at room temperature for 16 hrs. The reaction mixture was diluted with water (10 mL) and extracted with EA (20 mL×3), dried over Na₂SO₄ and concentrated to give crude 4-((3-(((tert-butyldimethylsilyl)oxy)methyl)-4-(trifluoromethoxy)phenoxy) methyl)-1-hexyl-1H-1,2,3-triazole (190-4) as yellow gum. MS Calcd.: 471.2; MS Found: 494.2 [M+Na]⁺.

Step 5. A mixture of 190-4 (600 mg, 1.232 mmol) and TBAF (1 M, 6.12 mL) in THF (10 mL) was stirred at room temperature for 12 hrs. Solvent was removed and the residue was purified by flash chromatography (PE/EA=10:90 to 40:60) to give (5-((1-hexyl-1H-1,2,3-triazol-4-yl)methoxy)-2-(trifluoromethoxy)phenyl)methanol (190-5) as a yellow solid. MS Calcd.: 373.2; MS Found: 374.3 [M+H]⁺.

Step 6. To a stirred mixture of 190-5 (160 mg, 0.428 mmol) in DCM (10 mL) at room temperature was added DMP (272 mg, 0.642 mmol) in portions. After addition, the resulting mixture was stirred for 4 hrs. Solvent was removed and the residue was purified by silica gel column chromatography (PE/EA=10:90 to 50:50) to give 5-((1-hexyl-1H-1,2,3-triazol-4-yl)methoxy)-2-(trifluoromethoxy)benzaldehyde (190-6) as yellow solid. MS Calcd.: 371.2; MS Found: 372.3 [M+H]⁺. ¹H NMR (400 MHz, CDCl₃): δ=10.3 (s, 1H), 7.60 (s, 1H), 7.52 (s, 1H), 7.28-7.25 (m, 2H), 5.24 (s, 2H), 4.36 (t, J=7.2 Hz, 2H), 1.93-1.88 (m, 2H), 1.34-1.25 (m, 6H), 0.88 (t, J=6.8 Hz, 3H).

Step 7. (2S,3R)-3-cyclopropyl-3-((R)-2-(1-(5-((1-hexyl-1H-1,2,3-triazol-4-yl)methoxy)-2-(trifluoro-methoxy)benzyl)piperidin-4-yl)chroman-7-yl)-2-methylpropanoic acid, Compound No, 190, was prepared from 190-6 and Intermediate A in the same way as Compound No, 193. MS Calcd.: 698.4; MS Found: 699.4 [M+H]⁺. ¹H NMR (400 MHz, CDCl₃): δ=7.60 (s, 1H); 7.25 (s, 1H); 7.15 (d, 1H); 6.92-6.90 (m, 2H); 6.61-6.59 (m, 2H); 5.20 (s, 2H); 4.34 (t, 2H); 3.91-3.64 (m, 3H); 3.17-3.15 (m, 1H); 3.03-3.01 (m, 1H); 2.80-2.70 (m, 3H); 2.22-2.15 (m, 2H); 2.03-1.84 (m, 5H); 1.81-1.55 (m, 5H); 1.29-1.26 (m, 6H); 1.09-1.03 (m, 1H); 0.95 (d, 3H); 0.89-0.83 (t, 3H); 0.61-0.53 (m, 1H); 0.39-0.32 (m, 1H); 0.27-0.22 (m, 1H); −0.01-0.11 (m, 1H).

### Example 10. Synthesis of Compound No. 130

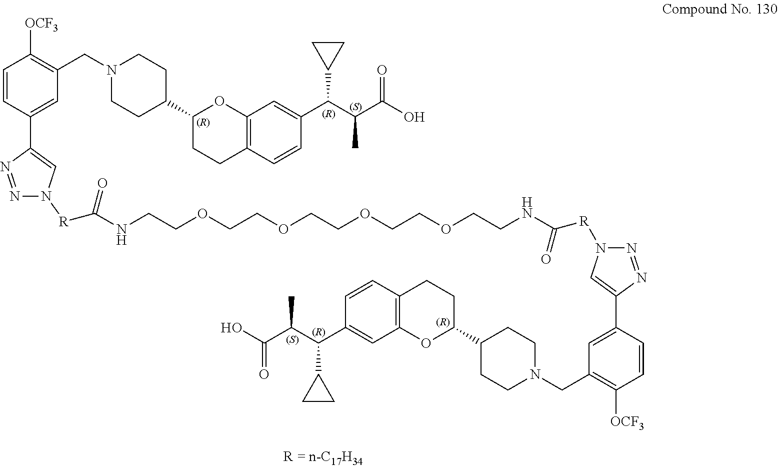

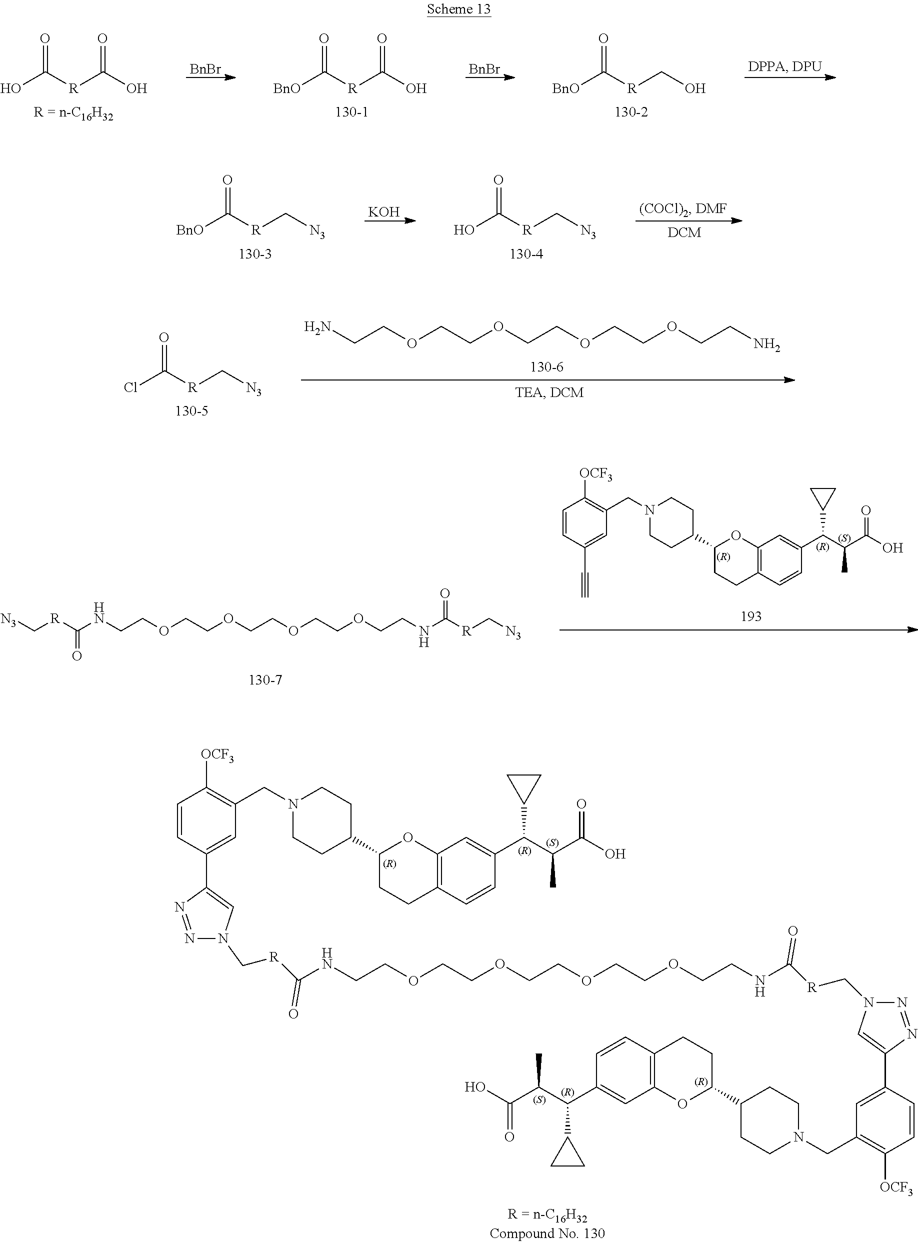

Step 1. To a solution of octadecanedioic acid (10 g, 0.032 mol) in DMF (150 mL) was added BnBr (5.5 g, 0.032 mol) and K₂CO₃ (6.6 g, 0.048 mol) at RT. The mixture was stirred at 8° C. for overnight. 0.5 N HCl was added to adjust pH=2. The resulting solution was extracted with EA (200 mL×3). The organic layer was washed with brine (200 mL×3), dried over Na₂SO₄ and filtered. The filtrate was concentrated, and the residue was purified by silica gel column chromatography (PE/EA=3/1, v/v) to afford 18-(benzyloxy)-18-oxooctadecanoic acid, 130-1, as a white solid. ¹H NMR (400 MHz, DMSO-d₆): δ=11.94 (s, 1H); 7.38-7.31 (m, 5H); 5.08 (s, 2H); 2.35-2.32 (m, 2H); 2.19-2.16 (m, 2H); 1.54-1.46 (m, 4H); 1.23 (s, 24H).

Step 2. To a solution of 130-1 (6.0 g, 0.015 mol) in THF (50 mL) was added BH₃/THF (1.0M, 45 mL, 0.045 mmol) at 0° C. for overnight. Then the reaction was quenched with MeOH (50 mL) and concentrated in vacuo to afford. The residue was purified by silica gel column chromatography (PE/EA=5/1, v/v) to afford benzyl 18-hydroxyoctadecanoate, 130-2, as a white solid. ¹H NMR (400 MHz, DMSO-d₆): δ=7.37-7.32 (m, 5H); 5.08 (s, 2H); 4.32-4.30 (m, 1H); 3.39-3.32 (m, 2H); 2.36-2.32 (m, 2H); 1.54-1.51 (m, 2H); 1.4-1.37 (m, 2H); 1.23 (s, 26H).

Step 3. To a solution of 130-2 (7.4 g, 0.019 mol) in DMF (100 mL) was added DPPA (7.8 g, 0.028 mol) and DBU (4.3 g, 0.028 mol) at RT. The mixture was stirred at 90° C. for overnight. The residue was poured into water (100 mL), extracted with EA (200 mL×2). The organic layer was washed with brine (100 mL×2), dried over Na₂SO₄ and filtered. The filtrate was concentrated, and the residue was purified by silica gel column chromatography (PE) to afford benzyl 18-azidooctadecanoate, 130-3, as a white solid. ¹H NMR (400 MHz, CDCl₃): δ=7.37-7.32 (m, 5H); 5.11 (s, 2H); 3.27-3.23 (m, 2H); 2.37-2.33 (m, 2H); 1.66-1.56 (m, 4H); 1.38-1.25 (m, 26H).

Step 4. To a solution of 130-3 (1.0 g, 2.41 mmol) in MeOH/H₂O (10 mL/5 mL) was added KOH (675 mg, 12.05 mmol) at ° C. The mixture was stirred at 0° C. for 2 h. Then the reaction was concentrated in vacuo, acidified to pH=1 by 1 N HCl and extracted by EA. The organic layer was combined, dried over Na₂SO₄ and filtered. The filtrate was concentrated afford 18-azidooctadecanoic acid, 130-4, as a white solid. H NMR (400 MHz, DMSO-d₆): δ=11.99 (s, 1H); 3.32-3.29 (m, 2H); 2.20-2.16 (m, 2H); 1.54-1.46 (m, 4H); 1.25-1.20 (m, 26H).

Step 5. To a solution of 130-4 (700.0 mg, 2.15 mmol) in dry DCM (10 mL) was added dropwise oxalyl chloride (0.9 mL, 10.75 mmol) and DMF (2 drops) under nitrogen atmosphere at ice bath. The reaction mixture was stirred at 0° C. for 2 hours. The reaction mixture was concentrated in vacuum to give 18-azidooctadecanoyl chloride, 130-5, as a yellow oil, which was taken onto the next step without any further purification.

Step 6. To a solution of 130-5 (700.0 mg, crude) in dry DCM (10 mL) was added TEA (652.0 mg, 6.45 mmol) and a solution of 3,6,9,12-tetraoxatetradecane-1,14-diamine (203.0 mg, 0.86 mmol) in dry DCM (5 mL) at 0° C. The reaction mixture was stirred at room temperature overnight. The reaction mixture was concentrated in vacuum. The residue was recrystallized with methanol to give N,N′-(3,6,9,12-tetraoxatetradecane-1,14-diyl)bis(18-azido octadecanamide), 130-7, as a white solid. 1H NMR (400 MHz, CDCl3): δ=6.38 (s, 2H); 3.66-3.61 (m, 12H); 3.55 (t, J=4.8 Hz, 4H); 3.45 (q, J=5.2 Hz, 4H); 3.25 (t, J=6.8 Hz, 4H); 2.17 (t, J=7.6 Hz, 4H); 1.63-1.56 (m, 8H); 1.41-1.25 (m, 52H). MS: m/z 851.7 (M+H+).

Step 7. To a solution of 130-7 (30.0 mg, 0.035 mmol) in EtOH/H2O/DCM (1/1/1 mL) was added 193 (42.0 mg, 0.077 mmol), L-Ascorbic acid sodium salt (2.8 mg, 0.014 mmol) and Copper(II) sulfate pentahydrate (1.7 mg, 0.007 mmol). The reaction mixture was stirred at room temperature for 24 hours. The reaction mixture was quenched with water and extracted with DCM (10 mL×3). The combined organic layers were washed with water (10 mL×2) and brine (10 mL), dried over Na₂SO₄ and concentrated. The residue was purified by preTLC (DCM/MeOH=20:1) and pre.HPLC to give (2S,3R)-3-((R)-2-(1-(5-(1-(52-(4-(2-((4-((R)-7-((1R,2S)-2-carboxy-1-cyclo propylpropyl)chroman-2-yl)piperidin-1-yl)methyl)-4-(trifluoro-methoxy)phenyl)-1H-1,2,3-triazol-1-yl)-18,35-dioxo-22,25,28,31-tetraoxa-19,34-diazado pentacontyl)-1H-1,2,3-triazol-4-yl)-2-(trifluoromethoxy)benzyl)piperidin-4-yl)chroman-7-yl)-3-cyclopropyl-2-methylpropanoic acid, 130, as a white solid. ¹H NMR (400 MHz, CDCl₃): δ=8.25 (d, 2H); 8.16 (s, 2H); 8.00 (s, 2H); 7.41 (d, 2H); 6.94 (d; 2H); 6.64 (d, 2H); 6.58 (brs, 4H); 4.39-4.40 (t, 4H); 4.30 (br, 4H); 3.80 (brs, 2H); 3.66 (s, 4H); 3.63 (s, 8H); 3.57-3.55 (m, 4H); 3.47-3.43 (m, 4H); 2.80-2.75 (m, 6H); 2.24-2.16 (m, 8H); 2.13-1.72 (m, 16H); 1.67-1.52 (m, 8H); 1.33-1.23 (m, 52H), 1.23-1.12 (m, 2H); 0.97-0.96 (d, 6H); 0.92-0.87 (m, 2H); 0.63-0.52 (m, 2H); 0.38-0.32 (m, 4H); 0.0 (m, 2H). MS: m/2z 967.9 (M/2+H⁺).

### Example 11. Synthesis of Compound No. 203

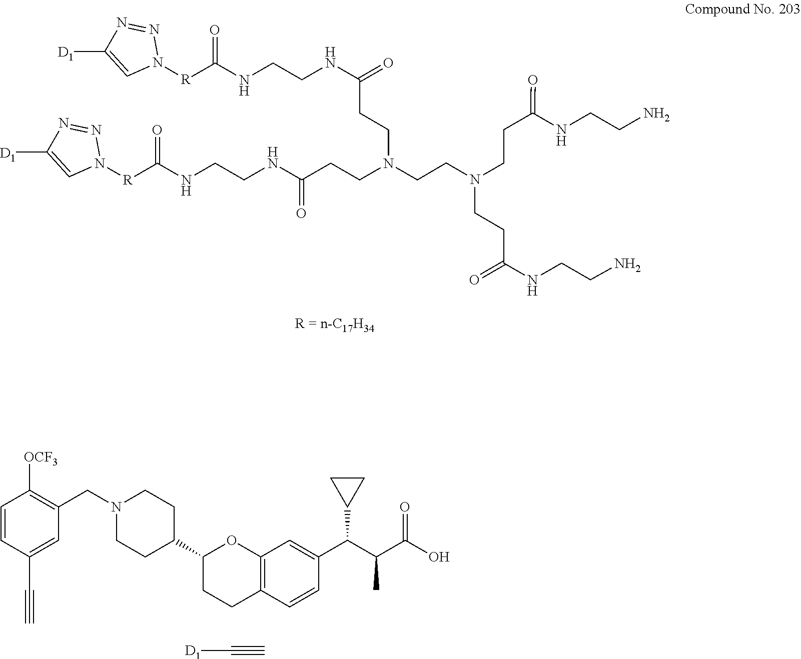

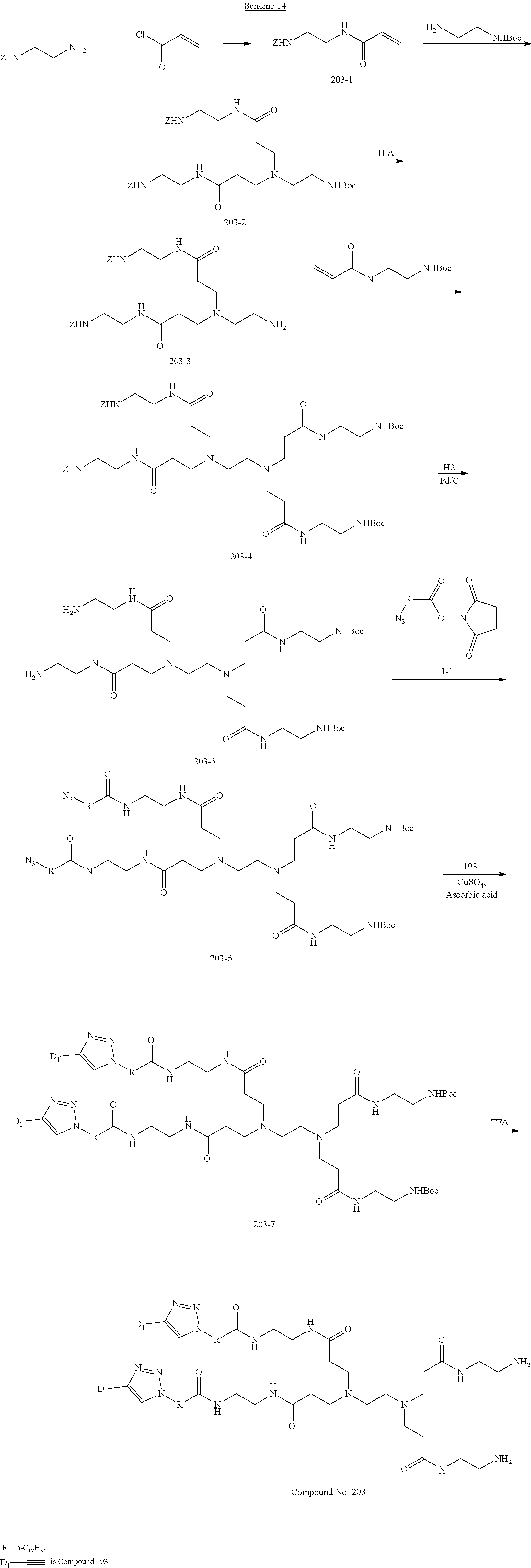

Step 1. To a mixture of benzyl (2-aminoethyl)carbamate (25.0 g, 0.108 mol) and TEA (32.7 g, 0.324 mol) in DCM (120 mL) at 0° C. was added acryloyl chloride (13.2 mL, 0.162 mol) dropwise during 10 min. After addition, the resulting mixture was stirred for 16 hrs, allowing the temperature to slowly warm to r. t. The reaction mixture was quenched with aq. NaHCO₃ and separated, extracted with DCM (50 mL×2). The combined organic phase was dried and concentrated. The residue was purified by flash chromatography (60% EA in PE) to give benzyl (2-acrylamidoethyl)carbamate, 203-1, as a pale-yellow solid. MS (ESI) m/z 249.1 [M+1]⁺.

Step 2. A pressure tube charged with tert-butyl (2-aminoethyl)carbamate (2.0 g, 12.48 mmol) and 203-1 (12.4 g, 49.94 mmol) in sat. aq. HBO₃ (10 mL) was sealed and heated at 100° C. for 2 days. The reaction mixture was diluted with water (10 mL), extracted with DCM (30 mL×4) and concentrated. The residue was purified by flash chromatography (10% MeOH in DCM, @214 nm) to give compound 203-2 (2.0 g, yield: 24.7%) as yellow gum. MS (ESI) m/z 657.1 [M+H]⁺.

Step 3. To a solution of 203-2 (2.0 g, 3.05 mmol) in DCM (10 mL) was added TFA (10 mL) dropwise at r. t. The resulting mixture became pale-yellow and clear. Stirred for 12 hrs at r. t. The reaction mixture was concentrated in vacuum. The crude was dissolved in methanol (10 mL), then to the solution was added NaOH aq (2 M, 10 mL). The reaction mixture was stirred at r. t for 3 hrs. The reaction mixture was diluted with water (20 mL), extracted with DCM (30 mL×3) and concentrated. The residue was purified by prep-HPLC to give dibenzyl (((3,3′-((2-aminoethyl)azanediyl)bis(propanoyl))bis(azanediyl))bis(ethane-2,1-diyl))dicarbamate, 203-3, (1.0 g, yield: 58.8%) as a colorless gum. MS (ESI) m/z 557.0 [M+H]⁺.

Step 4. A pressure tube charged with 203-3 (500 mg, 0.899 mmol) and tert-butyl (2-acrylamidoethyl)carbamate (960 mg, 4.496 mmol) in sat. aq. HBO₃ (5 mL) was sealed and heated at 100° C. for 2 days. The reaction mixture was diluted with water (10 mL), extracted with DCM (30 mL×4) and concentrated. The residue was purified by prep-HPLC to give 203-4 (200 mg, yield: 22.6%) as colorless gum. MS (ESI) m/z 985.2 [M+H]⁺.

Step 5. A flask charged with 203-4 (200 mg, 0.203 mmol) and Pd/C (100 mg, 50% w.t.) in MeOH (10 mL) was degassed and filled with hydrogen using a balloon. The resulting mixture was then hydrogenated at 25° C. for 16 hrs. The reaction was filter over celite and concentrated. The residue was purified by prep-HPLC to give 203-5 (150 mg, yield: 57.7%) as colorless oil. MS (ESI) m/z 717.6 [M+H]⁺.

Step 6. To a mixture of 203-5 (140 mg, 0.195 mmol) and TEA (98 mg, 0.975 mmol) in THE (10 mL) was added 1-1 (331 mg, 0.781 mmol). After addition, the resulting mixture was stirred at 45° C. for 16 hrs. Solvent was removed and the residue (in MeOH) was purified by prep-HPLC (NH₄HCO₃ method) to 203-6 (60 mg, yield: 23.1%) as white solid. MS (ESI) m/z 666.7 [M/2+H]⁺.

Step 7. To a solution of 193 (16.3 mg, 0.030 mmol) in THF (2 mL) was add 203-6 (20.0 mg, 0.015 mmol), L-Ascorbic acid sodium salt (0.2 M, 150 uL, H₂O solution) and Copper(II) sulfate pentahydrate (0.1 M, 150 uL, H₂O solution). The reaction mixture was heated to 50° C. and stirred for 16 hours. The combined reaction mixture was concentrated. The residue was purified by prep-HPLC (TFA method, C8 column) to give (2S,2'S,3R,3′R)-3,3′-((2R,2′R)-2,2′-(1,1′-(((1,1′-(26-(12-(3-((2-((tert-butoxycarbonyl)amino)ethyl)amino)-3-oxo propyl)-2,2-dimethyl-4,9-dioxo-3-oxa-5,8,12-triazatetradecan-14-yl)-18,23,29,34-tetraoxo-19,22,26,30,33-pentaazahenpentacontane-1,51-diyl)bis(1H-1,2,3-triazole-4,1-diyl)) bis(2-(trifluoro methoxy)-5,1-phenylene))bis(methylene))bis(piperidine-4,1-diyl))bis(chroman-7,2-diyl))bis (3-cyclopropyl-2-methylpropanoic acid), 203-7, (20 mg, crude) as a white solid.

Step 8. To a solution of 203-7 (10 mg, crude) in DCM (5 mL) was added TFA (1 mL). The reaction mixture was stirred at room temperature for 2 hours. The reaction mixture was detected completed by LCMS. The reaction mixture was concentrated in vacuum. The residue was purified by pre.HPLC to give (2S,2'S,3R,3′R)-3,3′-((2R,2′R)-2,2′-(1,1′-(((1,1′-(26-(2-(bis(3-((2-aminoethyl)amino)-3-oxopropyl)amino) ethyl)-18,23,29,34-tetraoxo-19,22,26,30,33-pentaazahenpentacontane-1,51-diyl)bis(1H-1,2,3-triazole-4,1-diyl))bis(2-(trifluoromethoxy)-5,1-phenylene))bis(methylene))bis (piperidine-4,1-diyl))bis(chroman-7,2-diyl))bis(3-cyclopropyl-2-methylpropanoic acid), Compound No. 203, as a white solid. MS: m/2z 1108.8 (M/2+H⁺).

### Example 12. Synthesis of Compound No. 1

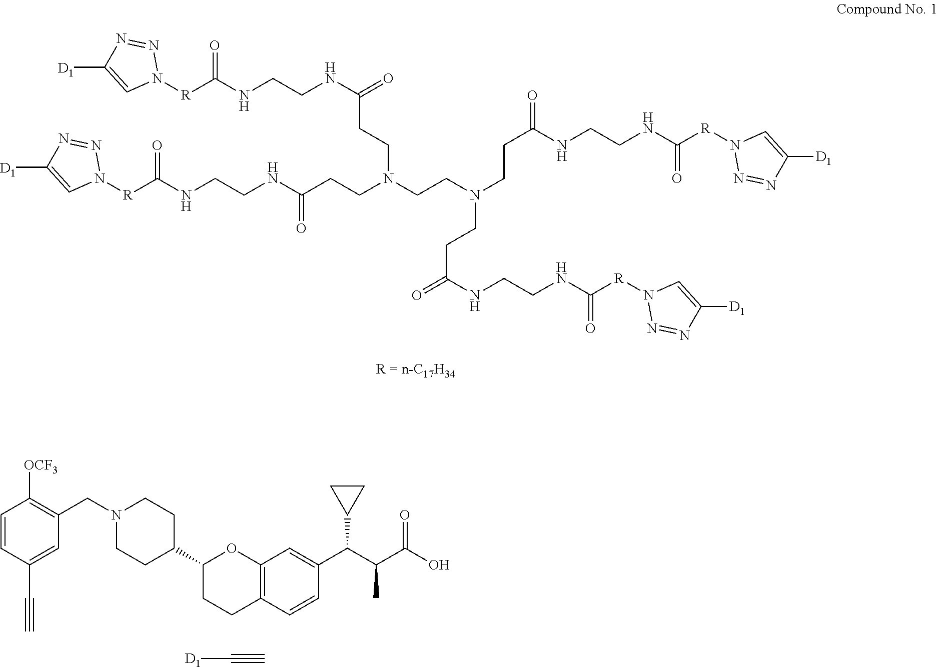

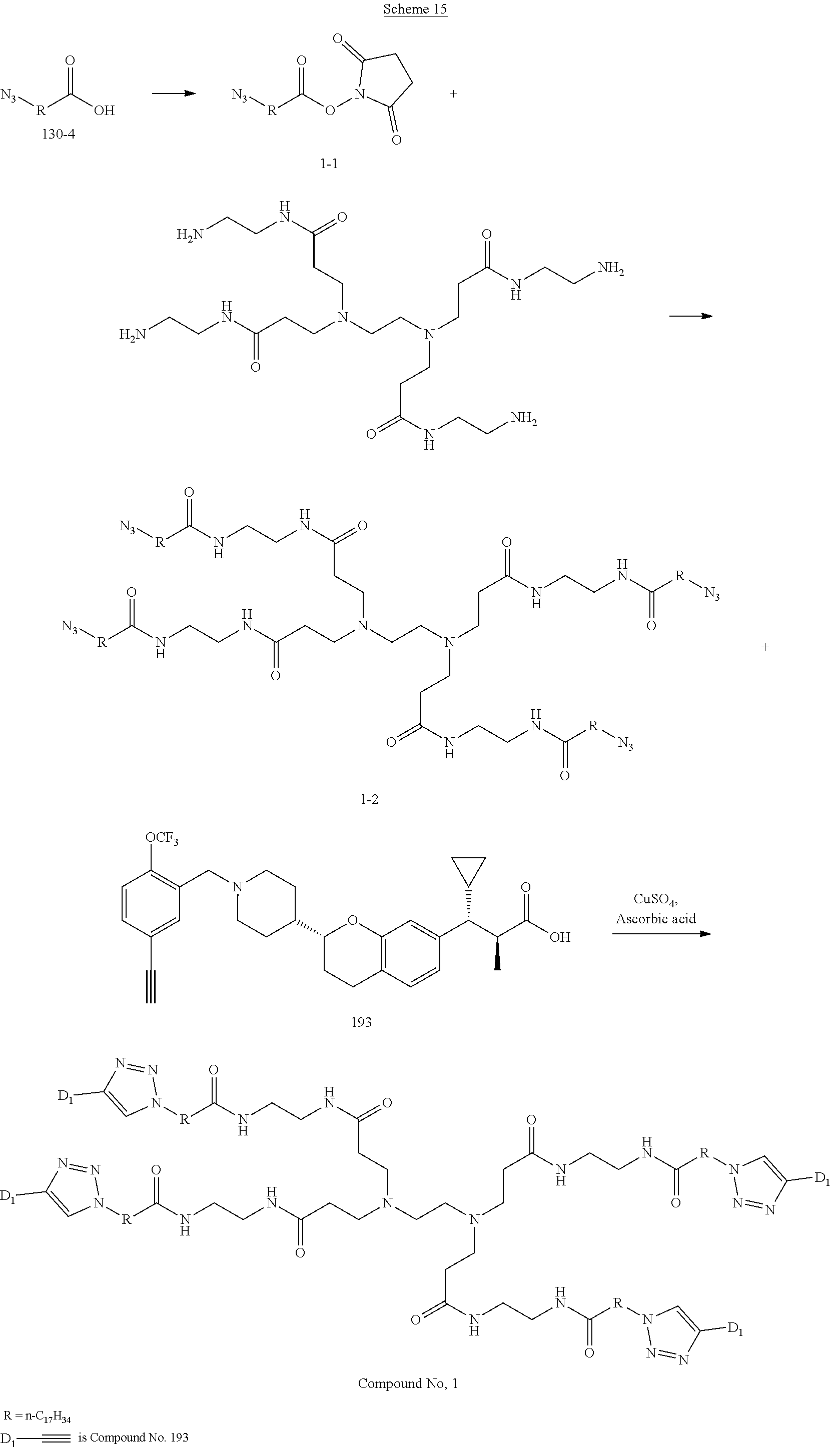

Step 1. To a mixture of 18-azido-octadecanoic acid, 130-4, (1 g, 3.07 mmol) and 1-hydroxy-pyrrolidine-2,5-dione (372 mg, 3.23 mmol) in DCM (50 mL) was added EDCI (650 mg, 3.383 mmol) in portions at r. t. (15° C.). After addition, the resulting mixture was stirred for 14 hrs at r. t. The reaction mixture was concentrated and the residue was purified by silica gel column chromatography (50% to 100% DCM in PE) to give 18-azido-octadecanoic acid 2,5-dioxo-pyrrolidin-1-yl ester, 1-1, as a white solid. H NMR (400 MHz, CDCl₃): δ=3.25 (t, J=7.2 Hz, 2H); 2.83 (brs, 4H); 2.60 (t, J=7.6 Hz, 2H); 1.76-1.72 (m, 2H); 1.62-1.56 (m, 2H); 1.41-1.30 (m, 2H); 1.25-1.3 (m, 24H).

Step 2. To a mixture of N-(2-amino-ethyl)-3-[[2-(2-amino-ethylcarbamoyl)-ethyl]-(2-{bis-[2-(2-amino-ethylcarbamoyl)-ethyl]-amino}-ethyl)-amino]-propionamide (191 mg, 0.369 mmol) and TEA (226 mg, 2.217 mmol) in DMF (15 mL) was added 1-1 (780 mg, 1.848 mmol) in portions. After addition, the resulting mixture was stirred at 50° C. for 16 hrs. The reaction mixture was diluted with water (30 mL) and filtered. The filter cake was washed with water (10 mL×1), MeOH (10 mL×2) and EA (10 mL×2) successively, and air-dried overnight to give 18-azido-octadecanoic acid [2-(3-{{2-[2-(18-azido-octadecanoylamino)-ethylcarbamoyl]-ethyl}-[2-(bis-{2-[2-(18-azido-octadecanoylamino)-ethylcarbamoyl]-ethyl}-amino)-ethyl]-amino}-propionyl amino)-ethyl]-amide, 1-2, as pale-yellow solid. ¹H NMR (400 MHz, CF₃CO₂D): δ=4.20 (br s, 4H); 3.75 (m, 16H); 3.63 (m, 8H); 3.44 (t, 8H); 3.13-3.11 (m, 8H); 2.67-2.63 (m, 8H); 1.80-1.75 (m, 16H); 1.47-1.36 (m, 104H).

Step 3. To a mixture of 193 (6.3 mg, 12.0 mmol) and 1-2 (5.5 mg, 3.15 mmol) in THE (1.8 mL) and water (0.6 mL) was added CuSO₄·5H₂O (0.1 M, 60 uL) and L-AASS (0.2 M, 60 uL). The resulting mixture was then stirred for 16 hrs at r. t. The reaction mixture was concentrated. The residue was treated with DMSO (4 mL)/TFA (0.8 mL), stirred for 2 h at r. t. and filtered. The filtrate was then purified by prep-HPLC (TFA method, C8 column) to give Compound No. 1 (TFA salt, 43 mg, yield 20%) as pale-yellow gum. m/z: 1305.4 (m/3+1), 979.4 (m/4+1), 783.7 (m/5+1), 653.1 (m/6+1). ¹H NMR (400 MHz, DMSO-d6): δ=9.75 (br, 4H); 8.64 (s, 4H); 8.34 (s, 4H); 8.16 (s, 4H); 8.00-7.98 (d, 4H); 7.83 (s, 4H); 7.59-7.57 (d, 4H); 6.97-6.94 (d, 4H); 6.65-6.63 (d, 4H); 6.51 (s, 4H); 4.43-4.39 (m, 16H); 3.81-3.80 (m, 4H); 3.65-3.45 (m, 8H); 3.5 (br, 4H); 3.25-3.05 (m, 8H); 3.1 (br, 16H); 3.05 (br, 8H); 2.76-2.59 (m, 12H); 2.44-2.40 (m, 8H); 2.1-2.0 (m, 12H); 1.95-1.81 (m, 24H); 1.75-1.56 (m, 12H); 1.50-1.41 (m, 8H); 1.31-1.15 (m, 104H); 1.07-1.00 (m, 4H); 0.80 (d, 12H); 0.53-0.47 (m, 4H); 0.26-0.20 (m, 8H); −0.09˜−0.11 (m, 4H).

### Example 13. Synthesis of Compound No 8

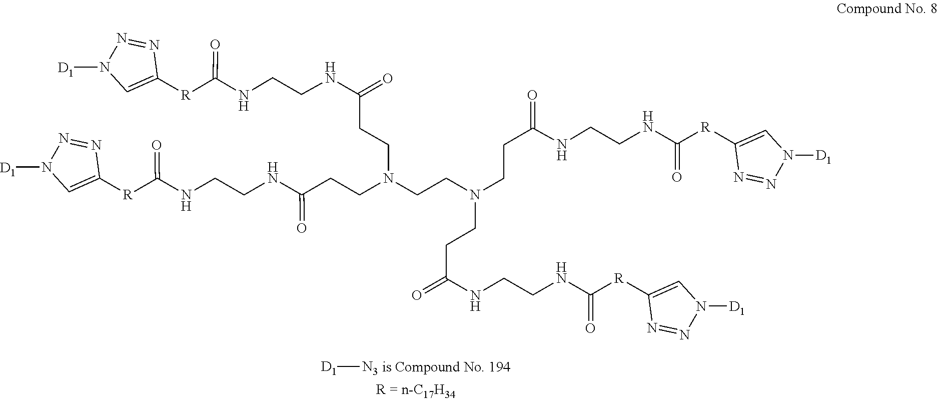

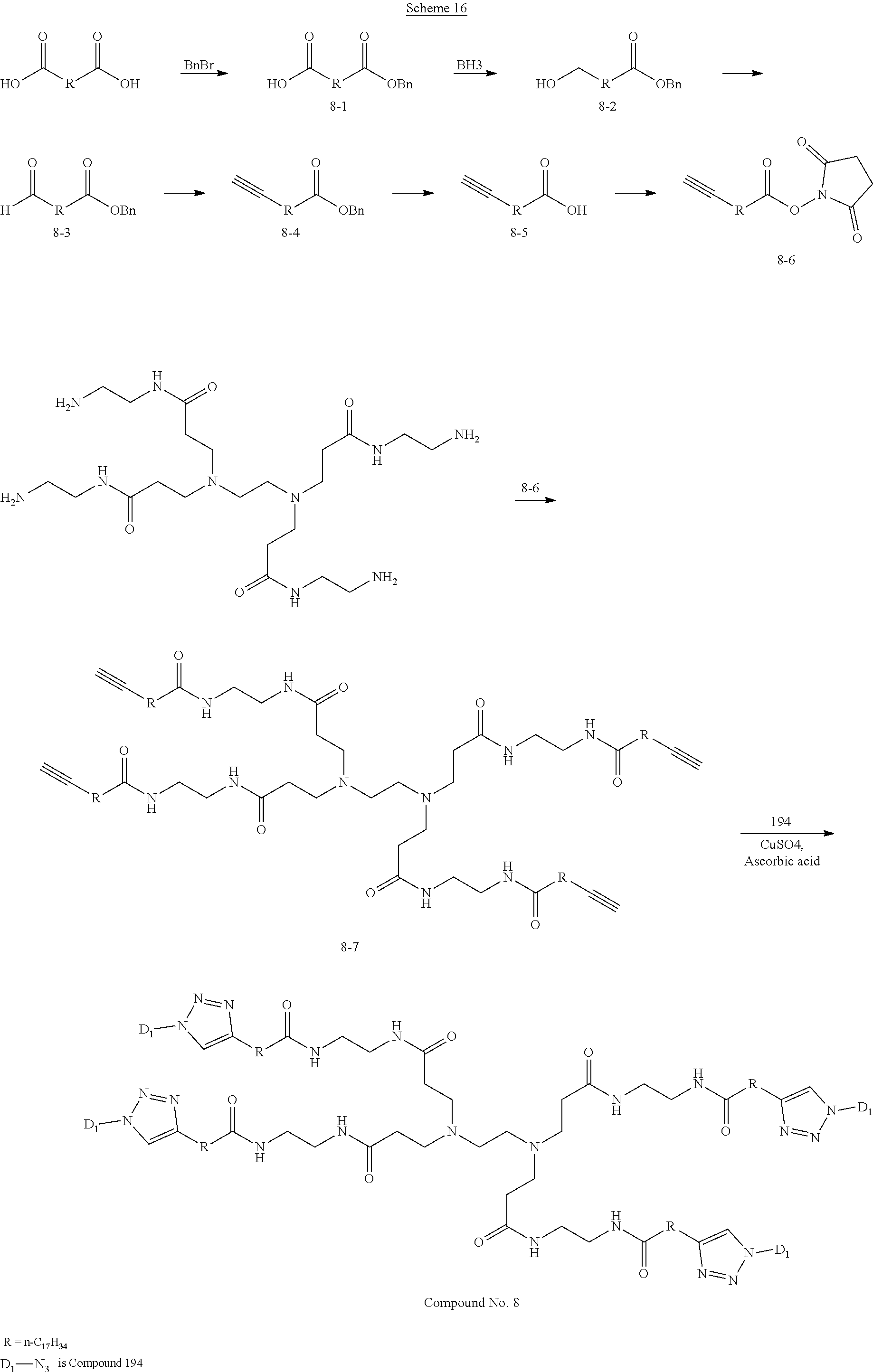

Step 1. To a solution of nonadecanedioic acid (23.0 g, 70.2 mmol) in DMF (400 mL) at room temperature was added K₂CO₃ (19.4 g, 140.4 mmol), BnBr (12.0 g, 70.2 mmol). The reaction mixture was stirred at 80° C. overnight under N₂. The reaction mixture was diluted with solvent and poured into water. The mixture was acidified to pH 3-4 with aqueous HCl and the mixture was extracted with EA (20 mL×4). The combined organic layers were dried over MgSO₄ and concentrated. The crude product was chromatographed on silica gel (Petroleum ether/EtOAc 10:1→5:1→0:1→MeOH/EtOAc 1:10) to give compound 8-1 (15.8 g, 54%) as a white solid. MS (ESI) m/z 417.2 [M−H]⁻. ¹H NMR (400 MHz, CDCl₃) δ 7.42-7.35 (m, 5H); 5.12 (s, 2H); 2.35 (t, J=6.8 Hz, 4H); 1.73-1.62 (m, 4H); 1.31-1.17 (m, 26H).

Step 2. To a solution of compound 8-1 (2.22 g, 5.3 mmol) in THE (25 mL) cooled to 0° C. was added dropwise BH₃ (5.3 mL, 10.6 mmol) under N₂. The reaction mixture was stirred at room temperature for 2 hours. The reaction mixture was quenched by the addition of MeOH at 0° C. The reaction mixture was concentrated under reduced pressure. The crude product was chromatographed on silica gel (Petroleum ether/EtOAc 10:1→7:1→4:1) to give compound 8-2 (1515 mg, 71%) as a white solid. MS (ESI) m/z 405.2 [M+H]⁺. 1H NMR (400 MHz, CDCl₃) δ 7.36-7.32 (m, 5H); 5.11 (s, 2H); 4.31 (t, 1H); 3.35 (dt, 2H); 2.34 (t, 2H), 1.53 (m, 2H); 1.39 (m, 2H), 1.55-1.25 (m, 26H).

Step 3. To a mixture of compound 8-2 (15.2 g, 3.7 mmol) in dry DCM (30 mL) was added PCC (16.0 g, 7.4 mmol) under N₂. The reaction mixture was stirred at room temperature overnight under N₂. The reaction mixture was concentrated under reduced pressure. The crude product was chromatographed on silica gel (Petroleum ether/EtOAc 1:0→10:1) to give compound 8-3 (587 mg, 39%) as a pale-yellow solid. MS (ESI) m/z 403.2 [M+H]⁺. 1H NMR (400 MHz, CDCl₃) δ 9.76 (t, J=2.4 Hz, 1H); 7.36-7.31 (m, 5H); 5.11 (s, 2H); 2.41 (dt, J=9.6 Hz, 2.4 Hz, 2H); 2.35 (t, J=10 Hz, 2H); 1.64-1.60 (m, 4H); 1.28-1.24 (m, 26H).

Step 4. To a mixture of compound 8-3 (587 mg, 1.5 mmol) in MeOH (10 mL) cooled to 0° C. was added Ohira-Bestmann reagent (437 mg, 2.25 mmol), K₂CO₃ (311 mg, 2.25 mmol) under N₂. The reaction mixture was stirred at room temperature overnight under N₂. The reaction mixture was concentrated under reduced pressure. The crude product was chromatographed on silica gel (Petroleum ether/EtOAc 1:0→20:1) to give compound 8-4 (373 mg, 79%) as a pale-yellow solid. ¹H NMR (400 MHz, CDCl₃) δ 3.66 (s, 3H); 2.30 (t, J=10.2 Hz, 2H); 2.18 (dt, J=9.4, 3.6 Hz, 2H); 1.93 (t, J=3.4 Hz, 1H); 1.64-1.59 (m, 2H); 1.52-1.47 (m, 2H); 1.40-1.36 (m, 2H); 1.36-1.25 (m, 24H).

Step 5. To a solution of compound 8-4 (373 mg, 1.2 mmol) in MeOH (10 mL), THE (10 mL), H₂O (10 mL) at room temperature was added KOH (202 mg, 3.6 mmol). The reaction mixture was stirred at 50° C. overnight. The reaction mixture was concentrated under reduced pressure. The residue was diluted with solvent and poured into water. The aqueous phase was acidified with aqueous HCl and extracted with EtOAc (15 mL×4). The combined organic layers were dried over MgSO₄ and concentrated. The crude product was chromatographed on silica gel (Petroleum ether/EtOAc 1:0→10:1) to give compound 8-5 (368 mg, 100%) as a white solid. MS (ESI) m/z 307.2 [M−H]⁻. ¹H NMR (400 MHz, CDCl₃) δ 2.35 (t, J=7.4 Hz, 2H); 2.20-2.15 (m, 2H); 1.93 (t, J=2.6 Hz, 1H); 1.66-1.50 (m, 4H); 1.40-1.25 (m, 26H).

Step 6. To a solution of icos-19-ynoic acid, 8-5, (2.0 g, 6.5 mmol) and 1-hydroxy-pyrrolidine-2,5-dione (1.5 g, 12.9 mmol) in DCM (40 mL) was added EDCI (2.5 g, 12.945 mmol) in portions at room temperature. The reaction mixture was stirred for 16 hours. The reaction mixture was quenched with water (40 mL) and extracted with DCM (30 mL×3). The combined organic layers were washed with water (40 mL×2) and brine (40 mL), dried over Na₂SO₄ and concentrated. The residue was purified by chromatography on silica gel (EA/PE=0-10%) to give 2,5-dioxopyrrolidin-1-yl icos-19-ynoate, 8-6, as a white solid. ¹H NMR (400 MHz, CDCl₃): δ=2.83 (brs, 4H); 2.60 (t, 2H); 2.18 (td, 2H); 1.93 (t, 1H); 1.74 (m, 2H); 1.45-1.50 (m, 2H); 1.35-1.40 (m, 4H); 1.25-1.30 (22H).

Step 7. To a solution of 3,3′,3″,3′″-(ethane-1,2-diylbis (azanetriyl))tetrakis(N-(2-aminoethyl) propanamide) (200.0 mg, 0.109 mmol, purified from commercial material) and TEA (220.0 mg, 1.09 mmol) in DMF (10 mL) was added 8-6 (266.0 mg, 0.655 mmol) in portions. The resulting mixture was heated to 60° C. and stirred for 16 hours. The reaction mixture was diluted with water (30 mL) and filtered. The filter cake was washed with water (10 mL×1), MeOH (10 mL×2) and EA (10 mL×2) successively, and air-dried overnight to give 8-7 (150 mg, yield: 82.0%) as a white solid. ¹H NMR (400 MHz, CF₃COOD): δ=4.23 (brs, 4H); 3.83 (m, 8H); 3.76 (m, 8H); 3.66 (m, 8H); 3.15 (m, 8H); 2.68 (t, 8H); 2.29-2.25 (m, 8H); 2.11 (s, 4H); 1.81 (br, 8H); 1.62 (br, 8H); 1.50 (br, 8H); 1.39 (br, 96H).

Step 8. To a solution of 194 (1.7 mg, 0.003 mmol) in DCM/EtOH/H2O (0.2/0.1/0.1 mL) was added 8-7 (1.9 mg, 0.001 mmol), L-Ascorbic acid sodium salt (0.2 M, 30 uL, H₂O solution) and Copper(II) sulfate pentahydrate (0.1 M, 30 uL, H₂O solution). The reaction mixture was stirred at room temperature for 16 hours. The combined reaction mixture was concentrated. The residue was treated with DMSO (2 mL)/TFA (0.8 mL), stirred for 2 h at room temperature and filtered. The filtrate was then purified by prep-HPLC (TFA method, C8 column) to give Compound No, 8 (TFA salt, 1.5 mg, yield: 3.4%) as a white solid. MS: m/3z 1305.1 (M/3+H⁺).

### Example 14. Synthesis of Compound No. 204

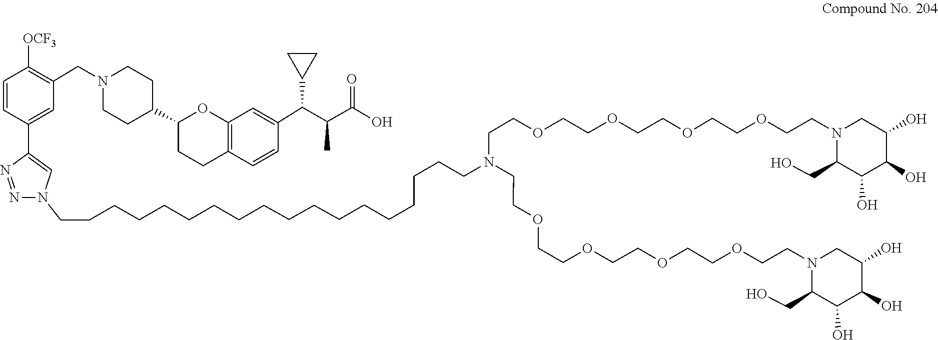 [SMILES: C[C@H](C(=O)O)[C@H](c1ccc2c(c1)O[C@@H](C1CCN(Cc3cc(-c4cn(CCCCCCCCCCCCCCCCCCN(CCOCCOCCOCCOCCN5C[C@H](O)[C@@H](O)[C@H](O)[C@H]5CO)CCOCCOCCOCCOCCN5C[C@H](O)[C@@H](O)[C@H](O)[C@H]5CO)nn4)ccc3OC(F)(F)F)CC1)CC2)C1CC1]

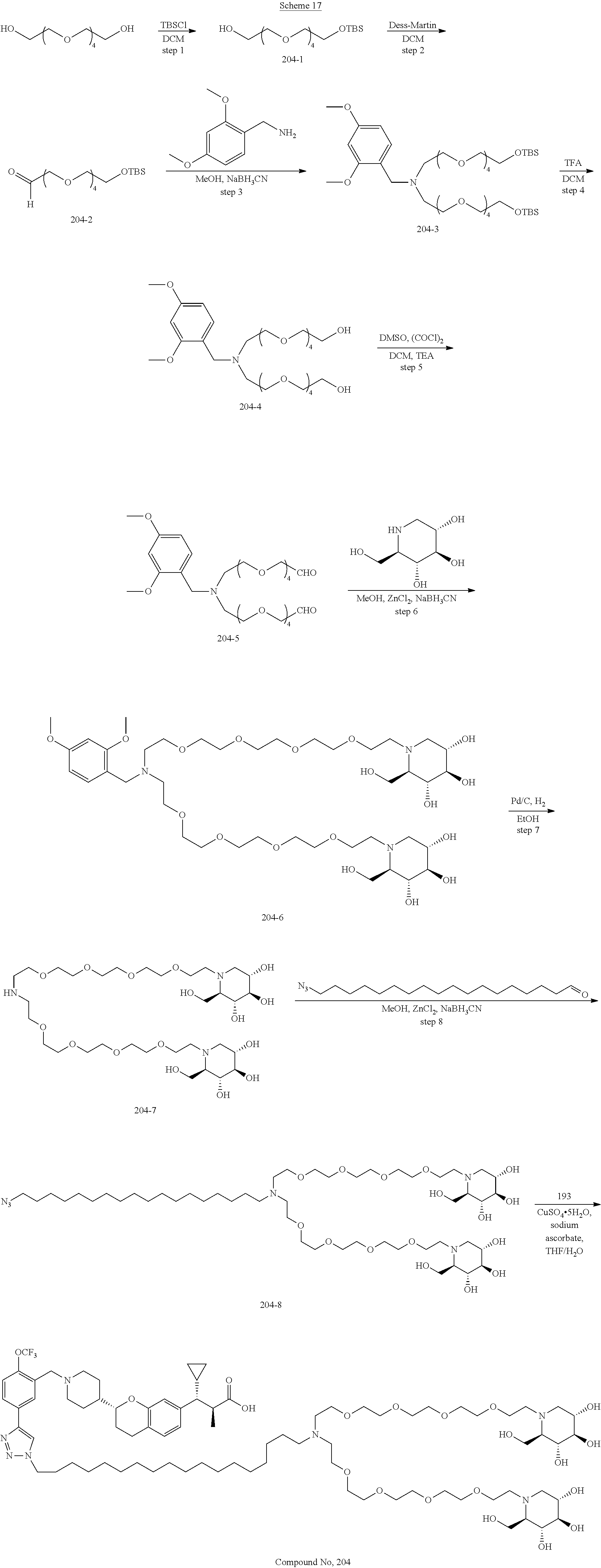

Compound 204 contains two miglitol residues. Miglitol is a known alpha-glucosidase inhibitor.

Step 1. To a solution of 3,6,9,12-tetraoxatetradecane-1,14-diol (100 g, 0.42 mol) and imidazole (42 g, 0.63 mol) in DCM (1 L) cooled to 0° C. was added TBSCI (63 g, 0.42 mol). The reaction mixture was stirred at room temperature overnight. Water (1 L) was added and the mixture was extracted with DCM (1 L×3), and the organic layer was dried by Na₂SO₄ and concentrated to give crude produce. The crude produce was purified by column chromatography on silica gel (PE/EA=1/1) to afford 2,2,3,3-tetramethyl-4,7,10,13,16-pentaoxa-3-silaoctadecan-18-ol, 204-1, (50 g, 34% yield) as a yellow oil.

Step 2. To a solution of 204-1 (50 g, 0.14 mmol) in DCM (500 mL) was added Dess-Martin (89 g, 0.21 mol) at 0° C. The reaction mixture was stirred at room temperature overnight. The mixture was quenched by the addition of saturated aqueous Na₂S₂O₄ and was filtered. The filtrate was extracted by DCM (1 L×3) and the organic layer was dried by Na₂SO₄ and concentrated to give crude produce. The crude produce was purified by column chromatography on silica gel (PE/EA=2/1) to afford 2,2,3,3-tetramethyl-4,7,10,13,16-pentaoxa-3-silaoctadecan-18-al, 204-2, as a yellow oil. ¹HNMR (400 MHz, CDCl₃): 9.67 (s, 1H); 4.09 (s, 2H); 3.71-3.57 (m, 14H); 3.51-3.48 (m, 2H); 0.83 (s, 9H); 0.10 (6H).

Step 3. To a solution of 204-2 (29 g, 0.08 mol) and (2,4-dimethoxyphenyl)methanamine (2.6 g, 0.015 mol) in MeOH (50 mL) was added NaBH₃CN (2.8 g, 0.045 mol). The resulting reaction mixture was stirred at room temperature overnight. The mixture was quenched by H₂O and was concentrated to give crude product. The crude produce was purified by column chromatography on silica gel (DCM/MeOH=10/1) to give 204-3 as a yellow oil. MS (ESI) m/z 836.6 [M+H]⁺

Step 4. To a solution of 204-3 (15 g, 0.018 mol) in DCM/TFA (150 mL/15 mL) was stirred at room temperature overnight. The mixture was concentrated and was purified by flash to give 204-4 as a yellow oil. MS (ESI) m/z 608.4[M+H]⁺

Step 5. To a solution of oxalyl chloride (6.5 g, 0.05 mol) in DCM (50 mL) was added DMSO (4.7 g, 0.06 mol) dropwise at −78° C. The resulting reaction mixture was stirred at −78° C. for 1 hr. Then a solution of 204-4 (5.2 g, 8.5 mmol) in DCM (10 mL) was added into the above mixture slowly. After stirred at −78° C. for 50 minutes, TEA (8.5 g, 85 mmol) was added and the reaction mixture was stirred at −78° C. for 1 hr. The mixture was quenched with water (200 mL) and layers separated. The aqueous phase was extracted with DCM (3×300 mL). Then the organic phase was combined and washed with brine (2×100 mL), dried over Na₂SO₄, and concentrated in vacuo to give 204-5 as yellow oil. MS (ESI) m/z 604.2 [M+H]⁺

Step 6. To a solution of 204-5 (2.5 g, 4.1 mmol) in MeOH was added (2R,3R,4R,5S)-2-(hydroxymethyl) piperidine-3,4,5-triol (2.6 g, 16.4 mmol) and NaBH₃CN (1.03 g, 16.4 mmol) and ZnCl₂ (2.2 g, 16.4 mmol). The resulting reaction mixture was stirred at 50° C. overnight. The mixture was quenched by H₂O and concentrated to give crude produce. The crude produce was purified by flash to give (2R,2′R,3R,3′R,4R,4′R,5S,5'S)-1,1′-(15-(2,4-dimethoxybenzyl)-3,6,9,12,18,21,24,27-octaoxa-15-azanonacosane-1,29-diyl)bis(2-(hydroxymethyl)piperidine-3,4,5-triol), 204-6, as a yellow oil. MS (ESI) m/z 449.9 [M/2+H]⁺. ¹H NMR (400 MHz, DMSO-d₆): 7.22 (d, J=8.0 Hz, 1H); 6.50 (s, 1H); 6.47 (d, J=8.04 Hz, 1H); 4.68-4.63 (m, 6H); 4.08-4.06 (m, 2H); 3.75 (s, 3H); 3.74 (s, 3H); 3.73-3.71 (m, 1H); 3.56-3.43 (m, 36H); 3.17-3.16 (m, 3H); 3.01-2.89 (m, 8H); 2.60-2.58 (m, 5H); 2.08-1.97 (m, 5H).

Step 7. To a solution of 204-6 (1.5 g, 1.6 mmol) in EtOH was added Pd/C (160 mg, 10%). The mixture was stirred at room temperature overnight under H₂. The mixture was filtered and concentrated to give crude produce. The crude produce was purified by prep-HPLC to give (2R,2′R,3R,3′R,4R,4′R,5S,5'S)-1,1′-(3,6,9,12,18,21,24,27-octaoxa-15-azanonacosane-1,29-diyl)bis(2-(hydroxymethyl)piperidine-3,4,5-triol), 204-7, as a yellow oil. MS (ESI) m/z 374.8 [M/2+H]⁺. ¹H NMR (400 MHz, DMSO-d₆): 9.46 (s, 1H); 8.57 (s, 1H); 5.54 (br, 5H); 3.91-3.65 (m, 14H); 3.56-3.47 (m, 27H); 3.45-3.29 (m, 11H); 3.18-3.14 (m, 4H); 3.04-2.98 (m, 3H).

Step 8. To a solution of 204-7 (600 mg, 0.80 mmol) and 18-azidooctadecanal (297 mg, 0.96 mmol) in MeOH (10 mL) was added NaBH₃CN (151 mg, 2.4 mmol) and ZnCl₂ (326 mg, 2.4 mmol). The resulting reaction mixture was stirred at 60° C. overnight. The mixture was quenched by H₂O and concentrated to give crude product. The crude product was purified by prep-HPLC to give (2R,2′R,3R,3′R,4R,4′R,5S,5'S)-1,1′-(15-(18-azidooctadecyl)-3,6,9,12,18,21,24,27-octaoxa-15-azanonacosane-1,29-diyl)bis(2-(hydroxymethyl)piperidine-3,4,5-triol), 204-8 (160 mg, 19% yield) as a yellow oil. MS (ESI) m/z 521.4 [M/2+H]⁺. ¹H NMR (400 MHz, DMSO-d₆): 9.49 (br, 2H); 5.75 (s, 5H); 3.94-3.73 (m, 24H); 3.57-3.52 (m, 24H; 3.43-3.29 (m, 11H; 3.19-2.93 (m, 8H; 1.70-1.43 (m, 2H; 1.24 (s, 24H).

Step 9. To a solution of 204-8 (160 mg, 0.15 mmol) and (2S,3R)-3-cyclopropyl-3-((R)-2-(1-(5-ethynyl-2-(trifluoromethoxy) benzyl)piperidin-4-yl)chroman-7-yl)-2-methylpropanoic acid (81 mg, 0.15 mmol) in THF/H₂O (2 mL/1 mL) was added CuSO₄·5H₂O (37 mg, 0.15 mmol) and sodium ascorbate (29.7 mg, 0.15 mmol). The reaction mixture was stirred at 50° C. overnight. The mixture was filtered and concentrated to give crude produce. The crude produce was purified by prep-HPLC to give (2S,3R)-3-cyclopropyl-2-methyl-3-((S)-2-(1-(2-(trifluoromethoxy)-4-(1-(1-((2R,3R,4R,5S)-3,4,5-trihydroxy-2-(hydroxymethyl)piperidin-1-yl)-15-(14-((2R,3R,4R,5S)-3,4,5-trihydroxy-2-(hydroxymethyl)piperidin-1-yl)-3,6,9,12-tetraoxatetradecyl)-3,6,9,12-tetraoxa-15-azatritriacontan-33-yl)-1H-1,2,3-triazol-4-yl)benzyl) piperidin-4-yl)chroman-7-yl)propanoic acid, Compound No. 204, (36 mg, 15% yield) as a white solid. ¹H NMR (400 MHz, DMSO-d₆): 9.66-9.36 (m, 3H); 8.65 (s, 1H); 8.33 (s, 1H); 8.00 (s, 1H); 7.60 (s, 1H); 6.96 (d, J=7.6 Hz, 1H); 6.65 (d, J=8.8 Hz, 1H); 6.51 (s, 1H); 5.73-5.26 (m, 7H); 4.44-4.40 (m, 3H); 3.93-3.72 (m, 11H); 3.56-3.44 (m, 35H); 3.20-2.98 (m, 18H); 2.84-2.58 (m, 3H); 2.35-2.32 (m, 1H); 2.13-1.53 (m, 13H); 1.22 (s, 26H); 1.09-0.99 (m, 1H); 0.81 (d, J=6.8 Hz, 1H; 0.56-0.45 (m, 1H); 0.28-0.16 (m, 2H). MS (ESI) m/z 792.3 [M/2+H]⁺.

### Example 15. Synthesis of Compound No. 205

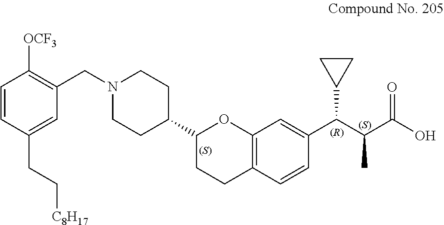

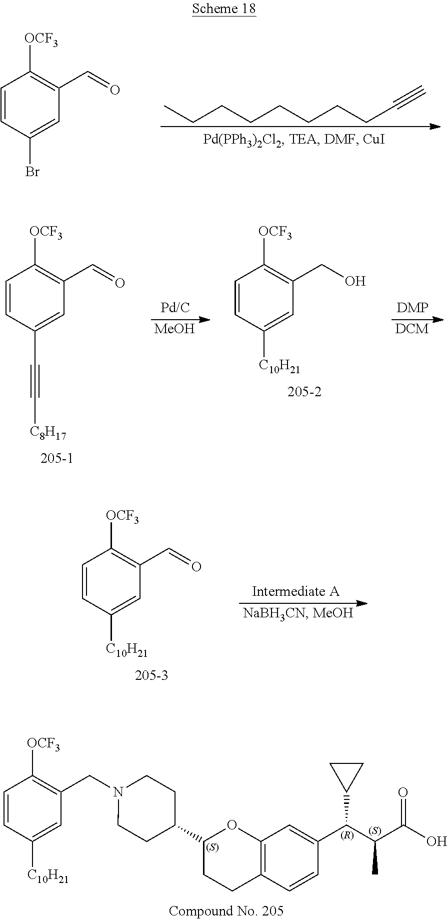

Step 1. To a mixture of 5-bromo-2-(trifluoromethoxy)benzaldehyde (1.0 g, 3.72 mmol) in DMF (10 mL) was added dec-1-yne (0.77 g, 5.58 mmol), TEA (0.75 g, 7.43 mmol), CuI (0.14 g, 0.74 mmol) and Pd(PPh₃)2C1₂ (0.52 g, 0.74 mmol). The mixture was stirred at 90° C. under N₂ for 3 h. The reaction mixture was diluted with water and extracted with EA. The organic phase was washed with brine, dried with Na₂SO₄, and concentrated. The residue was purified by column chromatography on silica gel (PE=100%) to give 5-(dec-1-yn-1-yl)-2-(trifluoromethoxy)benzaldehyde, 205-1, (1.0 g, yield: 83%) as a yellow oil. MS (ESI) m/z 344.1 [M+18]⁺.

Step 2. To a solution of 205-1 (500 mg, 1.53 mmol) in MeOH (10 mL) was added Pd/C (100 mg). The mixture was stirred at 40° C. under H₂ for 12 h. The reaction mixture was filtered and concentrated to give (5-decyl-2-(trifluoromethoxy)phenyl)methanol, 205-2, (410 mg, yield: 80.5%) as a yellow oil.

Step 3. To a solution of 205-2 (410 mg, 1.23 mmol) in DCM (10 mL) was added DMP (1.05 g, 2.47 mmol). The mixture was stirred at room temperature for 2 h. The reaction mixture was concentrated and the residue was purified by column chromatography on silica gel (PE=100%) to give 5-decyl-2-(trifluoromethoxy) benzaldehyde, 205-3, as a colorless oil. ¹H NMR (400 MHz, DMSO-d₆): 10.22 (s, 1H); 7.75 (s, 1H); 7.66 (dd, 1H); 7.47 (d, 1H); 2.68 (t, 2H); 1.58 (m, 2H); 1.28-1.19 (m, 14H); 0.86-0.82 (m, 3H).

Step 4. A mixture of 205-3 (28.9 mg, 0.088 mmol) and (2S,3R)-3-cyclopropyl-2-methyl-3-((R)-2-(piperidin-4-yl)chroman-7-yl)propanoic acid (10 mg, 0.029 mmol) in MeOH (2 mL) was stirred at room temperature for 2 h. Then NaBH₃CN (3.7 mg, 0.058 mmol) was added and the mixture was stirred at room temperature for 12 h. The reaction mixture was concentrated and the residue was purified by pre-HPLC to give (2S,3R)-3-cyclopropyl-3-((R)-2-(1-(5-decyl-2-(trifluoromethoxy)benzyl)piperidin-4-yl)chroman-7-yl)-2-methylpropanoic acid, Compound No. 205, as a white solid. ¹H NMR (400 MHz, CDCl₃): 7.51 (s, 1H); 7.29-7.21 (m, 2H); 6.95 (d, 1H); 6.65 (d, 1H); 6.58 (s, 1H); 4.24 (s, 2H); 3.81 (m, 1H); 3.66 (m, 2H); 2.83-2.66 (m, 3H); 2.60-2.58 (t, 2H); 2.25-1.63 (m, 10H); 1.62-1.57 (m, 2H); 1.30-1.26 (m, 14H); 1.11-1.07 (m, 1H); 0.98-0.96 (d, 3H); 0.89-0.86 (t, 3H); 0.62-0.58 (m, 1H); 0.38-0.33 (m, 2H); 0.05-0.03 (m, 1H). MS (ESI) m/z 658.4 [M+H]⁺.

### Example 16. Synthesis of Compound No. 206

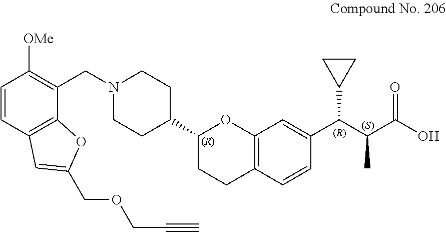

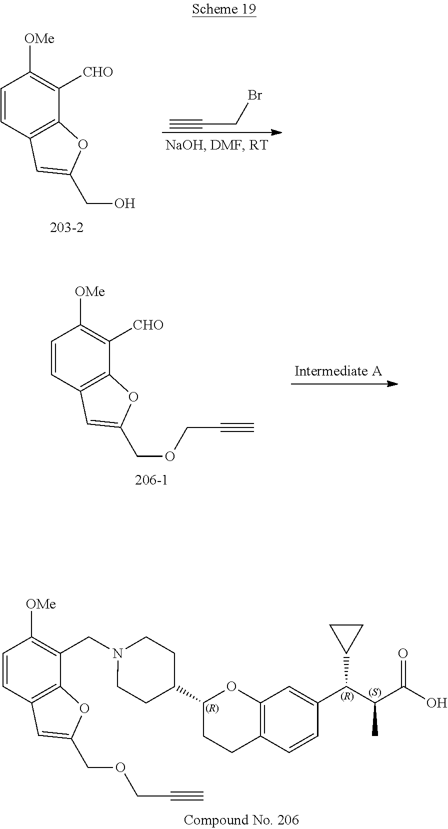

Step 1. To a solution of 203-2 (180 mg, 0.87 mmol) in DMF (5 mL) was added NaOH (87 mg, 2.17 mmol) and 3-bromoprop-1-yne (123 mg, 1.04 mmol). The reaction mixture was stirred at room temperature overnight. The mixture was added H₂O (10 mL) and extracted by EA (10 mL×3). The organic layer was dried with Na₂SO₄ and concentrated. The crude produce was purified by column chromatography on silica gel (PE/EA=5/1) to afford 6-methoxy-2-((prop-2-yn-1-yloxy) methyl)benzofuran-7-carbaldehyde, 206-1, as a yellow oil. MS (ESI) m/z 207.0 [M+H]⁺.

Step 2. To a solution of 206-1 (15 mg, 0.06 mmol) and (2S,3R)-3-cyclopropyl-2-methyl-3-((R)-2-(piperidin-4-yl)chroman-7-yl)propanoic acid (20.58 mg, 0.20 mmol) in DCM (3 mL) was added sodium borohydride (8.4 mg, 0.04 mmol). The reaction mixture was stirred at room temperature overnight. The mixture was concentrated and was purified by prep-HPLC to give (2S,3R)-3-cyclopropyl-3-((R)-2-(1-((6-methoxy-2-((prop-2-yn-1-yloxy)-methyl)benzofuran-7-yl)methyl)piperidin-4-yl)chroman-7-yl)-2-methylpropanoic acid, Compound No. 206, as a white solid. ¹H NMR (400 MHz, CDCl₃): 12.29 (s, 1H); 7.52-7.50 (d, 1H); 6.87-6.85 (d, 2H); 6.65 (s, 1H); 6.57-6.55 (d, 1H); 6.48 (s, 1H); 4.60 (s, 2H); 4.49 (s, 2H); 4.15 (d, J=2 Hz, 2H); 3.84 (s, 3H); 3.72-3.68 (m, 3H); 2.78-2.45 (m, 5H); 2.45 (t, J=2 Hz, 1H); 2.10-1.75 (m, 6H); 1.60-1.50 (m, 2H); 1.05-0.90 (m, 1H); 0.80 (d, 3H); 0.55-0.52 (m, 1H); 0.28-0.26 (m, 2H); 0.05˜−0.05 (m, 1H); MS (ESI) m/z 572.3 [M+H]⁺.

### Example 17. Synthesis of Compound No. 207

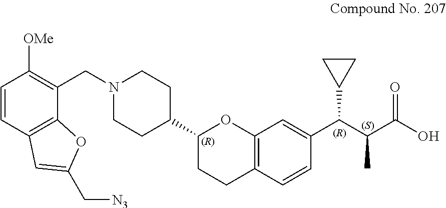

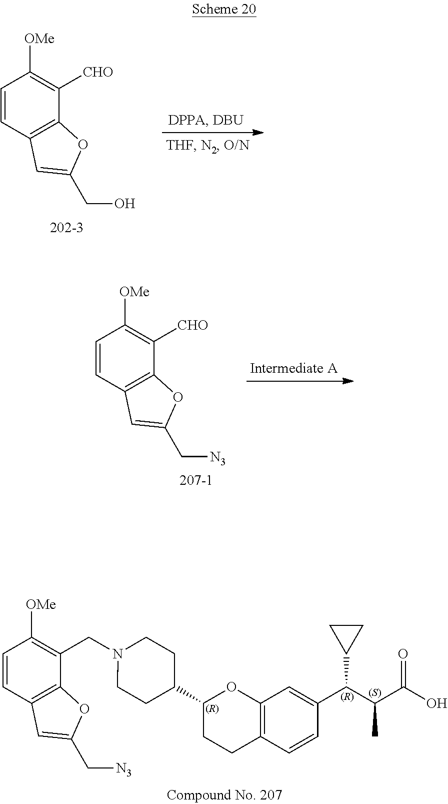

Step 1. To a solution of 203-2 (120 mg, 0.58 mmol) in DMF (5 mL) was added DPPA (400.48 mg, 1.45 mmol) and DBU (266 mg, 1.74 mmol). The resulting reaction mixture was stirred at room temperature overnight under N₂. The mixture was added H₂O (10 mL) and extracted by EA (20×3). The organic layer was dried with Na₂SO₄ and concentrated. The crude produce was purified by column chromatography on silica gel (PE/EA=5/1) to afford 2-(azidomethyl)-6-methoxybenzofuran-7-carbaldehyde, 207-1, as a yellow solid. MS (ESI) m/z 232.0 [M+H]⁺.

Step 2. To a solution of 207-1 (30 mg, 0.13 mmol), and (2S,3R)-3-cyclopropyl-2-methyl-3-((R)-2-(piperidin-4-yl)chroman-7-yl)propanoic acid (44.5 mg, 0.19 mmol) in DCM (3 mL) was added sodium borohydride (54.6 mg, 0.25 mmol). The reaction mixture was stirred at room temperature overnight. The mixture was concentrated and was purified by prep-HPLC to give (2S,3R)-3-((R)-2-(1-((2-(azidomethyl)-6-methoxybenzofuran-7-yl)methyl)-piperidin-4-yl)chroman-7-yl)-3-cyclopropyl-2-methylpropanoic acid, Compound Nol 207, as a white solid. ¹H NMR (400 MHz, CDCl₃): 12.46 (s, 1H); 7.54-7.52 (d, 1H); 6.90-6.85 (d, 2H); 6.64 (s, 1H); 6.57-6.55 (d, 1H); 6.49 (s, 1H); 4.49 (s, 2H); 4.36 (s, 2H); 3.85 (s, 3H); 3.75-3.66 (m, 3H); 2.76-2.56 (m, 5H); 2.00-1.72 (m, 6H); 1.62-1.52 (m, 2H); 1.0-0.90 (m, 1H); 0.86 (d, 3H); 0.55-0.50 (m, 1H); 0.30-0.20 (m, 2H); 0.05-˜−0.05 (m, 1H). MS (ESI) m/z 559.3 [M+H]⁺.

### Example 18. Synthesis of Compound No. 208

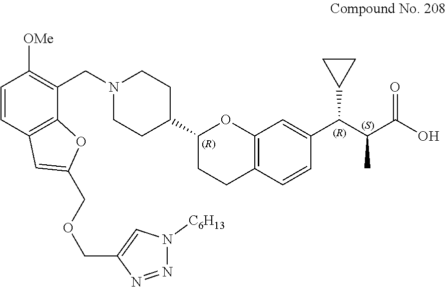

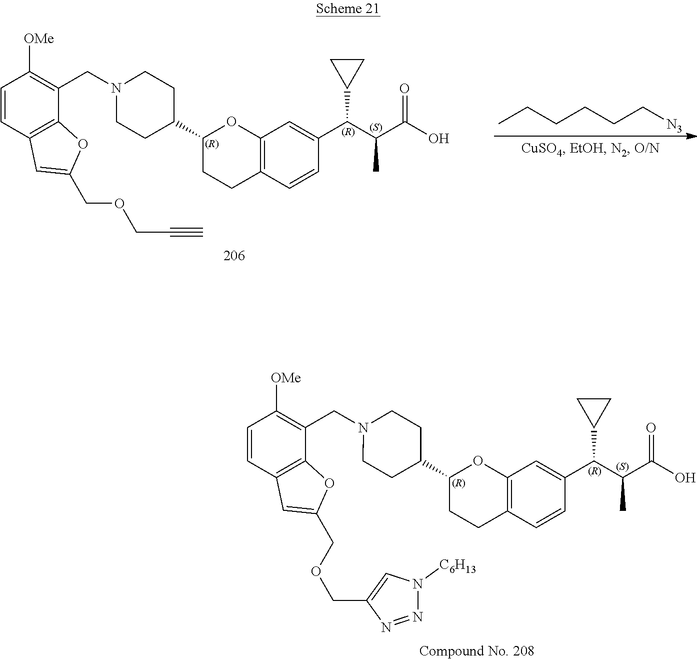

Step 1. To a solution of 206 (10 mg, 0.87 mmol) and 1-azidohexane (2.2 mg, 0.018 mmol) in EtOH/H₂O (1 mL/1 mL) was added CuSO₄·5H₂O (1 mg, 0.0017 mmol) and sodium ascorbate (1 mg, 0.0017 mmol). The resulting reaction mixture was stirred at room temperature overnight. The mixture was concentrated and the crude product was purified by prep-HPLC to give (2S,3R)-3-cyclopropyl-3-((R)-2-(1-((2-(((1-hexyl-1H-1,2,3-triazol-4-yl)methoxy)methyl)-6-methoxybenzofuran-7-yl)methyl)piperidin-4-yl)chroman-7-yl)-2-methylpropanoic acid, Compound No. 208, as a white solid. ¹H NMR (400 MHz, CDCl₃): 12.28 (s, 1H); 7.52-7.50 (d, 1H); 6.87-6.85 (d, 2H); 6.64 (s, 1H); 6.59-6.58 (d, 1H); 6.49 (s, 1H); 4.65 (s, 2H); 4.60 (s, 2H); 4.50 (s, 2H); 4.29 (t, 2H); 3.86 (s, 3H), 3.70-3.65 (m, 3H), 2.80-2.55 (m, 5H), 2.0-1.7 (m, 6H), 1.60-1.50 (m, 4H), 1.30-1.15 (m, 6H); 1.05-0.95 (m, 1H); 0.87 (d, 3H); 0.85-0.75 (m, 3H); 0.55-0.52 (m, 1H); 0.28-0.26 (m, 2H); 0.05˜−0.05 (m, 1H). MS (ESI) m/z 699.6 [M+H]⁺.

### Example 19. Synthesis of Compound No. 209

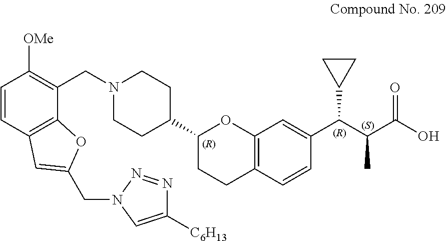

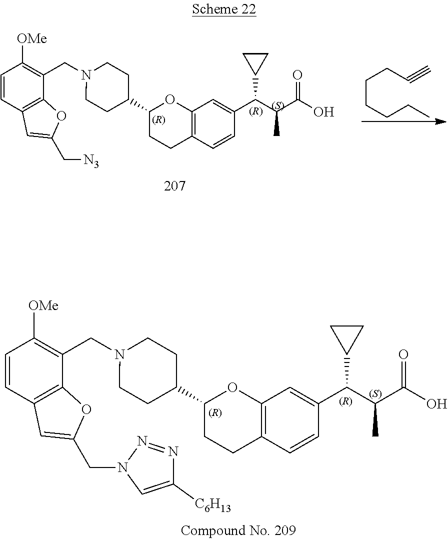

Step 1. To a solution of 207 (25 mg, 0.04 mmol) and oct-1-yne in EtOH/H₂O (1 mL/1 mL) was added CuSO₄ (5.5 mg, 0.022 mmol) and sodium ascorbate (4.35 mg, 0.022 mmol). The resulting reaction mixture was stirred at room temperature overnight. The mixture was concentrated and was purified by prep-HPLC to give (2S,3R)-3-cyclopropyl-3-((R)-2-(1-((2-((4-hexyl-1H-1,2,3-triazol-1-yl)methyl)-6-methoxybenzofuran-7-yl)methyl)piperidin-4-yl)chroman-7-yl)-2-methylpropanoic acid, Compound No. 209, as a white solid. ¹H NMR (400 MHz, CDCl₃): 12.46 (s, 1H); 7.51 (s, 1H); 7.51-7.49 (d, 1H); 6.88-6.87 (d, 1H); 6.87-6.82 (d, 1H); 6.68 (s, 1H); 6.62-6.60 (d, 1H), 6.49 (s, 1H), 5.58-5.42 (q, 2H), 4.43 (s, 2H), 3.85 (s, 3H), 3.72 (m, 1H), 3.62-3.49 (m, 2H); 2.76-2.74 (m, 2H); 2.74-2.60 (m, 4H); 2.50-1.50 (m, 10H); 1.37-1.19 (m, 6H); 1.10-0.99 (m, 1H); 0.94-0.90 (d, 3H); 0.82-0.80 (m, 3H); 0.57-0.47 (m, 1H); 0.36-0.23 (m, 2H); 0.02˜0.05 (m, 1H). MS (ESI) m/z 669.4 [M+H].

### Example 20. Synthesis of Compound No. 210

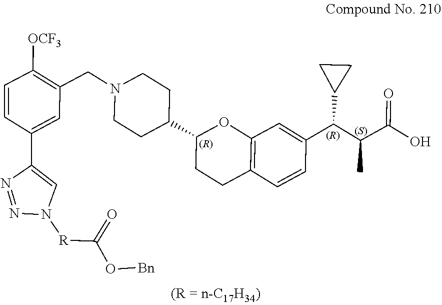

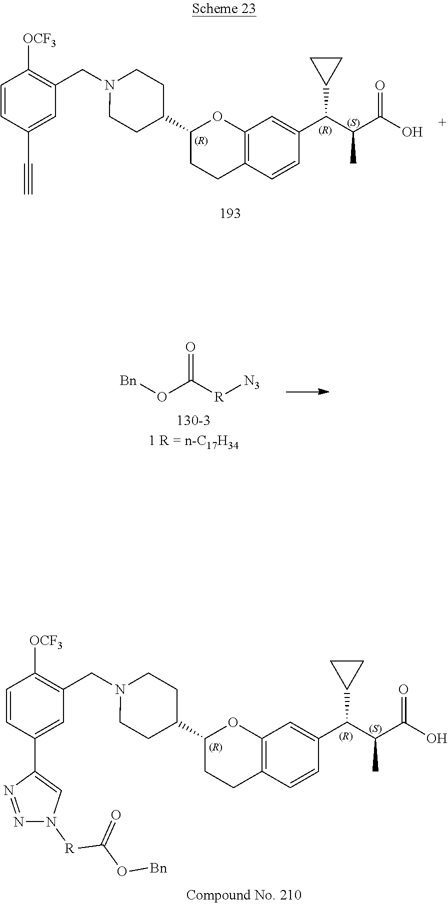

Step 1. To a solution of 193 (10.0 mg, 0.018 mmol) in EtOH/H₂O (1/1 mL) was added benzyl 18-azidooctadecanoate, 130-3 (7.7 mL, 0.018 mmol), L-Ascorbic acid sodium salt (1.4 mg, 0.007 mmol) and Copper(II) sulfate pentahydrate (1.0 mg, 0.004 mmol). The reaction mixture was stirred at room temperature for 16 hours. The reaction mixture was quenched with water and extracted with DCM (5 mL×3). The combined organic layers were washed with water (5 mL×2) and brine (5 mL), dried over Na₂SO₄ and concentrated. The residue was purified by prep HPLC to give (2S,3R)-3-((R)-2-(1-(5-(1-(18-(benzyloxy)-18-oxooctadecyl)-1H-1,2,3-triazol-4-yl)-2-(trifluoromethoxy)benzyl)piperidin-4-yl)chroman-7-yl)-3-cyclopropyl-2-methylpropanoic acid, Compound No. 210, as a white solid. H NMR (400 MHz, CDCl₃): δ=7.99 (s, 1H); 7.83-7.79 (m, 2H); 7.78 (s, 1H); 7.36-7.29 (m, 6H); 6.92 (d, 1H); 6.61 (s, 1H). 6.62-6.60 (d, 1H), 5.11 (s, 2H), 4.38 (t, 2H), 3.81-3.75 (m, 1H), 3.70-3.62 (q, 2H), 3.13-3.01 (m, 2H); 2.87-2.70 (m, 3H); 2.35 (t, 2H); 2.20-1.75 (m, 10H); 1.70-1.58 (m, 4H); 1.33-1.23 (m, 26H); 1.13-1.05 (m, 1H); 0.96 (d, 3H); 0.62-0.55 (m, 1H); 0.36-0.26 (m, 2H); −0.02-−0.03 (m, 2H). MS: m/z 957.7 (M+H⁺).

### Example 21. Synthesis of Compound No. 211

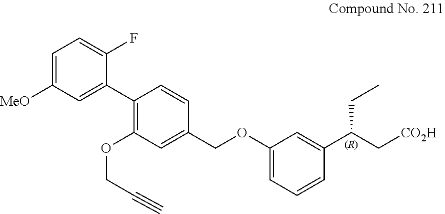

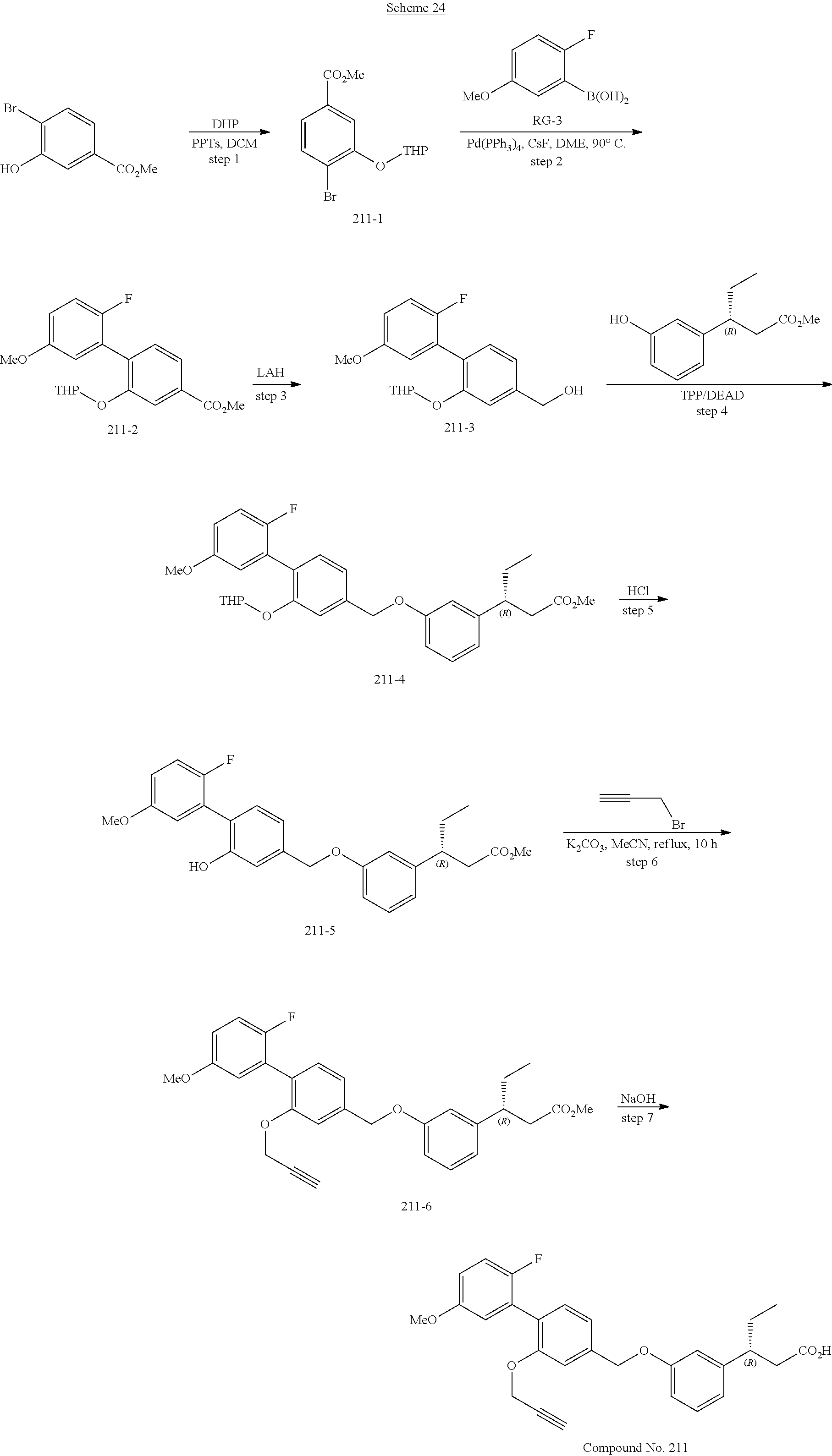

Step 1. To a solution of 4-bromo-3-hydroxy-benzoic acid methyl ester (123 mg, 0.53 mmol) in DCM (5 mL) at room temperature was added DHP (89 mg, 1.06 mmol), PPTs (13 mg, 0.053 mmol). The reaction mixture was stirred at room temperature overnight under N₂. The reaction mixture was quenched with water, extracted with DCM (15 mL×4). The combined organic layers were dried over MgSO₄ and concentrated. The residue was purified by preparative TLC (PE/EA=5:1) to give 4-bromo-3-(tetrahydro-pyran-2-yloxy)-benzoic acid methyl ester, 211-1, (163 mg, 97%) as a pale-yellow oil. MS (ESI) m/z 338.5 [M+Na]⁺. ¹H NMR (400 MHz, CDCl₃) δ 7.76 (d, J=2.4 Hz, 1H); 7.59 (d, J=11 Hz, 1H); 7.52 (dd, J=10.8, 2.4 Hz, 1H); 5.61 (t, J=3.4 Hz, 1H); 3.88 (s, 3H); 3.84 (dd, J=12, 4 Hz, 1H); 3.64-3.47 (m, 1H); 2.13-1.51 (m, 9H).

Step 2. To a mixture of 211-1 (66 mg, 0.2 mmol), phenylboronic acid RG-3 (68 mg, 0.4 mmol) and CsF (91 mg, 0.6 mmol) in dioxane (5 mL) at room temperature was added Pd(PPh₃)₄ (23 mg, 0.02 mmol). The reaction mixture was degassed and filled with N₂ 3 times and heated at 90° C. overnight. The reaction mixture was concentrated and the residue was purified by preparative TLC (PE/EA=5:1) to give 2′-fluoro-5′-methoxy-2-(tetrahydro-pyran-2-yloxy)-biphenyl-4-carboxylic acid methyl ester, 211-2, (70 mg, 93%) as a colorless oil. MS (ESI) m/z 383.3 [M+Na]⁺. ¹H NMR (400 MHz, CDCl₃) δ 7.89 (d, J=0.8 Hz, 1H); 7.74 (dd, J=7.8, 1.4 Hz, 1H); 7.36 (d, J=7.6 Hz, 1H); 7.04 (t, J=8.8 Hz, 1H); 6.90-6.84 (m, 2H); 5.52 (s, 1H); 3.92 (s, 3H); 3.81 (s, 3H); 3.72 (s, 1H); 3.63-3.59 (m, 1H); 1.77-1.50 (m, 6H).

Step 3. To a solution of 211-2 (2.1 g, 5.86 mmol) in THE (80 mL) was added LAH (668 mg, 17.6 mmol) portion wise at 0° C. The reaction mixture was stirred at room temperature overnight under N₂. The reaction mixture was quenched by the addition of H₂O (0.356 mL), NaOH (15%, 0.356 mL) and H₂O (2.025 mL) at 0° C. The reaction mixture was filtered and the filter cake was washed with EA. The combined organic phase was dried over MgSO₄ and concentrated. The residue was chromatographed on silica gel (Petroleum ether/EtOAc 50:10-40:10-30:10) to give [2′-fluoro-5′-methoxy-2-(tetrahydro-pyran-2-yloxy)-biphenyl-4-yl]-methanol, 211-3, (1885 mg, 97%) as a colorless oil. ¹H NMR (400 MHz, CDCl₃) δ 7.28 (t, J=7.6 Hz, 2H); 7.07 (d, J=7.6 Hz, 1H); 7.03 (t, J=9 Hz, 1H); 6.88 (t, J=2.8, 1H); 6.87-6.81 (m, 1H); 5.45 (s, 1H); 4.72 (d, J=5.6 Hz, 2H); 3.83 (dd, J=11, 2.2 Hz, 1H); 3.80 (s, 3H), 3.59-3.56 (m, 1H); 1.82-1.50 (m, 14H).

Step 4. A solution of 211-3 (230 mg, 0.69 mmol), (R)-3-(3-Hydroxy-phenyl)-pentanoic acid methyl ester (120 mg, 0.57 mmol) and PPh₃ (228 mg, 0.865 mmol) in DCM (15 mL) was degassed and filled with N₂ 3 times and cooled to 0° C. DEAD (151 mg, 0.865 mmol) was then added dropwise via syringe. The resulting mixture was then stirred overnight under N₂ allowing the temperature to slowly warm to r. t. Solvent was removed and the residue was purified by flash chromatography (18% EA in PE) to give (R)-3-(3-((2′-fluoro-5′-methoxy-2-(prop-2-yn-1-yloxy)-[1,1′-biphenyl]-4-yl)methoxy) phenyl)pentanoic acid, 211-4, (220 mg, yield: 73%) as a white gum. MS (ESI) m/z 540.1 [M+18]⁺.

Step 5. To a solution of 211-4 (220 mg, 0.42 mmol) in MeOH (15 mL) at room temperature under N₂ was added aqueous HCl (1M, 3 mL) dropwise. The reaction mixture was stirred at room temperature for 2 hrs. MeOH was evaporated under reduced pressure and the residue was diluted with water, extracted with EtOAc (20 mL×3). The combined organic layers were dried over MgSO₄ and concentrated in vacuo to give the crude (R)-methyl-3-(3-((2′-fluoro-2-hydroxy-5′-methoxy-[1,1′-biphenyl]-4-yl) methoxy)phenyl)pentanoate, 211-5, (200 mg crude) as white gum, which was used in the next step.

Step 6. A mixture of crude 211-5 (200 mg, 0.42 mmol), 3-bromoprop-1-yne (100 mg, 0.84 mmol) and K₂CO₃ (117 mg, 0.84 mmol) in ACN (20 mL) was stirred at 80° C. overnight under N₂. The reaction mixture was concentrated under reduced pressure to remove ACN. The residue was poured into water and extracted with EtOAc (15 mL×4). The combined organic layers were dried over Na₂SO₄ and concentrated to give crude (R)-methyl 3-(3-((2′-fluoro-5′-methoxy-2-(prop-2-yn-1-yloxy)-[1,1′-biphenyl]-4-yl) methoxy)phenyl)pentanoate, 211-6, (200 mg, crude) as a white gum. MS (ESI) m/z 494.0 [M+18]⁺.

Step 7. A mixture of 211-6 (200 mg, crude, 0.42 mmol) and NaOH (1N, 1.7 mL). in MeOH (2 mL) and THE (4 mL) was stirred at room temperature for 4 hrs. MeOH was removed and the residue was acidified with aqueous HCl until pH reached 3 and extracted with EtOAc (15 mL×3). The combined organic layers were dried over MgSO₄ and concentrated. The residue was purified by preparative HPLC (TFA method) to give Compound No. 211 (75 mg, 38% over 3 steps) as white gum. MS (ESI) m/z 461.2 [M−H]⁻. ¹H NMR (400 MHz, CDCl₃) δ 7.33-7.31 (m, 1H); 7.25-7.21 (t, 1H); 7.23 (s, 1H); 7.06-7.01 (t, 1H); 6.89-6.80 (m, 5H); 5.10 (s, 2H); 4.69 (d, J=2.4 Hz, 2H); 3.80 (s, 3H); 2.99-2.94 (m, 1H); 2.69-2.57 (m, 2H); 2.44 (t, J=2.4 Hz, 1H); 1.76-1.69 (m, 1H); 1.63-1.56 (m, 1H); 0.80 (t, 3H).

### Example 22. Synthesis of Compound No. 212

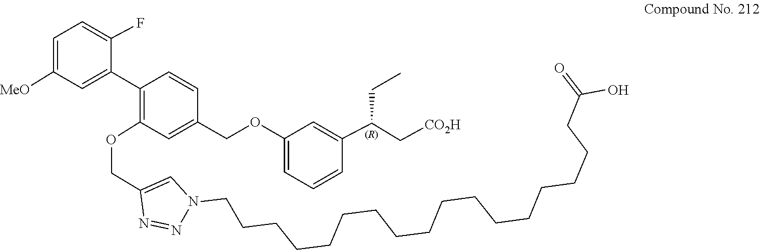

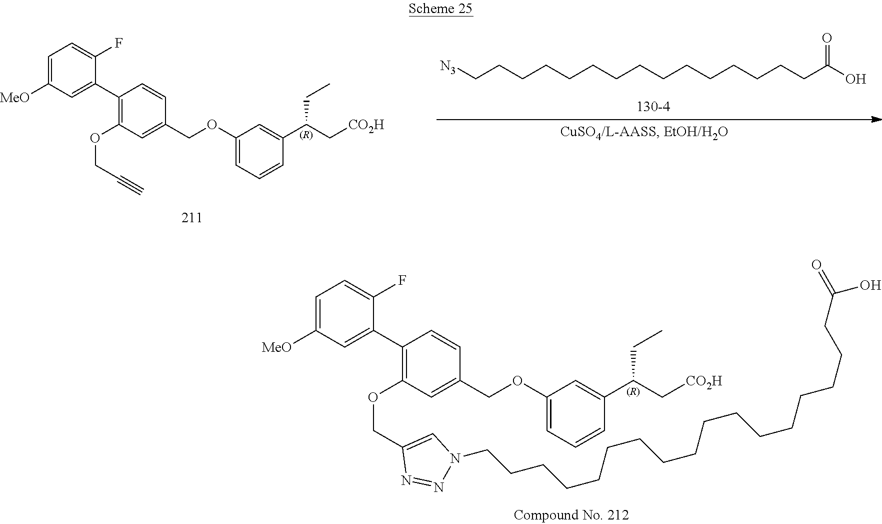

Step 1. To a mixture of 211 (15 mg, 0.032 mmol) and 130-4 (16 mg, 0.018 mmol) in EtOH (2 mL) and MeCN (1 mL) was added CuSO₄·5H₂O (0.1 M, 64 uL) and L-AASS (0.2 M, 64 uL). The resulting mixture was then stirred for 16 hrs at r. t. The reaction mixture was concentrated and the residue was then purified by prep-HPLC (TFA method) to give Compound No. 212 (10 mg, yield: 40%) as white solid. MS (ESI) m/z 788.6 [M+H]⁺. ¹H NMR (400 MHz, CDCl₃) δ 7.34 (s, 1H); 7.30-7.28 (d, 1H); 7.20 (t, 1H); 7.08 (s, 1H); 7.08-7.06 (d, 1H); 7.03-6.99 (t, 1H); 6.92 (s, 1H); 6.87-6.80 (m, 3H); 6.77-6.74 (m, 1H); 5.15 (s, 2H); 5.11 (s, 2H); 4.27 (t, 2H); 3.77 (s, 3H); 3.02-2.99 (m, 1H); 273-2.62 (m, 2H); 2.33 (t, J=6.8 Hz, 2H); 1.87-1.82 (m, 2H); 1.73-1.58 (m, 4H); 1.32-1.18 (m, 26H); 0.83 (t, J=7.2 Hz, 3H).

### Example 23. Synthesis of Compound No. 213

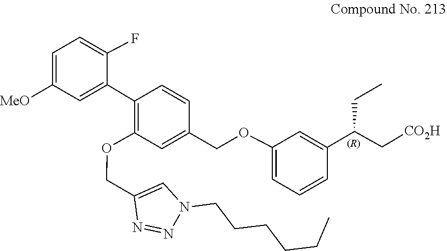

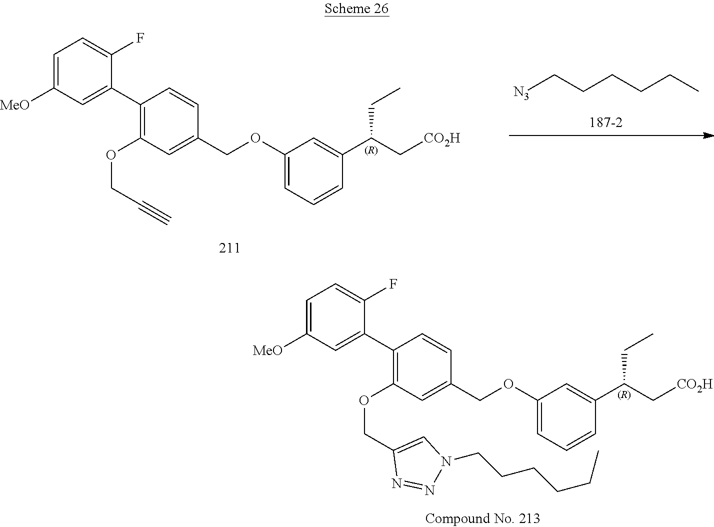

To a mixture of 211 (15 mg, 0.032 mmol) and 187-2 (0.2 M, 0.24 mL, 0.048 mmol) in EtOH (2 mL) and water (0.5 mL) was added CuSO₄·5H₂O (0.1 M, 60 uL) and L-AASS (0.2 M, 60 uL). The resulting mixture was then stirred for 16 hrs at r. t. The reaction mixture was concentrated and the residue was then purified by prep-IPLC (TFA method) to give Compound No. 213 (5 mg, yield: 26.3%) as yellow solid. MS (ESI) m/z 590.4 [M+H]⁺. ¹H NMR (400 MHz, CDCl₃) δ 7.33 (s, 1H); 7.29-7.26 (m, 1H); 7.18 (t, J=8.0 Hz, 1H); 7.05-6.95 (m, 4H); 6.86-6.80 (m, 3H); 6.73-6.70 (m, 1H); 5.20 (s, 2H); 5.13-5.06 (m, 2H); 4.26 (t, J=7.2 Hz, 2H); 3.76 (s, 3H); 3.05-2.99 (m, 1H); 272-2.62 (m, 2H); 1.86-1.82 (m, 2H); 1.72-1.62 (m, 2H); 1.32-1.20 (m, 8H); 0.88-0.81 (m, 6H).

### Example 24. Synthesis of Compound No. 214

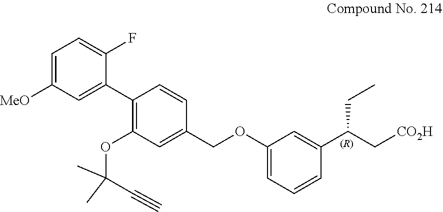

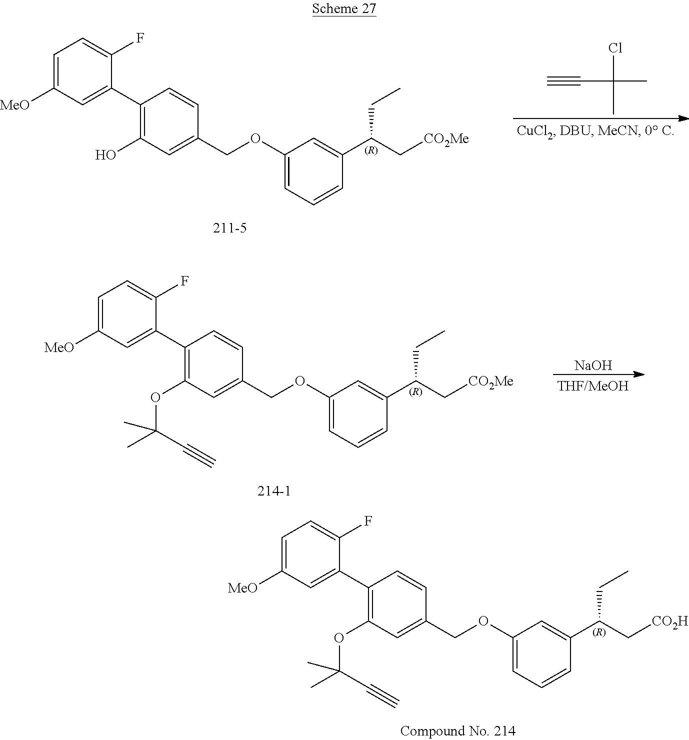

Step 1. To a 211-5 (80 mg, 0.182 mmol) in dry MeCN (5 mL) at 0° C. was added DBU (55 mg, 0.364 mmo) and CuCl₂·2H₂O (15 mg, 0.091 mmol). The resulting mixture was stirred for 15 min at 0° C. under N₂, then chloro-3-methyl-but-1-yne (28 mg, 0.274 mmol) was added via syringe. Then reaction mixture was stirred for 2 hrs at 0° C. and stirred for additional 2 hrs at r. t. Solvent was removed and the residue was purified by prep-TLC (EA/PE=1:4) to give (R)-methyl 3-(3-((2′-fluoro-5′-methoxy-2-((2-methylbut-3-yn-2-yl)oxy)-[1,1′-biphenyl]-4-yl) methoxy)phenyl)pentanoate 214-1 (65 mg, yield: 70%) as white gum. MS (ESI) m/z 505.3 [M+H]⁺.

Step 2. A mixture 214-1 (65 mg, 0.13 mmol) and NaOH (1N, 0.5 mL) in MeOH (2 mL) and THE (2 mL) was stirred at room temperature for 14 hrs. TLC indicated the completion of reaction. MeOH was removed and the residue was acidified with aqueous HCl until pH reached 3 and extracted with EtOAc (15 mL×3). The combined organic layers were dried over MgSO₄ and concentrated. The residue was purified by preparative HPLC (TFA method) to give Compound No. 214 (26 mg, yield: 42%) as white solid. MS (ESI) m/z 489.2 [M−H]⁻. ¹H NMR (400 MHz, CDCl₃) δ 7.70 (s, 1H); 7.32-7.31 (d, J=8.0 Hz, 1H); 7.21 (t, 1H); 7.17-7.16 (d, 1H); 7.00 (t, J=8.8 Hz, 1H); 6.89-6.78 (m, 5H); 5.09 (s, 2H); 3.79 (s, 3H); 2.99-2.95 (m, 1H); 2.63-2.60 (m, 2H); 2.47 (s, 1H); 1.73-1.69 (m, 1H); 1.62-1.57 (m, 1H); 1.46 (s, 6H); 0.78 (t, J=7.2 Hz, 3H).

### Example 25. Synthesis of Compound No. 215

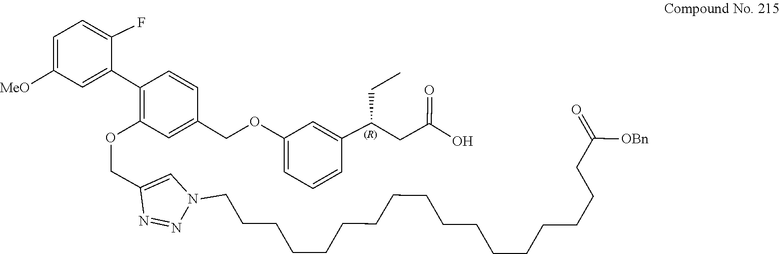

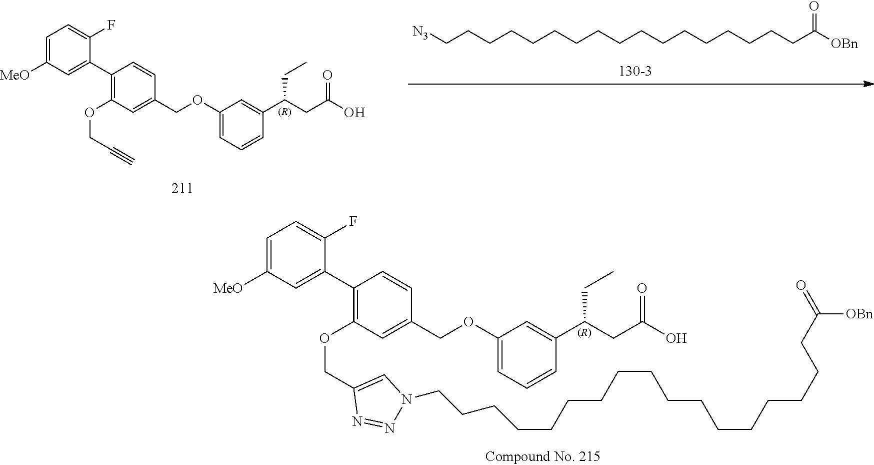

Step 1. To a solution of 211 (15.0 mg, 0.032 mmol) in EtOH/H₂O (2/2 mL) was added 130-3 (13.0 mg, 0.032 mmol), L-Ascorbic acid sodium salt (2.5 mg, 0.013 mmol) and Copper(II) sulfate pentahydrate (1.6 mg, 0.006 mmol). The reaction mixture was stirred at room temperature for 16 hours. The reaction mixture was quenched with water and extracted with DCM (5 mL×3). The combined organic layers were washed with water (5 mL×2) and brine (5 mL), dried over Na₂SO₄ and concentrated. The residue was purified by pre.HPLC to give (R)-3-(3-((2-((1-(18-(benzyloxy)-18-oxooctadecyl)-1H-1,2,3-triazol-4-yl)methoxy)-2′-fluoro-5′-methoxy[1,1′-biphenyl]-4-yl)methoxy)phenyl)pentanoic acid, Compound No. 215, (5.0 mg, yield: 17.6%) as a colorless colloidal. ¹H NMR (400 MHz, CDCl₃): δ=7.36-7.26 (m, 7H); 7.17 (t, 1H); 7.04-6.98 (m, 4H); 6.86-6.80 (m, 3H); 6.70 (dd, J=8.0, 2.0 Hz, 1H); 5.24 (s, 2H); 5.11 (s, 2H); 5.06 (q, 2H); 4.26 (t, 2H); 3.76 (s, 3H); 3.05-2.98 (m, 1H); 2.74-2.60 (m, 2H); 2.35 (t, J=7.6 Hz, 2H); 1.87-1.75 (m, 1H); 1.74-1.60 (m, 4H); 1.38-1.24 (m, 26H); 0.90-0.82 (m, 3H). MS: m/z 878.7 (M+H⁺).

### Example 26. Synthesis of Compound No. 216

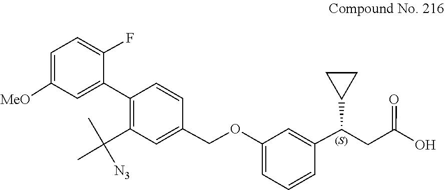

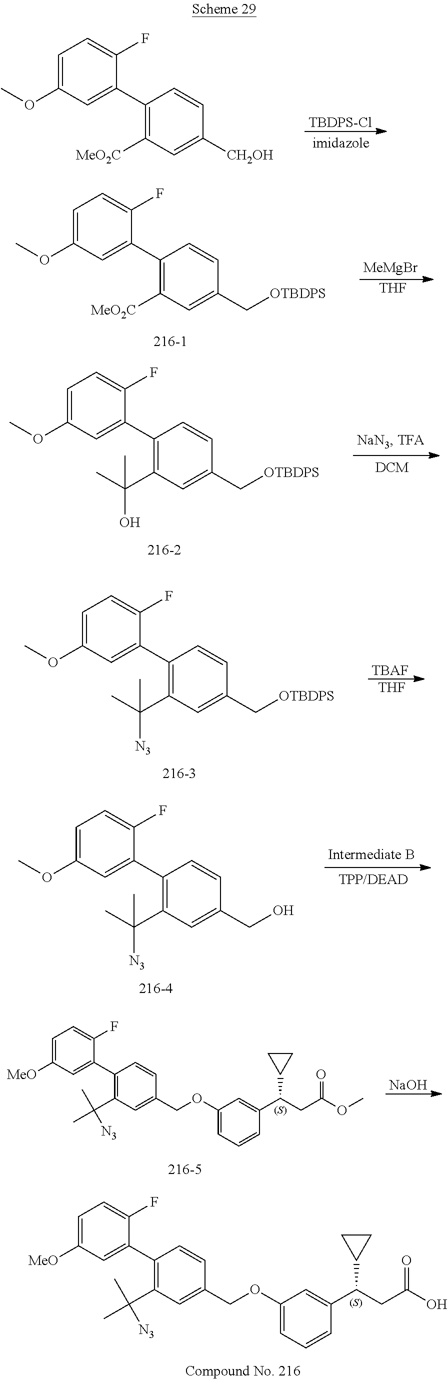

Step 1. To a solution of methyl 2′-fluoro-4-(hydroxymethyl)-5′-methoxy-[1,1′-biphenyl]-2-carboxylate (16.5 g, 0.057 mol) in DCM (200 mL) cooled to 0° C. was added imidazole (5.8 g, 0.085 mol) and TBDPSCl (18.76 g, 0.068 mol). The mixture was stirred at room temperature for 2 h. The reaction mixture was quenched with water and extracted with EA. The organic phase was washed with water and brine, dried with Na₂SO₄, filtered, and the filtrate was concentrated. The residue was purified by column chromatography on silica gel (EA/PE=10%) to give methyl 4-(((tert-butyldiphenylsilyl)oxy)methyl)-2′-fluoro-5′-methoxy-[1,1′-biphenyl]-2-carboxylate, 216-1, as a yellow oil.

Step 2. To a solution of methyl 216-1 (29 g, 0.055 mol) in THE (300 mL) cooled to 0° C. was added MeMgBr (54.9 mL, 0.165 mol) dropwise under N₂. The mixture was stirred at room temperature for 12 h. The reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=10%) to give 2-(4-(((tert-butyldiphenylsilyl) oxy)methyl)-2′-fluoro-5′-methoxy-[1,1′-biphenyl]-2-yl)propan-2-ol, 216-2, as a yellow oil.

¹H NMR (400 MHz, DMSO-d₆): 7.80 (s, 1H); 7.68 (dd, J=1.2 Hz, J=7.6 Hz, 4H); 7.51-7.43 (m, 6H); 7.23 (dd, J=1.6 Hz, J=8.0 Hz, 1H); 7.12 (t, 1H); 6.98-6.96 (d, 1H); 6.93-6.90 (m, 1H); 6.79-6.77 (m, 1H); 4.82-4.80 (d, 2H); 4.82 (br, 1H); 3.74 (s, 3H); 1.27 (s, 3H); 1.24 (s, 3H); 1.07 (s, 9H).

Step 3. To a solution of 216-2 (6.0 g, 0.011 mol) and NaN₃ (1.48 g, 0.023 mol) in DCM (60 mL) cooled to 0° C. was added TFA (3 mL) dropwise. The mixture was stirred at room temperature under N₂ for 12 h. The reaction mixture was quenched with water and adjusted to pH=8 with NaHCO₃ aqueous solution. The resulting mixture was extracted with DCM. The organic phase was washed with water and brine, dried with Na₂SO₄, filtered, and the filtrate was concentrated. The residue was purified by pre-TLC (EA/PE=2%) to give ((2-(2-azidopropan-2-yl)-2′-fluoro-5′-methoxy-[1,1′-biphenyl]-4-yl)methoxy)(tert-butyl) diphenyl silane, 216-3, as a colorless oil. ¹H NMR (400 MHz, DMSO-d₆): 7.69-7.67 (m, 4H); 7.61 (S, 1H); 7.51-7.43 (m, 6H); 7.34-7.32 (d, J=8.0 Hz, 1H); 7.18 (t, 1H); 7.08 (d, J=7.6 Hz, 1H); 6.99-6.94 (m, 1H); 6.84-6.82 (m, 1H); 4.87 (s, 2H); 3.75 (s, 3H); 1.49 (s, 3H); 1.44 (s, 3H); 1.07 (s, 9H).

Step 4. To a solution of 216-3 (1.4 g, 2.53 mmol) in THE (200 mL) cooled to 0° C. was added TBAF (5.1 mL, 5.06 mmol). The mixture was stirred at room temperature for 2 h. After the reaction was completed, the reaction mixture was quenched with water and extracted with EA. The organic phase was washed with water and brine, dried with Na₂SO₄, filtered, and the filtrate was concentrated. The residue was purified by column chromatography on silica gel (EA/PE=25%) to give (2-(2-azidopropan-2-yl)-2′-fluoro-5′-methoxy-[1,1′-biphenyl]-4-yl)methanol, 216-4, as a colorless oil. ¹H NMR (400 MHz, DMSO-d₆): 7.59 (s, 1H); 7.33 (d, 1H); 7.20 (t, 1H); 7.08 (d, 1H); 7.00-6.97 (m, 1H); 6.85-6.82 (m, 1H); 5.32 (t, J=7.6 Hz, 1H); 4.59 (d, J=7.6 Hz, 2H); 3.78 (s, 3H); 1.55 (s, 3H); 1.51 (s, 3H).

Step 5. To a mixture of 216-4 (50 mg, 0.16 mmol), Intermediate B (34.9 mg, 0.16 mmol) and TPP (83.2 mg, 0.32 mmol) in DCM (2 mL) was added DEAD (55.2 mg, 0.32 mmol) at 0° C. under N, and the mixture was stirred at room temperature for 12 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give (S)-methyl 3-(3-((2-(2-azidopropan-2-yl)-2′-fluoro-5′-methoxy-[1,1′-biphenyl]-4-yl)methoxy)phenyl)-3-cyclopropylpropanoate, 216-5, as a colorless oil.

Step 6. To a solution of 216-5 (40 mg, 0.077 mmol) in MeOH (2 mL) and H₂O (0.5 mL) was added NaOH (24.8 mg, 0.619 mmol), and the mixture was stirred at 40° C. for 12 h. The reaction mixture was diluted with water and adjusted to pH=6 with HCl (2N). The resulting mixture was extracted with EA and the organic phase was washed with water and brine, dried with Na₂SO₄, filtered, and the filtrate was concentrated. The residue was purified by pre-HPLC to give (S)-3-(3-((2-(2-azidopropan-2-yl)-2′-fluoro-5′-methoxy-[1,1′-biphenyl]-4-yl)methoxy)phenyl)-3-cyclopropylpropanoic acid, Compound No. 216, as a white solid. ¹H NMR (400 MHz, DMSO-d₆): 7.71 (s, 1H); 7.46-7.44 (d, J=7.6 Hz, 1H); 7.23-7.12 (m, 3H); 6.98-6.94 (m, 2H); 6.89-6.84 (m, 3H); 5.15 (s, 2H); 3.75 (s, 3H); 2.64-2.62 (m, 2H); 2.28-2.26 (m, 1H); 1.53 (s, 3H); 1.49 (s, 3H); 0.99 (br, 1H); 0.49-0.48 (m, 1H); 0.28-0.23 (m, 2H); 0.11-0.10 (m, 1H). MS (ESI) m/z 502.3 [M−H]⁻.

### Example 27. Synthesis of Compound No. 217

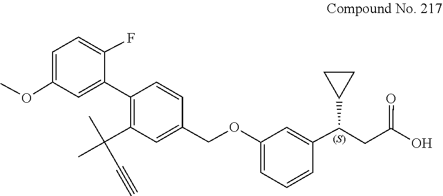

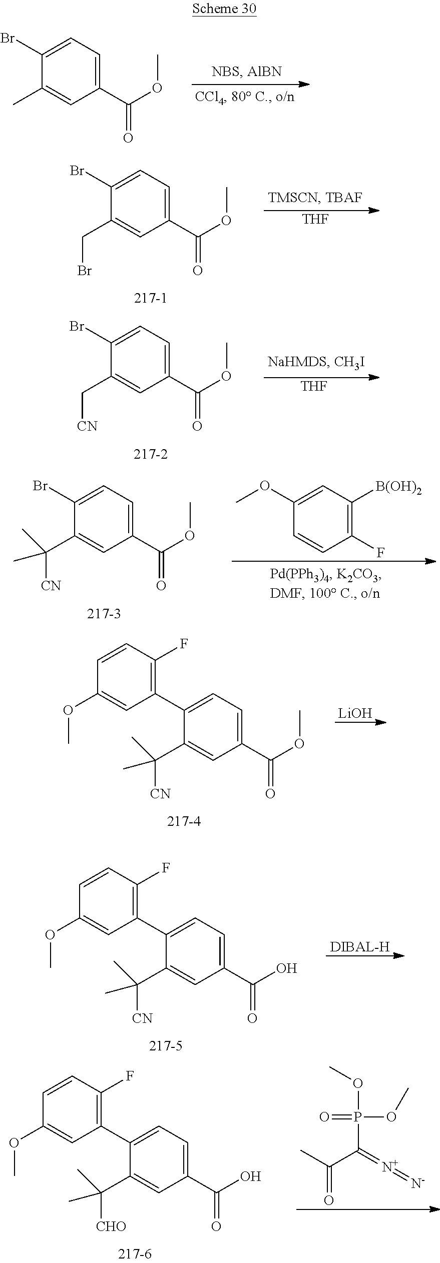

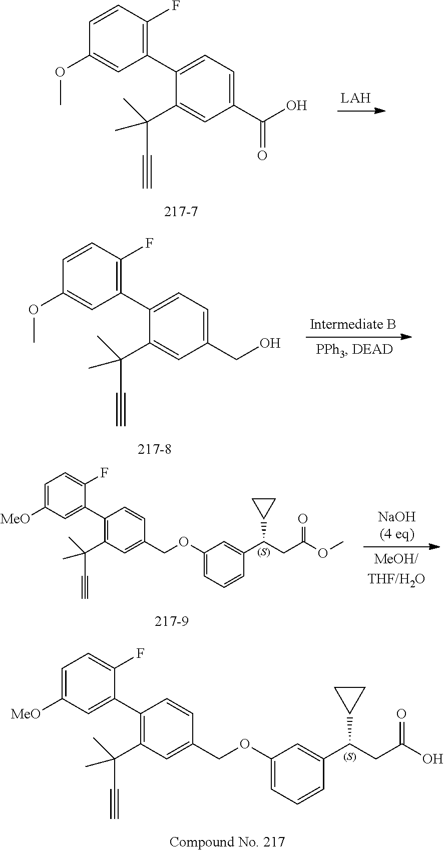

Step 1. To a solution of methyl 4-bromo-3-methyl benzoate (1.0 g, 4.49 mmol) in CCl₄ (30 mL) at room temperature was added NBS (838 mg, 1.05 mmol), AIBN (74 mg, 0.449 mmol). The reaction mixture was stirred at 80° C. overnight under N₂. The reaction mixture was diluted with DCM and water. The aqueous phase was extracted with DCM (15 mL×4). The combined organic layers were dried over MgSO₄ and concentrated. The crude product was chromatographed on silica gel (Petroleum ether/EtOAc=10:1) to give compound, 217-1 as a white solid. 1H NMR (400 MHz, CDCl₃) δ 8.12 (d, J=2 Hz, 1H); 7.82 (dd, J=8.2, 2.2 Hz, 1H); 7.67 (d, J=8.4 Hz, 1H); 4.62 (s, 2H); 3.93 (s, 3H).

Step 2. To a solution of TMSCN (3861 mg, 39 mmol) in dry THE (30 mL) at 0° C. was added TBAF (35.8 mL, 35.8 mmol) under N₂. After 1 hour, the reaction mixture was added 217-1 (10 g, 32.5 mmol) in ACN (200 mL). The reaction mixture was stirred at 80° C. under N₂ for 1 hour. The reaction mixture was concentrated under reduced pressure to remove THE and ACN. The crude product was chromatographed on silica gel (Petroleum ether/EtOAc=20:1 to 10:1 to 5:1 to 3:1) to give compound 217-2 (6.3 g, 70%) as a white solid. 1H NMR (400 MHz, CDCl₃) δ 8.17 (s, 1H); 7.89 (d, J=11.2 Hz, 1H); 7.71 (d, J=10.8 Hz, 1H); 3.94 (s, 3H); 3.89 (s, 2H).

Step 3. To a solution of 217-2 (4.65 g, 18.3 mmol) in THE (120 mL) at 0° C. under N₂ was added NaHMDS (2 M, 36.3 mL) dropwise during 15 min. Stirred for 30 min at 0° C. Then Mel (10.4 g, 73.2 mmol) was added dropwise at 0° C. The resulting mixture was stirred for 1 h at 0° C. and for 14 hrs at r. t. The reaction mixture was quenched by aq. NH₄Cl at 0° C. and separated. The water phase was extracted with EA (100 mL×2). The organic phase was combined and washed with brine, dried over Na₂SO₄ and concentrated to give crude 4-bromo-3-(cyano-dimethyl-methyl)-benzoic acid 217-3 (4.6 g, yield: 89%) as yellow solid. ¹H NMR (400 MHz, CDCl₃) δ 8.11 (d, J=1.6 Hz, 1H); 8.85-8.83 (dd, 1H); 7.76 (d, J=8.4 Hz, 1H); 3.94 (s, 3H); 1.93 (s, 6H).

Step 4. A mixture of 217-3 (2.3 g, 8.156 mmol), 2-fluoro-5-methoxyphenylboronic acid (2.08 g, 12.234 mmol), (PPh₃)4 (942 mg, 0.081 mmol) and K₂CO₃ (3.4 g, 24.47 mmol) in DMF (100 mL) was degassed and filled with N₂ 3 times and heated at 105° C. for 16 hrs. DMF was removed and the residue was diluted with water (100 mL), extracted with EA (60 mL×3), dried and concentrated. The residue was purified by flash chromatography (70EA in PE) to give 2-(cyano-dimethyl-methyl)-2′-fluoro-5′-methoxy-biphenyl-4-carboxylic acid, 217-4, (4.1 g, yield: 76%) as yellow solid. ¹H NMR (400 MHz, CDCl₃) δ 8.27 (d, J=1.2 Hz, 1H); 8.01 (dd, J=8.0 Hz, 1.6 Hz, 1H); 7.27-7.25 (dd, 1H); 7.08-7.03 (t, 1H); 6.95-6.91 (m, 1H); 6.86-6.83 (m, 1H); 3.96 (s, 3H); 3.80 (s, 3H); 1.76 (s, 3H); 1.65 (s, 3H).

Step 5. A mixture of 217-4 (3.1 g, 9.48 mmol) and LiOH·H₂O (800 mg, 18.96 mmol) in MeOH (10 mL), THE (30 mL) and water (10 mL) was stirred for 14 hrs at r. t. Solvent was removed and the residue was acidified with 1N HCl until the pH reached 2 to 3, extracted with EA (50 mL×3), dried and concentrated to give the crude 2-(cyano-dimethyl-methyl)-2′-fluoro-5′-methoxy-biphenyl-4-carboxylic acid, 217-5, as a brown solid, which was directly used in the next step.

Step 6. To a mixture of crude 217-5 (2.85 g, 9.1 mmol) in toluene (140 mL) at −78° C. was added DIBAL-H (1.0 M in hexane, 21 mL) dropwise during 15 min. After addition the resulting mixture was stirred for 1 h at −78° C. and stirred for additional 16 hrs at r. t. The reaction mixture was quenched by aq. NH₄Cl at 0° C. and further acidified with 1N HCl (about 50 mL), extracted with EA (50 mL×4), dried and concentrated. The residue was purified by flash chromatography (30% EA in PE) to give 2-(1,1-dimethyl-2-oxo-ethyl)-2′-fluoro-5′-methoxy-biphenyl-4-carboxylic acid, 217-6, as yellow solid. H NMR (400 MHz, CDCl₃) δ 9.43 (s, 1H); 8.28 (s, 1H); 8.09-8.06 (d, 1H); 7.28 (d, J=8.0 Hz, 1H); 7.03 (t, 1H); 6.93-6.89 (m, 1H); 6.64-6.62 (m, 1H); 3.80 (s, 3H), 1.413 (s, 3H); 1.407 (s, 3H).

Step 7. To a mixture of 217-6 (1 g, 3.16 mmol) in dry MeOH (60 mL) was added Bestmann reagent (1.23 g, 6.33 mmol) and K₂CO₃ (1.31 g, 9.48 mmol) at r. t. The resulting mixture became clear after stirring for 16 hrs at r. t. Solvent was removed and the residue was diluted with water (50 mL), acidified with 1N HCl until the pH reached 3 to 5, extracted with EA (50 mL×3), dried and concentrated. The residue was purified by flash chromatography (25% EA in PE) to give 2-(1,1-dimethyl-prop-2-ynyl)-2′-fluoro-5′-methoxy-biphenyl-4-carboxylic acid, 217-7, as white solid. ¹H NMR (400 MHz, CDCl₃) δ 8.58 (s, 1H); 8.01-7.99 (dd, 1H); 7.21 (d, J=8.0 Hz, 1H); 7.00 (t, 1H); 6.90-6.81 (m, 2H); 3.79 (s, 3H); 2.20 (s, 1H); 1.60 (s 3H); 1.54 (s, 3H).

Step 8. To a solution of 217-7 (785 mg, 2.516 mmol) in dry THE (40 mL) was added LAH (192 mg, 5.032 mmol) portion wise at 0° C. under N₂. The resulting mixture was then stirred and slowly heated at 60° C. for 2 hrs. The reaction mixture was quenched by the addition of H₂O (0.192 mL), NaOH (15%, 0.192 mL) and H₂O (0.576 mL) at 0° C. The reaction mixture was filtered and the filter cake was washed with EA. The combined organic phase was dried over MgSO₄ and concentrated. The residue was purified by flash chromatography (30% EA in PE) to give [2-(1,1-dimethyl-prop-2-ynyl)-2′-fluoro-5′-methoxy-biphenyl-4-yl]-methanol, 217-8, as a white gum. ¹H NMR (400 MHz, CDCl₃) δ 7.85 (s, 1H); 7.30-7.27 (dd, 1H); 7.08 (d, J=7.6 Hz, 1H); 6.97 (t, 1H); 6.86-6.81 (m, 2H); 4.75 (s, 2H); 3.77 (s, 3H); 2.18 (s, 1H); 1.60 (s 3H); 1.54 (s, 3H).

Step 9. To a solution of 217-8 (50.0 mg, 0.17 mmol) in dry DCM (5 mL) was added Intermediate B (37.0 mg, 0.17 mmol) and PPh₃ (88.0 mg, 0.34 mmol) under nitrogen atmosphere at 0° C. The mixture was stirred at 0° C. for 10 minutes, followed with addition of DEAD (58.0 mg, 0.34 mmol). The reaction mixture was allowed to warm up to room temperature and stirred overnight. The reaction mixture was quenched with water and extracted with DCM (20 mL×3). The combined organic layers were washed with water (20 mL×2) and brine (20 mL), dried over Na₂SO₄ and concentrated in vacuum. The residue was purified by flash chromatography (EA/PE=0-30%) to give (S)-methyl 3-cyclopropyl-3-(3-((2′-fluoro-5′-methoxy-2-(2-methylbut-3-yn-2-yl)-[1,1′-biphenyl]-4-yl) methoxy)phenyl)propanoate, 217-9, as a colorless oil. MS: m/z 518.1 (M+H₂O).

Step 10. To a solution of 217-9 (12.0 mg, 0.024 mmol) in MeOH/H₂O (5/2 mL) was added NaOH (9.6 mg, 0.240 mmol) at room temperature. The mixture was heated at 40° C. and stirred overnight. The reaction mixture was acidified by 1 M HCl to pH=3. The reaction mixture was extracted with EA (20 mL×3). The combined organic layers were washed with water (20 mL×2) and brine (20 mL), dried over Na₂SO₄ and concentrated in vacuum. The residue was purified by prepHPLC to give (S)-3-cyclopropyl-3-(3-((2′-fluoro-5′-methoxy-2-(2-methylbut-3-yn-2-yl)-[1,1′-biphenyl]-4-yl)methoxy)phenyl)propanoic acid, Compound No. 217, as a white solid. ¹H NMR (400 MHz, CDCl₃): δ=7.89 (s, 1H); 7.37-7.35 (d, J=7.2 Hz, 1H); 7.26-7.23 (d, 1H); 7.11-7.09 (d, J=6.8 Hz, 1H); 6.98 (t, J=8.8 Hz, 1H); 6.90-6.81 (m, 5H); 5.10 (s, 2H); 3.78 (s, 3H); 2.80-2.77 (m, 2H); 2.40-2.32 (m, 1H); 2.18 (s, 1H); 1.55 (s, 3H); 1.50 (s, 3H); 1.04-1.02 (m, 1H); 0.60-0.58 (m, 1H); 0.44-0.42 (m, 1H); 0.32-0.28 (m, 1H); 0.18-0.14 (m, 1H). MS: m/z 485.3 (M−H)⁺.

### Example 28. Synthesis of Compound No. 218

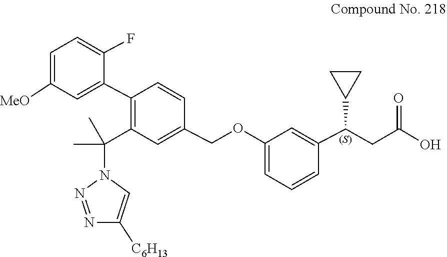

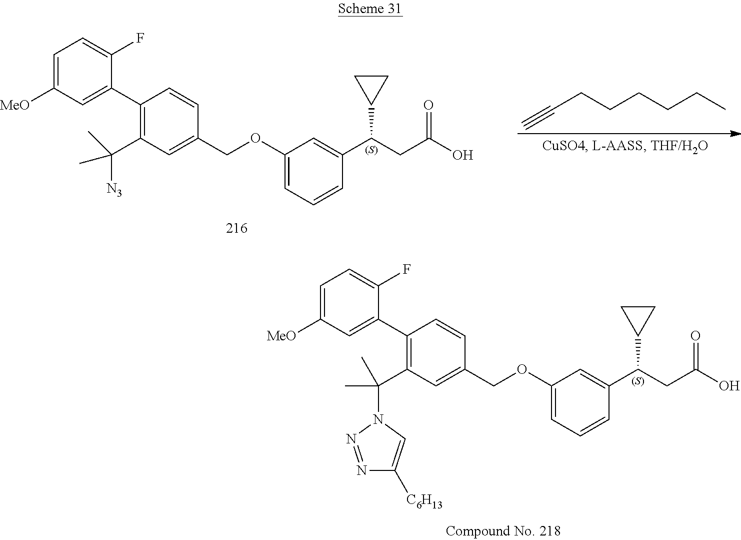

To a solution 216 (10 mg, 0.020 mmol) and oct-1-yne (4.4 mg, 0.040 mmol) in MeOH (1 mL) and H₂O (0.1 mL) was added CuSO₄ 5H₂O (2.5 mg, 0.010 mmol) and L-Ascorbic Acid Sodium Salt (2.0 mg, 0.010 mmol). The mixture was stirred at 50° C. for 48 h. The reaction mixture was filtered and the filtrate was concentrated. The residue was purified by pre-HPLC to give (S)-3-cyclopropyl-3-(3-((2′-fluoro-2-(2-(4-hexyl-1H-1,2,3-triazol-1-yl)propan-2-yl)-5′-methoxy-[1,1′-biphenyl]-4-yl)methoxy)phenyl)propanoic acid, Compound No. 218, (2.0 m g, yield: 16.4%) as a yellow solid. ¹H NMR (400 MHz, DMSO-d₆): 7.52 (s, 1H); 7.45-7.43 (d, 1H); 7.43 (s, 1H); 7.22-7.18 (m, 1H); 7.06-7.02 (m, 2H); 6.93 (s, 1H), 6.86-6.84 (d, 2H); 6.83-6.79 (m, 1H); 5.94-5.93 (m, 1H); 5.13 (s, 2H); 3.60 (s, 3H); 2.60-2.38 (m, 4H); 2.35-2.25 (m, 1H); 1.85 (s, 3H); 1.78 (s, 3H); 1.53-1.44 (m, 2H); 1.27-1.67 (m, 6H); 1.00-0.96 (m, 1H); 0.86-0.84 (m, 3H); 0.49-0.45 (m, 1H); 0.26-0.24 (m, 2H); 0.10-0.07 (m, 1H). MS (ESI) m/z 612.4 [M−H]⁻.

### Example 29. Synthesis of Compound No. 219

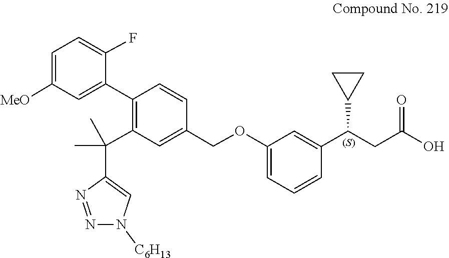

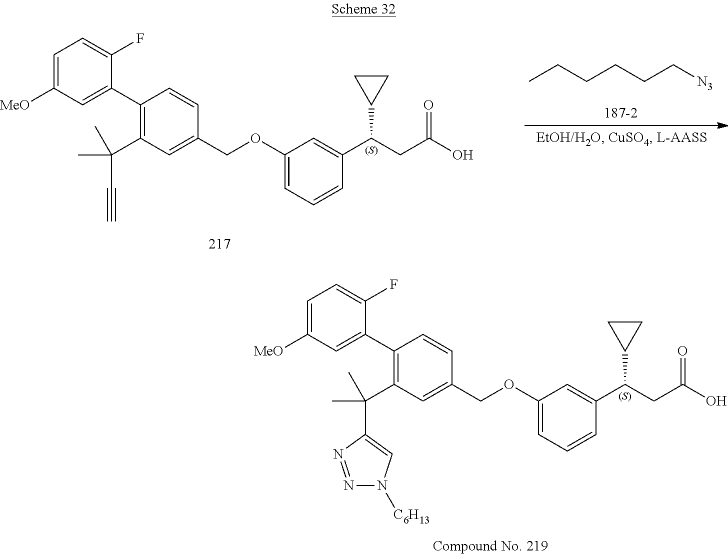

Step 1. To a mixture of 217 (40 mg, 0.082 mmol) and 187-2 (0.15 M in EtOH, 1.1 mL, 0.164 mmol) in EtOH (4 mL) and water (1 mL) was added L-AASS (0.2 M, 0.41 mL) and CuSO₄·5H₂O (0.1 M, 0.41 mL) at r. t. The resulting mixture was then stirred for 16 hrs at r. t. Solvent was removed and the residue was purified by prep-HPLC (TFA method) to give (S)-3-cyclopropyl-3-(3-{2′-fluoro-2-[1-(1-hexyl-1H-[1,2,3]triazol-4-yl)-1-methyl-ethyl]-5′-methoxy-biphenyl-4-ylmethoxy}-phenyl)-propionic acid, Compound No. 219, as a white solid. MS (ESI) m/z 614.4 [M+H]⁺. H NMR (400 MHz, DMSO-d₆): δ=11.98 (br, 1H); 7.69 (s, 1H); 7.36-7.34 (d, 1H); 7.32 (s, 1H); 7.25-7.21 (t, 1H); 7.02-6.94 (m, 3H); 6.91-6.86 (m, 2H); 6.82-6.78 (m, 1H); 5.96-5.93 (m, 1H); 5.14 (s, 2H); 4.14-4.05 (m, 2H); 3.61 s, 3H); 2.65-2.58 (m, 2H); 2.34-2.28 (m, 1H); 1.76-1.69 (m, 2H); 1.60 (s, 3H); 1.47 (s, 3H); 1.37-1.20 (m, 6H); 1.04-1.00 (m, 1H); 0.88-0.84 (m, 3H); 0.52-0.46 (m, 1H); 0.34-0.22 (m, 2H); 0.16-0.10 (m, 1H).

### Example 30. Synthesis of Compound No. 220

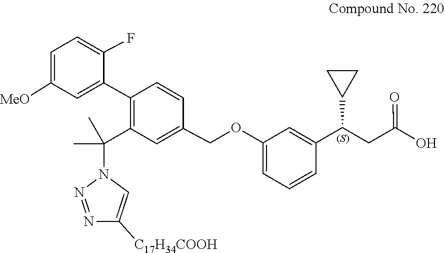

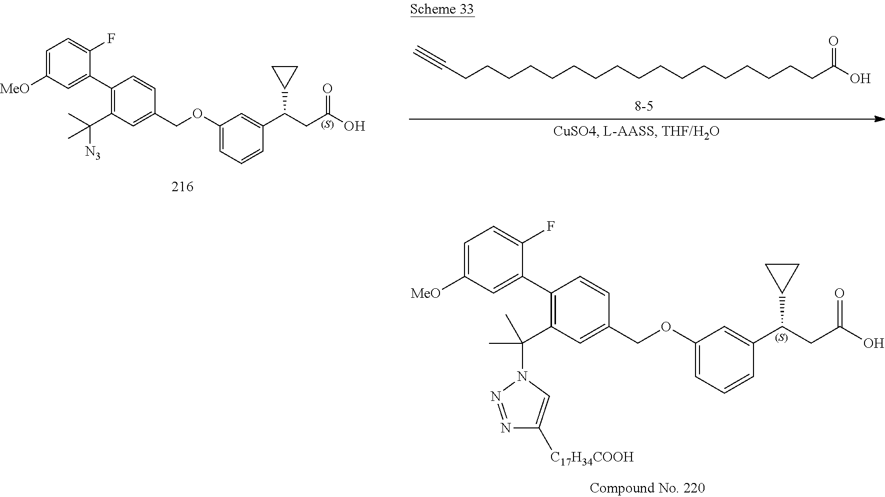

To a solution of 216 (30 mg, 0.060 mmol) and 8-5 (36.9 mg, 0.119 mmol) in THE (1 mL) was added CuSO₄·5H₂O (7.5 mg, 0.030 mmol) in H₂O (0.5 mL) and L-Ascorbic Acid Sodium Salt (5.9 mg, 0.030 mmol) in H₂O (0.5 mL). The mixture was stirred at 90° C. for 2 h. The reaction mixture was filtered and the filtrate was concentrated. The residue was purified by pre-HPLC to give (S)-18-(1-(2-(4-((3-(2-carboxy-1-cyclopropylethyl) phenoxy)methyl)-2′-fluoro-5′-methoxy-[1,1′-biphenyl]-2-yl)propan-2-yl)-1H-1,2,3-triazol-4-yl)octadecanoic acid, Compound No. 220, as a white solid. H NMR (400 MHz, DMSO-d₆): 11.93 (br, 2H); 7.51 (s, 1H); 7.45-7.42 (d, 1H); 7.42 (s, 1H); 7.24-7.20 (t, 1H); 7.06-7.02 (t, 1H); 7.04-7.02 (d, 1H); 6.94 (s, 1H); 6.89-6.85 (m, 2H); 6.82-6.78 (m, 1H); 5.96-5.93 (m, 1H); 5.14 (s, 2H); 3.60 (s, 3H); 2.69-2.51 (m, 2H); 2.33-2.27 (m, 1H); 2.20-2.11 (m, 4H); 1.85 (s, 3H); 1.78 (s, 3H); 1.49-1.36 (m, 4H); 1.36-1.24 (s, 26H); 1.04-0.98 (m, 1H); 0.51-0.48 (m, 1H); 0.33-0.24 (m, 2H); 0.12-0.09 (m, 1H). MS (ESI) m/z 810.7 [M−H]⁻.

### Example 31. Synthesis of Compound No. 221

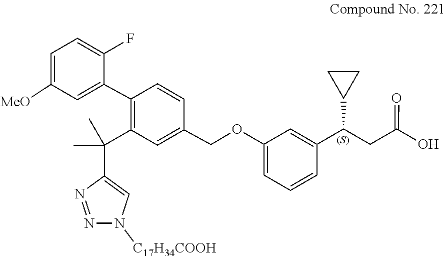

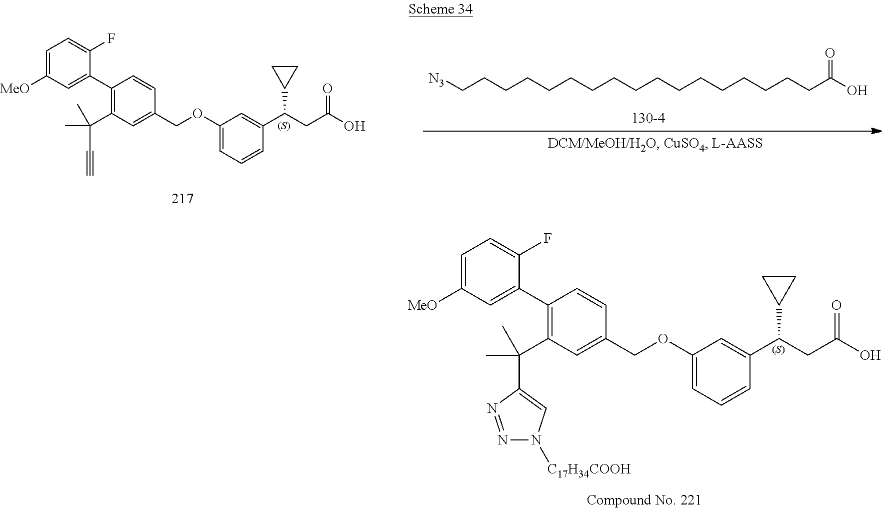

Step 1. To a mixture of 217, 50 mg, 0.103 mmol) and 130-4 (67 mg, 0.205 mmol) in DCM (3 mL) and MeOH (1 mL) was added L-AASS (0.2 M in water, 0.5 mL) and CuSO₄·5H₂O (0.1 M, 0.5 mL) at r. t. The resulting mixture was then stirred for 16 hrs at r. t. The reaction mixture was separated and the water phase was extracted with DCM (2 mL×3). The organic phase was combined, dried and concentrated. The residue was purified by prep-HPLC (TFA method) to give (S)-18-[4-(1-{4-[3-(2-Carboxy-1-cyclopropyl-ethyl)-phenoxymethyl]-2′-fluoro-5′-methoxy-biphenyl-2-yl}-1-methyl-ethyl)-[1,2,3]triazol-1-yl]-octadecanoic acid, Compound No. 221, (21 mg, yield: 25%) as a white solid. MS (ESI) m/z 812.6 [M+H]⁺. ¹H NMR (400 MHz, DMSO-d₆): δ=11.91 (br, 1H); 7.68 (s, 1H); 7.35-7.33 (d, 1H); 7.30 (s, 1H); 7.24-7.20 (t, 1H); 7.01-6.93 (m, 3H); 6.90-6.85 (m, 2H); 6.81-6.77 (m, 1H); 5.96-5.93 (m, 1H); 5.14 (s, 2H); 4.11-4.05 (m, 2H); 3.60 (s, 3H); 2.68-2.62 (m, 2H); 2.33-2.26 (m, 1H); 2.19-2.16 (m, 2H); 1.73-1.69 (m, 2H); 1.60 (s, 3H); 1.49 (s, 3H); 1.28-1.15 (m, 26H); 1.03-1.00 (m, 1H); 0.51-0.49 (m, 1H); 0.35-0.220 (m, 2H); 0.14-0.08 (m, 1H).

### Example 32. Synthesis of Compound No. 222

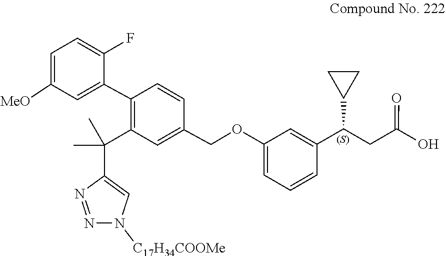

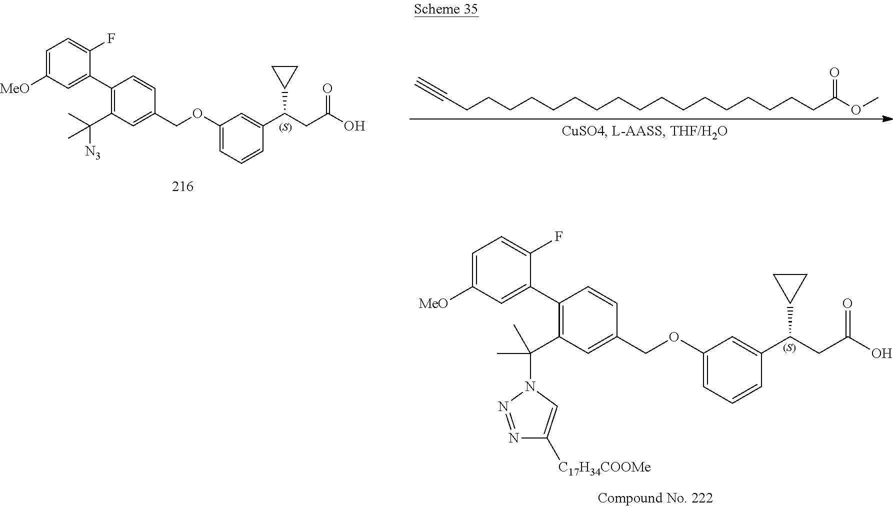

To a solution of 216 (50 mg, 0.099 mmol) and methyl icos-19-ynoate (64.2 mg, 0.199 mmol) in THF (1 mL) was added CuSO₄·5H₂O (12.4 mg, 0.050 mmol) in H₂O (0.5 mL) and L-Ascorbic Acid Sodium Salt (9.8 mg, 0.050 mmol) in H₂O (0.5 mL). The mixture was stirred at 90° C. for 4 h. The reaction mixture was filtered and the filtrate was concentrated. The residue was purified by pre-HPLC to give (S)-3-cyclopropyl-3-(3-((2′-fluoro-5′-methoxy-2-(2-(4-(18-methoxy-18-oxo octadecyl)-1H-1,2,3-triazol-1-yl)propan-2-yl)-[1,1′-biphenyl]-4-yl)methoxy) phenyl)propanoic acid, Compound No. 222, (4.0 m g, yield: 4.9%) as a white solid. ¹H NMR (400 MHz, DMSO-d₆): 12.00 (br, 1H), 7.51 (s, 1H); 7.45-7.42 (d, 1H); 7.42 (s, 1H); 7.23-7.201 (t, 1H); 7.06-7.02 (t, 1H); 7.04-7.02 (d, 1H); 6.94 (s, 1H); 6.88-6.85 (m, 2H); 6.82-6.78 (m, 1H); 5.95-5.92 (m, 1H); 5.13 (s, 2H); 3.60 (s, 3H); 3.57 (s, 3H); 2.50-2.40 (m, 2H); 2.33-2.26 (m, 4H); 2.15-2.11 (m, 1H); 1.85 (s, 3H); 1.78 (s, 3H); 1.52-1.49 (m, 4H); 1.33-1.17 (s, 26H); 1.03-0.96 (m, 1H); 0.50-0.46 (m, 1H); 0.31-0.22 (m, 2H); 0.13-0.10 (m, 1H). MS (ESI) m/z 826.6 [M+H]⁺.

### Example 33. Synthesis of Compound No. 223

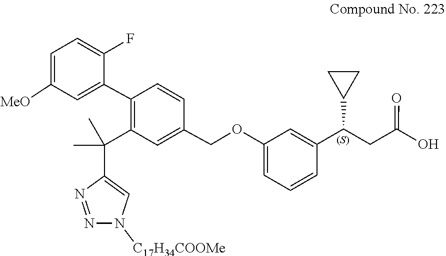

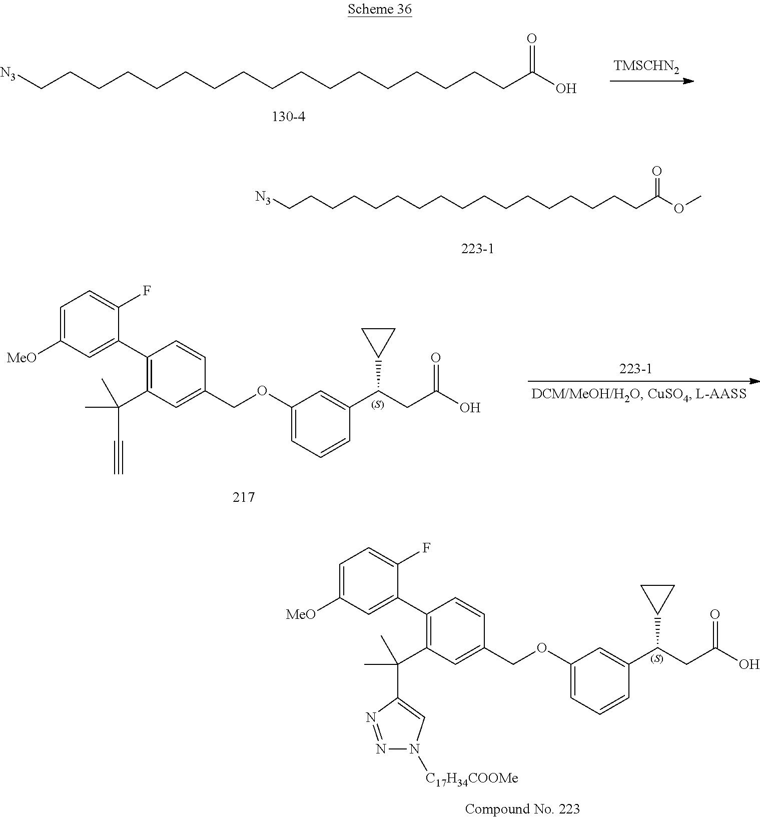

Step 1. To a mixture of 130-4 (104 mg, 0.32 mmol) in DCM (4 mL) and MeOH (4 mL) at 0° C. was added TMSCHN₂ (2.0 M in hexane, 0.5 mL) dropwise. The resulting mixture was stirred for 14 hrs at r. t. Solvent was removed to give crude 18-azido-octadecanoic acid methyl ester 223-1 (82 mg, yield: 74%) as a white solid.

Step 2. To a mixture of 217 (30 mg, 0.062 mmol) and 223-1 (42 mg, 0.123 mmol) in DCM (4 mL) and MeOH (1 mL) was added L-AASS (0.2 M in water, 0.3 mL) and CuSO₄·5H₂O (0.1 M in water, 0.3 mL) at r. t. The resulting mixture was then stirred for 16 hrs at r. t. The reaction mixture was separated and the water phase was extracted with DCM (2 mL×3). The organic phase was combined, dried and concentrated. The residue was purified by prep-HPLC (TFA method) to give (S)-18-[4-(1-{4-[3-(2-Carboxy-1-cyclopropyl-ethyl)-phenoxymethyl]-2′-fluoro-5′-methoxy-biphenyl-2-yl}-1-methyl-ethyl)-[1,2,3] triazol-1-yl]-octadecanoic acid methyl ester, Compound No. 223, (12 mg, yield: 23%) as a white solid. MS (ESI) m/z 826.6 [M+H]⁺. ¹H NMR (400 MHz, DMSO-d₆): δ=11.97 (br, 1H); 7.68 (s, 1H); 7.35-7.33 (m, 1H); 7.30 (s, 1H); 7.24-7.20 (t, 1H); 7.00-6.88 (m, 3H); 6.88-6.81 (m, 2H); 6.80-6.77 (m, 1H); 5.96-5.93 (m, 1H); 5.13 (s, 2H); 4.13-4.03 (m, 2H); 3.60 (s, 3H); 3.57 (s, 3H); 2.69-2.64 (m, 2H); 2.30-2.24 (m, 3H); 1.75-1.69 (m, 2H); 1.60 (s, 3H); 1.52-1.46 (m, 2H); 1.46 (s, 3H); 1.28-1.18 (m, 26H); 1.05-1.00 (m, 1H); 0.51-0.47 (m, 1H); 0.34-0.23 (m, 2H); 0.14-0.09 (m, 1H).

### Example 34. Synthesis of Compound No. 224

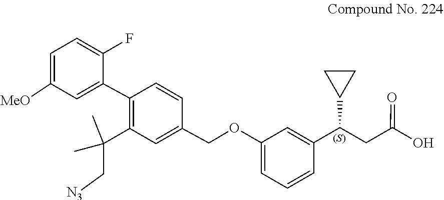

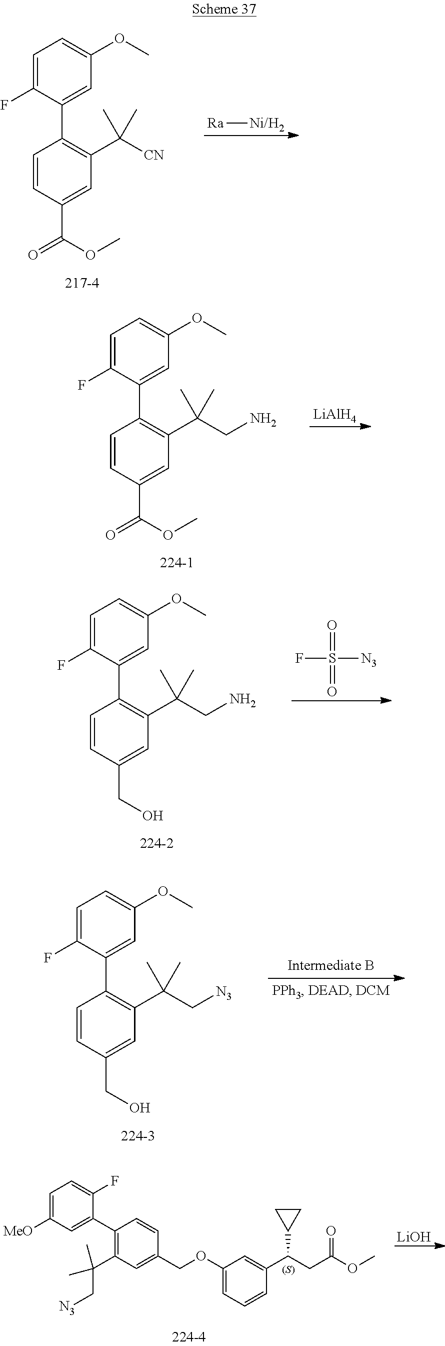

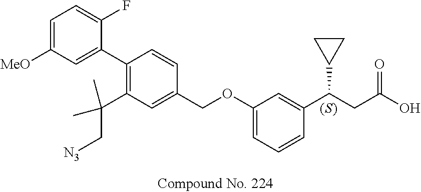

Step 1. A mixture of 2-(cyano-dimethyl-methyl)-2′-fluoro-5′-methoxy-biphenyl-4-carboxylic acid (836 mg, 2.6 mmol) and Raney-Ni (˜200 mg, 20% wt.) in MeOH (30 mL) was degassed and filled with hydrogen using a balloon. The resulting mixture was then hydrogenated for 16 hrs at r. t. Solvent was removed and the residue was purified by flash chromatography (5% MeOH in DCM) to give 2-(2-amino-1,1-dimethyl-ethyl)-2′-fluoro-5′-methoxy-biphenyl-4-carboxylic acid methyl ester 224-1 (780 mg, yield: 93%) as pale-yellow oil. MS (ESI) m/z 332.0 [M+H]⁺.

Step 2. To a mixture of 224-1 (128 mg 26 mmol) in THE (10 mL) at 0° C. under N₂ atmosphere was added LAH (198 mg, 5.2 mmol) in 3 portions. The resulting mixture was stirred for 15 min at 0° C. and stirred for additional 2 hrs at r. t. The reaction mixture was quenched by water (0.2 mL), aq NaOH (15%, 0.2 mL) and water (0.6 mL) at 0° C., diluted with EA (20 mL) and filtered. The filtrate was dried over Na₂SO₄ and concentrated to give crude [2-(2-Amino-1,1-dimethyl-ethyl)-2′-fluoro-5′-methoxy-biphenyl-4-yl]-methanol, 224-2, (110 mg, yield: 95%) as pale-yellow oil. MS (ESI) m/z 304.1 [M+H]⁺.

Step 3. To a mixture 224-2 (420 mg, 1.386 mmol) in DMF (30 mL) at r. t. was added fluorosulfuryl azide (˜0.5 M in MTBE, 2.78 mL) and KHCO₃ (3.0 M, 1.848 mL) dropwise. After addition the resulting mixture was stirred for 4 hrs at r. t. Diluted with water (50 mL), extracted with EA (30 mL×3), dried and concentrated. The residue was purified by flash chromatography (30% EA in PE) to give [2-(2-azido-1,1-dimethyl-ethyl)-2′-fluoro-5′-methoxy-biphenyl-4-yl]-methanol, 224-3, (210 mg, yield: 46% over 2 steps) as white solid. MS (ESI) m/z 325.2 [M-27+Na]⁺.

Step 4. A solution of 224-3 (245 mg, 0.744 mmol), Intermediate B (164 mg, 0.744 mmol) and PPh₃ (390 mg, 1.488 mmol) in DCM (15 mL) was degassed and filled with N₂ 3 times and cooled to 0° C. DEAD (260 mg, 1.488 mmol) was then added dropwise via syringe. The resulting mixture was stirred for 12 hrs under N₂, allowing the temperature to slowly warm to r. t. Solvent was removed and the residue was purified by flash chromatography (20% EA in PE) to give (R)-3-{3-[2-(2-Azido-1,1-dimethyl-ethyl)-2′-fluoro-5′-methoxy-biphenyl-4-ylmethoxy]-phenyl}-3-cyclopropyl-propionic acid methyl ester, 224-4, (140 mg, yield: 35%) as white gum. MS (ESI) m/z 532.4 [M+18]⁺.

Step 5. A mixture of 224-4 (140 mg, 0.263 mmol) and LiOH·H₂O (111 mg, 2.63 mmol) in water (5 mL), MeOH (5 mL) and THE (10 mL) was stirred at 50° C. for 14 hrs. MeOH was removed and the residue was acidified with aqueous HCl until pH reached 3 and extracted with EtOAc (15 mL×3). The combined organic layers were dried over MgSO₄ and concentrated. The residue was purified by preparative HPLC (TFA method) to give, Compound No. 224, as white solid. MS (ESI) m/z 535.4 [M+18]⁺. ¹H NMR (400 MHz, DMSO-d₆) δ 12.01 (br, 1H); 7.64 (s, 1H); 7.38-7.36 (d, 1H); 7.24-7.19 (m, 2H); 7.05-6.99 (m, 2H); 6.96 (s, 1H); 6.91-6.82 (m, 3H); 5.14 (s, 2H); 3.77 (s, 3H); 3.46 (s, 2H); 2.70-2.61 (m, 2H); 2.31-2.25 (m, 1H); 1.23 (s, 3H); 1.15 (s, 3H); 1.04-0.99 (m, 1H); 0.53-0.49 (m, 1H); 0.34-0.23 (m, 2H); 0.13-0.10 (m, 1H).

### Example 35. Synthesis of Compound No. 225

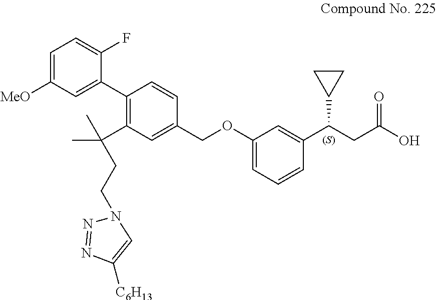

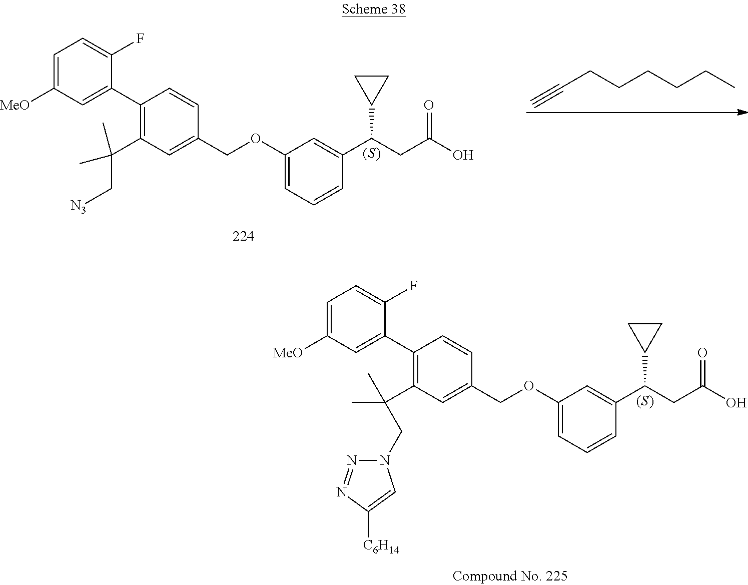

To a mixture of 224 (32 mg, 0.062 mmol) and oct-1-yne (10.2 mg, 0.0928 mmol) in THE (4 mL) was added L-AASS (0.2 M in water, 60 uL) and CuSO₄·5H₂O (0.1 M in water, 60 uL) at r. t. The resulting mixture was then stirred for 16 hrs at 60° C. Solvent was removed and the residue was purified by prep-HPLC (TFA method) to give (S)-3-cyclopropyl-3-(3-{2′-fluoro-2-[2-(4-hexyl-[1,2,3]triazol-1-yl)-1,1-dimethyl-ethyl]-5′-methoxy-biphenyl-4-ylmethoxy}-phenyl)-propionic acid, Compound No. 225, as a white solid. MS (ESI) m/z 628.5 [M+H]⁺. 1H NMR (400 MHz, DMSO-d₆) δ 12 (brs, 1H); 7.55 (s, 1H); 7.37 (d, J=8 Hz, 1H); 7.21 (t, J=8 Hz, 1H); 7.18 (t, 9 Hz, 1H); 7.13 (d, J=2.4 Hz, 1H); 7.03 (d, J=8 Hz, 1H); 7.00-6.96 (m, 1H); 6.93 (s, 1H); 6.87-6.84 (m, 2H); 6.75-6.73 (m, 1H); 5.09 (s, 2H); 4.43 (s, 2H); 3.73 (s, 3H); 2.68-2.58 (m, 2H); 2.51-2.48 (m, 2H); 2.29-2.23 (m, 1H); 1.49-1.45 (m, 2H); 1.22-1.21 (m, 9H); 1.05 (s, 3H); 1.03-0.97 (m, 1H); 0.85-0.81 (m, 3H); 0.50-0.47 (m, 1H); 0.31-0.21 (m, 2H); 0.12-0.09 (m, 1H).

### Example 36. Synthesis of Compound No. 226

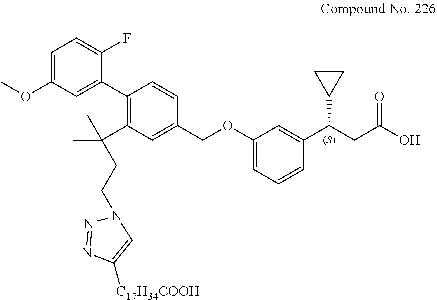

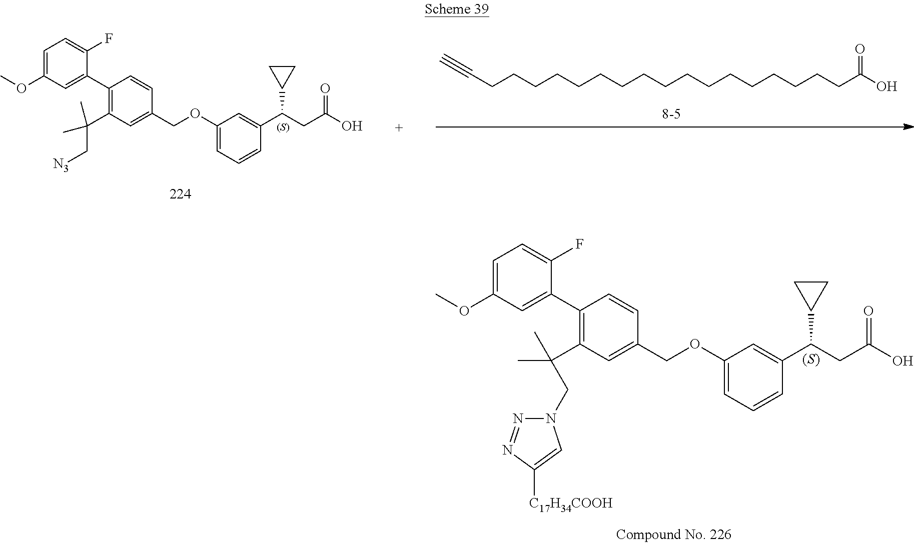

To a solution of 8-5 (27.0 mg, 0.087 mmol) in THF/H₂O (2/0.5 mL) was added 224 (30.0 mg, 0.058 mmol), L-Ascorbic acid sodium salt (46 mg, 0.232 mmol) and Copper(II) sulfate pentahydrate (29 mg, 0.116 mmol). The reaction mixture was heated to 65° C. and stirred for 16 hours. The reaction mixture was quenched with water and extracted with DCM (10 mL×3). The combined organic layers were washed with water (10 mL×2) and brine (10 mL), dried over Na₂SO₄ and concentrated. The residue was purified by pre.TLC (PE/EA=4:1) and pre.HPLC to give (S)-18-(1-(2-(4-((3-(2-carboxy-1-cyclopropylethyl) phenoxy)methyl)-2′-fluoro-5′-methoxy- [1,1′-biphenyl]-2-yl)-2-methylpropyl)-1H-1,2,3-triazol-4-yl)octadecanoic acid, Compound No. 226, as a colorless gum. ¹H NMR (400 MHz, DMSO-d₆): δ=11.95 (s, 2H); 7.55 (s, 1H); 7.36 (d, J=8 Hz, 1H); 7.20 (t, J=8 Hz, 1H); 7.17 (t, J=9 Hz, 1H); 7.11 (d, J=1 Hz, 1H); 7.02 (d, J=8 Hz, 1H); 6.99-6.95 (m, 1H); 6.93 (s, 1H); 6.86-6.84 (m, 2H); 6.74-6.71 (m, 1H); 5.09 (s, 2H); 4.42 (s, 2H); 3.73 (s, 3H); 2.66-2.62 (m, 2H); 2.50-2.47 (m, 2H); 2.27-2.25 (m, 1H); 2.19-2.16 (t, 2H); 1.47-1.46 (m, 4H); 1.29-1.20 (m, 29H); 1.05 (s, 3H); 1.01-0.99 (m, 1H); 0.51-0.47 (m, 1H); 0.31-0.22 (m, 2H); 0.12-0.08 (m, 1H). MS: m/z 826.7 (M+H⁺).

### Example 37. Synthesis of Compound No. 227

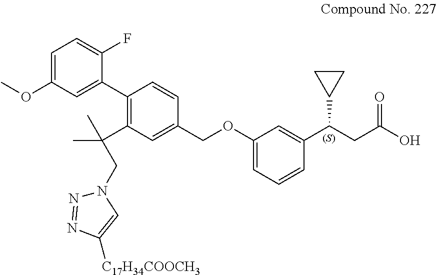

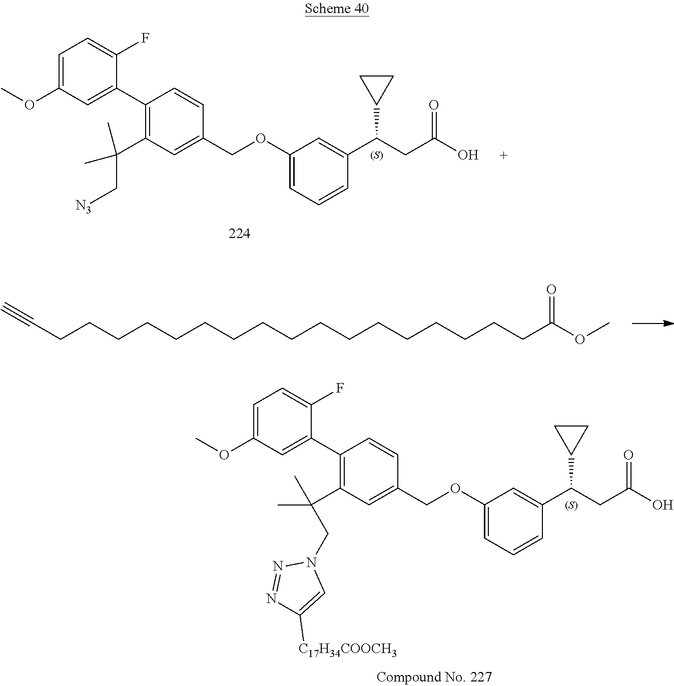

To a solution of methyl icos-19-ynoic acid ester (28.0 mg, 0.087 mmol) in THF/H₂O (2/0.5 mL) was added 224 (30.0 mg, 0.058 mmol), L-Ascorbic acid sodium salt (46.0 mg, 0.232 mmol) and Copper(II) sulfate pentahydrate (29.0 mg, 0.116 mmol). The reaction mixture was heated to 65° C. and stirred for 16 hours. The reaction mixture was quenched with water and extracted with DCM (10 mL×3). The combined organic layers were washed with water (10 mL×2) and brine (10 mL), dried over Na₂SO₄ and concentrated. The residue was purified by pre.TLC (PE/EA=4:1) and pre.HPLC to give (S)-3-cyclopropyl-3-(3-((2′-fluoro-5′-methoxy-2-(1-(4-(18-methoxy-18-oxooctadecyl)-1H-1,2,3-triazol-1-yl)-2-methylpropan-2-yl)-[1,1′-biphenyl]-4-yl)methoxy)phenyl)propanoic acid, Compound No. 227, as a colorless gum. ¹H NMR (400 MHz, DMSO-d₆): δ=11.98 (brs, 1H); 7.55 (s, 1H); 7.36 (d, J=7.6 Hz, 1H); 7.20 (t, J=8 Hz, H); 7.17 (t, J=9 Hz, 1H); 7.11 (d, J=2 Hz, 1H); 7.02 (d, J=8 Hz, 1H); 6.99-6.95 (m, 1H); 6.93 (s, 1H); 6.86-6.83 (m, 2H); 6.73-6.71 (m, 1H); 5.09 (s, 2H); 4.42 (s, 2H); 3.72 (s, 3H); 3.57 (s, 3H); 2.68-2.58 (m, 2H); 2.50-2.47 (m, 2H); 2.29-2.25 (m, 3H); 1.51-1.44 (m, 4H); 1.22-1.20 (m, 29H); 1.05 (s, 3H); 1.02-0.98 (m, 1H); 0.50-0.48 (m, 1H); 0.30-0.22 (m, 2H); 0.13-0.08 (m, 1H). MS: m/z 840.6 (M+H⁺).

### Example 38. Synthesis of Compound No. 228

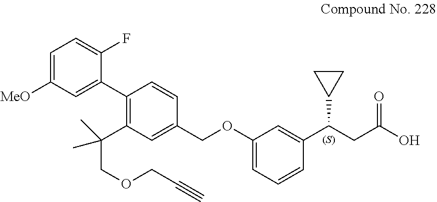

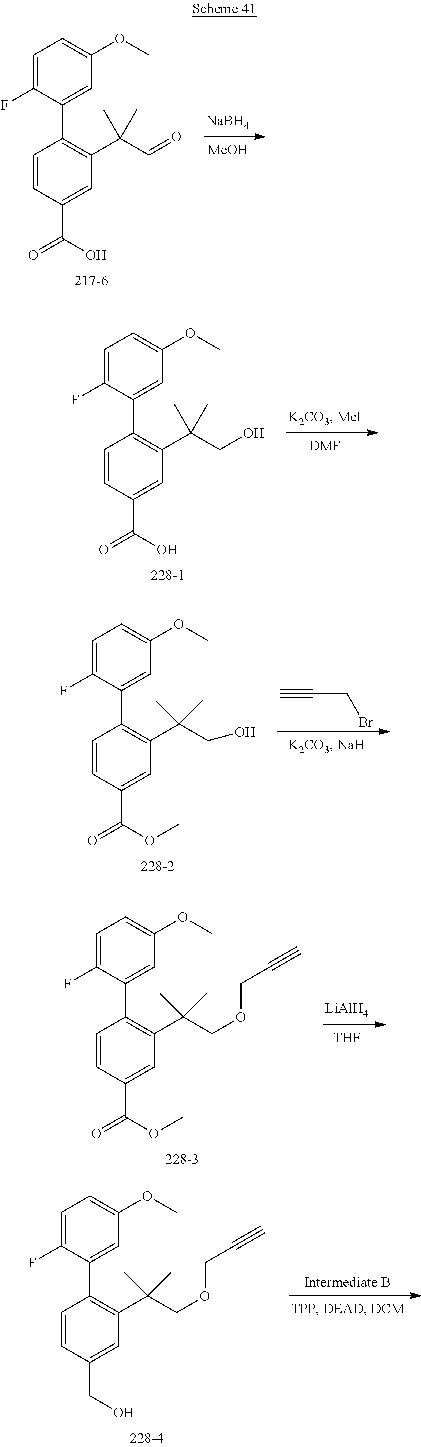

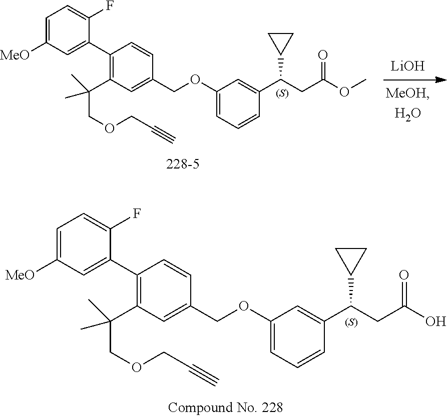

Step 1. To a mixture of 2′-fluoro-5′-methoxy-2-(2-methyl-1-oxopropan-2-yl)-[1,1′-biphenyl]-4-carboxylic acid (3.2 g, 0.010 mol) in MeOH (50 mL) cooled to 0° C. was added NaBH₄ (0.77 g, 0.020 mol) over 15 min. The mixture was stirred at 0° C. for 2 h. The reaction mixture was quenched with NH₄Cl aqueous solution and adjusted to pH=2 with HCl (2N). The resulting mixture was extracted with EA and the organic phase was washed with water and brine, dried with Na₂SO₄, filtered, and the filtrate was concentrated to give 2′-fluoro-2-(1-hydroxy-2-methylpropan-2-yl)-5′-methoxy-[1,1′-biphenyl]-4-carboxylic acid, 228-1, (3.0 g, yield: 93.2%) as a yellow oil.

Step 2. To a solution of 228-1 (3.0 g, 9.4 mmol) in DMF (30 mL) was added K₂CO₃ (2.61 g, 18.9 mmol) and Mel (2.68 g, 18.9 mmol). The mixture was stirred at room temperature for 2 h. The reaction mixture was quenched with water and extracted with EA. The organic phase was washed with water and brine, dried with Na₂SO₄, filtered, and the filtrate was concentrated. The residue was purified by column chromatography on silica gel (EA/PE=20%) to give methyl 2′-fluoro-2-(1-hydroxy-2-methylpropan-2-yl)-5′-methoxy-[1,1′-biphenyl]-4-carboxylate, 228-2, (1.5 g, yield: 47.9%) as a yellow solid. ¹H NMR (400 MHz, DMSO-d₆): 8.14 (s, 1H); 7.79 (dd, J=1.2 Hz, J=7.6 Hz, 1H); 7.19 (t, J=9.0 Hz, 1H); 7.12 (d, J=7.6 Hz, 1H); 7.01-6.97 (m, 1H); 6.88-6.86 (m, 1H); 4.70 (t, J=5.4 Hz, 1H); 3.88 (s, 3H); 3.75 (s, 3H); 3.47-3.43 (m, 1H); 3.36-3.31 (m, 1H); 1.18 (s, 3H); 1.00 (s, 3H).

Step 3. To a solution 228-2 (1.5 g, 4.5 mmol) in DMF (15 mL) cooled to 0° C. was added NaH (0.36 g, 9.0 mmol). The mixture was stirred at 0° C. for 30 min. Then 3-bromoprop-1-yne (1.08 g, 9.0 mmol) was added and the mixture was stirred at room temperature for 2 h. The reaction mixture was quenched with NH₄Cl aqueous solution and extracted with EA. The organic phase was washed with water, brine, dried with Na₂SO₄. The crude product was purified by column chromatography on silica gel (EA/PE=10%) to give methyl 2′-fluoro-5′-methoxy-2-(2-methyl-1-(prop-2-yn-1-yloxy)propan-2-yl)-[1,1′-biphenyl]-4-carboxylate, 228-3, as a yellow oil.

Step 4. To a solution of methyl 228-3 (450 mg, 1.22 mmol) in THE (10 mL) cooled to 0° C. was added LiAlH₄ (92.4 mg, 2.43 mmol) over 30 min. Then the mixture was stirred at room temperature for 1 h. After the reaction was completed, the reaction mixture was quenched with water (0.1 mL) at 0° C. Then 15% of NaOH aqueous solution (0.1 mL) and water (0.3 mL) were added. The resulting mixture was diluted with EA, dried with MgSO₄, filtered, and the filtrate was concentrated to give (2′-fluoro-5′-methoxy-2-(2-methyl-1-(prop-2-yn-1-yloxy)propan-2-yl)-[1,1′-biphenyl]-4-yl)methanol, 228-4, as a colorless oil. ¹H NMR (400 MHz, DMSO-d₆): 7.45 (s, 1H); 7.19-7.13 (m, 2H); 6.98-6.94 (m, 1H); 6.91 (d, J=10.8 Hz, 1H); 6.78-6.76 (m, 1H); 5.19 (t, J=5.8 Hz, 1H); 4.52 (d, J=6.0 Hz, 2H); 4.01 (d, J=2.0 Hz, 2H); 3.75 (m, 3H); 3.42-3.35 (m, 3H); 1.18 (s, 3H); 1.07 (s, 3H).

Step 5. To a mixture of 228-4 (300 mg, 0.88 mmol), Intermediate B (193.0 mg, 0.88 mmol) and TPP (344.7 mg, 1.32 mmol) in DCM (5 mL) was added DEAD (228.9 mg, 1.32 mmol) at 0° C., and the mixture was stirred at room temperature under N₂ for 12 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give (S)-methyl 3-cyclopropyl-3-(3-((2′-fluoro-5′-methoxy-2-(2-methyl-1-(prop-2-yn-1-yloxy)propan-2-yl)-[1,1′-biphenyl]-4-yl)methoxy)phenyl)propanoate, 228-5, as a colorless oil.

Step 6. To a solution of 228-5 (310 mg, 0.57 mmol) in MeOH (5 mL) and H₂O (0.5 mL) was added LiGH (239.3 mg, 5.70 mmol), and the mixture was stirred at 45° C. for 4 h. After the reaction was completed, the reaction mixture was diluted with water and adjusted to pH=3 with HCl (2N). The resulting mixture was extracted with EA and the organic phase was washed with water and brine, dried with Na₂SO₄, filtered, and the filtrate was concentrated to give (S)-3-cyclopropyl-3-(3-((2′-fluoro-5′-methoxy-2-(2-methyl-1-(prop-2-yn-1-yloxy)propan-2-yl)-[1,1′-biphenyl]-4-yl)methoxy)phenyl)propanoic acid, Compound No. 228, as a white solid. H NMR (400 MHz, DMSO-d₆): 11.96 (s, 1H); 7.60 (s, 1H); 7.33-7.31 (d, J=8.0 Hz, 1H); 7.23-7.14 (m, 2H); 7.00-6.94 (m, 3H); 6.89-6.79 (m, 3H); 5.11 (s, 2H); 4.01 (s, 2H); 3.75 (s, 3H); 3.45-3.31 (m, 3H); 2.69-2.63 (m, 2H); 2.29-2.23 (m, 1H); 1.19 (s, 3H); 1.08 (s, 3H); 1.02-0.98 (m, 1H); 0.52-0.47 (m, 1H); 0.32-0.22 (m, 2H); 0.13-0.09 (m, 1H). MS (ESI) m/z 529.5 [M−H]⁻.

### Example 39. Synthesis of Compound No. 229

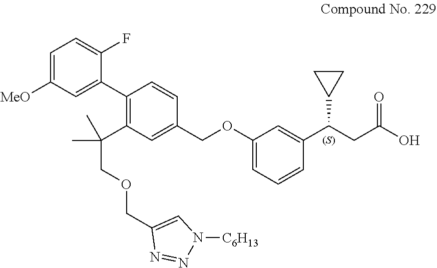

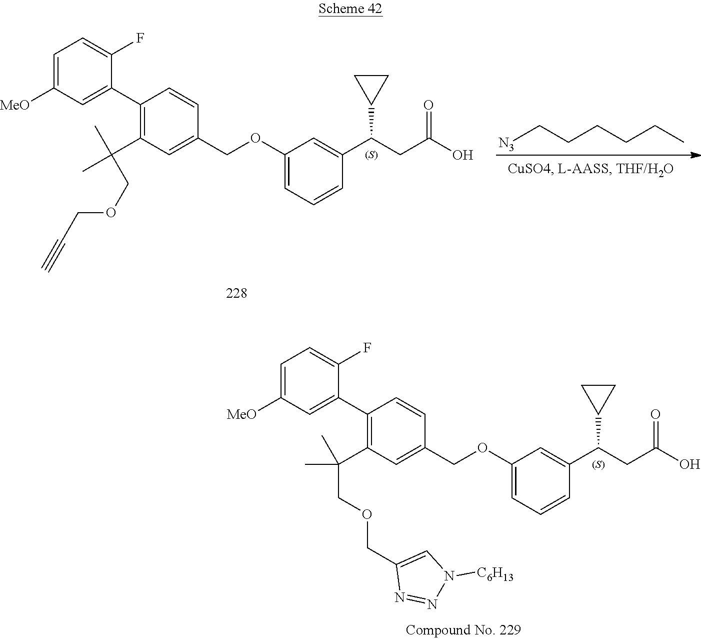

To a solution of 228 (30 mg, 0.057 mmol) and 1-azidohexane (0.75 mL, 0.113 mmol) in THE (1 mL) was added CuSO₄·5H₂O (7.1 mg, 0.028 mmol) in H₂O (0.5 mL) and L-Ascorbic Acid Sodium Salt (5.6 mg, 0.028 mmol) in H₂O (0.5 mL). The mixture was stirred at 90° C. for 4 h. After the reaction was completed, the reaction mixture was filtered and the filtrate was concentrated. The residue was purified by pre-HPLC to give (S)-3-cyclopropyl-3-(3-((2′-fluoro-2-(1-((1-hexyl-1H-1,2,3-triazol-4-yl)methoxy)-2-methylpropan-2-yl)-5′-methoxy-[1,1′-biphenyl]-4-yl)methoxy)phenyl) propanoic acid, Compound No. 229, as a white solid. ¹H NMR (400 MHz, DMSO-d₆): 11.96 (br, 1H); 7.92 (s, 1H); 7.55 (s, 1H); 7.31-7.30 (d, J=7.6 Hz, 1H); 7.23-7.13 (m, 2H); 6.98-6.94 (m, 3H); 6.88-6.84 (m, 2H); 6.77-6.75 (m, 1H); 5.08 (s, 2H); 4.39 (s, 2H); 4.27 (t, J=9.0 Hz, 2H); 3.73 (s, 3H); 3.41-3.33 (ABq, 2H); 2.67-2.63 (m, 2H); 2.28-2.25 (m, 1H); 1.79-1.74 (m, 2H); 1.23-1.17 (m, 6H); 1.15 (s, 3H); 1.05-0.99 (m, 4H); 0.82 (t, J=6.8 Hz, 3H); 0.50-0.48 (m, 1H); 0.31-0.22 (m, 2H); 0.13-0.11 (m, 1H). MS (ESI) m/z 658.5 [M+H]⁺.

### Example 40. Synthesis of Compound No. 230

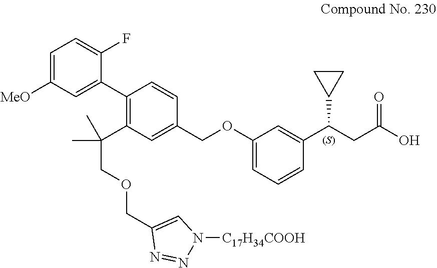

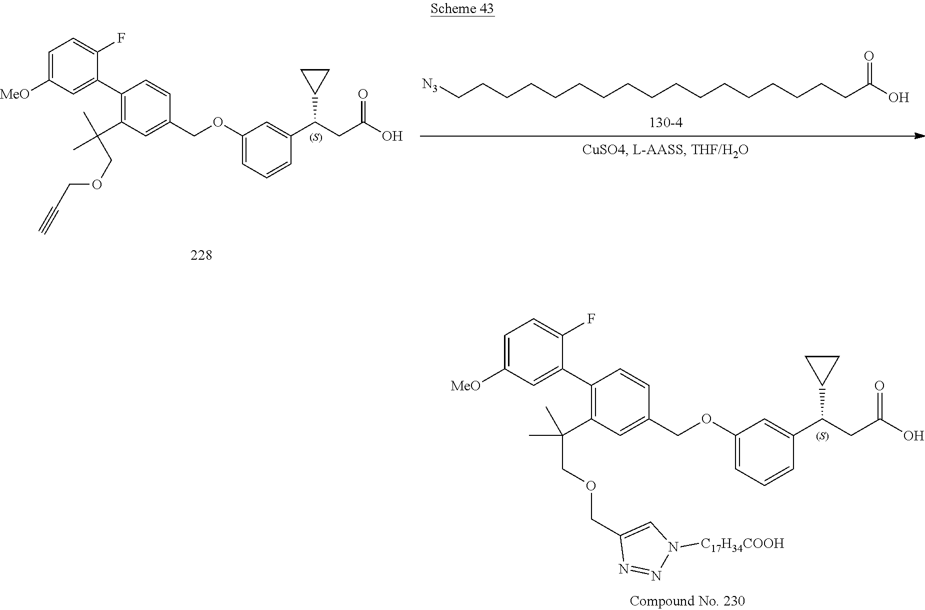

Compound No. 230 was made in the same way as 229 using azidoacid 130-4: ¹H NMR (400 MHz, DMSO-d₆): 12.06 (br, 1H); 7.92 (s, 1H); 7.55 (s, 1H); 7.31-7.30 (d, 1H); 7.19 (t, 1H); 7.15 (t, 1H); 6.98-6.94 (m, 3H), 6.88-6.84 (m, 2H); 6.76-6.74 (m, 1H); 5.08 (s, 2H); 4.39 (s, 2H); 4.28-4.25 (t, 2H); 3.73 (s, 3H); 3.40-3.33 (m, 2H); 2.67-2.63 (m, 2H); 2.26-2.20 (m, 1H); 2.19-2.15 (t, 2H); 1.77-1.74 (m, 2H); 1.51-1.48 (m, 2H); 1.22-1.19 (m, 26H); 1.15 (s, 3H); 1.04 (s, 3H); 1.01-0.97 (m, 1H); 0.50-0.45 (m, 1H); 0.32-0.22 (m, 2H); 0.10-0.06 (m, 1H).

### Example 41. Synthesis of Compound No. 231

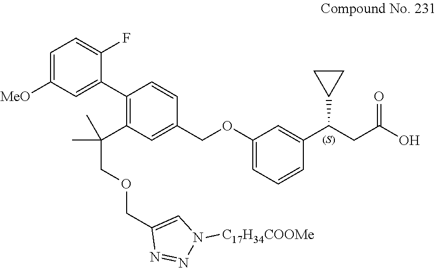

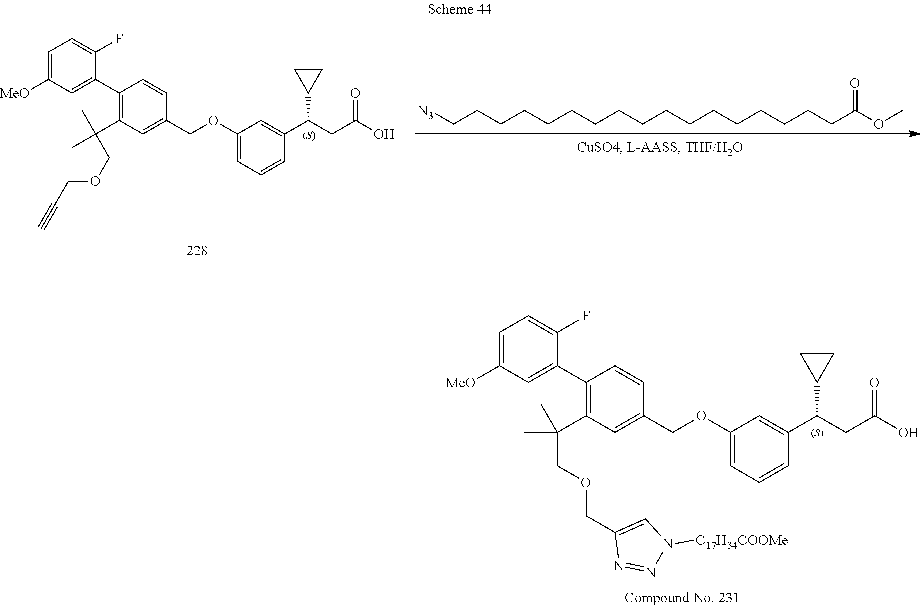

To a solution of 228 (30 mg, 0.057 mmol) and methyl 18-azidooctadecanoate (28.8 mg, 0.085 mmol) in THF (1 mL) was added CuSO₄·5H₂O (7.1 mg, 0.028 mmol) in H₂O (0.5 mL) and L-Ascorbic Acid Sodium Salt (5.6 mg, 0.028 mmol) in H₂O (0.5 mL). The mixture was stirred at 90° C. for 4 h. The reaction mixture was filtered and the filtrate was concentrated. The residue was purified by pre-HPLC to give (S)-3-cyclopropyl-3-(3-((2′-fluoro-5′-methoxy-2-(1-((1-(18-methoxy-18-oxooctadecyl)-1H-1,2,3-triazol-4-yl)methoxy)-2-methylpropan-2-yl)-[1,1′-biphenyl]-4-yl)methoxy)phenyl)propanoic acid, Compound No. 231, as a white solid. H NMR (400 MHz, DMSO-d₆): 12.06 (br, 1H); 7.92 (s, 1H); 7.53 (s, 1H); 7.31 (d, 1H), 7.19 (t, 1H); 7.15 (t, 1H); 6.98-6.93 (m, 3H), 6.85-6.84 (d, 2H); 6.76-6.74 (m, 1H); 5.07 (s, 2H); 4.39 (s, 2H); 4.27 (t, 2H); 3.75 (s, 3H); 3.57 (s, 3H); 3.40-3.33 (m, 2H); 2.63-2.51 (m, 2H); 2.33-2.30 (m, 1H); 2.27 (t, 2H); 1.78-1.71 (m, 2H); 1.51-1.48 (m, 2H); 1.22-1.19 (m, 26H); 1.15 (s, 3H); 1.04 (s, 3H); 1.01-0.97 (m, 1H); 0.47-0.44 (m, 1H); 0.32-0.22 (m, 2H); 0.10-0.06 (m, 1H). MS (ESI) m/z 870.8 [M+H]⁺.

### Example 42. Synthesis of Compound No. 232

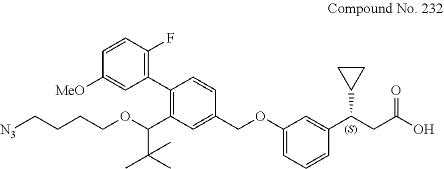

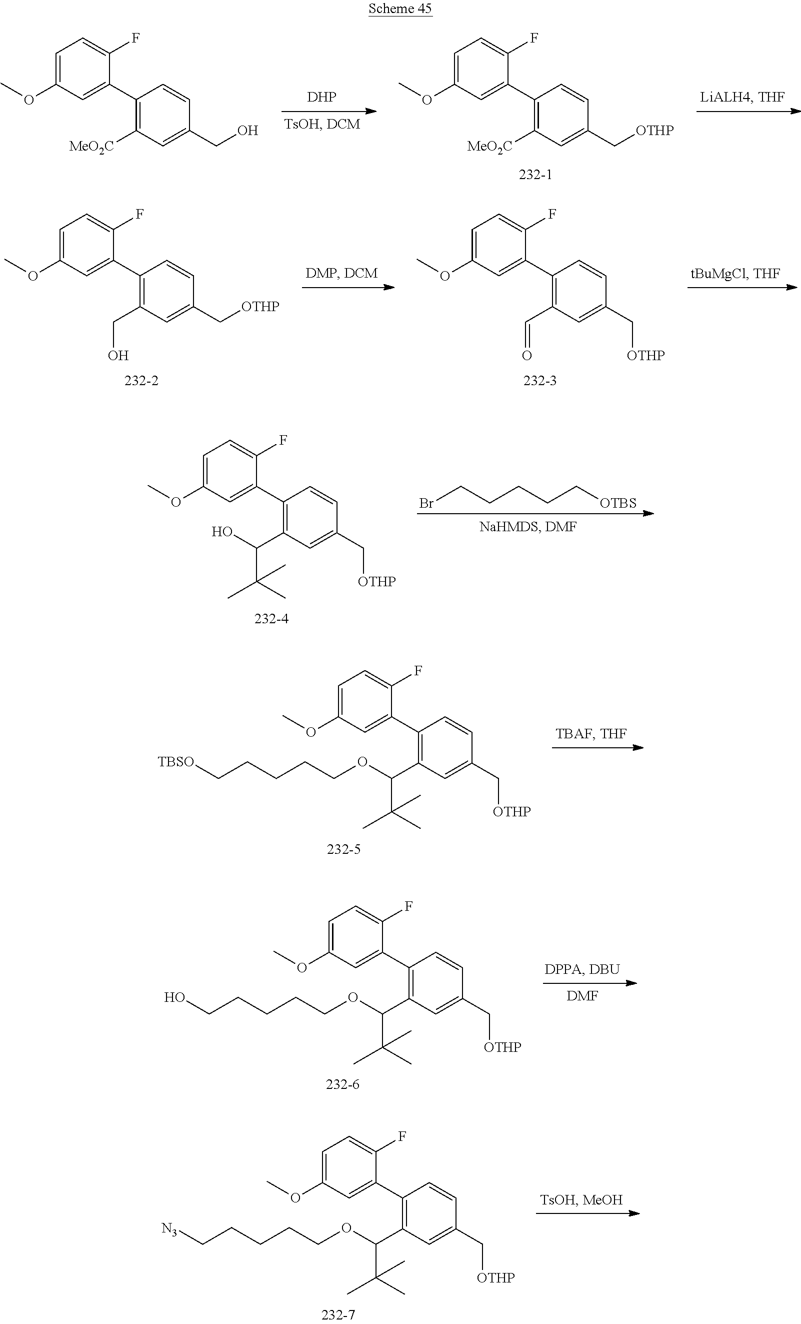

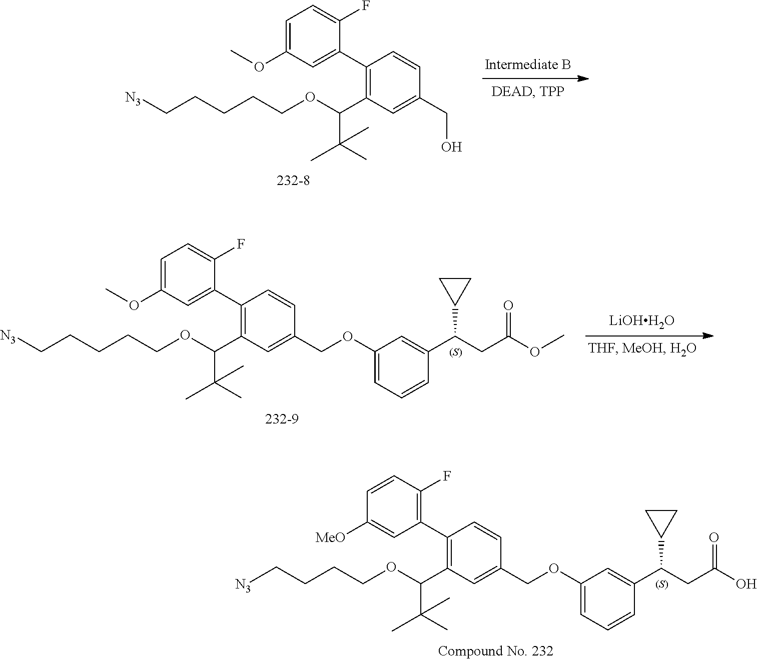

Step 1. To a solution of methyl 2′-fluoro-4-(hydroxymethyl)-5′-methoxy-[1,1′-biphenyl]-2-carboxylate (13.5 g, 0.047 mol) in DCM (200 mL) cooled to 0° C. was added DHP (7.82 g, 0.093 mol) and TsOH (1.77 g, 0.0093 mol). The mixture was stirred at room temperature for 5 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give methyl 2′-fluoro-5′-methoxy-4-(((tetrahydro-2H-pyran-2-yl)oxy)methyl)-[1,1′-biphenyl]-2-carboxylate, 232-1, as a yellow oil.

Step 2. To a solution of 232-1 (15.5 g, 0.041 mol) in THE (150 mL) cooled to 0° C. was added LiAlH₄ (2.36 g, 0.062 mol) over 30 min. Then the mixture was stirred at room temperature for 1 h. The reaction mixture was quenched with water (2.4 mL) at 0° C., followed by addition of 15% of NaOH aqueous solution (2.4 mL) and water (7.2 mL). The resulting mixture was diluted with EA, dried with MgSO₄, filtered, and the filtrate was concentrated to give (2′-fluoro-5′-methoxy-4-(((tetrahydro-2H-pyran-2-yl)oxy)methyl)-[1,1′-biphenyl]-2-yl)methanol, 232-2, as a yellow oil.

Step 3. To a solution of 232-2 (14.0 g, 0.041 mol) in DCM (200 mL) was added Dess-Martin Periodinane (25.7 g, 0.061 mol), and the mixture was stirred at room temperature for 2 h. After the reaction was completed, the reaction mixture was concentrated and the residue was purified by column chromatography on silica gel (EA/PE=20%) to give 2′-fluoro-5′-methoxy-4-(((tetrahydro-2H-pyran-2-yl)oxy)methyl)-[1,1′-biphenyl]-2-carbaldehyde, 232-3, as a yellow oil. ¹H NMR (400 MHz, DMSO-d₆): 9.84 (d, J=3.6 Hz, 1H); 7.91 (d, J=1.6 Hz, 1H); 7.75 (dd, J=2.0 Hz, J=8.0 Hz, 1H); 7.51 (d, J=7.6 Hz, 1H); 7.27 (t, J=9.2 Hz, 1H); 7.08-7.00 (m, 2H); 4.82-4.74 (m, 2H); 4.60 (d, J=12.4 Hz, 1H); 3.84-3.80 (m, 4H) 3.54-3.49 (m, 1H) 1.78-1.67 (m, 2H); 1.59-1.48 (m, 4H).

Step 4. To a solution of 232-3 (5 g, 0.015 mol) in THE (50 mL) cooled to −70° C. was added tBuMgCl (21.4 mL, 0.036 mol) over 20 min. Then the mixture was allowed to warm to room temperature and stirred under N₂ for 12 h. The reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=20%) to give 1-(2′-fluoro-5′-methoxy-4-(((tetrahydro-2H-pyran-2-yl)oxy)methyl)-[1,1′-biphenyl]-2-yl)-2,2-dimethylpropan-1-ol, 232-4, as a yellow oil.

Step 5. To a solution of 232-4 (1 g, 2.49 mmol) in DMF (10 mL) cooled to 0° C. was added NaHMDS (5.0 mL, 9.95 mmol) over 10 min under N₂. The mixture was stirred at room temperature for 2 h. Then the reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=10%) to give tert-butyl((5-(1-(2′-fluoro-5′-methoxy-4-(((tetrahydro-2H-pyran-2-yl)oxy)methyl)-[1,1′-biphenyl]-2-yl)-2,2-dimethylpropoxy) pentyl)oxy)dimethylsilane, 232-5, as a yellow oil.

Step 6. To a solution of 232-5 (1 g, 1.66 mmol) in THE (10 mL) cooled to 0° C. was added TBAF (4.98 mL, 4.98 mmol), and the mixture was stirred at room temperature for 2 h. After the reaction was completed, the reaction mixture was quenched with water and extracted with EA. The organic phase was washed with water and brine, dried with Na₂SO₄, filtered, and the filtrate was concentrated. The residue was purified by column chromatography on silica gel (EA/PE=20%) to give 5-(1-(2′-fluoro-5′-methoxy-4-(((tetrahydro-2H-pyran-2-yl)oxy) methyl)-[1,1′-biphenyl]-2-yl)-2,2-dimethylpropoxy) pentan-1-ol, 232-6, as a colorless oil.

Step 7. To a solution of 232-6 (420 mg, 0.86 mmol) in DMF (5 mL) was added DBU (261.6 mg, 1.72 mmol) and DPPA (473.4 mg, 1.72 mmol), and the mixture was stirred at 90° C. for 2 h. After the reaction was completed, the reaction mixture was quenched with water and extracted with EA. The organic phase was washed with water and brine, dried with Na₂SO₄, filtered, and the filtrate was concentrated. The residue was purified by column chromatography on silica gel (EA/PE=10%) to give 2-((2-(1-((5-azidopentyl)oxy)-2,2-dimethylpropyl)-2′-fluoro-5′-methoxy-[1,1′-biphenyl]-4-yl)methoxy)tetrahydro-2H-pyran, 232-7, as a colorless oil.

Step 8. To a solution of 232-7 (100 mg, 0.19 mmol) in MeOH (2 mL) was added TsOH (111.1 mg, 0.58 mmol), and the mixture was stirred at room temperature for 2 h. After the reaction was completed, the reaction mixture was quenched with water and extracted with EA. The organic phase was washed with water and brine, dried with Na₂SO₄, filtered, and the filtrate was concentrated. The residue was purified by column chromatography on silica gel (EA/PE=25%) to give (2-(1-((5-azidopentyl)oxy)-2,2-dimethylpropyl)-2′-fluoro-5′-methoxy-[1,1′-biphenyl]-4-yl)methanol, 232-8, as a yellow oil. ¹H NMR (400 MHz, DMSO-d₆): 7.45 (d, J=16.4 Hz, 1H); 7.32-7.11 (m, 3H); 6.99-6.95 (m, 1H); 6.77-6.71 (m, 1H); 5.23 (t, J=5.6 Hz, 1H); 4.55 (d, J=5.2 Hz, 2H); 4.19-3.95 (m, 1H); 3.75 (s, 3H); 3.42-3.19 (m, 4H); 1.55-1.40 (m, 6H); 0.66 (s, 9H).

Step 9. To a mixture of 232-8 (60 mg, 0.14 mmol), Intermediate B (36.9 mg, 0.17 mmol) and TPP (73.3 mg, 0.28 mmol) in DCM (1 mL) was added DEAD (48.7 mg, 0.28 mmol) at 0° C., and the mixture was stirred at room temperature for 12 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give (3S)-methyl 3-(3-((2-(1-((5-azidopentyl)oxy)-2,2-dimethylpropyl)-2′-fluoro-5′-methoxy-[1,1′-biphenyl]-4-yl)methoxy)phenyl)-3-cyclopropylpropanoate, 232-9, as a colorless gum. ¹H NMR (400 MHz, CDCl₃): 7.60 (d, J=12.0 Hz, 1H); 7.41 (t, J=8.2 Hz, 1H); 7.25-7.16 (m, 2H); 7.07-6.99 (m, 1H); 6.87-6.83 (m, 4H); 6.74-6.69 (m, 1H); 5.11 (s, 2H); 4.24-4.01 (s, 1H); 3.78 (s, 3H); 3.61 (s, 3H); 3.43-3.38 (m, 1H); 3.30-3.25 (m, 3H); 2.79-2.68 (m, 2H); 2.38-2.32 (m, 1H); 1.64-1.44 (m, 4H); 1.03-0.98 (m, 1H); 0.70 (s, 9H); 0.59-0.56 (m, 1H); 0.43-0.39 (m, 1H); 0.27-0.23 (m, 1H); 0.15-0.12 (m, 1H).

Step 10. To a solution of 232-9 (50 mg, 0.079 mmol) in MeOH (2 mL) and H₂O (0.5 mL) was added LiOH (33.3 mg, 0.792 mmol), and the mixture was stirred at 50° C. for 5 h. After the reaction was completed, the reaction mixture was concentrated. The residue was purified by pre-HPLC to give (3S)-3-(3-((2-(1-(4-azidobutoxy)-2,2-dimethylpropyl)-2′-fluoro-5′-methoxy-[1,1′-biphenyl]-4-yl)methoxy)phenyl)-3-cyclopropylpropanoic acid, Compound No. 232, as a white solid. ¹H NMR (400 MHz, DMSO-d₆): 11.96 (s, 1H); 7.58 (d, 1H); 7.46-7.41 (m, 1H); 7.27-7.17 (m, 3H); 7.00-6.96 (m, 1H); 6.89 (s, 1H); 6.86-6.83 (m, 2H); 6.80-6.73 (m, 1H); 5.17 (s, 2H); 4.20-3.96 (s, s, two rotamers, 1H); 3.75 (s, 3H); 3.38-3.19 (m, 4H); 2.68-2.57 (m, 2H); 2.28-2.22 (m, 1H); 1.53-1.37 (m, 6H); 1.00-0.96 (m, 1H); 0.64 (s, 9H); 0.50-0.45 (m, 1H); 0.30-0.19 (m, 2H); 0.09-0.06 (m, 1H). MS (ESI) m/z 616.5 [M−H]⁻.

### Example 43. Synthesis of Compound No. 233

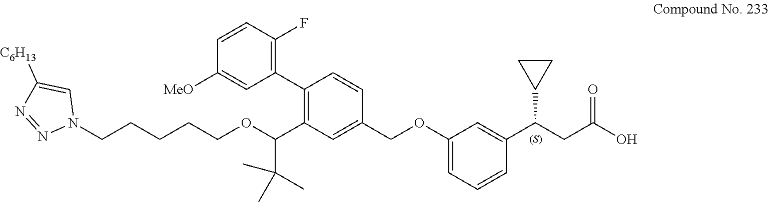

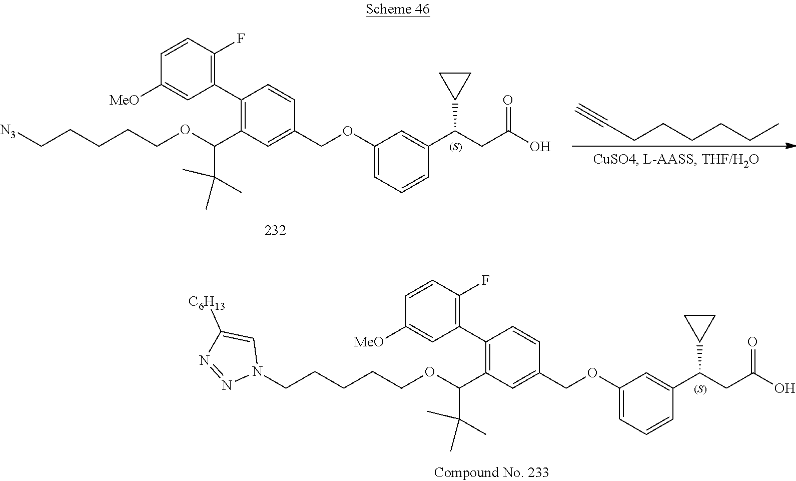

Compound No. 233 was prepared in the same way as for compound 226 to give a while solid. ¹H NMR (400 MHz, DMSO-d₆): 11.97 (br, 1H); 7.77 (s, 1H); 7.54-7.50 (d, J=16.8 Hz, 1H); 7.45-7.40 (m, 1H); 7.26-7.16 (m, 3H); 7.00-6.96 (m, 1H); 6.90 (s, 1H): 6.86-6.71 (m, 3H); 5.17 (s, 2H); 4.29-4.26 (t, J=7.0 Hz, 2H); 4.17, 3.93 (s, s, rotamers, 1H); 3.74 (s, 3H); 3.29-3.16 (m, 4H); 2.65-2.52 (m, 4H); 2.28-2.22 (m, 1H); 1.79-1.74 (m, 2H); 1.58-1.44 (m, 6H); 1.29-1.25 (m, 6H); 0.99-0.96 (m, 1H); 0.85-0.82 (m, 3H); 0.61 (s, 9H); 0.48-0.44 (m, 1H); 0.28-0.21 (m, 2H); 0.09-0.05 (m, 1H). MS (ESI) m/z 728.7 [M+H]⁺.

### Example 44. Synthesis of Compound No. 234

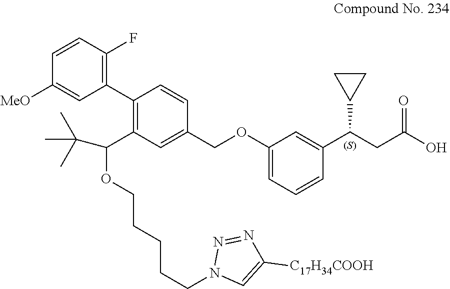

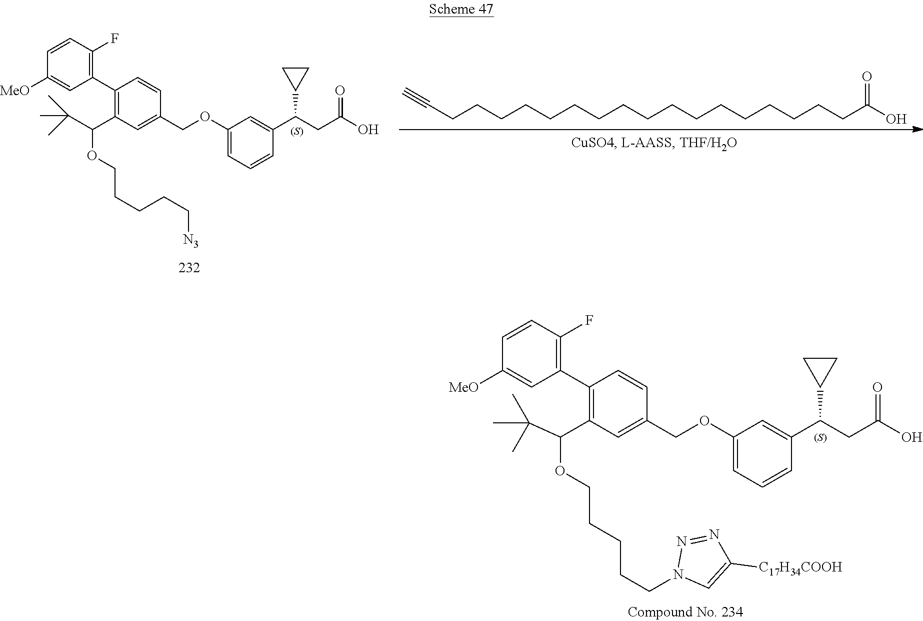

Compound 234 was prepared in the same way as for compound 226 to give a while solid. ¹H NMR (400 MHz, DMSO-d₆): 11.96 (br, 2H); 7.76 (s, 1H); 7.54-7.50 (d, J=16.8 Hz, 1H); 7.46-7.40 (m, 1H); 7.24-7.16 (m, 3H); 7.00-6.96 (m, 1H); 6.90 (s, 1H); 6.86-6.72 (m, 3H); 5.17 (s, 2H); 4.29-4.26 (t, J=6.8 Hz, 2H); 4.17, 3.93 (s, s, rotamers, 1H); 3.74 (s, 3H); 3.30-3.14 (m, 2H); 2.64-2.52 (m, 4H); 2.27-2.21 (m, 1H); 2.17 (t, J=7.4 Hz, 1H); 1.79-1.75 (m, 2H); 1.56-1.47 (m, 6H); 1.23-1.21 (m, 28H); 0.99-0.94 (m, 1H); 0.61 (s, 9H); 0.49-0.46 (m, 1H); 0.27-0.20 (m, 2H); 0.09-0.04 (m, 1H). MS (ESI) m/z 926.8 [M+H]⁺.

### Example 45. Synthesis of Compound No. 235

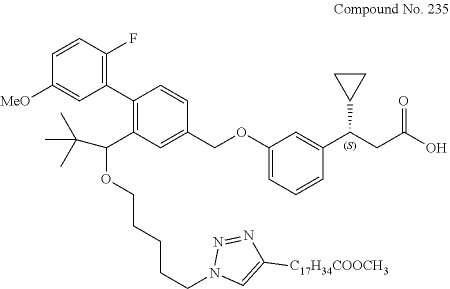

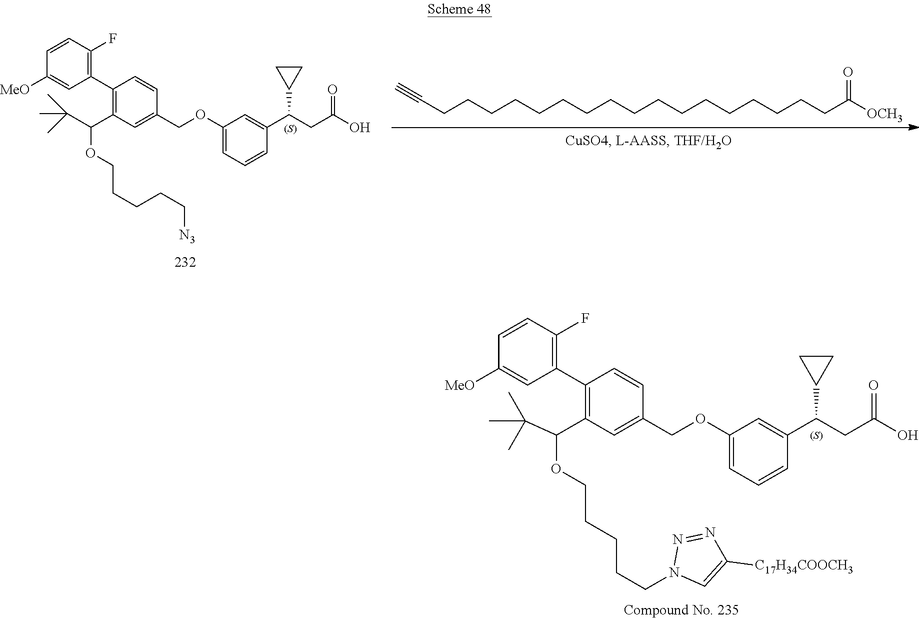

Compound No. 235 was prepared in the same way as for compound 227 to give a while solid. ¹H NMR (400 MHz, DMSO-d₆): 11.96 (br, 1H); 7.76 (s, 1H); 7.54-7.50 (d, 1H); 7.45-7.40 (m, 1H); 7.24-7.15 (m, 2H); 7.00-6.96 (m, 1H); 6.90 (s, 1H); 6.86-6.71 (m, 3H), 5.17 (s, 2H); 4.27 (t, J=6.8 Hz, 2H); 4.17, 3.93 (s, s, rotamers, 1H); 3.74 (s, 3H); 3.57 (s, 3H); 3.34-3.15 (m, 2H); 2.64-2.49 (m, 4H); 2.29-2.21 (m, 3H); 1.79-1.75 (m, 2H); 1.56-1.48 (m, 6H); 1.26-1.17 (m, 28H); 0.99-0.96 (m, 1H); 0.61 (s, 9H); 0.49-0.43 (m, 1H); 0.28-0.18 (m, 2H); 0.08-0.04 (m, 1H). MS (ESI) m/z 940.9 [M+H]⁺.

### Example 46. Synthesis of Compound No. 236

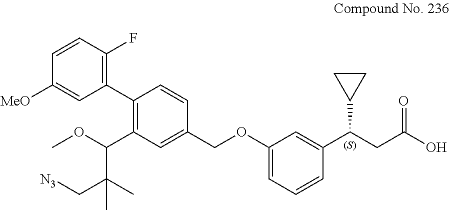

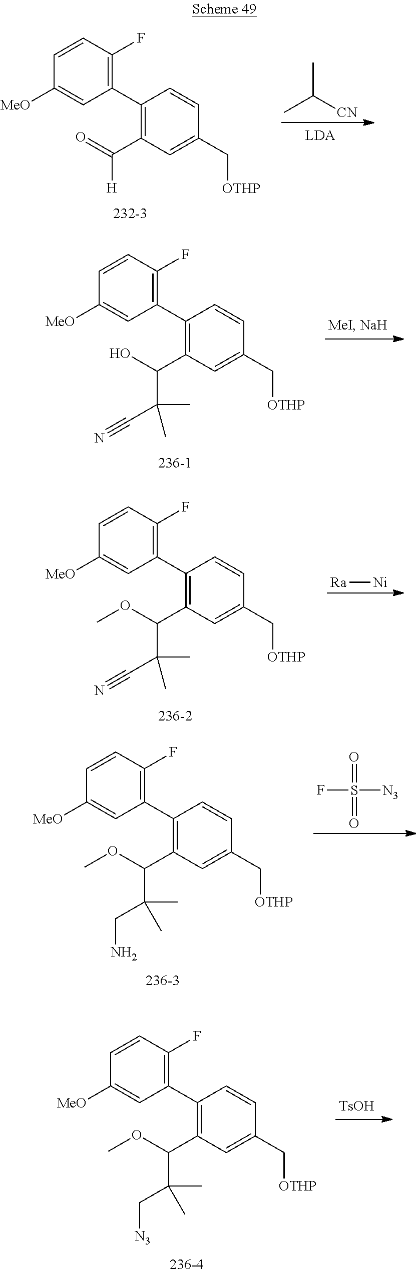

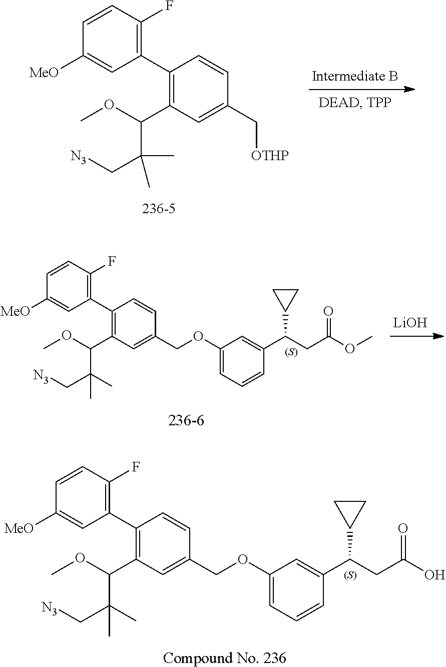

Step 1. To a solution of isobutyronitrile 1 (281 mg, 4.07 mmol) in dry THF (15 mL) at −78° C. was added LDA (2.0 M in hexane, 2.14 mL) dropwise over 20 min. The resulting mixture was stirred for 2 hrs at −78° C. Then a mixture of 2′-fluoro-5′-methoxy-4-(tetrahydro-pyran-2-yloxymethyl)-biphenyl-2-carbaldehyde 232-3 in THE (15 mL) was added dropwise over 20 min. The reaction mixture was then stirred for 16 hrs, allowing the temperature to slowly warm to r. t. The mixture was quenched by aq. NH₄Cl at 0° C. and extracted with EA (30 mL×3), dried and concentrated. The residue was purified by flash chromatograph (20% EA in PE) to give 3-[2′-fluoro-5′-methoxy-4-(tetrahydro-pyran-2-yloxymethyl)-biphenyl-2-yl]-3-hydroxy-2,2-dimethyl-propionitrile, 236-1, as a yellow solid. ¹H NMR (400 MHz, DMSO-d₆) δ 7.88-7.79 (m, 1H); 7.37-7.33 (m, 1H); 7.27-7.17 (m, 2H); 7.10-6.97 (m, 1H); 6.90-6.84 (m, 1H); 6.17-6.15 (m, 1H); 4.75-4.72 (m, 2H); 4.54-4.36 (m, 2H); 3.85-3.80 (m, 1H); 3.76-3.75 (m, 3H); 3.52-3.48 (m, 1H); 1.77-1.64 (m, 2H); 1.57-1.44 (m, 4H); 1.25-1.15 (m, 6H).

Step 2. To a mixture of 236-1 (500 mg, 1.453 mmol) in DMF (8 mL) at 0° C. was added NaH (116 mg, 60%, 2.905 mmol) in portions under N₂. The resulting mixture was stirred for 30 min at 0° C. Then Mel (412 mg, 2.905 mmol) was added. Stirred for additional 2 hrs at r. t. The reaction mixture was quenched with aq. NH₄Cl at 0° C. and extracted with EA (40 mL×3), dried and concentrated. The residue was purified by flash chromatograph (10% EA in PE) to give 3-[2′-fluoro-5′-methoxy-4-(tetrahydro-pyran-2-yloxymethyl)-biphenyl-2-yl]-3-methoxy-2,2-dimethyl-propionitrile, 236-2, as a colorless oil.

Step 3. A mixture of 236-2 (311 mg, 0.7 mmol) and Raney-Ni (˜60 mg, 20% wt) in MeOH (30 mL) was hydrogenated with a balloon at 25° C. for 16 hrs. The mixture was filtered over celite and concentrated. The residue was purified by flash chromatograph (5% to 10% of MeOH in DCM) to give 3-[2′-fluoro-5′-methoxy-4-(tetrahydro-pyran-2-yloxymethyl)-biphenyl-2-yl]-3-methoxy-2,2-dimethyl-propylamine, 236-3, as a pale-yellow oil. MS (ESI) m/z 432.1 [M+H]⁺.

Step 4. To a mixture of 236-3 (280 mg, 0.648 mmol) in DMF (30 mL) at r. t. was added fluorosulfuryl azide (˜0.5 M in MTBE, 1.4 mL) and KHCO₃ (3.0 M, 0.9 mL) dropwise. After addition the resulting mixture was stirred for 4 hrs at r. t. The mixture was diluted with water (50 mL), extracted with EA (30 mL×3), dried and concentrated. The residue was purified by flash chromatography (20% EA in PE) to give 2-[2-(3-azido-1-methoxy-2,2-dimethyl-propyl)-2′-fluoro-5′-methoxy-biphenyl-4-ylmethoxy]-tetrahydro-pyran, 236-4, as a yellow oil.

Step 5. To a mixture of 236-4 (300 mg, 0.656 mmol) in MeOH (6 mL) was added TsOH·H₂O (374 mg, 1.969 mmol). The resulting mixture was stirred for 2 hrs at r. t. The reaction mixture was diluted with water (20 mL), extracted with EA (30 mL×4) and concentrated. The residue was purified by flash chromatography (25% EA in PE) to give [2-(3-azido-1-methoxy-2,2-dimethyl-propyl)-2′-fluoro-5′-methoxy-biphenyl-4-yl]-methanol, 236-5, as a yellow oil.

Step 6. A solution of 236-5 (210 mg, 0.563 mmol), Intermediate B (160 mg, 0.76 mmol) and PPh₃ (295 mg, 1.126 mmol) in DCM (8 mL) was degassed and filled with N₂ 3 times and cooled to 0° C. DEAD (196 mg, 1.126 mmol) was then added dropwise via syringe. The resulting mixture was then stirred for 12 hrs under N₂, left the temperature slowly warm to r. t. Solvent was removed and the residue was purified by flash chromatography (10% EA in PE) to give 3-{3-[2-(3-azido-1-methoxy-2,2-dimethyl-propyl)-2′-fluoro-5′-methoxy-biphenyl-4-ylmethoxy]-phenyl}-3-cyclopropyl-propionic acid methyl ester, 236-6, as a colorless oil. Note: weak MS. ¹H NMR (400 MHz, DMSO-d₆) δ 7.55-7.48 (m, 2H); 7.25-7.17 (m, 3H); 7.02-6.99 (m, 1H); 6.91 (s, 1H); 6.86-6.81 (m, 3H); 5.20 (s, 2H); 4.41-4.16 (m, 1H); 3.75 (s, 3H); 3.51 (s, 3H); 3.35-3.32 (m, 1H); 3.22-3.14 (m, 3H); 2.95-2.92 (m, 1H); 2.76-2.68 (m, 2H); 2.26-2.22 (m, 1H); 1.01-0.98 (m, 1H); 0.65 (s, 3H); 0.49-0.46 (m, 1H); 0.38 (s, 3H); 0.29-0.26 (m, 1H); 0.20-0.15 (m, 1H); 0.10-0.05 (m, 1H).

Step 7. A mixture of 236-6 (180 mg, 0.31 mmol) and LiOH·H₂O (128 mg, 3.13 mmol) in water (3 mL), MeOH (3 mL) and THE (3 mL) was stirred at 55° C. for 14 hrs. MeOH and THF were removed and the residue was acidified with aqueous HCl until pH reached 3 and extracted with EtOAc (15 mL×3). The combined organic layers were dried over Na₂SO₄ and concentrated to give crude (3S)-3-(3-((2-(3-azido-1-methoxy-2,2-dimethylpropyl)-2′-fluoro-5′-methoxy-[1,1′-biphenyl]-4-yl)methoxy)phenyl)-3-cyclopropylpropanoic acid, Compound No. 236, as a yellow oil, which was purified with preparative HPLC to give a while solid. MS (ESI) m/z 560.4 [M−H]⁻. ¹H NMR (400 MHz, DMSO-d₆): 11.98 (br, 1H); 7.56-7.48 (m, 2H); 7.27-7.17 (m, 3H); 7.02-6.99 (m, 1H); 6.90-6.82 (m, 4H); 5.20 (s, 2H); 4.42, 4.16 (s, s, rotamers, 1H); 3.75 (s, 3H); 3.36-3.30 (d, J=23 Hz, 1H); 3.23, 3.15 (s, s, rotamers, 3H); 2.96-2.93 (d, J=23 Hz, 1H); 2.65-2.61 (m, 2H); 2.26-2.22 (m, 1H); 1.00-0.97 (m, 1H); 0.67, 0.39 (s, s, rotamers, 3H); 0.50-0.45 (m, 1H); 0.29-0.20 (m, 2H); 0.09-0.07 (m, 1H).

### Example 47. Synthesis of Compound No. 237

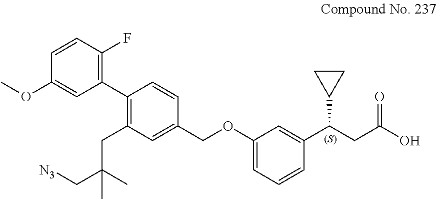

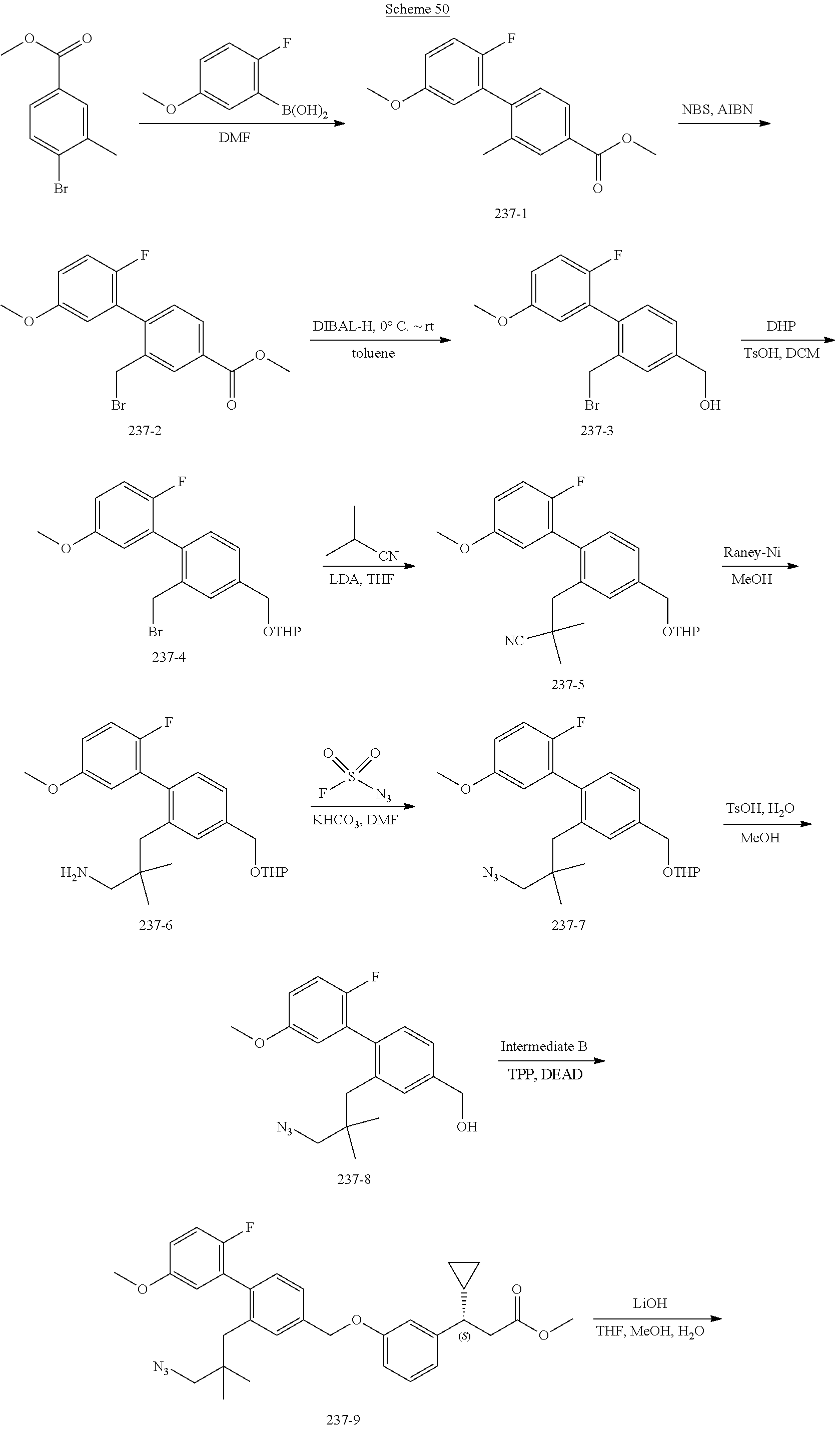

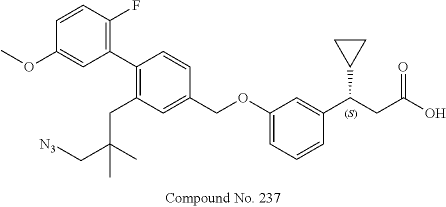

Step 1. A mixture of 4-bromo-3-methyl-benzoic acid methyl ester (19 g, 83.2 mol), phenylboronic acid 2 (21.2 g, 124 mol), (PPh3)4 (4.8 g, 4.16 mmol) and K2CO3 (22.96 g, 166.4 mol) in DMF (200 mL) was degassed and filled with N₂ 3 times and heated at 100° C. for 16 hrs. DMF was removed and the residue was diluted with water (100 mL), extracted with EA (60 mL×3), dried and concentrated. The residue was purified by flash chromatography (10% of EA in PE) to give 2-bromomethyl-2′-fluoro-5′-methoxy-biphenyl-4-carboxylic acid methyl ester, 237-1, (20 g, yield: 88%) as a red oil. MS (ESI) m/z 275.0 [M+H]⁺.

Step 2. To a mixture of 237-1 (20 g, 72.99 mol) and AIBN (2.38 g, 14.52 mol) in CCl4 (250 mL) at r. t. was added NBS (10.3 g, 58.08 mol) in small portions. The resulting mixture was heated to reflux (85° C.) for 20 hrs. Solvent was removed and the residue was purified by flash chromatography (5% to 10% of EA in PE) to give 2-bromomethyl-2′-fluoro-5′-methoxy-biphenyl-4-carboxylic acid methyl ester, 237-2, as yellow oil. MS (ESI) m/z 352.9 [M+H]⁺.

Step 3. To a mixture of 237-2 (27 g, about 73 mol) in toluene (300 mL) at 0° C. was added DIBAL-H (1.0 M in hexane, 150 mL) dropwise over 30 min. The resulting mixture was stirred for 16 hrs, allowing the temperature to slowly warm to r. t. The mixture was quenched with aq. NH₄Cl at 0° C. and the precipitate was removed by filtration. Organic phase was separated and the water phase was extracted with EA (100 mL×3). The organic phase was combined, dried and concentrated to give crude (2-bromomethyl-2′-fluoro-5′-methoxy-biphenyl-4-yl)-methanol, 237-3, as orange oil. MS (ESI) m/z 342.1 [M+18]⁺.

Step 4. To a mixture of 237-3 (13.6 g, 41.9 mmol) and TsOH·H₂O (796 mg, 4.19 mmol) in DCM (150 mL) at r. t. was added DHP (5.28 g, 62.85 mmol) dropwise. The mixture was stirred at room temperature for 12 h, and quenched with water (100 mL). The organic layer was separated, extracted with DCM (50 mL×3). The organic phase was combined, dried and concentrated. The residue was purified by flash chromatography (5% to 10% of EA in PE) to give 2-(2-bromomethyl-2′-fluoro-5′-methoxy-biphenyl-4-ylmethoxy)-tetrahydro-pyran, 237-4, as a white oil.

Step 5. To a solution of isobutyronitrile (5.06 g, 0.073 mol) in THE (100 mL) cooled to −70° C. was added LDA (36.7 mL, 0.073 mol) dropwise under N₂ and the mixture was stirred at −70° C. for 2 h. Then a solution of 237-4 (15.0 g, 0.037 mmol) in THE (50 mL) was added at −70° C. and the mixture was stirred at room temperature for 12 h. The reaction mixture was quenched with NH₄Cl aqueous solution and extracted with EA. The organic phase was washed with water and brine, dried with Na₂SO₄, filtered, and the filtrate was concentrated. The residue was purified by column chromatography on silica gel (EA/PE=25%) to give 3-(2′-fluoro-5′-methoxy-4-(((tetrahydro-2H-pyran-2-yl)oxy)methyl)-[1,1′-biphenyl]-2-yl)-2,2-dimethylpropanenitrile, 237-5, as a yellow solid. H NMR (400 MHz, DMSO-d₆): 7.51 (s, 1H); 7.35 (d, J=8.0 Hz, 1H); 7.25-7.19 (m, 2H); 7.01-6.98 (m, 1H); 6.88-6.86 (m, 1H); 4.72 (dd, J=4.4 Hz, J=7.6 Hz, 1H); 4.51 (d, J=12.4 Hz, 1H); 3.85-3.79 (m, 1H); 3.75 (s, 3H); 3.52-3.48 (m, 1H); 2.96-2.93 (m, 1H); 2.75-2.72 (m, 1H); 1.80-1.65 (m, 2H); 1.58-1.48 (m, 4H); 1.12 (s, 3H); 1.03 (s, 3H).

Step 6. To a solution of 237-5 (5.0 g, 12.6 mmol) in MeOH (150 mL) was added Raney Ni (5 g) and the mixture was stirred at 45° C. under H₂ for 12 h. After the reaction was completed, the reaction mixture was filtered, and the filtrate was concentrated to give 3-(2′-fluoro-5′-methoxy-4-(((tetrahydro-2H-pyran-2-yl)oxy)methyl)-[1,1′-biphenyl]-2-yl)-2,2-dimethylpropan-1-amine, 237-6, as a yellow oil. MS (ESI) m/z 402.3 [M+H]⁺.

Step 7. To a solution of 237-6 (2 g, 4.99 mmol) in DMF (10 mL) cooled to 0° C. was added KHCO₃ aqueous solution (6.65 mL, 19.95 mmol) and sulfurazidic fluoride (11.0 mL, 5.49 mmol) in MTBE. The mixture was stirred at room temperature for 1 h. The reaction mixture was quenched with water and extracted with EA. The organic phase was washed with water and brine, dried with Na₂SO₄, filtered, and the filtrate was concentrated. The residue was purified by column chromatography on silica gel (EA/PE=10%) to give 2-((2-(3-azido-2,2-dimethylpropyl)-2′-fluoro-5′-methoxy-[1,1′-biphenyl]-4-yl)methoxy)tetrahydro-2H-pyran, 237-7, as a yellow oil. MS (ESI) m/z 450.2 [M+Na]⁺.

Step 8. To a solution of 237-7 (2.0 g, 4.68 mmol) in MeOH (20 mL) was added TsOH (1.78 g, 9.37 mmol) and the mixture was stirred at room temperature for 1 h. The reaction mixture was diluted with water and extracted with EA. The organic phase was washed with water and brine, dried with Na₂SO₄, filtered, and the filtrate was concentrated. The residue was purified by column chromatography on silica gel (EA/PE=20%) to give (2-(3-azido-2,2-dimethylpropyl)-2′-fluoro-5′-methoxy-[1,1′-biphenyl]-4-yl)methanol, 237-8, as a yellow oil. ¹H NMR (400 MHz, DMSO-d6): 7.26-7.14 (m, 4H); 6.98-6.94 (m, 1H); 6.83-6.80 (m, 1H); 5.22 (t, J=5.8 Hz, 1H); 4.53 (d, J=5.2 Hz, 2H); 3.75 (s, 3H); 3.02 (d, J=3.2 Hz, 2H); 2.66-2.50 (m, 2H); 0.62 (s, 6H). MS (ESI) m/z 366.2 [M+Na]⁺.

Step 9. To a mixture of 237-8 (1.4 g, 4.08 mmol), Intermediate B (1.35 g, 6.12 mmol) and TPP (1.60 g, 6.12 mmol) in DCM (15 mL) cooled to 0° C. was added DEAD (1.07 g, 6.12 mmol) under N₂. The mixture was stirred at room temperature for 12 h. The reaction mixture was quenched with water and extracted with DCM. The organic phase was washed with water and brine, dried with Na₂SO₄, filtered, and the filtrate was concentrated. The residue was purified by column chromatography on silica gel (EA/PE=10%) to give (S)-methyl 3-(3-((2-(3-azido-2,2-dimethylpropyl)-2′-fluoro-5′-methoxy-[1,1′-biphenyl]-4-yl)methoxy) phenyl)-3-cyclopropylpropanoate, 237-9, as a colorless oil. MS (ESI) m/z 546.2 [M+H]⁺.

Step 10. To a solution of 237-9 (1.2 g, 2.20 mmol) in MeOH (5 mL) and THE (5 mL) was added LiGH (0.92 g, 22.02 mmol) and H₂O (1 mL), and the mixture was stirred at 45° C. for 8 h. After the reaction was completed, the reaction mixture was diluted with water and adjusted to pH=5 with HCl (2N). The resulting mixture was extracted with EA and the organic phase was washed with water and brine, dried with Na₂SO₄, filtered, and the filtrate was concentrated to give (S)-3-(3-((2-(3-azido-2,2-dimethylpropyl)-2′-fluoro-5′-methoxy-[1,1′-biphenyl]-4-yl)methoxy)phenyl)-3-cyclopropylpropanoic acid, Compound No. 237, as a white solid. ¹H NMR (400 MHz, DMSO-d6): 11.97 (br, 1H); 7.40-7.36 (m, 2H); 7.24-7.18 (m, 3H); 6.99-6.96 (m, 1H); 6.91 (s, 1H); 6.87-6.83 (m, 3H); 5.14 (s, 2H); 3.75 (s, 3H); 2.99 (d, J=2.4 Hz, 2H); 2.69-2.58 (m, 4H); 2.29-2.23 (m, 1H); 1.01-0.97 (m, 1H); 0.59 (s, 6H); 0.52-0.46 (m, 1H); 0.32-0.20 (m, 2H); 0.12-0.08 (m, 1H). MS (ESI) m/z 530.1 [M−H]˜.

### Biological Example 1. Material and General Methods

IP1 accumulation assay was used to evaluate the potency of compounds. HEK293 cells stably expressing GPR40 were cultured in 5% CO₂ incubator (ThermoFisher) in maintenance media (Dulbecco's modified Eagle's medium with 4.5 g/L of glucose, 10% fetal bovine serum, 100 μg/mL Hygromycin, and Penicillin (100 U/mL)/Streptomycin (100 μg/mL)) till 100% confluency. Cells were harvested freshly, spun down at 300×g for 5 min, and resuspended in pre-warmed 1× stimulant buffer from Cisbio IP-One HTRF Detection kit (Cisbio). Cell density was adjusted to 2.0×10⁶ cells/mL. DMSO was used as blank control and AMG-1638 (CAS #: 1142214-62-7) as positive control. Compounds were prepared at 10 mM in DMSO and 5 nL of 3× serially diluted compounds (10 concentrations) were added to each well of the 384-LDV assay plate (Corning) by using ECHO 550 (Labcyte). Five μL of cells in suspension were transferred into each well by using Multidrop Combi Reagent Dispenser (ThermoFisher). Assay plate was then sealed and incubated at 37° C. for 2 hours. IP-d2 reagent and anti-IP1 reagent were prepared following the manual (Cisbio). Five μL of IP1-d2 and then 5 μL of anti-IP1 antibody was added to each well sequentially. Assay plate was incubated at room temperature for 60 min and then read at 665 nm/615 nm on an Envision plate reader (PerkinElmer). The ratio of values obtained at 665 nm and 615 nm was used for calculation of IP1 accumulation: % Effect=(Ratioₛₐₘₚₗₑ−Ratio_{blank})/(Ratio_{AMG-1638}−Ratio_{blank})×100. Dose curve was fitted and EC₅₀ of each compound was calculated by using XLFit.

Selected compounds of the present disclosure were tested according to Biological Example 1 and the EC₅₀ values are shown in the table below, in the table below, “*” refers to 1 nM≤EC₅₀<50 nM; “**” refers to 50 nM≤EC₅₀<250 nM; “***” refers to 250 nM≤EC₅₀<1000 nM; “****” refers to 1000 nM≤EC₅₀<5000 nM; and “*****” refers to 5000 nM≤EC₅₀:

| Compound | EC50 | Compound | EC50 | Compound | EC50 |
|---|---|---|---|---|---|
| No | (nM) | No | (nM) | No | (nM) |
| 193 | * | 207 | ***** | 225 | ** |
| 194 | * | 209 | **** | 226 | ** |
| 201 | * | 210 | ** | 227 | ***** |
| 187 | ** | 211 | **** | 228 | ** |
| 188 | * | 212 | ***** | 229 | *** |
| 195 | * | 213 | **** | 230 | *** |
| 189 | * | 216 | * | 231 | ***** |
| 190 | ** | 217 | * | 232 | * |
| 130 | **** | 218 | *** | 233 | * |
| 203 | ** | 219 | *** | 234 | *** |
| 1 | ** | 220 | **** | 235 | ***** |
| 8 | ** | 221 | *** | 236 | * |
| 204 | * | 224 | * | 237 | * |
| 205 | ** | | | | |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

1. A compound of Formula I, or a pharmaceutically acceptable salt or ester thereof: 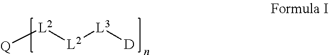
wherein:
Q is a carrier covalently bonded to L¹;
n is an integer of 1-500;
L¹ at each occurrence is independently a structure of Formula L-1: 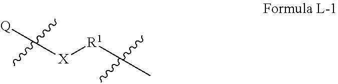
wherein: X is a bond, —CR¹⁰³R¹⁰⁴—, N(R¹⁰⁰)—, —O—, —S—, —SO₂—, —C(═O)—, —C(═O)—N(R¹⁰⁰)—, —S(═O)₂—N(R¹⁰⁰)—, —P(═O)(OR¹⁰²)—N(R¹⁰⁰)—, —C(═O)—O—, —S(═O)₂—O—, or —P(═O)(OR¹⁰²)—O—; and R¹ is a saturated or partially unsaturated aliphatic group or an aromatic group, e.g., a C₁₀₋₅₀ alkyl, wherein the longest chain length of the aliphatic group is at least 10 carbons;
L² at each occurrence is independently —N(R¹⁰⁰)—, —O—, —S—, —SO₂—, —C(═O)—, or a moiety selected from: 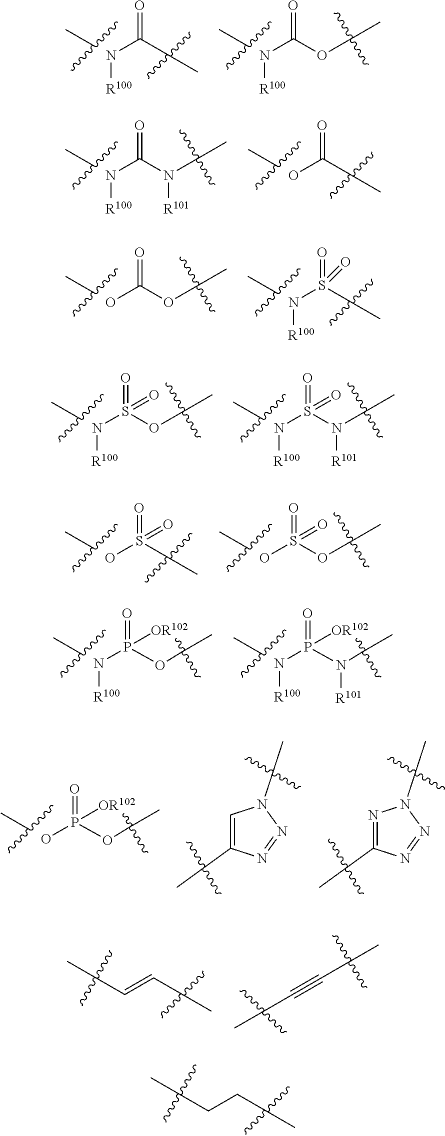
L³ at each occurrence is independently a bond, optionally substituted alkylene, optionally substituted heteroalkylene, optionally substituted carbocyclylene, optionally substituted heterocyclylene, optionally substituted arylene, or optionally substituted heteroarylene, and D is a residue of a GPR40 agonist;
wherein R¹⁰⁰, R¹⁰¹ and R¹⁰² at each occurrence is independently hydrogen, optionally substituted alkyl, or optionally substituted cycloalkyl, wherein R¹⁰³ and R¹⁰⁴ are independently hydrogen, halogen, optionally substituted alkyl, or optionally substituted cycloalkyl; or R¹⁰³ and R¹⁰⁴ are joined to form a C(═O) or an optionally substituted cyclic structure.

2. The compound of claim 1, or a pharmaceutically acceptable salt or ester thereof, wherein Q is a hydrophilic carrier.

3. The compound of claim 1, or a pharmaceutically acceptable salt or ester thereof, wherein Q is a residue of a dendrimer selected from a poly(amide amine) dendrimer, a poly(propylene amine) dendrimer, or a poly (amide amine)-poly(propylene amine) dendrimer.

4. (canceled)

5. (canceled)

6. (canceled)

7. (canceled)

8. (canceled)

9. The compound of claim 1, or a pharmaceutically acceptable salt or ester thereof, wherein Q has a Formula Q-1, Q-2, Q-3, Q-4, Q-5A or Q-5B: 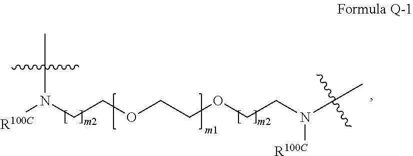
wherein: m1 is an integer of 0-100, and each m2 is independently an integer of 0-5, and each R^{100C} is independently hydrogen, optionally substituted alkyl, or optionally substituted cycloalkyl,
wherein at least one of the terminal NR^{100C} of Formula Q-1 forms a covalent bond with an L¹; 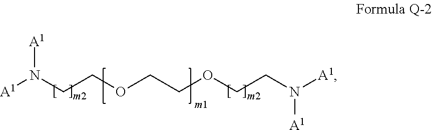
wherein each A¹ is independently F-1, F-2, or F-3, 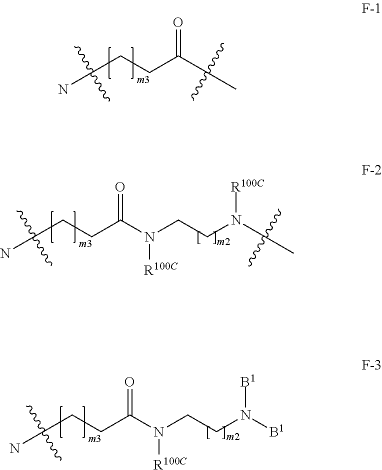
wherein each B¹ group in F-3 is independently F-1 or F-2, or a moiety having at least one repeating units of 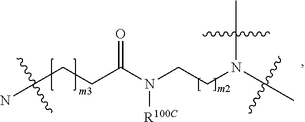
wherein the moiety terminates with a structure comprising 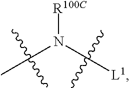
(L¹ is showing to show connectivity if L¹-L²-L³-D is bond at the terminal);
wherein:
m1 is an integer of 0-100, such as 0-10 (e.g., 1, 2, 3, 4, 5, 6, 7, 8, 9, or 10), 10-50, 50-100, etc.;
each m2 is an integer of 0-5 (e.g., 1, 2, or 3);
each m3 is independently an integer of 0-5 (e.g., 0, 1, 2, or 3);
R^{100C} at each occurrence is independently hydrogen, optionally substituted alkyl, or optionally substituted cycloalkyl; and
wherein at least one of the A¹ forms a covalent bond with an L¹ through a terminal carbonyl group or —N—R^{100C} group; 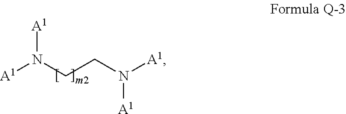
wherein each A¹ is independently F-1, F-2, or F-3, 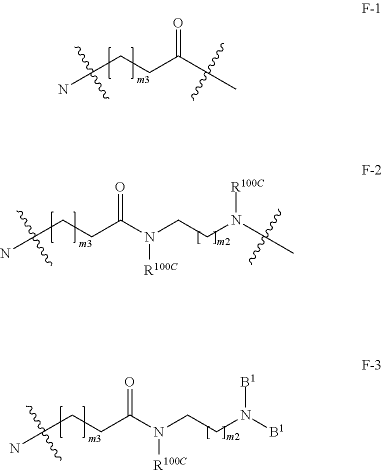
wherein each B¹ group in F-3 is independently F-1 or F-2, or a moiety having at least one repeating units of 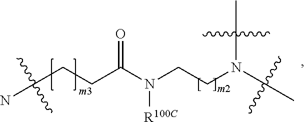
wherein the moiety terminates with a structure comprising 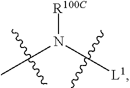
(L¹ is showing to show connectivity if L¹-L²-L³-D is bond at the terminal);
wherein:
each m2 is an integer of 0-5 (e.g., 1, 2, or 3):
each m3 is independently an integer of 0-5 (e.g., 0, 1, 2, or 3):
R^{100C} at each occurrence is independently hydrogen, optionally substituted alkyl, or optionally substituted cycloalkyl; and
wherein at least one of the A¹ forms a covalent bond with an L¹ through a terminal carbonyl group or —N—R^{100C} group; 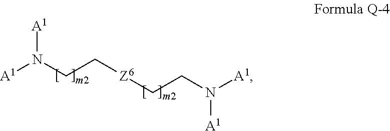
wherein:
Z⁶ is O, NR^{100D} a polyethylene glycol (PEG) chain, optionally substituted alkylene, optionally substituted heteroalkylene, optionally substituted carbocyclylene, optionally substituted heterocyclylene, optionally substituted arylene, or optionally substituted heteroarylene, each A¹ is independently F-1, F-2, or F-3, 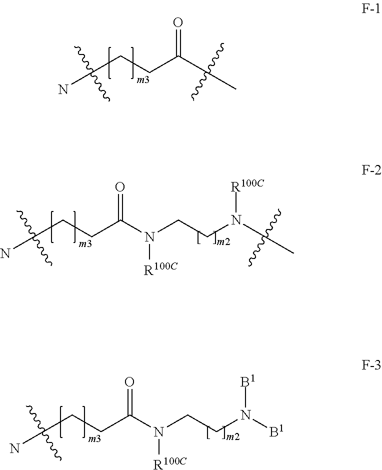
wherein each B¹ group in F-3 is independently F-1 or F-2, or a moiety having at least one repeating units of 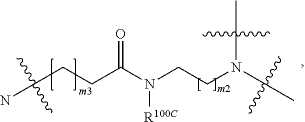
wherein the moiety terminates with a structure comprising 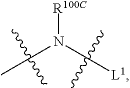
(L¹ is showing to show connectivity if L¹-L²-L³-D is bond at the terminal);
wherein:
each m2 is an integer of 0-5 (e.g., 1, 2, or 3);
each m3 is independently an integer of 0-5 (e.g., 0, 1, 2, or 3);
R^{100C} at each occurrence is independently hydrogen, optionally substituted alkyl, or optionally substituted cycloalkyl;
R^{100D} is hydrogen, optionally substituted alkyl, or optionally substituted cycloalkyl; and wherein at least one of the A¹ forms a covalent bond with an L¹ through a terminal carbonyl group or —N—R^{100C} group; 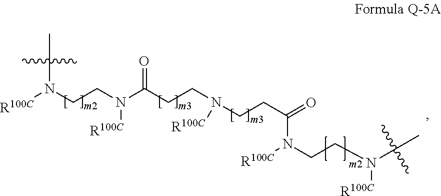
wherein at least one of the terminal NR^{100C} of Formula Q-5A forms a covalent bond with an L¹; or 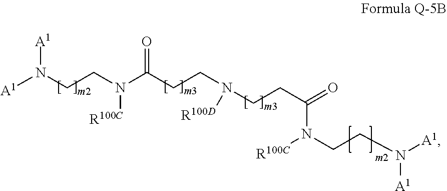
wherein each A¹ is independently F-1, F-2, or F-3, 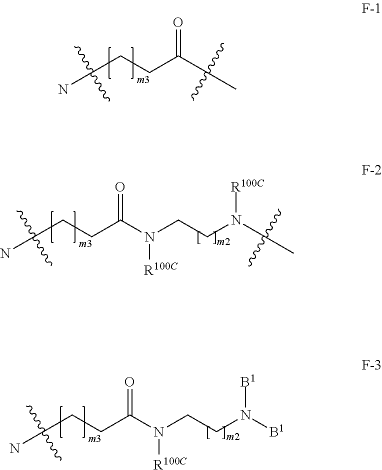
wherein each B¹ group in F-3 is independently F-1 or F-2, or a moiety having at least one repeating units of 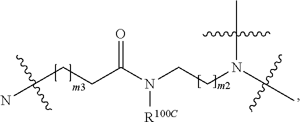
wherein the moiety terminates with a structure comprising 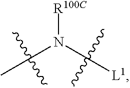
(L¹ is showing to show connectivity if L¹-L²-L³-D is bond at the terminal):
wherein:
each m2 is independently an integer of 0-5 (e.g., 1, 2, or 3);
each m3 is independently an integer of 0-5 (e.g., 0, 1, 2, or 3);
R^{100C} at each occurrence is independently hydrogen, optionally substituted alkyl, or optionally substituted cycloalkyl;
R^{100D} is hydrogen, optionally substituted alkyl, or optionally substituted cycloalkyl; and wherein at least one of the A¹ forms a covalent bond with an L¹ through a terminal carbonyl group or —N—R^{100C} group.

10-13. (canceled)

14. The compound of claim 1, or a pharmaceutically acceptable salt or ester thereof, wherein n is an integer of 1-64, e.g., 1-4, 2-8, 4-16, etc.

15. The compound of claim 1, or a pharmaceutically acceptable salt or ester thereof, wherein L¹ at each occurrence is independently a residue of Formula L-1: 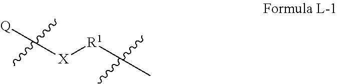
wherein:
i) if Q forms a covalent bond with X through a —C(═O)— group, then X is —N(R¹⁰⁰)—; or
ii) if Q forms a covalent bond with X through a —NR¹⁰⁰— group, then X is —C(═O)—, —C(═O)—N(R¹⁰⁰)—, —SO₂— or —C(═O)—O—, preferably, X is —C(═O)—.

16. The compound of claim 1, or a pharmaceutically acceptable salt or ester thereof, wherein R¹ at each occurrence is independently a C₁₀₋₅₀ alkyl, e.g., a straight chain or branched C₁₀₋₃₀ alkyl, or a C₁₀₋₅₀ alkenyl, e.g., a straight chain or branched C₁₀₋₃₀ alkenyl.

17. The compound of claim 1, or a pharmaceutically acceptable salt or ester thereof, wherein L² at each occurrence is independently

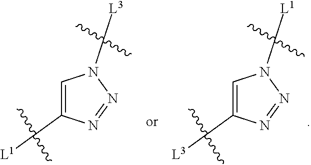

18. The compound of claim 1, or a pharmaceutically acceptable salt or ester thereof, wherein L³ at each occurrence is independently a bond, optionally substituted C₁₋₁₀ alkylene, or optionally substituted C₁₋₁₀ heteroalkylene having 1-5 heteroatoms independently selected from O and N.

19. (canceled)

20. The compound of claim 1, or a pharmaceutically acceptable salt or ester thereof, wherein D at each occurrence is independently selected from: 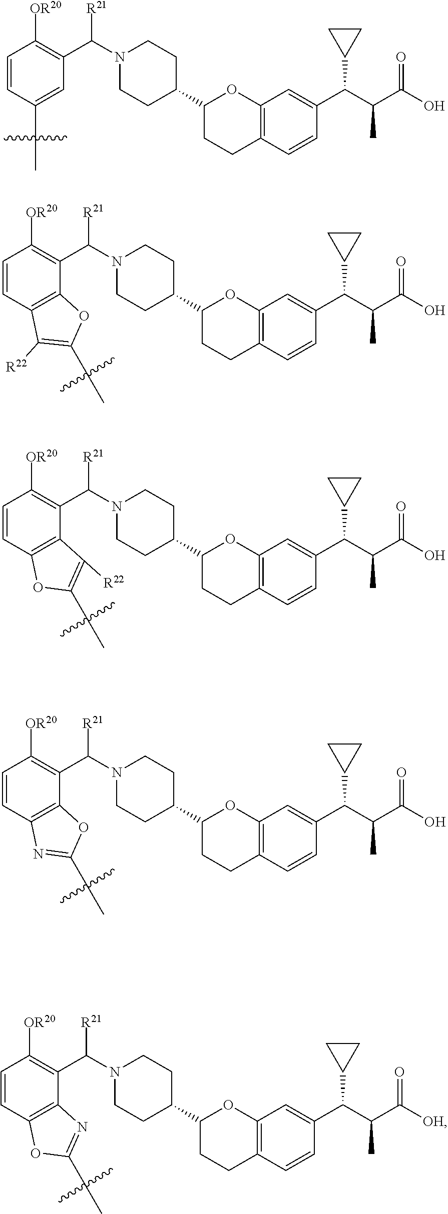
wherein: R²⁰ is C₁₋₆ alkyl or fluorine substituted C₁₋₆ alkyl, R²¹ is hydrogen or a C₁₋₆ alkyl, and R²² is hydrogen, halogen, CN, C₁₋₆ alkyl or fluorine substituted C₁₋₆ alkyl or a C₃₋₆ cycloalkyl.

21. A compound of Formula II, or a pharmaceutically acceptable salt or ester thereof: 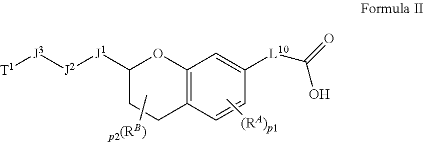
wherein:
L¹⁰ is an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, optionally substituted heterocyclyl, optionally substituted aryl, and optionally substituted heteroaryl, or two substituents are joined to form an optionally substituted ring structure;
R^{A} at each occurrence is independently halogen, CN, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{A} are joined to form an optionally substituted ring structure; p1 is 0, 1, or 2;
R^{B} at each occurrence is independently halogen, hydroxyl, amino, substituted amino, optionally substituted C₁₋₆ alkyl, optionally substituted C₃₋₆ cycloalkyl, optionally substituted C₁₋₆ alkoxy, or optionally substituted C₃₋₆ cycloalkoxy, or two R^{B} are joined to form an optionally substituted ring structure; p2 is 0, 1, 2, 3, or 4;
J¹ is a bond, an optionally substituted aryl or heteroaryl ring, —C₁₋₆alkylene-N(R¹⁰⁰)—, 3-14 membered optionally substituted heterocyclylene containing at least one ring nitrogen atom, or —C₁₋₆alkylene-(3-14 membered optionally substituted heterocyclylene containing at least one ring nitrogen atom)-;
J² is a bond or an alkylene, optionally substituted with 1-3 substituents independently selected from halogen, optionally substituted C₁₋₆ alkyl, optionally substituted C₂₋₆ alkenyl, optionally substituted C₂₋₆ alkynyl, optionally substituted C₁₋₆ heteroalkyl, optionally substituted C₃₋₆ cycloalkyl, optionally substituted C₁₋₆ alkoxy, optionally substituted C₃₋₆ cycloalkoxy, or two substituents are joined to form an optionally substituted ring structure;
J³ is an optionally substituted cycloalkyl, heterocyclyl, aryl or heteroaryl ring,
T¹ is:
1) —C₅₋₅₀ alkylene-T^{A}, wherein T^{A} is hydrogen or a structure having a hydrophilic moiety, e.g., a moiety having one or more ethylene glycol unit, one or more ethylene diamine unit, one or more ethylene amino ether or alcohol unit, one or more groups that are charged or can become charged at pH about 7, etc.;
2) -T^{B}-C₅₋₅₀ alkylene-T^{A}; wherein T^{A} is defined above, T^{B} is —N(R¹⁰⁰)—, —O—, —S—, —SO₂—, —C(═O)—, or a moiety selected from: 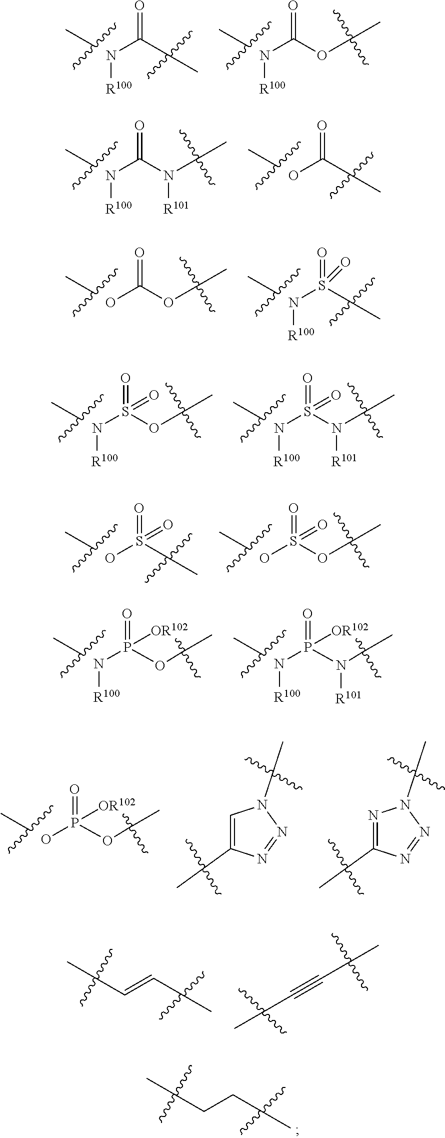
3) a moiety having a formula of -T^{C}-T^{B}-T^{D}-C₅₋₅₀ alkylene-T^{A}, wherein T^{C} and T^{D} are independently a bond, optionally substituted alkylene, optionally substituted heteroalkylene, optionally substituted carbocyclylene, optionally substituted heterocyclylene, optionally substituted arylene, or optionally substituted heteroarylene, and T^{A} and T^{B} are defined above; or
4) a moiety having a formula of -T^{C}-G, wherein T^{C} is defined above, and G is hydrogen, OH, N₃ or, 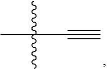
wherein R¹⁰⁰, R¹⁰¹ and R¹⁰² at each occurrence is independently hydrogen, optionally substituted alkyl, or optionally substituted cycloalkyl.

22. The compound of claim 21, or a pharmaceutically acceptable salt or ester thereof, which has a Formula II-1:

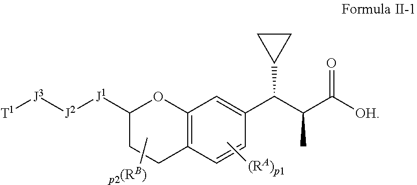

23. (canceled)

24. (canceled)

25. (canceled)

26. (canceled)

27. The compound of claim 21, or a pharmaceutically acceptable salt or ester thereof, which has a Formula II-1-A:

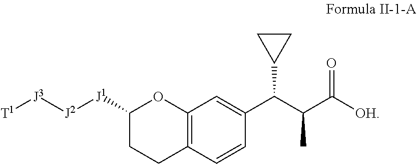

28. (canceled)

29. (canceled)

30. The compound of claim 29, or a pharmaceutically acceptable salt or ester thereof, wherein J¹ is selected from: 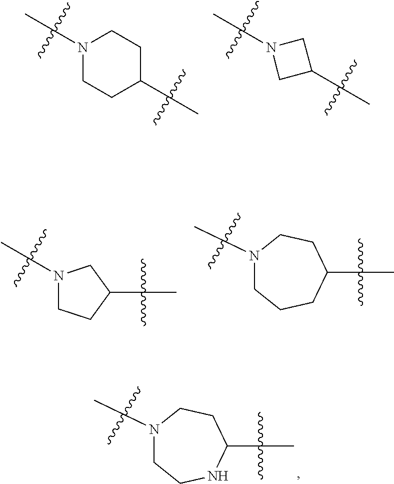 [SMILES: *C1CCN(*)CC1 ; *C1CN(*)C1 ; *C1CCN(*)C1 ; *C1CCCN(*)CC1 ; *C1CCN(*)CCN1]
each of which is optionally substituted with 1-2 substituents independently selected from F, OH, NH₂, NH(C₁₋₄ alkyl), N(C₁₋₄ alkyl)(C₁₋₄ alkyl), C₁₋₄ alkyl optionally substituted with 1-3 fluorine, or C₁₋₄ alkoxy optionally substituted with 1-3 fluorine.

31. (canceled)

32. (canceled)

33. The compound of claim 21, or a pharmaceutically acceptable salt or ester thereof, wherein J² is CH₂ or —CH(CH₃)—.

34. (canceled)

35. (canceled)

36. (canceled)

37. (canceled)

38. (canceled)

39. The compound of claim 21, or a pharmaceutically acceptable salt or ester thereof, wherein J³ is selected from: 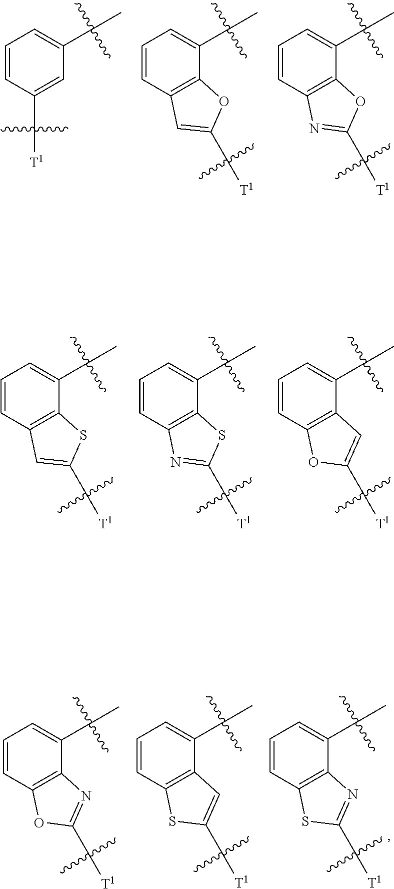
wherein each of which is optionally substituted with 1-3 substituents independently selected from F, Cl, CN, OH, C₁₋₆ alkyl optionally substituted with F (e.g., CF₃), cyclopropyl, cyclobutyl, C₁₋₆ alkoxy optionally substituted with F (e.g., —O—CF₃), or C₃₋₆ cycloalkoxy.

40. The compound of claim 21, or a pharmaceutically acceptable salt or ester thereof, which has a Formula II-1-A-1, II-1-A-2, II-1-A-3, II-1-A-4, or II-1-A-5: 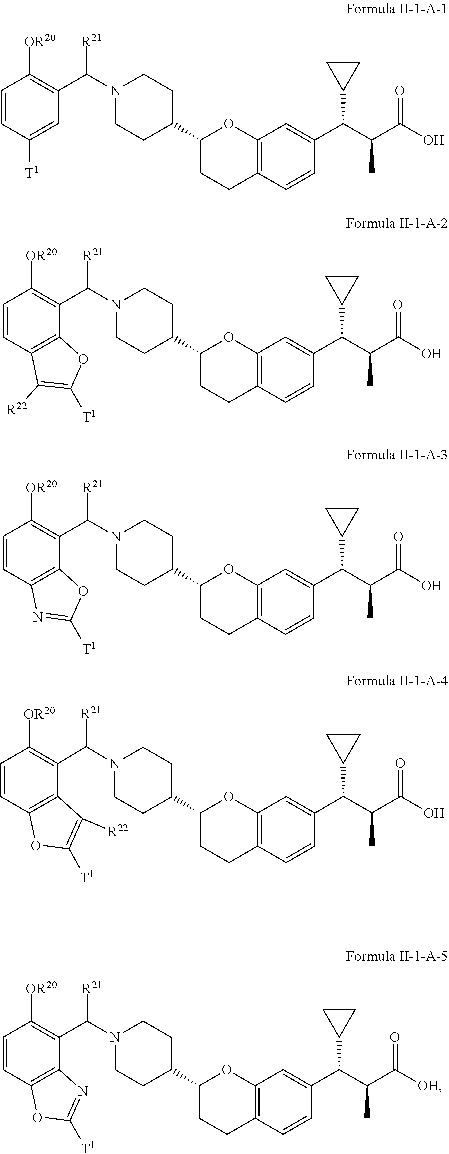
wherein:
R²⁰ is C₁₋₆ alkyl or fluorine substituted C₁₋₆ alkyl, R²¹ is hydrogen or C₁₋₆ alkyl, and R²² is hydrogen, halogen, CN, C₁₋₆ alkyl or fluorine substituted C₁₋₆ alkyl or a C₃₋₆ cycloalkyl.

41. (canceled)

42. (canceled)

43. The compound of claim 21, or a pharmaceutically acceptable salt or ester thereof, wherein T^{B} is N(R¹⁰⁰)—, —O—, —C(═O)—, or a moiety selected from:

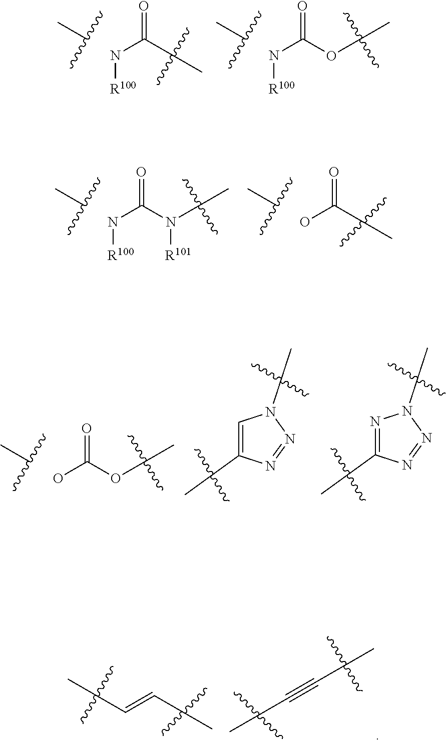

44. (canceled)

45. (canceled)

46. The compound of claim 21, or a pharmaceutically acceptable salt or ester thereof, wherein T^{C} and T^{D} are independently a bond, optionally substituted C₁₋₁₀ alkylene, optionally substituted C₁₋₁₀ heteroalkylene having 1-5 heteroatoms independently selected from O and N, e.g., —CH₂—O—CH₂—.

47. (canceled)

48. (canceled)

49. (canceled)

50. (canceled)

51. (canceled)

52. (canceled)

53. (canceled)

54. The compound of claim 21, or a pharmaceutically acceptable salt or ester thereof, wherein T^{A} is —OH, amine, amidine, guanidine, phosphate, sulfate, carboxylic acid, sugar alcohol, amino alcohol, a short peptide, monosaccharide, disaccharide, polysaccharide, or a basic optionally substituted heterocycle or heteroaryl.

55. (canceled)

56. A compound selected from Compound Nos. 1-237, or a pharmaceutically acceptable salt or ester thereof.

57. A pharmaceutical composition comprising the compound of claim 1 or a pharmaceutically acceptable salt or ester thereof and optionally a pharmaceutically acceptable carrier.

58. A method of treating or preventing a disorder, condition or disease that may be responsive to the agonism of the G-protein-coupled receptor 40 in a subject in need thereof comprising administration of a therapeutically effective amount of the compound of claim 1 or a pharmaceutically acceptable salt or ester thereof.

59. A method of treating type 2 diabetes mellitus in a subject in need of treatment comprising administering to the subject a therapeutically effective amount of the compound of claim 1 or a pharmaceutically acceptable salt or ester thereof.

60. The method of claim 59, further comprising administering to the subject one or more additional therapeutic agents, wherein the one or more additional therapeutic agents are selected from PPAR gamma agonists and partial agonists; biguanides; protein tyrosine phosphatase-1B (PTP-1B) inhibitors: dipeptidyl peptidase IV (DPP-IV) inhibitors: insulin or an insulin mimetic: sulfonylureas: α-glucosidase inhibitors; agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (vi) acyl CoA:cholesterol acyltransferase (ACAT) inhibitors, (vii) CETP inhibitors, (viii) PCSK9 inhibitor or antibodies; (ix) apolipoproteins inhibitors; and (x) phenolic anti-oxidants; PPARα/γ dual agonists; PPARδ agonists; PPAR α/δ partial agonists; antiobesity compounds; ileal bile acid transporter inhibitors; anti-inflammatory agents; glucagon receptor antagonists; glucokinase activators; GLP-1 and GLP-1 analogs; GLP-1 receptor agonists; GLP-1/GIP receptor dual agonists; GLP-1/GIP/insulin receptor triple agonists; GLP-1/GIP/glucagon receptor triple agonists; HSD-1 inhibitors; HSD-17 inhibitors; SGLT-2 inhibitors; SGLT-1/SGLT-2 inhibitors: FXR agonists; DGAT1 and/or DGAT2 inhibitors; FGF19 and analogs; FGF21 and analogs; GDF15 and analogs; ANGPTL3 antibody or inhibitor; ANGPTL3/8 antibody; ANGPTL4 inhibitor; Oxyntomodulin.

61. (canceled)

62. A conjugate of a GPR40 agonist covalently linked to a carrier through a linker, wherein:
the linker contains an aliphatic group with the longest chain length of at least 10 carbons; and
the carrier has a hydrophilic moiety selected from an alcohol, an amine, an amide, an amino alcohol, an amino ether, water soluble ether, polyethylene glycol chain, a carboxylic acid, an amino acid, a peptide, a charged group, or a group that can become charged at pH 7, or a combination thereof.

63. A method of preparing the compound of claim 1, the method comprising coupling a compound of S-1 and S-2 to form the compound of Formula I: 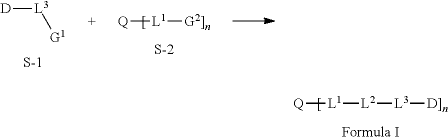
wherein G¹ and G² are suitable coupling partners to form the L² linkage of Formula I.

64. A compound of S-1: 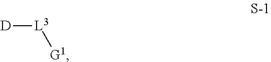
wherein
L³ is a bond, optionally substituted alkylene, optionally substituted heteroalkylene, optionally substituted carbocyclylene, optionally substituted heterocyclylene, optionally substituted arylene, or optionally substituted heteroarylene;
D is a residue of a GPR40 agonist; and
G¹ is acetylene, 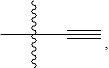 [SMILES: *C#C]
an azide (—N₃), or OH.

65. A compound of the following formulae III-1, III-2, III-3, III-4, III-5, III-6, III-7, III-8, or III-9, or pharmaceutically acceptable salts or ester thereof: 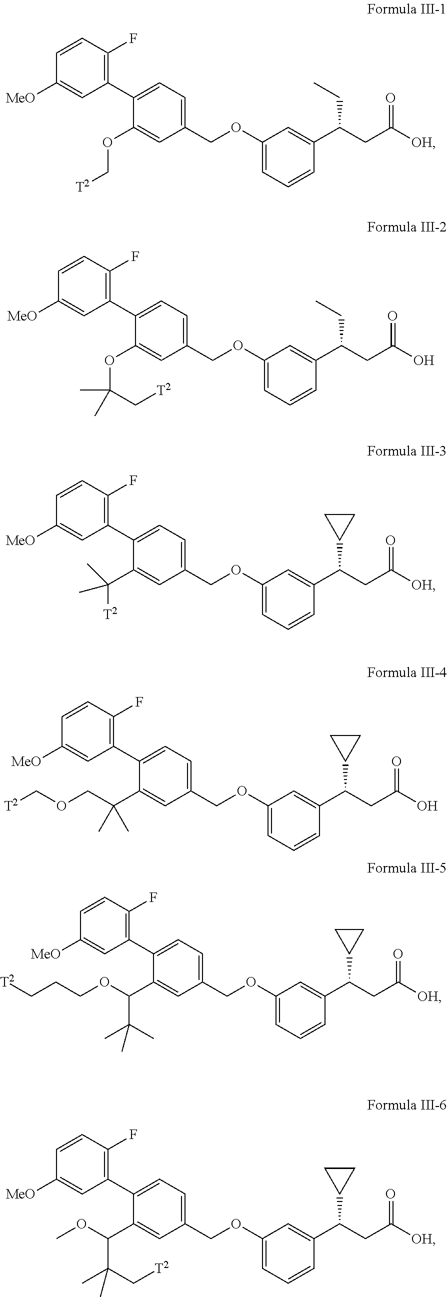 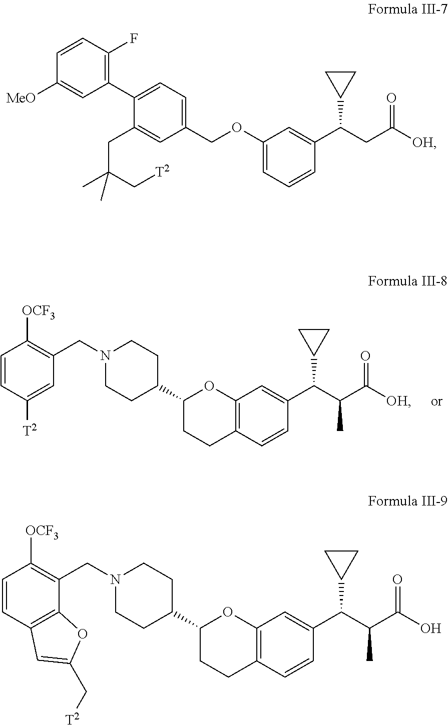
wherein T² is selected from:
1) —C₅₋₅₀ alkylene-T^{A1}, wherein T^{A1} is hydrogen or a moiety that includes one or more functional groups suitable for a coupling reaction, such as a coupling reaction for forming a carbon-carbon bond, carbon-heteroatom bond, or heteroatom-heteroatom bond, such as those forming an amide, ether, thioether, carbamate, carbonate, ester, phosphonate, sulfonate, sulfonamide, or urea linkage, for example, T^{A1} is OH, SH, SO₃H, NH₂, NHR¹⁰⁰, COOH, COOR¹⁰², CONR¹⁰⁰R¹⁰¹, or a leaving group;
2) -T^{B}-C₅₋₅₀ alkylene-T^{A1}; wherein T^{A1} is defined above, T^{B} is —N(R¹⁰⁰)—, —O—, —S—, —SO₂—, —C(═O)—, or a moiety selected from: 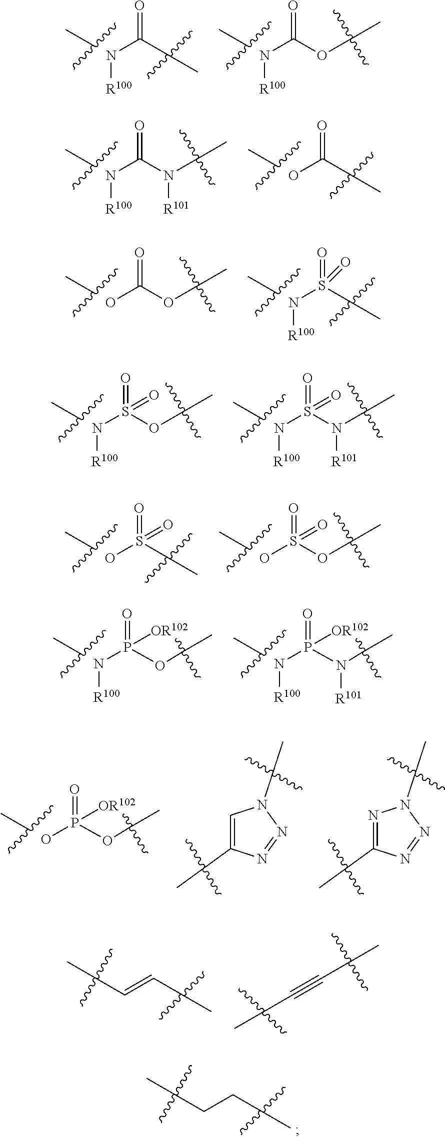
3) a moiety having a formula of -T^{C}-T^{B}-T^{D}-C₅₋₅₀ alkylene-T^{A1}, wherein T^{C} and T^{D} are independently a bond, optionally substituted alkylene, optionally substituted heteroalkylene, optionally substituted carbocyclylene, optionally substituted heterocyclylene, optionally substituted arylene, or optionally substituted heteroarylene, and T^{A1} and T^{B} are defined above, or
4) a moiety having a formula of -T^{C}-G, wherein T^{C} is defined above, and G is hydrogen, OH, N₃ or acetylene,
wherein R¹⁰⁰, R¹⁰¹ and R¹⁰² at each occurrence is independently hydrogen, optionally substituted alkyl, or optionally substituted cycloalkyl.
